SEQUENCE LISTING (32 sequences)

SEQ ID NO: 1 - DNA, 12 bp, Artificial Sequence
  Chemically Synthesized
ttagggttaggg

SEQ ID NO: 2 - DNA, 20 bp, Artificial Sequence
  Chemically Synthesized
tgacggaggcgccgctgcca

SEQ ID NO: 3 - DNA, 28 bp, Artificial Sequence
  Chemically Synthesized
tcaggcgctcagctccgtttcggtttca

SEQ ID NO: 4 - DNA, 26 bp, Artificial Sequence
  Chemically Synthesized
aagcgccattggagccccgcacttcc

SEQ ID NO: 5 - DNA, 34 bp, Artificial Sequence
  Chemically Synthesized
aattcactagtgaattcagatctggtaccgtacg

SEQ ID NO: 6 - DNA, 34 bp, Artificial Sequence
  Chemically Synthesized
tcgacgtacggtaccagatctgaattcactagtg

SEQ ID NO: 7 - DNA, 20 bp, Artificial Sequence
  Chemically Synthesized
gattaaggcagctataagca

SEQ ID NO: 8 - DNA, 20 bp, Artificial Sequence
  Chemically Synthesized
gttgaggaaaggcgagtacg

SEQ ID NO: 9 - DNA, 20 bp, Artificial Sequence
  Chemically Synthesized
tttggtctacactcgcgtga

SEQ ID NO: 10 - DNA, 20 bp, Artificial Sequence
  Chemically Synthesized
gtggtaggttgataagacaa

SEQ ID NO: 11 - DNA, 23 bp, Artificial Sequence
  Chemically Synthesized
caaaggacagcatcgctaatgcc

SEQ ID NO: 12 - DNA, 22 bp, Artificial Sequence
  Chemically Synthesized
gctccaatctgtcgcaactgct

SEQ ID NO: 13 - DNA, 22 bp, Artificial Sequence
  Chemically Synthesized
gaccgacagatgcctaaagtgg

SEQ ID NO: 14 - DNA, 22 bp, Artificial Sequence
  Chemically Synthesized
tgtcggtgaaggttgctggctt

SEQ ID NO: 15 - DNA, 23 bp, Artificial Sequence
  Chemically Synthesized
gagaaatcgtccaactcctcgag

SEQ ID NO: 16 - DNA, 22 bp, Artificial Sequence
  Chemically Synthesized
tctgagaggtgcagacacagct

SEQ ID NO: 17 - DNA, 22 bp, Artificial Sequence
  Chemically Synthesized
ggatgcttaccgaagtggagga

SEQ ID NO: 18 - DNA, 24 bp, Artificial Sequence
  Chemically Synthesized
ggtaacactgctcaaaatcggatg

SEQ ID NO: 19 - DNA, 22 bp, Artificial Sequence
  Chemically Synthesized
gtctcctctgacttcaacagcg

SEQ ID NO: 20 - DNA, 22 bp, Artificial Sequence
  Chemically Synthesized
accaccctgttgctgtagccaa

SEQ ID NO: 21 - DNA, 10 bp, Artificial Sequence
  Chemically Synthesized
accaaaccaa

SEQ ID NO: 22 - DNA, 33 bp, Artificial Sequence
  Chemically Synthesized
agatcggaagagcacacgtctgaactccagtca

SEQ ID NO: 23 - DNA, 33 bp, Artificial Sequence
  Chemically Synthesized
agatcggaagagcgtcgtgtagggaaagagtgt

SEQ ID NO: 24 - DNA, 33 bp, Artificial Sequence; identical to SEQ ID NO: 22
  Chemically Synthesized

SEQ ID NO: 25 - DNA, 33 bp, Artificial Sequence; identical to SEQ ID NO: 23
  Chemically Synthesized

SEQ ID NO: 26 - DNA, 27 bp, Artificial Sequence
  Chemically Synthesized
acgtgacgtggtttcagtgtttacacc

SEQ ID NO: 27 - DNA, 27 bp, Artificial Sequence
  Chemically Synthesized
agccccgcacttccaccaccagctcct

SEQ ID NO: 28 - DNA, 20 bp, Artificial Sequence
  Chemically Synthesized
agtcccgcgcaaacattatt

SEQ ID NO: 29 - DNA, 20 bp, Artificial Sequence
  Chemically Synthesized
atacggacggatcagggtac

SEQ ID NO: 30 - DNA, 20 bp, Artificial Sequence
  Chemically Synthesized
aacgaactggccttaccagt

SEQ ID NO: 31 - DNA, 40 bp, Artificial Sequence
  Chemically Synthesized
agtcccgcgcaaacattattatacggacggatcagggtac

SEQ ID NO: 32 - DNA, 40 bp, Artificial Sequence
  Chemically Synthesized
taatacgactcactatagggaacgaactggccttaccagt

(19) United States
(12) Patent Application Publication
(10) Pub. No.: US 2022/0323553 A1
(43) Pub. Date: Oct. 13, 2022

(54) Epigenomic editing and reactivation of targets for the treatment of Fragile X syndrome

(71) Applicant: THE TRUSTEES OF THE UNIVERSITY OF PENNSYLVANIA, Philadelphia, PA (US)

(72) Inventors: Jennifer E. Phillips-Cremins, Philadelphia, PA (US); Linda Zhou, Philadelphia, PA (US); Chunmin Ge, Philadelphia, PA (US)

(21) Appl. No.: 17/689,867

(22) Filed: Mar. 8, 2022

Related U.S. Application Data
(60) Provisional application No. 63/158,089, filed on Mar. 8, 2021.

(51) Int. Cl. A61K 38/46; A61P 43/00; C12N 9/22; C12N 15/11; A61K 31/7105; A61K 39/00

(52) U.S. Cl. CPC A61K 38/465; A61P 43/00; C12N 9/22; C12N 15/11; A61K 31/7105; A61K 39/0005; C12N 2310/20; A61K 2039/53

(57) ABSTRACT

The present invention generally relates to compositions and methods for modulating heterochromatin content or the level or activity of a gene or gene product that has been silenced by the formation of heterochromatin regions and the use thereof for the prevention and treatment of fragile X syndrome and diseases and disorders associated with fragile X syndrome.

### CROSS REFERENCE TO RELATED APPLICATIONS

This application claims priority to U.S. Provisional Application No. 63/158,089, filed Mar. 8, 2021, which is hereby incorporated by reference herein in its entirety.

### STATEMENT REGARDING FEDERALLY SPONSORED RESEARCH OR DEVELOPMENT

This invention was made with government support under MH120269 awarded by the National Institutes of Health. The government has certain rights in the invention.

### REFERENCE TO A “SEQUENCE LISTING,” A TABLE, OR A COMPUTER PROGRAM LISTING APPENDIX SUBMITTED AS AN ASCII TEXT FILE

The Sequence Listing written in the ASCII text file: 046483-6212-00US_SequenceListing.txt; created on Mar. 8, 2022, and 6,624 bytes in size, is hereby incorporated by reference.

### BACKGROUND OF THE INVENTION

Fragile X syndrome (FXS) is the most common form of inherited intellectual disability, affecting 1 in 4,000 males and 1 in 8,000 females. The disease is made manifest early in life and presents as a range of mild to severe defects in communication skills, cognitive ability, and physical appearance, as well as hypersensitivity to stimuli, seizures, and anxiety (Santoro et al., 2012, Annu Rev Pathol 7, 219-245). FXS is caused by expansion of a CGG STR tract in the 5′ untranslated (5′UTR) region of the FMR1 gene (La Spada et al., 1994, Ann Neurol 36, 814-822). FMR1 CGG length correlates with disease severity, and can be stratified into <40 (normal-length), 41-55 (intermediate), 55-200 (pre-mutation), and 200+(mutation-length) (Mirkin, 2007, Nature 447, 932-940; Nelson et al., 2013, Neuron 77, 825-843; La Spada et al., 2010, Nat Rev Genet 11, 247-258; McMurray, 2010, Nat Rev Genet 11, 786-799; Pearson et al., 2005, Nat Rev Genet 6, 729-742). Individuals with pre-mutation length CGG tracts can exhibit neurodevelopmental problems in their early years, and acquire late stage neurodegeneration due to Fragile X-associated tremor/ataxia syndrome (FXTAS) (Hagerman et al., 2016, Nat Rev Neurol 12, 403-412). Moreover, in a process known as anticipation, mutation-length STRs grow longer as they are inherited across generations, leading to earlier onset and increased severity of FXS symptoms (Richards et al., 1992, Nat Genet 1, 257-260). These data highlight the critical role of precise STR tract lengths in a wide range of pathologic features during the onset and progression of human disease.

Increases in STR tract length correlate with pathologically altered gene expression levels in a number of repeat expansion disorders (Orr et al., 2007, Annu Rev Neurosci 30, 575-621). In FXTAS, CGG expansion from normal-length to pre-mutation causes a 2-8-fold increase in FMR1 expression, leading to pathologic nuclear inclusion bodies⁸. By contrast, expansion from pre-mutation to mutation-length causes transcriptional inhibition of FMR1 and consequent severe reduction in levels of the Fragile X Mental Retardation Protein (FMRP) it encodes (Zoghbi et al., 2012, Cold Spring Harb Perspect Biol 4; Contractor et al., 2015, Neuron 87, 699-715). Evidence to date suggests that transcriptional silencing occurs solely due to local DNA methylation and heterochromatinization of the FMR1 CGG tract and its adjacent promoter (Sutcliffe et al., 1992, Hum Mol Genet 1, 397-400; Zhou et al., 2016, MolAutism 7, 42). Some genome-wide reports support this model by suggesting that pathologic changes to epigenetic modifications are restricted locally to FMR1 in FXS (Alisch et al., 2013, BMC Med Genet 14, 18). However, DNA demethylation by 5-aza-2′-deoxycytidine treatment or direct targeting of dCas9-Tet1 only partially reinstates FMR1 transcription, and patient samples with longer CGG tracts are more refractory to FMR1 de-repression (Coffee et al., 1999, Nat Genet 22, 98-101; Coffee et al., 2002, Am J Hum Genet 71, 923-932; Liu et al., 2018, Cell 172, 979-992 e976). Moreover, recovery of FMRP levels through the use of human FMR1 cDNA (Musumeci et al., 2007, Exp Neurol 203, 233-240), artificial chromosomes (Peier et al., 2000, Hum Mol Genet 9, 1145-1159), or viral vectors (Gholizadeh et al., 2014, Neuropsychopharmacology 39, 3100-3111; Zeier et al., 2009, Gene Ther 16, 1122-1129; Arsenault et al., 2016, Hum Gene Ther 27, 982-996) can only reduce, but not fully reverse, FXS defects in synaptic plasticity, anxiety, seizure susceptibility, and macro-orchidism. Together, these data suggest that a subset of long-term pathologic features of FXS are made manifest independent from FMRP's downstream effects.

Thus, there is a need in the art for improved compositions and methods for treating Fragile X Syndrome. This invention satisfies this unmet need.

### SUMMARY OF THE INVENTION

In some embodiments, the invention relates to a composition for activating, reactivating or de-repressing at least one H3K9me3-heterochromatin mark containing gene, wherein the gene is repressed or silenced in a heterochromatic genomic region.

In some embodiments, the composition increases the level of transcription, or translation of the silenced gene. In some embodiments, the composition increases the level of gene product for the silenced gene.

In some embodiments, the composition is a chemical compound, a protein, a peptidomemetic, an epigenomic editor, or a nucleic acid molecule.

In some embodiments, the composition is an epigenomic editor comprising catalytically dead Cas9 (dCas9) operably linked to a composition for removing a methylation mark. In some embodiments, the composition removing a methylation mark is VP64, NF-κB p65, Ten-Eleven Translocation (TET) protein, histone lysine demethylase (KDM) or a DNA demethylase.

In some embodiments, the composition further comprises a guide RNA specific for at least one silenced gene in a heterochromatin comprising genomic region. In some embodiments, the silenced gene in a heterochromatin comprising genomic region of FMR1, FMR1NB, FMR1-AS1, C5orf38, CTD-2194D22.4, LOC100506858, IRX2, LOC105374620, LINC01377, LINC01019, LINC01017, IRX1, LINC02114, DPP6, LINC01287, LOC101929998, CSMD1, FAM135B, LOC101927815, COL22A1, KCNK9, TRAPPC9, MYOM2, LOC101927845, LINC01591, LOC101927915, SPANXN4, SPANXN3, SLITRK4, SPANXN2, UBE2NL, SPANXN1, SLITRK2, TMEM257, MIR892C, MIR890, MIR888, MIR892A, MIR892B, MIR891B, MIR891A, CXorf51B, CXorf51A, MIR513C, MIR513B, MIR513A1, MIR513A2, MIR506, MIR507, MIR508, MIR514B, MIR509-2, MIR509-3, MIR509-1, MIR510, MIR514A1, MIR514A2, MIR514A3, TCERG1L, MIR378C, TCERG1L-AS1, LINC01164, TMEM132C, TMEM132D, LOC100996671, LOC101927592, LINC00508, LOC100996679, GLT1D1, RIMBP2, LINC00939, LOC101927464, LOC100128554, LINC00944, LINC00943, LOC440117, LOC101927616, LOC101927637, LOC105370068, FLJ37505, LINC00507, CRAT8, LOC101927694, MIR4419B, MIR3612, SLC15A4, LOC283352, LOC101927735, LOC100190940, FZD10-AS1, FZD10, PIWIL1, RBFOX1, MIR8065, TMEM114, DNAH9, SHISA6, PTPRT, LOC101927159, LINC01441, LINC01440, CBLN4, or MC3R.

In some embodiments, the composition is 5-aza-2′-deoxycytidine.

In some embodiments, the composition is for overexpression of one or more H3K9me3-heterochromatin mark containing gene, wherein the gene is repressed or silenced in a heterochromatic genomic region.

In some embodiments, the composition comprises a heterologous nucleic acid molecule comprising the silence gene. In some embodiments, the heterologous nucleic acid molecule encodes FMR1, FMR1NB, FMR1-AS1, C5orf38, CTD-2194D22.4, LOC100506858, IRX2, LOC105374620, LINC01377, LINC01019, LINC01017, IRX1, LINC02114, DPP6, LINC01287, LOC101929998, CSMD1, FAM135B, LOC101927815, COL22A1, KCNK9, TRAPPC9, MYOM2, LOC101927845, LINC01591, LOC101927915, SPANXN4, SPANXN3, SLITRK4, SPANXN2, UBE2NL, SPANXN1, SLITRK2, TMEM257, MIR892C, MIR890, MIR888, MIR892A, MIR892B, MIR891B, MIR891A, CXorf51B, CXorf51A, MIR513C, MIR513B, MIR513A1, MIR513A2, MIR506, MIR507, MIR508, MIR514B, MIR509-2, MIR509-3, MIR509-1, MIR510, MIR514A1, MIR514A2, MIR514A3, TCERG1L, MIR378C, TCERG1L-AS1, LINC01164, TMEM132C, TMEM132D, LOC100996671, LOC101927592, LINC00508, LOC100996679, GLT1D1, RIMBP2, LINC00939, LOC101927464, LOC100128554, LINC00944, LINC00943, LOC440117, LOC101927616, LOC101927637, LOC105370068, FLJ37505, LINC00507, CRAT8, LOC101927694, MIR4419B, MIR3612, SLC15A4, LOC283352, LOC101927735, LOC100190940, FZD10-AS1, FZD10, PIWIL1, RBFOX1, MIR8065, TMEM114, DNAH9, SHISA6, PTPRT, LOC101927159, LINC01441, LINC01440, CBLN4, or MC3R.

In some embodiments, the composition comprises a nucleic acid molecule comprising a nucleotide sequence of an Fmr1 gene comprising an intermediate or pre-mutation length CGG tandem repeat. In some embodiments, the intermediate or pre-mutation length CGG tandem repeat comprises 40 to 200 tandem CGG repeats.

In some embodiments, the composition comprises a noncoding RNA molecule comprising a premutation length CGG tandem repeat. In some embodiments, the intermediate or pre-mutation length CGG tandem repeat comprises 40 to 200 tandem CGG repeats. In some embodiments, the composition comprises an RNA vaccine comprising a noncoding RNA molecule comprising a premutation length CGG tandem repeat. In some embodiments, the intermediate or pre-mutation length CGG tandem repeat comprises 40 to 200 tandem CGG repeats.

In some embodiments, the composition comprises a composition for reducing a full mutation length CGG tandem repeat of Fmr1 to an intermediate or pre-mutation length. In some embodiments, the composition comprises a complex comprising a guide RNA targeted to the Fmr1 gene, and a CRISPR-associated (Cas) protein. In some embodiments, the intermediate or pre-mutation length CGG tandem repeat comprises 40 to 200 tandem CGG repeats.

In some embodiments, the composition comprises a composition for reducing the level of Fmr1 mRNA, wherein the Fmr1 mRNA comprises a full mutation length CGG tandem repeat. In some embodiments, the composition comprises a complex comprising a guide RNA targeted to the Fmr1 mRNA, and a CRISPR-associated (Cas) protein. In some embodiments, the CRISPR-associated (Cas) protein is Cas13.

In some embodiments, the invention relates to a composition for inhibiting at least one of heterochromatin formation, RNA mediated heterochromatin formation and RNA-DNA interactions. In some embodiments, the inhibitor is a small interfering RNA (siRNA), a microRNA, an antisense nucleic acid, a ribozyme, an expression vector encoding a transdominant negative mutant, an antibody, an antibody fragment, a peptide, a chemical compound or a small molecule. In some embodiments, the inhibitor is compound 1a, compound 1f or ETP69.

In some embodiments, the inhibitor decreases the level of mRNA or protein of at least one CGG tandem repeat containing gene. In some embodiments, the CGG tandem repeat containing gene is FMR1, SHISA6, IRX2, TCERG1L, PTPRT, DPP6, or TMEM257. In some embodiments, the inhibitor comprises an antisense oligonucleotide (ASO) targeting FMR1, SHISA6, IRX2, TCERG1L, PTPRT, DPP6, or TMEM257.

In some embodiments, the inhibitor decreases the level of mRNA or protein of at least one histone H3-K9 methyltransferase gene. In some embodiments, the histone H3-K9 methyltransferase gene is ESET, G9a, Eu-HMTase, SUV39H1 or SUV39H2.

In some embodiments, the invention relates to a method of activating, reactivating or de-repressing at least one H3K9me3-heterochromatin mark containing gene, wherein the gene is repressed or silenced in a heterochromatic genomic region, the method comprising contacting a sample comprising a heterochromatic nucleic acid molecule with a composition for activating, reactivating or de-repressing at least one H3K9me3-heterochromatin mark containing gene, wherein the gene is repressed or silenced in a heterochromatic genomic region. In some embodiments, the gene encodes at least one of FMR1, FMR1NB, FMR1-AS1, C5orf38, CTD-2194D22.4, LOC100506858, IRX2, LOC105374620, LINC01377, LINC01019, LINC01017, IRX1, LINC02114, DPP6, LINC01287, LOC101929998, CSMD1, FAM135B, LOC101927815, COL22A1, KCNK9, TRAPPC9, MYOM2, LOC101927845, LINC01591, LOC101927915, SPANXN4, SPANXN3, SLITRK4, SPANXN2, UBE2NL, SPANXN1, SLITRK2, TMEM257, MIR892C, MIR890, MIR888, MIR892A, MIR892B, MIR891B, MIR891A, CXorf51B, CXorf51A, MIR513C, MIR513B, MIR513A1, MIR513A2, MIR506, MIR507, MIR508, MIR514B, MIR509-2, MIR509-3, MIR509-1, MIR510, MIR514A1, MIR514A2, MIR514A3, TCERG1L, MIR378C, TCERG1L-AS1, LINC01164, TMEM132C, TMEM132D, LOC100996671, LOC101927592, LINC00508, LOC100996679, GLT1D1, RIMBP2, LINC00939, LOC101927464, LOC100128554, LINC00944, LINC00943, LOC440117, LOC101927616, LOC101927637, LOC105370068, FLJ37505, LINC00507, CRAT8, LOC101927694, MIR4419B, MIR3612, SLC15A4, LOC283352, LOC101927735, LOC100190940, FZD10-AS1, FZD10, PIWIL1, RBFOX1, MIR8065, TMEM114, DNAH9, SHISA6, PTPRT, LOC101927159, LINC01441, LINC01440, CBLN4, and MC3R.

In some embodiments, the invention relates to a method of inhibiting at least one of heterochromatin formation, RNA mediated heterochromatin formation and RNA-DNA interactions, the method comprising contacting a sample with a composition for inhibiting at least one of heterochromatin formation, RNA mediated heterochromatin formation and RNA-DNA interactions.

In some embodiments, the method comprises decreasing the level of mRNA or protein of at least one CGG tandem repeat containing gene. In some embodiments, the CGG tandem repeat containing gene is FMR1, SHISA6, IRX2, TCERG1L, PTPRT, DPP6, or TMEM257.

In some embodiments, the method comprises decreasing the level of mRNA or protein of at least one histone H3-K9 methyltransferase gene. In some embodiments, the histone H3-K9 methyltransferase gene is ESET, G9a, Eu-HMTase, SUV39H1 or SUV39H2.

In some embodiments, the invention relates to a method of treating or preventing a fragile X syndrome or a disease or disorder associated with fragile X syndrome in a subject in need thereof, the method comprising administering a composition for activating, reactivating or de-repressing at least one H3K9me3-heterochromatin mark containing gene, wherein the gene is repressed or silenced in a heterochromatic genomic region, to a subject in need thereof. In some embodiments, the gene encodes FMR1, FMR1NB, FMR1-AS1, C5orf38, CTD-2194D22.4, LOC100506858, IRX2, LOC105374620, LINC01377, LINC01019, LINC01017, IRX1, LINC02114, DPP6, LINC01287, LOC101929998, CSMD1, FAM135B, LOC101927815, COL22A1, KCNK9, TRAPPC9, MYOM2, LOC101927845, LINC01591, LOC101927915, SPANXN4, SPANXN3, SLITRK4, SPANXN2, UBE2NL, SPANXN1, SLITRK2, TMEM257, MIR892C, MIR890, MIR888, MIR892A, MIR892B, MIR891B, MIR891A, CXorf51B, CXorf51A, MIR513C, MIR513B, MIR513A1, MIR513A2, MIR506, MIR507, MIR508, MIR514B, MIR509-2, MIR509-3, MIR509-1, MIR510, MIR514A1, MIR514A2, MIR514A3, TCERG1L, MIR378C, TCERG1L-AS1, LINC01164, TMEM132C, TMEM132D, LOC100996671, LOC101927592, LINC00508, LOC100996679, GLT1D1, RIMBP2, LINC00939, LOC101927464, LOC100128554, LINC00944, LINC00943, LOC440117, LOC101927616, LOC101927637, LOC105370068, FLJ37505, LINC00507, CRAT8, LOC101927694, MIR4419B, MIR3612, SLC15A4, LOC283352, LOC101927735, LOC100190940, FZD10-AS1, FZD10, PIWIL1, RBFOX1, MIR8065, TMEM114, DNAH9, SHISA6, PTPRT, LOC101927159, LINC01441, LINC01440, CBLN4, or MC3R.

In some embodiments, the invention relates to a method of treating or preventing a fragile X syndrome or a disease or disorder associated with fragile X syndrome in a subject in need thereof, the method comprising administering a composition for inhibiting at least one of heterochromatin formation, RNA mediated heterochromatin formation and RNA-DNA interactions, to a subject in need thereof.

In some embodiments, the method comprises decreasing the level of mRNA or protein of at least one CGG tandem repeat containing gene. In some embodiments, the CGG tandem repeat containing gene is FMR1, SHISA6, IRX2, TCERG1L, PTPRT, DPP6, or TMEM257.

In some embodiments, the method comprises decreasing the level of mRNA or protein of at least one histone H3-K9 methyltransferase gene. In some embodiments, the histone H3-K9 methyltransferase gene is ESET, G9a, Eu-HMTase, SUV39H1 or SUV39H2.

In some embodiments, the invention relates to a method of diagnosing a subject as having fragile X syndrome or a disease or disorder associated with fragile X syndrome, the method comprising detecting a decreased level of mRNA or protein of at least one H3K9me3-heterochromatin mark containing gene, wherein the gene is repressed or silenced in a heterochromatic genomic region. In some embodiments, the method comprises detecting a decreased level of mRNA or protein for FMR1, FMR1NB, FMR1-AS1, C5orf38, CTD-2194D22.4, LOC100506858, IRX2, LOC105374620, LINC01377, LINC01019, LINC01017, IRX1, LINC02114, DPP6, LINC01287, LOC101929998, CSMD1, FAM135B, LOC101927815, COL22A1, KCNK9, TRAPPC9, MYOM2, LOC101927845, LINC01591, LOC101927915, SPANXN4, SPANXN3, SLITRK4, SPANXN2, UBE2NL, SPANXN1, SLITRK2, TMEM257, MIR892C, MIR890, MIR888, MIR892A, MIR892B, MIR891B, MIR891A, CXorf51B, CXorf51A, MIR513C, MIR513B, MIR513A1, MIR513A2, MIR506, MIR507, MIR508, MIR514B, MIR509-2, MIR509-3, MIR509-1, MIR510, MIR514A1, MIR514A2, MIR514A3, TCERG1L, MIR378C, TCERG1L-AS1, LINC01164, TMEM132C, TMEM132D, LOC100996671, LOC101927592, LINC00508, LOC100996679, GLT1D1, RIMBP2, LINC00939, LOC101927464, LOC100128554, LINC00944, LINC00943, LOC440117, LOC101927616, LOC101927637, LOC105370068, FLJ37505, LINC00507, CRAT8, LOC101927694, MIR4419B, MIR3612, SLC15A4, LOC283352, LOC101927735, LOC100190940, FZD10-AS1, FZD10, PIWIL1, RBFOX1, MIR8065, TMEM114, DNAH9, SHISA6, PTPRT, LOC101927159, LINC01441, LINC01440, CBLN4, or MC3R.

In some embodiments, the invention relates to a composition for inhibiting an interaction between a nucleic acid molecule comprising a Fmr1 full-mutation length CGG repeat and at least one distal or trans nucleic acid molecule comprising a CGG repeat, comprising a nucleic acid molecule that binds to a CGG repeat. In some embodiments, the composition comprises a recombinant nucleic acid molecule comprising a pre-mutation length CGG repeat. In some embodiments, the pre-mutation length CGG repeat comprises 99 CGG repeats.

In some embodiments, the composition comprises a recombinant nucleic acid molecule for expression of an antisense oligonucleotide that directly hybridizes to a nucleic acid molecule comprising a CGG repeat.

In some embodiments, the invention relates to a method of inhibiting an interaction between a nucleic acid molecule comprising a Fmr1 full-mutation length CGG repeat and at least one distal or trans nucleic acid molecule comprising a CGG repeat, the method comprising administering to a subject in need thereof composition for inhibiting an interaction between a nucleic acid molecule comprising a Fmr1 full-mutation length CGG repeat and at least one distal or trans nucleic acid molecule comprising a CGG repeat, comprising a nucleic acid molecule that binds to a CGG repeat, an inhibitor of heterochromatin formation, an inhibitor of RNA mediated heterochromatin formation, or an inhibitor of RNA-DNA interactions.

In some embodiments, the Fmr1 gene of the subject comprises at least 200 CGG repeats.

In some embodiments, the method comprises administering a recombinant nucleic acid molecule comprising a pre-mutation length CGG repeat. In some embodiments, the pre-mutation length CGG repeat comprises 99 CGG repeats.

In some embodiments, the method comprises administering a recombinant nucleic acid molecule for expression of an antisense oligonucleotide that directly hybridizes to a nucleic acid molecule comprising a CGG repeat. In some embodiments, the inhibitor is compound 1a, compound 1f or ETP69.

In some embodiments, the invention relates to a method of treating or preventing a disease or disorder associated with genomic instability or a triplet repeat expansion in a subject in need thereof, the method comprising administering a composition comprising a noncoding RNA molecule comprising a premutation length CGG tandem repeat for activating, reactivating or de-repressing at least one H3K9me3-heterochromatin mark containing gene, wherein the gene is repressed or silenced in a heterochromatic genomic region, to a subject in need thereof. In some embodiments, the intermediate or pre-mutation length CGG tandem repeat comprises 40 to 200 tandem CGG repeats. In some embodiments, the composition comprises an RNA vaccine comprising a noncoding RNA molecule comprising a premutation length CGG tandem repeat. In some embodiments, the intermediate or pre-mutation length CGG tandem repeat comprises 40 to 200 tandem CGG repeats.

In some embodiments, the disease or disorder associated with genomic instability or a triplet repeat expansion is selected from the group consisting of parkinsonism, ataxia, dementia, autonomic dysfunctions, myopathy, ubiquitin-positive inclusion bodies, middle cerebellar peduncle hyperintensity, leukoencephalopathy, myotonic dystrophy (DM), Huntington disease, spinocerebellar ataxia, Friedreich ataxia, fragile X syndrome, fragile X-associated primary ovarian insufficiency (FXPOI), fragile X-associated tremor/ataxia syndrome (FXTAS), syndromic and non-syndromic forms of intellectual disability (ID), autism, developmental delay, Jacobsen syndrome, and Baratela-Scott syndrome.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following detailed description of embodiments of the invention will be better understood when read in conjunction with the appended drawings. It should be understood that the invention is not limited to the precise arrangements and instrumentalities of the embodiments shown in the drawings.

FIGS. 1a-1i depict exemplary results demonstrating that a >5 Megabase-sized domain of H3K9me3 heterochromatin spreads across the FMR1 locus in a CGG STR length-dependent manner in fragile X syndrome. FIG. 1a depicts a schematic of iPS cell lines used to model FXTAS and FXS, including normal-length, pre-mutation length, short mutation-length, and long mutation-length. Colors are associated with each CGG length to identify STR sequence-dependent disease progression across all figures.

FIGS. 1b-1e depict results of Nanopore long-read analysis of total number of CGGs present (FIG. 1b), longest continuous CGG tract (FIG. 1c), number of AGG interrupters within CGG STR (FIG. 1d), and total number of continuous CGG tracks within the STR in the 5′UTR of FMR1 (FIG. 1e). FIG. 1f depicts results of FMR1 mRNA levels as evaluated by RNA-seq. Horizontal lines represent the central tendency (mean) between n=2 biological replicates. FIG. 1g depicts results of H3K9me3 ChIP-seq across all five lines is shown for an 8 Mb region around FMR1. Gene track is plotted below ChIP-seq tracks. FMR1, SLITRK2, and SLITRK4 are highlighted in red, blue, and green respectively. FIG. 1h depicts results of Hi-C data across all five lines is shown as a heatmap of counts representing interaction frequency for an 8 Mb region around FMR1. Compartment score, H3K9me3 ChIP-seq, and CTCF ChIP-seq in iPS-derived NPCs is displayed below the heatmaps for all five conditions. FIG. 1i depicts results of SLITRK2 and SLITRK4 mRNA levels as evaluated by RNA-seq. Horizontal lines represent the central tendency (mean) between n=2 biological replicates.

FIG. 2 depicts exemplary results of nanopore long-read sequencing over the FMR1 gene. Visual representation of Nanopore long reads that span the transcription start site and first 200 bp of FMR1. For each of the 5 samples, the sequence of each read is shown in colors corresponding to base pairs shown in the legend.

FIG. 3 depicts exemplary results of the mappability statistics of Hi-C Samples.

FIG. 4 depicts exemplary results demonstrating that CpG methylation evaluated from Nanopore long-reads is increased over the FMR1 transcription start site and CGG STR in fragile X syndrome. Nanopolish was used to calculate CpG methylation frequency around FMR1 from the Nanopore long-reads (see Methods in Example 1).

FIG. 5 depicts exemplary results of the mappability statistics of ChIP-seq Samples.

FIGS. 6a-6n depict exemplary results demonstrating that disruption to the 3D genome upon acquisition of a Mb-sized heterochromatin domain across the FMR1 locus as the CGG STR tract expands from short mutation-length to long mutation-length in fragile X syndrome. FIG. 6a depicts Hi-C data across all five lines, shown as a heatmap of counts representing interaction frequency for an 8 Mb region around FMR1. Compartment score, H3K9me3 ChIP-seq, and CTCF ChIP-seq in iPS-derived NPCs is displayed below the heatmaps for all five conditions. Box 1, 2, and 3 are highlighted and referenced elsewhere in the figure. FIG. 6b depicts the H3K9me3 ChIP signal across the entire loci shown in FIG. 6a is binned into 40 bins and plotted for each cell line. Each dot represents one bin. FIG. 6c depicts the compartment score across the loci shown in FIG. 6a is binned into 40 bins and plotted for each cell line. Each dot represents one bin. FIG. 6d depicts CTCF and H3K9me3 ChIP-seq including FMR1 and up to 6 MB upstream are overlaid on each other for each cell line. FIG. 6e depicts the insulation score up to 6 MB upstream from FMR1 is shown for 5 cell lines. Grey vertical lines represent location of FMR1 gene. FIG. 6f depicts a zoom in on Box 1 from FIG. 6a on a 1 Mb region centered on FMR1. Blue highlights demonstrate location of gained contains/boundary disruption in disease. FIG. 6g depicts a barplot showing directionality index, a metric quantifying domain boundary strength, is plotted at the FMR1 gene in 5 cell lines. FIG. 6h depicts the insulation score in a 1 MB region containing FMR1 is shown for 5 cell lines. Grey vertical lines represent location of FMR1 gene. FIG. 6i and FIG. 6j depict zoom-ins to Box2 and Box3 from FIG. 6a showing FMR1-SLITRK2 or FMR1-SLITRK4 gene-gene interactions, respectively. FIG. 6k and FIG. 6l depict a boxplot showing the interaction frequency measured with Hi-C between FMR1 and either SLITRK2 (FIG. 6k) or with SLITRK4 (FIG. 6l). Genomic intervals containing FMR1, SLITRK2, SLITRK4 are binned into 20 kb bins and each dot represents interactions between one set of bins. A total of n=15 bins are shown for SLITRK2 and n=6 bins are shown for SLITRK4. Boxes show the range from lower to upper quartiles, with median line, and whispers extend to minimum and maximum data points with 1.5 times the interquartile range. FIG. 6m and FIG. 6n depict the expression of SLITRK2 and SLITRK4, respectively, across 5 cell lines. Data is shown for 2 replicates. Dots represent replicates in expression plot, and lines represent mean expression across replicates.

FIG. 7 depicts exemplary results demonstrating that a series of loops connecting FMR1 to SLITRK2 are lost in fragile X syndrome. Heatmaps of Hi-C cis interaction frequency in a 2 Mb window around FMR1 (red, x-axis) interacting with a 2.6 Mb window around SLITRK2 (blue, y-axis) across five iPS cell lines differentiated to NPCs with normal-length, pre-mutation, short mutation-length, or long mutation-length CGG STR tract in FMR1. A/B compartment score computed from Hi-C data, CTCF ChIP-seq, and H3K9me3 ChIP-seq tracts are shown below heatmaps for each cell line. Grey arrows denote locations of loops which are lost in short and long mutation-length F×S lines. Turquoise highlights indicate the location of CTCF sites that are lost in parallel with looping interactions.

FIG. 8 depicts exemplary results demonstrating that two long mutation-length fragile X syndrome samples differ in the spread and density of H3K9me3 domain. Heatmaps of Hi-C cis interaction frequency in a 10 Mb window around FMR1 (red gene) SLITRK2 (blue gene), and SLITRK4 (green gene) across five iPS cell lines differentiated to NPCs with normal-length, pre-mutation, short mutation-length, or long mutation-length CGG STR tract in FMR1. A/B compartment score computed from Hi-C data, CTCF ChIP-seq, and H3K9me3 ChIP-seq tracts are shown below heatmaps for each cell line. Grey arrows denote locations of long-range ˜5 Mb loops between FMR1 and SLITRK4 in WT. Turquoise highlights over FMR1 and SLITRK4 are shown and intersect at the location of the grey arrow.

FIGS. 9a-9c depict exemplary results demonstrating that a megabase-scale heterochromatin domain is deposited across the FMR1 locus in FXS iPS cells. FIG. 9a depicts H3K9me3 ChIP-seq in one replicate of five iPS cell lines in an 8 MB region around FMR1. Genes are shown below ChIP-seq tracks. FMR1, SLITRK2, and SLITRK4 are highlighted in red, blue, and green respectively. FIG. 9b depicts a zoom-in on data from FIG. 9a shown in an 75 KB window around the FMR1 gene. FIG. 9c depicts H3K9me3 ChIP-seq from FIG. 9a shown with all 5 cell lines overlaid directly on top of each other.

FIGS. 10a-10d depict exemplary results demonstrating that local 3D genome alterations such as TAD boundary disruption and loop loss occur around the FMR1 gene upon gene silencing and mutation-length CGG expansion. FIG. 10a depicts 5C Heatmaps of ˜5 MB of the X chromosome surrounding the FMR1 gene in WT B cells, B cells from FXS patients with 900 CGG repeats in the 5′UTR of FMR1, and B cells with 650 CGG repeats from a different patient. H3K27ac, CTCF, H3K9me3, and H3K27me3 ChIP seq tracks from these B-cells are aligned underneath heatmaps. All data is from 1 replicate. FIG. 10b depicts 5C data in each FXS B-cell line is divided by 5C data in WT B-cells to show fold change maps aligned with epigenetic modification tracks in FIG. 10a. FIG. 10c depicts a zoom in on to 1.5 MB around FMR1 (location marked in grey rectangle in FIG. 10a). FIG. 10d depicts a zoom in on to 80 KB around FMR1.

FIGS. 11a-11j depict exemplary results demonstrating that distal heterochromatin domains on somatic chromosomes repress critical synaptic plasticity genes in fragile X syndrome. FIG. 11a depicts results of three classes of H3K9me3 ChIP-seq domains identified genome-wide across five iPS-derived NPC lines. H3K9me3 domains are defined as either (i) invariant across all genotypes, (ii) gained in FXS but not consistently in all disease lines, or (iii) consistently gained in all three F×S lines. Categorization was based on presence or absence of domains identified by RSEG. FIG. 11b depicts Hi-C data across all five lines, shown as a heatmap of counts representing interaction frequency for a 1.6 Mb region around one of the somatic H3K9me3 domains encompassing SHISA6. Compartment score, H3K9me3 ChIP-seq, and CTCF ChIP-seq in iPS-derived NPCs is displayed below the heatmaps for all five conditions. Lines representing RSEG H3K9me3 domain calls are show above H3K9me3 ChIP-seq track. SHISA6 gene is highlighted in orange. FIG. 11c depicts the insulation score across Hi-C matrices in the same 1.6 Mb region as FIG. 11b for all five cell lines. Red, orange, green, blue, purple correspond to normal-length, pre-mutation, short mutation-length, long mutation-length sample 1, and long mutation-length sample 2, respectively. SHISA6 gene is highlighted in orange. FIG. 11d depicts SHISA6 mRNA levels as evaluated by RNA-seq. Horizontal lines represent the central tendency (mean) between n=2 biological replicates. FIG. 11e depicts pooled H3K9me3 and CTCF ChIP-seq data across all n=12 consistently gained H3K9me3 domains in FXS. FIG. 11f depicts the insulation score for the strongest domain boundary in each of the H3K9me3 domains consistently gained in all three F×S lines for WT_NPC_15 (red) and FXS_NPC_378 (purple) cell lines. There are 12 sets of one red and one purple bar plot, each set corresponding to one H3K9me3 domain. FIG. 11g depicts mRNA levels as evaluated by RNA-seq for n=26 protein coding genes in consistently gained domains in FXS. Genes are only shown if they were expressed in at least one cell line. Each point represents expression of one gene averaged across n=2 biological replicates. * indicates p<0.05 when compared to WT_NPC_15. Pvalues were calculated using a one-tailed Mann Whitney U test. FIG. 11h depicts the expression of all genes in consistently gained domains in FXS across tissues in the GTEX dataset. Genes were only shown if expression was not 0 across all tissues, resulting in n=67 genes. Genes were clustered using K-means clusters into 4 groups, and clusters were labelled based on the tissue types dominating each cluster.

FIGS. 11i-11j depict a gene ontology (GO) analysis using WebGESTALT for n=26 protein coding genes expressed in iPS-derived NPCs and localized to H3K9me3 domains consistently gained in FXS (FIG. 11i) and n=20 protein coding genes expressed in iPS-derived NPCs and localized to H3K9me3 domains gained in FXS but not consistently in all F×S lines as defined in panel FIG. 11a (FIG. 11j).

FIG. 12 depicts exemplary results of distal FXS H3K9me3 domains in iPS-derived NPCs. H3K9me3 and CTCF ChIPseq are plotted at n=11 distal FXS H3K9me3 domains in five NPC lines (normal-length (15 CGG), pre-mutation (133 CGG), short mutation-length (306 CGG), long mutation-length sample 1 (326 CGG), long mutation-length sample 2 (378 CGG)). Note: two of the 11 domains are separated by only 200kb, so both are shown on one plot in the chr8:134.6-142.7 interval (noted by arrows).

FIG. 13 depicts exemplary results of distal FXS H3K9me3 domains in iPS cells. H3K9me3 ChIP-seq is shown around distal FXS H3K9me3 domains for five iPS cell lines (normal-length (15 CGG), pre-mutation (133 CGG), short mutation-length (306 CGG), long mutation-length sample 1 (326 CGG), long mutation-length sample 2 (378 CGG)). Lines representing RSEG H3K9me3 domain calls are shown above H3K9me3 ChIP-seq track. Of n=12 total H3K9me3 domains gained, one is at FMR1 (FIG. 9), and the remaining 11 are shown here. Note: two of the 11 domains are separated by only 200kb, so both are shown on one plot in the chr8:134.6-142.7 interval.

FIG. 14 depicts exemplary results demonstrating that genome folding is severely disrupted upon acquisition of distal H3K9me3 domains in FXS. Hi-C Interaction frequency heatmaps around distal FXS H3K9me3 domains. Compartment score, H3K9me3 ChIP-seq, and CTCF ChIP-seq from NPCs is displayed below the heatmaps. Lines representing RSEG H3K9me3 domain calls are shown above the H3K9me3 ChIP-seq track. Of n=12 total H3K9me3 domains, one is at FMR1 (FIG. 1e) and the remaining 11 are shown here. Note: two of the 10 domains are separated by only 200kb, so both are shown on one plot in the chr8:134.6-142.7 interval.

FIG. 15 depicts exemplary results of expression of genes in n=12 FXS H3K9me3 domains. Of the n=12 domains, 10 contained protein coding genes expressed in NPCs. For each domain, expression for genes in that domain is shown for normal-length (15 CGG), pre-mutation (133 CGG), short mutation-length (306 CGG), long mutation-length sample 1 (326 CGG), long mutation-length sample 2 (378 CGG)). Two biological replicates are shown for each sample. Horizontal line represents the mean of two replicates for each of five lines.

FIGS. 16a-16c depict exemplary results of Gene Ontology for upregulated and downregulated genes in FXS. Gene ontology for n=25 protein coding expressed genes in invariant H3K9me3 domains as defined in FIG. 11a (FIG. 16a), genes upregulated in both long diseases (FIG. 16b), or genes downregulated in both long mutation-length F×S lines but not located within one of n=12 consistently gained FXS H3K9me3 domains (FIG. 16c). GO was performed using WebGESTALT with settings Over-Representation Analysis, geneontology, Biological Process, Cellular Component, Molecular function, with “genome-protein coding” as the reference. A P-value cutoff of p<0.01 and enrichment >4 was used.

FIGS. 17a-17b depict exemplary results of RNA-seq data in FXS. FIG. 17a depicts an M-A plot showing RNA-seq data in iPS-derived NPCs (normal-length (15 CGG), pre-mutation (133 CGG), short mutation-length (306 CGG), long mutation-length sample 1 (326 CGG), long mutation-length sample 2 (378 CGG)). Genes in red are called significant by DEseq2 using a likelihood ratio test at a threshold of p<0.005. FIG. 17b depicts the total number of up- and down-regulated genes for all lines (pre-mutation (133 CGG), short mutation-length (306 CGG), long mutation-length sample 1 (326 CGG), long mutation-length sample 2 (378 CGG)) compared to normal-length (15 CGG).

FIG. 18 depicts exemplary results demonstrating tissue specific expression of genes in FXS H3K9me3 domains. Expression of all genes in H3k9me3 domains consistently gained across all 3 F×S lines in n=54 tissues in GTEX dataset. Genes were only shown if expression was not zero across all tissues, resulting in n=67 genes. Genes were clustered using K-means clusters into 4 groups, and clusters were labelled based on the tissue types dominating each cluster. This is the same as FIG. 2h, but with all labels for each axis shown legibly in a larger image footprint.

FIGS. 19a-19H depict exemplary results demonstrating that FXS heterochromatin domains form a spatial subnuclear hub of trans interactions between distal genes exhibiting ultra-high frequency of CGG STRs. FIG. 19a depicts Hi-C inter-chromosomal interaction heatmaps binned at 1 Mb resolution between FMR1 H3K9me3 domains and H3K9me3 domains on chromosome 8 and chromosome 5. The window for each region includes the H3K9me3 domain gained in FXS and 5 Mb of flanking genome. H3K9me3 ChIP-seq is shown on the x-axis for chromosome X and for the y-axis for the distal region. Blue bars and green arrows highlight trans interactions. FIG. 19b depicts inter-chromosomal contacts between all FXS H3K9me3 domains and the FMR1 domain on chromosome X. Each dot represents one gained H3K9me3 domain. Lines connecting the dots show the progress of that domain across 5 cell lines with increasing CGG STR length. Bar represents mean trans contacts across all domains. FIG. 19c depicts pairwise Hi-C interactions among all of the distal H3K9me3 domains gained in FXS for long mutation-length (FXS_378, upper triangle) and normal-length (FXS_15, lower triangle). Domains annotated by chromosome. The window for each region includes the H3K9me3 domain gained and 3 Mb of flanking genome. H3K9me3 ChIP-seq signal for all domains for both FXS_378 and FXS_15 are plotted above Hi-C heatmaps. Blue boxes highlight FXS gained trans interactions. FIG. 19d depicts the location of the gained H3K9me3 domain at FMR1 and n=11 distal gained H3K9me3 domains is highlighted in a red box on a chromosome ideogram obtained from the UCSC genome browser. In FIGS. 19e-19f, H3K9me3 and CGG STRs are shown for the IRX2 gene (FIG. 19e) and the PTPRT gene (FIG. 19f). FIG. 19g depicts the average number of CGG STR tracks within the first 2 kb of genes in FXS domains is compared to genes in a null distribution consisting of 1000 draws of n=12 size-matched, randomly-sampled intervals located within genotype-independent H3K9me3 domains that remain constant throughout normal-length, short mutation, pre-mutation, and disease lines. To count the of CGGs, all CGG tracks that were at least (CGG)n>=2 were analyzed and the total number of CGG occurrences within the first 2 kb of genes whose promoters were located within either the null or test set of domains was summed. A one-tailed randomization test was used to compute a P-value as the area under the null distribution curve to the right of the red line. FIG. 19h depicts the number of fragile sites within the H3K9me3 domains in FXS compared to a null distribution consisting of 1000 draws of n=12 size-matched, randomly-sampled intervals located within genotype-independent H3K9me3 domains that remain constant throughout normal-length, short mutation, pre-mutation, and disease lines. as called by RSEG. Fragile sites were obtained from the HumCFS database.

FIG. 20 depicts exemplary results of inter-chromosomal interactions between FMR1 and distal FXS H3K9me3 domains. Hi-C interactions between FMR1 and each of the distal H3K9me3 domains gained are shown for normal-length (15 CGG), pre-mutation (133 CGG), short mutation-length (306 CGG), and two long mutation-length samples with increasing continuous CGG length FXS NPCs. The window for each region includes the H3K9me3 domain gained and 5 Mb of flanking genome. H3K9me3 ChIP-seq FMR1 is shown on the x axis (chrX) and for the distal region is shown on the Y axis. All data is from one replicate per cell line. Hi-C data is binned at 1 MB resolution. The domains are identified by which chromosome they are on, and the FMR1 domain is identified as “chrX.” Blue bars and green arrows highlight trans interactions.

FIG. 21 depicts exemplary results of inter-chromosomal interactions between distal H3K9me3 domains in FXS_NPC_15 and FXS_NPC_133. Pairwise Hi-C interactions among all of the distal H3K9me3 domains gained in FXS for pre-mutation-length (FXS_133, upper triangle) and normal-length (FXS_15, lower triangle). Domains annotated by chromosome. The window for each region includes the H3K9me3 domain gained and 3 Mb of flanking genome. H3K9me3 ChIP-seq signal for all domains for both FXS_15 and FXS_133 are plotted above Hi-C heatmaps. Blue boxes highlight FXS gained trans interactions.

FIG. 22 depicts exemplary results of inter-chromosomal interactions between distal H3K9me3 domains in FXS_NPC_15 and FXS_NPC_306. Pairwise Hi-C interactions among all of the distal H3K9me3 domains gained in FXS for short mutation-length (FXS_306, upper triangle) and normal length (FXS_15, lower triangle). Domains annotated by chromosome. The window for each region includes the H3K9me3 domain gained and 3 Mb of flanking genome. H3K9me3 ChIP-seq signal for all domains for both FXS_15 and FXS_306 are plotted above Hi-C heatmaps. Blue boxes highlight FXS gained trans interactions.

FIG. 23 depicts exemplary results of inter-chromosomal interactions between distal H3K9me3 domains in FXS_NPC_15 and FXS_NPC_326. Pairwise Hi-C interactions among all of the distal H3K9me3 domains gained in FXS for long mutation-length (FXS_326, upper triangle) and normal length (FXS_15, lower triangle). Domains annotated by chromosome. The window for each region includes the H3K9me3 domain gained and 3 Mb of flanking genome. H3K9me3 ChIP-seq signal for all domains for both FXS_15 and FXS_326 are plotted above Hi-C heatmaps. Blue boxes highlight FXS gained trans interactions.

FIG. 24 depicts exemplary results of inter-chromosomal interactions between distal H3K9me3 domains in FXS_NPC_306 and FXS_NPC_378. Pairwise Hi-C interactions among all of the distal H3K9me3 domains gained in FXS for long mutation-length (FXS_378, upper triangle) and short mutation-length (FXS_306, lower triangle). Domains annotated by chromosome. The window for each region includes the H3K9me3 domain gained and 3 Mb of flanking genome. H3K9me3 ChIP-seq signal for all domains for both FXS_378 and FXS_306 are plotted above Hi-C heatmaps. Blue boxes highlight FXS gained trans interactions.

FIG. 25 depicts exemplary results of inter-chromosomal interactions between distal H3K9me3 domains in FXS_NPC_326 and FXS_NPC_378. Pairwise Hi-C interactions among all of the distal H3K9me3 domains gained in FXS for short mutation-length (FXS_326, upper triangle) and long mutation-length (FXS_378, lower triangle). Domains annotated by chromosome. The window for each region includes the H3K9me3 domain gained and 3 Mb of flanking genome. H3K9me3 ChIP-seq signal for all domains for both FXS_378 and FXS_326 are plotted above Hi-C heatmaps. Blue boxes highlight FXS gained trans interactions.

FIGS. 26a-26b depict exemplary results of CGG, TTAGGG (telomeric repeat), and fragile sites with respect to FXS H3K9me3 domains. In FIG. 26a, for each of the H3K9me3 domains consistently present in all three F×S lines, the H3K9me3 ChIP-seq in WT_NPC_15, FXS_NPC_326, and FXS_NPC_378 is shown. Underneath, CGG repeat tracks are shown in red, and TTAGGGTTAGGG (SEQ ID NO:1) are shown in blue. Fragile sites are shown with orange bars. FIG. 26b depicts zoom-ins on CGG STR tracks at genes in each of the FXS H3K9me3 domains.

FIGS. 27a-27e depict exemplary results of statistical tests demonstrating unique genetic features of FXS H3K9me3 domains compared to the rest of the genome. FIGS. 27a-b depict the average number of CGG STR tracks within the first 2 kb of genes in FXS H3K9me3 domains is compared to the null distribution of the number of CGG STR tracks in the first 2 kb of genes in expected null distributions consisting of (FIG. 27a) 1000 draws of size-matched, randomly sampled intervals not within H3K9me3 domains. P-values computed as a one-tailed area under the curve to the right of the red line. ** indicates P-values <0.05. To count the number of CGGs, all CGG STRs that were at least (CGG)n>=2 were considered “CGG”, and the total number of CGG occurrences within the first 2 kb of genes whose promoters were located within either the null or test set of domains was summed. FIG. 27b depicts the number of fragile sites within the H3K9me3 domains in FXS compared to a null distribution of 1000 draws of size-matched genomic intervals that are not in H3K9me3 domains as called by RSEG. Fragile sites were obtained from the HumCFS database. FIGS. 27c-27d depict the length of 6110 CGG STRs was profiled across 544 individuals (data from Annear et al., 2021, Sci Rep 11, 2515). CGGs in that study were stratified into whether they were located in one of n=12 FXS specific H3K9me3 domains, in a genotype invariant H3K9me3 domain, or in the 1 MB flanking regions around the n=12 FXS specific H3K9me3 domains. The hg19 reference genome length of the CGGs in each category (FIG. 27c) and the median length of the CGGs in each category across the population (FIG. 27d) are plotted. FIG. 27e depicts the length of all CGGs of at least n=2 units across the genome was profiled in the 5 cell lines used in this study using ExpansionHunter. The number of variations in CGG length between the cell lines and the reference genome are plotted for each cell line, stratified by where the CGG is located.

FIG. 28 depicts exemplary results of Nanopore long-read sequencing over the FMR1 gene in edited cell lines. Visual representation of Nanopore long reads that span the transcription start site and first 200 bp of FMR1 for the two edited cell lines and their parent lines in this study. The sequence of each read is shown in colors corresponding to base pairs shown in the legend.

FIGS. 29a-29j depict exemplary results demonstrating that engineering the FMR1 CGG STR to pre-mutation length reverses a subset of distal heterochromatin domains and reprograms 3D genome misfolding in fragile X syndrome. FIG. 29a depicts a schematic of CRISPR engineered STR cut-out IPSCs. Isogenic set 1 (purple) consists of long mutation-length F×S line (FXS_iPSC_376) engineered to normal-length (FXS_iPSC_376_cut 4) where CGG repeats were removed so only 4 remained. Isogenic set 2 (blue) consists of a second long mutation-length F×S line (FXS_iPSC_326) engineered to pre-mutation length (FXS_iPSC_326_cut_180) where CGG repeats were removed so only 180 remained. FIG. 29b depicts the number of CGGs present in the FMR1 5′UTR per cell line. Each dot represents the number of CGGS in one long Nanopore read. Bar represents mean across all reads. FIG. 29c depicts H3K9me3 ChIP-seq in WT iPSC and Isogenic Set 1 and Set 2 iPSC for a 8 Mb region around FMR1. Genes are shown below ChIP-seq tracks. FMR1, SLITRK2, and SLITRK4 are highlighted in red, blue, and green respectively. FIG. 29d depicts FMR1 and SLITRK2 mRNA levels (n=3 replicates) from qRT-PCR shown for WT, Isogenic Set 1, and Set 2 iPSCs. Each dot represents one replicate, with the horizontal line representing the mean. FIG. 29e depicts Hi-C interaction frequency heatmaps in an 8 Mb region around FMR1 for iPSC lines in isogenic set 2. H3K9me3 and CTCF ChIP-seq is displayed below the heatmaps. In FIGS. 29f-29g, H3K9me3 ChIP-seq signal is shown for each of n=12 heterochromatin domains consistently gained across all three F×S lines as well as heterochromatin domains present only in the FXS parent line for IPSC in isogenic set 1 (FIG. 29f) and isogenic set 2 (FIG. 29g). Each line of the heat map represents one region. Red boxes annotate reprogrammed domains that lose H3K9me3 signal upon FMR1 CGG STR shortening. FIG. 29h depicts the average H3K9me3 signal in isogenic set 2 (FXS_iPSC_326 and FXS_326_CUT_180) for each FXS H3K9me3 domain, stratified up by whether the domain was reprogrammed or resistant upon shortening of the mutation-length CGG to pre-mutation length. FIG. 29i depicts pairwise Hi-C interactions among all of the distal H3K9me3 domains gained in FXS for long mutation-length (FXS_iPSC_326, upper triangle) and pre-mutation length (FXS_180, lower triangle). Domains annotated by chromosome. The window for each region includes the H3K9me3 domain gained and 3 Mb of flanking genome. H3K9me3 ChIP-seq signal for all domains for both lines are plotted above Hi-C heatmaps. Blue boxes highlight FXS trans interactions that are resistant to reprogramming. Green boxes highlight FXS trans interactions that are reprogrammed upon CGG shortening to pre-mutation length. FIG. 29j depicts the average number of CGG STR tracks within the first 2 kb of genes in either reprogrammed or resistant H3K9me3 domains is compared to genes in a null distribution consisting of 1000 draws of n=12 size-matched, randomly-sampled intervals located within genotype-independent H3K9me3 domains that remain constant throughout normal-length, short mutation, pre-mutation, and disease lines. To count CGGs, all CGG tracks that were at least (CGG)n;>2 were analyzed and the total number of CGG occurrences within the first 2 kb of genes whose promoters were located within either the null or test set of domains was summed. A one-tailed randomization test was used to compute a P-value as the area under the null distribution curve to the right of the red line.

FIGS. 30a-30d depict exemplary details of CRISPR edited iPSC cell lines. FIG. 30a depicts a schematic of iPS cell lines and the CRISPR edited deletions used in this study. FIGS. 30b-d depict Nanopore long-read analysis of longest continuous CGG trat (FIG. 30b), number of AGG interrupters within CGG STR (FIG. 30c), and total number of continuous CGG tracks within the STR in the 5′UTR of FMR1 (FIG. 30d).

FIG. 31 depicts exemplary results of mappability statistics of 5C samples.

FIG. 32 depicts exemplary results of 5C, H3K9me3, and CTCF in Fragile X Syndrome in iPSC upon CGG repeat cut to normal length. 5C in a ˜6 MB region around FMR1 is shown for a disease cell line, FXS_iPSC_387, and an isogenic line where the repeats were cut out from >500 to around 4. H3K9me3 ChIP-seq and CTCF for each are shown below.

FIG. 33 depicts exemplary results of distal H3K9me3 domains in Fragile X Syndrome in iPSC upon CGG repeat cut. H3K9me3 ChIP-seq is shown around distal FXS specific H3K9me3 domains in one replicate for of each cell line in Isogenic set 1 and set 2. Lines representing RSEG H3K9me3 domain calls are shown above H3K9me3 ChIP-seq track. Red represents locations of the CGG repeat, blue represents TTAGGGTTAGGG (SEQ ID NO:1), and yellow represents fragile sites, obtained from HumFCS database.

FIG. 34 depicts exemplary results of inter-chromosomal interactions between FMR1 and distal H3K9me3 domains in FXS_iPSC_326 and isogenic cut out line FXS_326_CUT_180 Hi-C interactions between FMR1 and the distal H3K9me3 domains (see FIG. 19) are shown for long mutation-sample FXS_iPSC_326 and the edited cell lines with CGGs cut to 180, (FXS_326_CUT_180). The window for each region includes the H3K9me3 domain gained and upto 20 MB of flanking genome. H3K9me3 ChIP-seq FMR1 is shown on the x axis (chrX) and for the distal region is shown on the Y axis. All data is from one replicate per cell line. Hi-C data is binned at 1 MB resolution. The domains are identified by which chromosome they are on, and the FMR1 domain is identified as “chrX.”

FIGS. 35a-35i depicts exemplary results demonstrating that overexpression of a pre-mutation length CGG STR tract de-represses pathologically silenced expression and attenuates FXS H3K9me3 domains. FIG. 35a depicts a schematic showing experimental workflow. FIGS. 35b-e depict mRNA levels as assessed by qRT-PCR for FMR1 (FIG. 35b), SLITRK2 (FIG. 35c), DPP6 (FIG. 35d), and SHISA6 (FIG. 35e) in long mutation-length FXS iPSCs which either did receive (GFP+) or did not receive (GFP−) the CGGx99 plasmid. Error bars represent the standard error of the mean for the indicated number of technical replicates. FIGS. 35f-h depict H3K9me3 CUT&RUN in either the GFP- or GFP+ cells at the gained H3K9me3 domains at FMR1 (FIG. 35f), DPP6 (FIG. 35g), and SHISA6 (FIG. 35h). FIG. 35i depicts a schematic model. Numerous heterochromatin domains interact via long-range trans interactions to form an inter-chromosomal subnuclear hub with the FMR1 locus in fragile X syndrome. When CGG STRs are normal-length, FMR1 and other chromosomes do not cluster and do not interact in trans. As the CGG tract expands to pre-mutation length, FMR1 mRNA levels increase. When the CGG STR tract expands to short mutation-length, FMR1 expression drastically decreases. Distal fragile sites acquire large H3K9me3 domains that cluster together spatially in trans. When CGG repeats expand to long mutation-length (450), FMR1 mRNA levels are fully repressed, and the distal heterochromatin domains gain H3K9me3 signal intensity. Upon cutout from long mutation-length to pre-mutation, a subset of distal domains lose H3K9me3 signal and the long-range trans interactions with FMR1 are abolished. By contrast, cutout of long mutation-length to normal-length CGG triplets does not reverse heterochromatin domains, trans interactions remain connected, and genes remain repressed. Finally, the role for the pre-mutation length CGG tract is made evident upon introduction of an exogenous 99 CGG triplet STR transgene to FXS iPSCs. The presence of transcribed CGG plasmid leads to reduction of heterochromatin across all gained H3K9me3 domains and reactivates FMR1 and distal gene expression, suggesting that long-range 3D Epigenome miswiring in FXS is driven by the DNA or RNA CGG STR sequence.

FIGS. 36a-36b depict exemplary results of the effect of CGG-99x on gene expression and H3K9me3 domain strength. FIG. 36a depicts the expression of FMR1 (FIG. 36b) via qRT-PCR in cells which either did receive (GFP+) or did not receive (GFP−) the CGGx99 plasmid. Two biological replicates separate from that in FIG. 35b are shown. FIG. 36b depicts H3K9kme3 ChIP-seq in consistently gained H3K9me3 domains in FXS.

FIG. 37 depicts exemplary sequences of guide RNAs used for Cas9-targeted Nanopore sequencing.

FIG. 38A-FIG. 38J: A >5 Megabase-sized domain of H3K9me3 heterochromatin spreads across the FMR1 locus in a CGG STR length-dependent manner in fragile X syndrome. (A) Schematic of iPSC lines used to model FXTAS and FXS, including normal-length, pre-mutation, and full mutation-length. (B) Representative Nanopore long reads across the FMR1 5′UTR. Colors reflect nucleotides (yellow: A, blue: T, green: C, red: G, dark green, CGG). (C) Number of CGG triplets in the FMR1 5′UTR from single-molecule Nanopore long reads, called using STRique. (D) FMR1 expression normalized to GAPDH via qRT-PCR. Horizontal lines represent the mean between n=2 biological replicates. (E) Proportion of 19 CpG dinucleotides methylated in the 500 bp FMR1 promoter per allele computed using nanopolish and single-molecule Nanopore long reads. Distribution across all alleles per condition is shown. (F) Proportion of CGG triplets within the 5′ UTR STR tract that are methylated, called using STRique. Each dot is a single-molecule long read representing one allele. (G) Hi-C heatmaps representing interaction frequency an 8 Mb region around FMR1 in across all five iPSC-NPC lines. A/B compartment score, input-normalized H3K9me3 ChIP-seq, and CTCF ChIP-seq is displayed below the heatmaps. FMR1, SLITRK2, and SLITRK4 are highlighted in red, blue, and green respectively. (H) Summed interactions between FMR1 and the TAD immediately upstream or downstream is shown as a difference from WT_19. (I) Hi-C fold-change interaction frequency maps. Gained and lost contacts compared to WT_19 are highlighted in red and blue, respectively. (J) SLITRK2 and SLITRK4 mRNA levels as evaluated by RNA-seq. Data was normalized using DESeq's median-of-ratios method. Horizontal lines represent the mean between n=2 biological replicates.

FIG. 39A-FIG. 39I: Heterochromatin domains and synaptic gene silencing on autosomes in fragile X syndrome. (A) Three classes of H3K9me3 domains identified genome-wide across five iPSC-NPC lines: (i) FXS-consistent: consistently gained in all three F×S lines, (ii) FXS-variable: gained in only a single F×S line, or (iii) Genotype-invariant: present in all genotypes. (B) Heatmaps of Hi-C interaction frequency for a 1.6 Mb region around an autosomal H3K9me3 domains encompassing SHISA6. Compartment score, input normalized H3K9me3 ChIP-seq, and CTCF ChIP-seq in iPSC-NPCs is displayed below the heatmaps for all five conditions. Horizontal lines represent H3K9me3 domain calls. SHISA6 highlighted in orange. (C) Insulation score measuring boundary strength across Hi-C matrices in the same 1.6 Mb region as (B) for all five cell lines. (D) SHISA6 mRNA levels as evaluated by RNA-seq. Horizontal lines represent the central tendency (mean) between n=2 biological replicates. (E) Pooled H3K9me3 and CTCF ChIP-seq data across FXS-consistent H3K9me3 domains. (F) Boundary strength in each of the distal FXS-consistent H3K9me3 domains for WT_NPC_19 (red) and FXS_NPC_389 (purple) lines. (G) mRNA levels as evaluated by RNA-seq for n=27 protein-coding genes in FXS-consistent H3K9me3 domains. Genes are shown if they were expressed in at least one iPSC-NPC line. Each point represents expression of one gene averaged across n=2 biological replicates. * indicates p<0.05 when compared to WT_NPC_19. Pvalues were calculated using a one-tailed Mann Whitney U test. (H) Gene ontology (GO) analysis using WebGESTALT for n=34 protein-coding genes localized to FXS-consistent H3K9me3 domains as defined in panel (A). (I) Expression of all genes in FXS-consistent H3K9me3 domains across GTEX tissues. Genes (n=68) were shown if expression non-zero across tissues.

FIG. 40A-FIG. 40G: Autosomal heterochromatin domains overlay unstable STRs, and spatially connect with FMR1 via inter-chromosomal interactions in FXS. (A) Hi-C inter-chromosomal interaction heatmaps binned at 1 Mb resolution between H3K9me3 domains +/−5 Mb on FMR1 (x-axis) and either chromosome 8 or 5 (y-axis). Green arrows highlight trans interactions. (B) Inter-chromosomal interactions between each of the N=10 FXS H3K9me3 domain on autosomes and FMR1 on chromosome X. Trans interaction frequency between each autosomal H3K9me3 domain and FMR1 across 5 iPSC-NPC lines with increasing CGG STR length. Bar represents mean X-autosome trans contacts for all H3K9me3 domains. (C) Pairwise Hi-C trans interactions among autosomal H3K9me3 domains and the X chromosome in FXS_386 (upper triangle) and normal-length (WT_19, lower triangle). H3K9me3 domains +/−3 Mb annotated by chromosome. Input normalized H3K9me3 Chip-seq signal for all domains for both FXS_389 and WT_19 are plotted above Hi-C heatmaps. Blue boxes highlight FXS-gained trans interactions. (D-E) The number of FXS-consistent H3K9me3 (D) domains or (E) boundaries overlapping unstable STR tracts (i.e. reproducible expansion/contraction events in 2/3 of our FXS iPSCs verified by long-read sequencing) compared to null distributions consisting of 10,000 draws of n=10 size-matched, randomly-sampled genotype-invariant H3K9me3 domains or boundaries of domains. Empirical, one-tailed P-value shown as computed in (26). (F-G) Example unstable STR expansion events for (F) RBFOX1 and (G) CSDM1 genes. Short-read alignments to the hg38 reference genome for all five iPSC-NPC lines.

FIG. 41A-FIG. 41H: Engineering the FMR1 CGG STR to pre-mutation length attenuates a subset of H3K9me3 domains and de-represses pathologically silenced expression in fragile X syndrome. (A) Schematic of long-premutation-length and short-premutation-length cut-back iPSC lines generated with CRISPR/Cas9 genome editing from multiple parental mutation-length lines. (B-C) Input normalized H3K9me3 CUT&Run profiles are shown in (B) 6 Mb and (C) 200 kb regions on chrX around FMR1 for each engineered iPSC line. Horizontal lines above the signal indicate H3K9me3 RSEG domain calls. FMR1, SLITRK2, and SLITRK4 are highlighted in red, blue, and green, respectively. (D) FMR1 and SLITRK2 mRNA levels normalized to GAPDH (n=2 replicates) from qRT-PCR shown for each iPSC line in (A). Each dot represents one replicate, with the horizontal line representing the mean. (E) Hi-C interaction frequency heatmaps in an 8 Mb region around FMR1 for FXS_386 and its cutout FXS_386_cut196. H3K9me3 and CTCF profiles are displayed below the heatmaps. (F) H3K9me3 CUT&RUN signal for distal FXS-consistent and FXS-variable H3K9me3 domains. One H3K9me3 domain per row. Red boxes annotate domains with reduced signal upon FMR1 CGG length engineering, where reprogrammed was defined as losing at least half of the H3K9me3 domain in the cutout compared to the parent iPSCs. (G) Average H3K9me3 signal for each FXS-consistent H3K9me3 domain, stratified up by whether the domain was reprogrammed or resistant upon engineering of the mutation-length CGG to pre-mutation length. P-value, Two-tailed Mann-Whitney U test. (H) Pairwise Hi-C interactions among all H3K9me3 domains in FXS_386 (upper triangle) and FXS_386_cut196 (lower triangle). Input normalized H3K9me3 signal is annotated by chromosome and plotted as the domain +/−3 Mb of flanking genome. Blue and green boxes highlight FXS trans interactions that are resistant and amenable to reprogramming upon CGG shortening to pre-mutation length, respectively.

FIG. 42A-FIG. 42G: Inter-chromosomal interactions among heterochromatin domains in FXS are detectable in single-cells. (A-B) DNA FISH images with Oligopaints probes for the H3K9me3 domain on chromosome X (magenta) interacting with (A) the H3K9me3 domain on chromosome 12 (yellow) or (B) all H3K9me3 domains in WT_19, FXS_386, and FXS_386_cut196 iPSC nuclei. (C) Proportion of cells with chrX and chr12 H3K9me3 domains within 0-250 nm, 251-500 nm, and >500 nm distance. (D) Distances between the H3K9me3 domains on chrX and chr12 from individual iPSC nuclei. Pvalues were calculated using a two-tailed Mann Whitney U test. * indicates p<le-6. (E) Average distance per cell (one point per individual cell) between the H3K9me3 domain on chrX and all other domains. Pvalues were calculated using a two-tailed Mann Whitney U test.* indicates p<le-12. (F) Distribution of the number of individual foci representing each autosomal and chrX domain. Pvalues were calculated using a two-tailed Mann Whitney U test. *=p<le-12. (G) Schematic model of long-range inter-chromosomal interaction hubs of heterochromatin domains silencing long synaptic genes and unstable STRs in FXS.

FIG. 43A-FIG. 43D: Morphology and expected homogeneous marker expression in human induced pluripotent stem cells (iPSCs) and iPSC-derived neural progenitor cells (iPSC-derived NPCs). (A) Phase contrast images of iPSC colony morphology. (B) Immunofluorescence staining of human iPSC lines for OCT4 (green) and NESTIN (cyan) co-localized with DAPI (blue) as a nuclear marker. (C) Phase contrast images of iPSC-derived NPC rosettes. (D) Immunofluorescence staining of human iPSC-derived NPCs for OCT4 (green) and NESTIN (cyan) co-localized with DAPI (blue) as a nuclear marker. Scale bars, 250 μm.

FIG. 44A-FIG. 44C: Clinical-grade genotyping of CGG STR tracts in iPSC lines. (A) Capillary gel electrophoresis traces from the AmplideX® mPCR FMR1 Kit for WT_19, PM 136, FXS_373, FXS_386, and FXS_389. (B) Estimated average CGG tract lengths from Amplidex traces are listed in a table. (C) Capillary gel electrophoresis traces from the AmplideX® mPCR FMR1 Kit for FXS_373 paired with FXS_373_CUT_180 and FXS_386 paired with FXS_386_CUT_196.

FIG. 45A-FIG. 45E: Visual representation of bonito and guppy base-called forward and reverse reads spanning upstream and downstream regions of FMR1 across iPSC lines. (A) Forward reads called by both Guppy and Bonito base mapping. Nucleotides are represented by colors as shown in the legend. (B-C) Reverse reads (B) without fmlrc and (C) with fmlrc base pair correction with both Guppy and Bonito read mapping. (D-E) CGG STR lengths for all four conditions across all 5 iPSC lines in FIG. 1. Black bar represents median CGG length.

FIG. 46A-FIG. 46D: DNA methylation analysis of Nanopore long-read sequencing. (A) Schematic representation of the FMR1 gene with annotations demonstrating the location of the CGG tract and promoter associated CpGs which are analyzed in panels (C) and (D). (B) Visual representation of bonito base-called reverse reads (i.e., alleles) spanning upstream and downstream regions of FMR1 across iPSC lines. CGGs are highlighted in green and other nucleotides are grey. (C) Visual representation of methylation status of CpGs within the FMR1 CGG tract by the STR-specific tool STRique. (D) Visual representation of methylation status of the 19 CpGs present in the FMR1 promoter called using nanopolish. In panels C-D, DNA methylation is annotated per read, with read order kept consistent across B-D.

FIG. 47A-FIG. 47H: 3D genome folding disruption and acquisition of a Mb-sized heterochromatin domain at the FMR1 locus in fragile X syndrome. (A) Hi-C data across all five iPSC-NPC lines is shown as a heatmap of interaction frequency for an 8 Mb region around FMR1. A/B compartment score, input normalized H3K9me3 Chip-seq, and CTCF Chip-seq in iPSC-derived NPCs is displayed below the heatmaps. (B-C) Input normalized H3K9me3 ChTP signal and A/B compartment score across the locus shown in (A) binned into 40 bins and plotted for each iPSC-NPC line. (D) CTCF and input normalized H3K9me3 ChIP-seq for 6 Mb upstream of FMR1 are overlaid for each iPSC-NPC line. (E, G) Zoom-ins to Box1 and Box2 from (A) showing (E) FMR1-SLITRK2 or (G) FMR1-SLITRK4 interactions. Arrows point to loops. CTCF motif orientation show by track with blue and red arrows. (F,H) Boxplot showing the interaction frequency measured with Hi-C between FMR1 and either (F) SLITRK2 or (H) SLITRK4.

FIG. 48A-FIG. 48H: Comparison of CGG STR length, H3K9me3 signal, and 3D genome features genome-wide in two iPSC clones derived from the same FXS parent line. (A) Number of CGG triplets in the FMR1 5′UTR based on Nanopore long read sequencing. (B) Percent of CGGs in the FMR1 5′UTR which are methylated based on STRique analysis of single-molecule Nanopore long-reads. (C) Representative images of Nanopore long-reads across FMR1. (D) Input normalized H3K9me3 ChIP-seq is shown for a 6 Mb locus around FMR1. (E) Hi-C data in a 10 Mb locus around FMR1. Tracks for input normalized H3K9me3 and CTCF ChIP-seq are plotted underneath heat maps. (F) FMR1 and SLITRK2 expression using qRT-PCR. G) Input normalized H3K9me3 ChIP-seq signal for each of n=10 FXS-consistent H3K9me3 domains on autosomes (i.e. domains consistently gained across all three FXS iPSC-NPC lines), n=12 FXS-variable H3K9me3 domains gained only in the FXS_386 iPSC-NPC (compared to FXS_373 and FXS_389, two genetically different backgrounds), and n=58 genotype-invariant H3K9me3 domains on autosomes (i.e. domains present in all normal-length, pre-mutation, and FXS iPSC-NPC lines). Each row represents one H3K9me3 domain. (H) Hi-C heatmaps around n=10 FXS-consistent H3K9me3 domains on autosomes. Input normalized H3K9me3 ChIP-seq, and CTCF ChIP-seq tracks are displayed below the heatmaps. Lines representing RSEG H3K9me3 domain calls are shown above H3K9me3 signal.

FIG. 49A-FIG. 49C: A Mb-scale heterochromatin domain is deposited across the FMR1 locus in FXS iPSC. (A) Input normalized H3K9me3 Chip-seq profile in five iPSC lines in an 8 Mb region around FMR1. FMR1, SLITRK2, and SLITRK4 genes are highlighted in red, blue, and green respectively. (B) Zoom-in on data from (A) is shown in a 75 kb window around the FMR1 gene. (C) Input normalized H3K9me3 ChIP-seq from (A) is overlaid for all 5 iPSC lines.

FIG. 50A-FIG. 50F: Linear and 3D genome alterations occur around the FMR1 gene upon mutation-length CGG expansion in EBV-transformed lymphoblastoid B-cell lines. (A) FMR1, SLITRK2, and SLITRK4 gene expression via RNA-seq is shown for one normal-length EBV-transformed lymphoblastoid cell line (WT_B) and two FXS EBV-transformed lymphoblastoid cell lines (FXS_B_900, FXS_B_650) isolated from two FXS patients. (B) Hi-C heatmaps of a 10 Mb region around FMR1 are shown for WT_B and FXS_B_900. Tracks representing A/B compartment score, CTCF ChIP-seq, and input normalized H3K9me3 ChIP-seq are shown below heatmaps. CTCF peak calls and H3K9me3 domain calls displayed as horizontal lines above their respective signal. (C-D) Zoom in to (C) 1.5 Mb and (D) 80 kb around FMR1 (location marked in grey rectangle in (E). (E) 5C interaction frequency heatmaps of ˜5 Mb of the X chromosome surrounding the FMR1 gene in WT_B, FXS_B_900, and FXS_B_650. CTCF and input normalized H3K9me3 ChTP seq tracks displayed underneath heatmaps. (F) 5C interaction frequency heatmaps from FXS EBV-transformed lymphoblastoid cell lines are divided by 5C interaction frequency heatmaps from the normal-length EBV-transformed lymphoblastoid line.

FIG. 51: Reproducible gain of autosomal FXS-consistent H3K9me3 domains and loss of CTCF occupancy upon mutation-length CGG STR expansion in iPSC-derived NPCs. Input normalized H3K9me3 and CTCF Chip-seq are plotted together at n=10 autosomal FXS-consistent H3K9me3 domains across five iPSC-NPC lines (normal-length (WT_19), pre-mutation (PM_136), and three mutation-length F×S lines (FXS_373, FXS_386, FXS_389).

FIG. 52: Genome folding is severely disrupted at sites of distal FXS-consistent H3K9me3 domain acquisition in FXS. Hi-C interaction frequency heatmaps around distal FXS-consistent H3K9me3 domains in iPSC-derived NPC lines. Tracks for A/B compartment score, input normalized H3K9me3 ChIP-seq, and CTCF ChIP-seq from iPSC-NPCs are displayed below heatmaps. RSEG H3K9me3 domain calls are shown above the input normalized H3K9me3 ChIP-seq track as horizontal lines.

FIG. 53A-FIG. 53D: Expression and ontology of genes affected by FXS H3K9me3 domain acquisition. (A) RNA-seq data showing expression of genes located in the FXS-consistent H3K9me3 domains in iPSC-NPC cells. Of the N=11 domains, N=10 autosomal and N=1 on the X chromosome, we only examine genes that are expressed in at least one of the iPSC-NPC lines. Two biological replicates are shown for each sample. Horizontal line represents the mean of two replicates for each of five lines. (B+D) Gene ontology analysis using WebGESTALT with settings Over-Representation Analysis, geneontology, Biological Process, Cellular Component, Molecular function, with “genome-protein coding” as the reference. We only examine protein-coding genes, and used a P-value cutoff of p<0.01 and enrichment >4. (B) Gene ontology analysis for n=409 protein-coding genes co-localized with N=58 genotype-invariant H3K9me3 domains. (C) Total number of up- and down-regulated genes for pre-mutation (PM 136), and three FXS mutation-length iPS-NPCs (FXS_373, FXS_386, FXS_389) compared to normal-length (WT_19) as determined by DESeq. (D) Gene ontology analysis for n=31 protein-coding genes co-localized with N=24 FXS-variable H3K9me3 domains present in only 1 of 3 FXS iPSC-NPC lines.

FIG. 54: FXS-consistent H3k9me3 domains are acquired in iPSCs. Input normalized H3K9me3 ChIP-seq is shown around distal FXS-consistent H3K9me3 domains for five iPSC (normal-length (WT_19), pre-mutation (PM 136), and three mutation-length F×S lines (FXS_373, FXS_386, FXS_389). H3K9me3 domain calls from RSEG software are shown above H3K9me3 ChIP-seq track as horizontal lines. Of N=11 total H3K9me3 domains gained in FXS, one is at FMR1 on the X chromosome, and the remaining 10 are shown here.

FIG. 55A-FIG. 55B: H3K9me3 domains are gained distal autosomes in FXS in EBV-transformed lymphoblastoid B-cell lines. (A-B) Input normalized H3K9me3 ChIP-seq in one WT and two FXS EBV-transformed lymphoblastoid cell lines for (A) the N=10 autosomal FXS-consistent H3K9me3 domains identified in iPSCs and iPSC-NPCs and (B) the N=5 autosomal FXS H3K9me3 domains identified in EBV-transformed lymphoblastoid cell lines. H3K9me3 domain calls are shown as horizontal lines above the ChIP-seq signal. Of the N=10 H3K9me3 domains from iPSC-NPCs, N=2 (in red text) are gained in FXS vs. normal-length EBV-transformed lymphoblastoid cells.

FIG. 56: Inter-chromosomal interactions between FMR1 and distal FXS-consistent H3K9me3 domains in iPSC-NPC. Hi-C interactions between FMR1 and each of the distal FXS-specific H3K9me3 domains in normal-length (WT_19), pre-mutation (PM_136), and three mutation-length (FXS_373, FXS_386, FXS_389). The window for each region includes the H3K9me3 domain and +/−5 Mb of flanking genome. Input normalized H3K9me3 ChIP-seq is shown for chr X (x-axis) and for the distal region (y-axis). Hi-C data is binned at 1 Mb resolution. Blue bars and green arrows highlight trans interactions.

FIG. 57: Inter-chromosomal interactions between FXS-consistent H3K9me3 domains in WT_19 and PM_136 iPSC-NPC. Pairwise Hi-C trans interactions between FXS-consistent H3K9me3 domain loci on autosomes (N=10) and the X chromosome are compared for pre-mutation-length iPSC-NPC (PM_136, upper triangle) and normal-length iPSC-NPC (WT_19, lower triangle). H3K9me3 domains +/−3 Mb annotated by chromosome. Input normalized H3K9me3 Chip-seq signal for all domains for both PM_136 and WT_19 are plotted alongside Hi-C heatmaps. Blue boxes highlight FXS-gained trans interactions.

FIG. 58: Inter-chromosomal interactions between FXS-consistent H3K9me3 domains in WT_19 and FXS_373 iPSC-NPC. Pairwise Hi-C trans interactions between FXS-consistent H3K9me3 domain loci on autosomes (N=10) and the X chromosome are compared for FXS full-mutation-length iPSC-NPC (FXS_373, upper triangle) and normal-length iPSC-NPC (WT_19, lower triangle). H3K9me3 domains +/−3 Mb annotated by chromosome. Input normalized H3K9me3 Chip-seq signal for all domains for both FXS_373 and WT_19 are plotted alongside Hi-C heatmaps. Blue boxes highlight FXS-gained trans interactions.

FIG. 59: Inter-chromosomal interactions between FXS-consistent H3K9me3 domains in WT_19 and FXS_386 iPSC-NPC. Pairwise Hi-C trans interactions between FXS-consistent H3K9me3 domain loci on autosomes (N=10) and the X chromosome are compared for FXS full-mutation-length iPSC-NPC (FXS_386, upper triangle) and normal-length iPSC-NPC (WT_19, lower triangle). H3K9me3 domains +/−3 Mb annotated by chromosome. Input normalized H3K9me3 Chip-seq signal for all domains for both FXS_386 and WT_19 are plotted alongside Hi-C heatmaps. Blue boxes highlight FXS-gained trans interactions.

FIG. 60: Inter-chromosomal interactions between FXS-consistent H3K9me3 domains in FXS_376 and FXS_389 iPSC-NPC. Pairwise Hi-C trans interactions between FXS-consistent H3K9me3 domain loci on autosomes (N=10) and the X chromosome are compared for two different FXS full-mutation-length iPSC-NPCs (FXS_389, upper triangle, and FXS_376, lower triangle). H3K9me3 domains +/−3 Mb annotated by chromosome. Input normalized H3K9me3 Chip-seq signal for all domains for both FXS_389 and FXS_376 are plotted alongside Hi-C heatmaps. Blue boxes highlight FXS-gained trans interactions.

FIG. 61: Inter-chromosomal interactions between FXS-consistent H3K9me3 domains in FXS_386 and FXS_389 iPSC-NPC. Pairwise Hi-C trans interactions between FXS-consistent H3K9me3 domain loci on autosomes (N=10) and the X chromosome are compared for two FXS full-mutation-length iPSC-NPCs (FXS_386, upper triangle, and FXS_389, lower triangle). H3K9me3 domains +/−3 Mb annotated by chromosome. Input normalized H3K9me3 Chip-seq signal for all domains for both FXS_386 and FXS_389 are plotted alongside Hi-C heatmaps. Blue boxes highlight FXS-gained trans interactions.

FIG. 62A-FIG. 62G: Three methods of measuring genome integrity support largely normal karyotype in iPSC and iPSC-NPC lines. (A-E) De novo genome assemblies across all chromosomes per iPSC/iPSC-NPC lines constructed from Hi-C data and PCR-free whole genome sequencing reads using W2rapContigger, Juicer, and 3D-DNA in NPC. Lines on the jupiter plots demonstrate mapping between de novo genome assembly (left half of circle) with the hg38 reference genome (right half of circle). FXS-consistent H3K9me3 domains are denoted with black stripes and black stars along the de novo assembled chromosomes (left half). Grey stripes denote masked areas. (F) Copy number variation in 5kb bins all chromosomes calculated from Hi-C data in iPSC-NPC using NeoLoopFinder. (G) Genome coverage in 5 kb bins across all chromosomes calculated from PCR free whole genome sequencing data in iPSCs.

FIG. 63A-FIG. 63E: FXS-consistent H3K9me3 domains have similar chromosomal locations and are enriched for contracting/expanding STRs in Zhou et al FXS iPSC (A) The location of the FXS-consistent H3K9me3 domain at FMR1 and n=10 distal gained H3K9me3 domains is highlighted in a red box on a chromosome ideogram (obtained from the UCSC genome browser). (B) Schematic demonstrating method of identifying STRs that contract/expand in FXS iPSC lines by comparing to n=90 PCR-free whole genome sequencing datasets from unaffected individuals. (C). The location of all Zhou et al FXS iPSC-specific STRs within FXS-consistent H3K9me3 domains is annotated as a red line under input normalized H3K9me3 ChIP tracks for each iPSC-NPC line used in this study. (D) The number of Zhou et al FXS iPSC-specific unstable STR events (i.e. normal-length range expansion/contractions exclusively in three FXS iPSC lines verified by long-read sequencing) co-localized with autosomal FXS-consistent H3K9me3 domains or with (E) boundaries (350kb flanking regions) of FXS-consistent H3K9me3 domains (purple lines) compared to a null distribution consisting of 10,000 draws of n=10 size-matched, randomly-sampled intervals taken from the genomewide background. Empirical, one-tailed P-value shown in D-E are computed as in (26).

FIG. 64A-FIG. 64D: Unstable STR lengths called by GangSTR are validated using short- and long-read sequencing. (A) For each STR, the length of that tract across 5 iPSC lines from PCR-free whole genome sequencing short reads is shown. Each dot represents data from one read. (B) For the STRs, short read sequencing reads in 5 iPSC lines that are mapped over the STR are shown, and deviations from hg38 are highlighted in zoom boxes. Deviations which are significantly expanded/contracted (26) compared to N=90 unaffected individuals are highlighted in zoom boxes, while deviations that are not significantly expanded/contracted are highlighted in hatched zoom boxes. (C) Oxford nanopore long read sequencing in FXS_386 that map to the STR are shown. (D) For each individual STR, the tract length for each of the 5 iPSC lines is plotted in colored lines on top of distribution of tract lengths in n=90 non-diseased individuals. All STR tract lengths were called using GangSTR.

FIG. 65A-FIG. 65D: Additional unstable STR lengths called by GangSTR are validated using short- and long-read sequencing. (A) For each STR, the length of that tract across 5 iPSC lines from PCR-free whole genome sequencing short reads is shown. Each dot represents data from one read. (B) For the STRs, short read sequencing reads in 5 iPSC lines that are mapped over the STR are shown, and deviations from hg38 are highlighted in zoom boxes. Deviations which are significantly expanded/contracted (26) compared to N=90 unaffected individuals are highlighted in zoom boxes, while deviations that are not significantly expanded/contracted are highlighted in hatched zoom boxes. (C) Oxford nanopore long read sequencing in FXS_386 that map to the STR are shown. (D) For each individual STR, the tract length for each of the 5 iPSC lines is plotted in colored lines on top of distribution of tract lengths in n=90 non-diseased individuals. All STR tract lengths were called using GangSTR.

FIG. 66A-FIG. 66X: Six single-cell-derived FXS clones with sgRNA+Cas9 engineered CGG STR tracts. (A-D) Schematics illustrating CRISPR-engineered CGG STR tract lengths across four independent FXS iPSC lines with corresponding DNA agarose gel images used to assess CGG tract length of all engineered clones. (E-H) Bar graphs depicting CGG STR tract lengths for each parent line and corresponding CRISPR-engineered FXS clone. (I, K, Q, S) Input normalized H3K9me3 CUT&Run profiles encompassing the FMR1 locus on chromosome X shown for 200 kb around FMR1 across all iPSC lines. (J, L, R, T) Input normalized H3K9me3 CUT&Run profiles encompassing the FMR1 locus on chromosome X shown for 6 Mb around FMR1 across all iPSC lines. (M-X) Relative FMR1 or SLITRK2 mRNA levels quantified by RT-qPCR and normalized to GAPDH.

FIG. 67A-FIG. 67B: Effect of FMR1 5′UTR CGG STR cut-back on autosomal FXS-consistent H3K9me3 domains. (A) Input normalized H3K9me3 CUT&Run profiles are shown for 5 FXS-consistent H3K9me3 domains where the signal was not reprogrammed in any of the CGG STR engineered iPSC lines. (B) Input normalized H3K9me3 CUT&Run profiles are shown for 5 FXS-consistent H3K9me3 domains where the signal was reprogrammed in at least one CGG STR engineered iPSC line. Reprogrammed domains are highlighted in red. Loci were determined to be “reprogrammed” if the H3K9me3 domain size in the engineered line was under half the size as in the original parent line (26).

FIG. 68A-FIG. 68J: Quantifying the effect of FMR1 5′UTR CGG STR cut-back on autosomal FXS H3K9me3 domains. (A-B) Input normalized H3K9me3 CUT&Run signal for each of n=10 distal FXS H3K9me3 domains consistently gained across all three FXS parent iPSC lines (FXS-consistent H3K9me3 domains) and for H3K9me3 domains present in only one FXS parent iPSC line (FXS-variable H3K9me3 domains). Each row represents one H3K9me3 domain flanked by +/−3 Mb. (A) FXS_371 parent iPSCs and cut-back to intermediate-length 60 CGGs. (B) FXS_389 parent iPSCs and cut-back to intermediate-length 40 CGGs. (C-J) Average input normalized H3K9me3 signal for each of (C-D) n=10 distal FXS-consistent H3K9me3 domains, (E-G) distal FXS-variable H3K9me3 domains, and (H-J) genotype-invariant H3K9me3 domains. Domains are stratified by whether the heterochromatin signal was reprogrammed or resistant upon cut-back of (A-B, D, G, J) mutation-length FXS_371 and FXS_389 iPSCs to 40-60 CGGs (intermediate-length), (C, F, I) mutation-length FXS_371 and FXS_373 iPSCs to 100 CGGs (short-pre-mutation length), and (E, H) mutation-length FXS_386 and FXS_373 iPSCs to 180-195 CGGs (long-pre-mutation length).

### DETAILED DESCRIPTION

The present invention relates to systems and methods for modulating heterochromatin content or the level or activity of a gene or gene product that has been silenced by the formation of heterochromatin regions and the use thereof for the prevention and treatment of fragile X syndrome and diseases and disorders associated with fragile X syndrome including, but not limited to, reproductive, epithelial, neural adhesion, and synaptic plasticity defects.

In some embodiments, the composition also comprises methods of diagnosing a subject as having fragile X syndrome and diseases and disorders associated with fragile X syndrome including, but not limited to, reproductive, epithelial, neural adhesion, and synaptic plasticity defects. In some embodiments the method comprises detecting a decreased level of at least one gene product of a gene that has been silenced by the formation of heterochromatin regions.

### Definitions

Unless defined otherwise, all technical and scientific terms used herein have the same meaning as commonly understood by one of ordinary skill in the art to which this invention belongs.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of 20%, +10%, +5%, ±1%, or ±0.1% from the specified value, as such variations are appropriate to perform the disclosed methods.

The term “activate,” as used herein, means to induce or increase an activity or function, for example, about ten percent relative to a control value. Preferably, the activity is induced or increased by 50% compared to a control value, more preferably by 75%, and even more preferably by 95%. “Activate,” as used herein, also means to increase a molecule, a reaction, an interaction, a gene, an mRNA, and/or a protein's expression, stability, function or activity by a measurable amount or to increase entirely. Activators are compounds that, e.g., bind to, partially or totally induce stimulation, increase, promote, induce activation, activate, sensitize, or up regulate a protein, a gene, and an mRNA stability, expression, function and activity, e.g., agonists.

As used herein in reference to a display library, a “barcode” refers to a unique molecular identifier to distinguish cells expressing distinct display molecules. For example, the barcode may be a unique DNA sequence within a cell that corresponds to a display molecule expressed by said cell. This barcode may be detected using methods including, but not limited to, next generation sequencing

“Coding sequence” or “encoding nucleic acid” as used herein may refer to the nucleic acid (RNA or DNA molecule) that comprise a nucleotide sequence which encodes an antigen set forth herein. The coding sequence may further include initiation and termination signals operably linked to regulatory elements including a promoter and polyadenylation signal capable of directing expression in the one or more cells of an individual or mammal to whom the nucleic acid is administered. The coding sequence may further include sequences that encode signal peptides.

A “constitutive” promoter is a nucleotide sequence which, when operably linked with a polynucleotide which encodes or specifies a gene product, causes the gene product to be produced in a cell under most or all physiological conditions of the cell.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease, or disorder, the frequency with which such a sign or symptom is experienced by a patient, or both, is reduced.

The term “expression” as used herein is defined as the transcription of a particular nucleotide sequence driven by its promoter and/or the translation of said nucleotide sequence into an amino acid sequence.

The term “gene” means the segment of DNA involved in producing a polypeptide chain. It may include regions preceding and following the coding region (leader and trailer) as well as intervening sequences (introns) between individual coding segments (exons).

As used herein, an “inducible” promoter is a nucleotide sequence which, when operably linked with a polynucleotide which encodes or specifies a gene product, causes the gene product to be produced substantially only when an inducer which corresponds to the promoter is present.

The term “inhibit,” as used herein, means to suppress or block an activity or function, for example, about ten percent relative to a control value. Preferably, the activity is suppressed or blocked by 50% compared to a control value, more preferably by 75%, and even more preferably by 95%. “Inhibit,” as used herein, also means to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down regulate a protein, a gene, and an mRNA stability, expression, function and activity, e.g., antagonists.

As used herein, an “instructional material” includes a publication, a recording, a diagram, or any other medium of expression which can be used to communicate the usefulness of a compound, composition, vector, or delivery system of the invention in the kit for effecting alleviation of the various diseases or disorders recited herein. Optionally, or alternately, the instructional material can describe one or more methods of alleviating the diseases or disorders in a cell or a tissue of a mammal. The instructional material of the kit of the invention can, for example, be affixed to a container which contains the identified compound, composition, vector, or delivery system of the invention or be shipped together with a container which contains the identified compound, composition, vector, or delivery system. Alternatively, the instructional material can be shipped separately from the container with the intention that the instructional material and the compound be used cooperatively by the recipient.

“Measuring” or “measurement,” or alternatively “detecting” or “detection,” means assessing the presence, absence, quantity or amount (which can be an effective amount) of a given substance.

The term “modulate,” as used herein, refers to mediating a detectable increase or decrease in a desired response. For example, a small molecule may be used to increase or decrease the level of interaction between two proteins.

As used herein, the term “next generation sequencing” refers to sequencing methods that allow for massively parallel sequencing of clonally amplified molecules and of single nucleic acid molecules. Next generation sequencing is synonymous with “massively parallel sequencing” for most purposes. Non-limiting examples of next generation sequencing include sequencing-by-synthesis using reversible dye terminators, and sequencing-by-ligation.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al, Mol. Cell. Probes 8:91-98 (1994)). The term nucleic acid is used interchangeably with gene, cDNA, and mRNA encoded by a gene.

“Operably linked” as used herein may mean that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

As used herein in reference to interactions, “promote” refers to inducing or increasing an interaction between two species. For example, a small molecule may promote or increase interactions between two proteins.

“Promoter” as used herein may mean a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which can be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the promoters from GAL1 (galactose), PGK (phosphoglycerate kinase), ADH (alcohol dehydrogenase), AOX1 (alcohol oxidase), HIS4 (histidinol dehydrogenase), metallothionein, 3-phosphoglycerate kinase, such as enolase, glyceraldehyde-3-phosphate dehydrogenase, hexokinase, pyruvate decarboxylase, phospho-fructokinase, glucose-6-phosphate isomerase, 3-phosphoglycerate mutase, pyruvate kinase, triosephosphate isomerase, phospho-glucose isomerase, and glucokinase.

The term “regulating” as used herein can mean any method of altering the level or activity of a substrate. Non-limiting examples of regulating with regard to a protein include affecting expression (including transcription and/or translation), affecting folding, affecting degradation or protein turnover, and affecting localization of a protein. Non-limiting examples of regulating with regard to an enzyme further include affecting the enzymatic activity. “Regulator” refers to a molecule whose activity includes affecting the level or activity of a substrate. A regulator can be direct or indirect. A regulator can function to activate or inhibit or otherwise modulate its substrate.

The terms “subject”, “individual”, “patient” and the like are used interchangeably herein, and refer to any animal, or cells thereof whether in vitro or in situ, amenable to the methods described herein. In some non-limiting embodiments, the patient, subject or individual is a human. In various embodiments, the subject is a human subject, and may be of any race, sex, and age.

“Vector” as used herein may mean a nucleic acid sequence containing an origin of replication. A vector may be a plasmid, bacteriophage, bacterial artificial chromosome or yeast artificial chromosome. A vector may be a DNA or RNA vector. A vector may be either a self-replicating extrachromosomal vector or a vector which integrates into a host genome.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The invention is based, in part, on the finding of ten 3-10 Mb sized H3K9me3 domains on distal chromosomes that silence a cohort of distal genes directly, and further the identification that the distal silenced genes have CGG short tandem repeat tracks, similar to that of Fmr1.

In some embodiments, the invention provides compositions and methods for activating or reactivating or de-repressing a H3K9me3-heterochromatin mark containing gene. In some embodiments, the invention provides compositions and methods for modulating one or more epigenomic marker. For example, in some embodiments, the composition reduces the level of epigenomic methylation of at least one H3K9me3-heterochromatin mark containing gene or H3K9me3-heterochromatin mark containing gene regulator. In one embodiment, the composition blocks RNA mediated heterochromatin formation. In one embodiment, the composition inhibits RNA-DNA interactions which may induce heterochromatin.

In various embodiments, the invention relates to compositions for modulation, activation, reactivation or de-repression of one or more H3K9me3-heterochromatin mark containing gene. H3K9me3-heterochromatin mark containing genes that can be modulated, activated, reactivated, or de-repressed include, but are not limited to, FMR1, FMR1NB, FMR1-AS1, C5orf38, CTD-2194D22.4, LOC100506858, IRX2, LOC105374620, LINC01377, LINC01019, LINC01017, IRX1, LINC02114, DPP6, LINC01287, LOC101929998, CSMD1, FAM135B, LOC101927815, COL22A1, KCNK9, TRAPPC9, MYOM2, LOC101927845, LINC01591, LOC101927915, SPANXN4, SPANXN3, SLITRK4, SPANXN2, UBE2NL, SPANXN1, SLITRK2, TMEM257, MIR892C, MIR890, MIR888, MIR892A, MIR892B, MIR891B, MIR891A, CXorf51B, CXorf51A, MIR513C, MIR513B, MIR513A1, MIR513A2, MIR506, MIR507, MIR508, MIR514B, MIR509-2, MIR509-3, MIR509-1, MIR510, MIR514A1, MIR514A2, MIR514A3, TCERG1L, MIR378C, TCERG1L-AS1, LINC01164, TMEM132C, TMEM132D, LOC100996671, LOC101927592, LINC00508, LOC100996679, GLT1D1, RIMBP2, LINC00939, LOC101927464, LOC100128554, LINC00944, LINC00943, LOC440117, LOC101927616, LOC101927637, LOC105370068, FLJ37505, LINC00507, CRAT8, LOC101927694, MIR4419B, MIR3612, SLC15A4, LOC283352, LOC101927735, LOC100190940, FZD10-AS1, FZD10, PIWIL1, RBFOX1, MIR8065, TMEM114, DNAH9, SHISA6, PTPRT, LOC101927159, LINC01441, LINC01440, CBLN4, and MC3R.

In some embodiments, the present invention relates to the prevention or treatment of a disease or disorder by administration of a composition for activating, reactivating or de-repressing a H3K9me3-heterochromatin mark containing gene. In some embodiments, the disease or disorder is fragile X syndrome, fragile X-associated primary ovarian insufficiency or a disease or disorder associated with fragile X syndrome including, but not limited to, reproductive, epithelial, neural adhesion, and synaptic plasticity defects.

In some embodiments, the present invention relates to the prevention or treatment of a disease or disorder by administration of a composition for inhibiting at least one heterochromatin formation, RNA mediated heterochromatin formation and RNA-DNA interactions. In some embodiments, the disease or disorder is fragile X syndrome, fragile X-associated primary ovarian insufficiency or a disease or disorder associated with fragile X syndrome including, but not limited to, reproductive, epithelial, neural adhesion, and synaptic plasticity defects.

### Activators

In various embodiments, the present invention includes compositions and methods of activating, reactivating or de-repressing a H3K9me3-heterochromatin mark containing gene. In some embodiments, the composition for activating, reactivating or de-repressing a H3K9me3-heterochromatin mark containing gene, increases the amount of polypeptide, the amount of mRNA, the amount of protein activity, or a combination thereof of the gene product.

It will be understood by one skilled in the art, based upon the disclosure provided herein, that an increase in the level of a H3K9me3-heterochromatin mark containing gene encompasses the increase in gene expression, including transcription, translation, or both. The skilled artisan will also appreciate, once armed with the teachings of the present invention, that an increase in the level of a H3K9me3-heterochromatin mark containing gene includes an increase in gene product activity. Thus, increasing the level or activity of a H3K9me3-heterochromatin mark containing gene includes, but is not limited to, increasing transcription, translation, or both, of a H3K9me3-heterochromatin mark containing gene; and it also includes increasing any activity of a H3K9me3-heterochromatin mark containing gene product as well.

Activation or reactivation of a H3K9me3-heterochromatin mark containing gene can be assessed using a wide variety of methods, including those disclosed herein, as well as methods well-known in the art or to be developed in the future. That is, a person of skill in the art would appreciate, based upon the disclosure provided herein, that increasing the level or activity of a H3K9me3-heterochromatin mark containing gene can be readily assessed using methods that assess the level of a nucleic acid comprising a H3K9me3-heterochromatin mark containing gene product (e.g., mRNA) and/or the level of polypeptide comprising a H3K9me3-heterochromatin mark containing gene product in a biological sample.

An activator of a H3K9me3-heterochromatin mark containing gene can include, but should not be construed as being limited to, a chemical compound, a protein, a peptidomemetic, an epigenomic editor, and a nucleic acid molecule, including a DNA molecule, and an RNA molecule.

In some embodiments, activator of a H3K9me3-heterochromatin mark containing gene can include a small molecule chemical compound. Exemplary small molecule compounds that can be used to remove DNA methylation, and therefore activate or re-activate on or more H3K9me3-heterochromatin mark containing gene include, but are not limited to, 5-aza-2′-deoxycytidine.

One of skill in the art would readily appreciate, based on the disclosure provided herein, that a H3K9me3-heterochromatin mark containing gene activator encompasses a chemical compound that increases the level, activity, or the like of a H3K9me3-heterochromatin mark containing gene. Additionally, a H3K9me3-heterochromatin mark containing gene activator encompasses a chemically modified compound, and derivatives, as is well known to one of skill in the chemical arts.

Epigenomic Editors

The present disclosure is directed, in part, to targeting and modulating the epigenetic “state” (e.g., methylation state) of one or more genes. In some embodiments, the compositions of the invention include the use of epigenomic editors to remove at least one H3K9me3-heterochromatin mark from at least one H3K9me3-heterochromatin mark containing gene to activate, re-activate or de-repress the gene.

In some embodiments, epigenetic modification is done with a chimeric RNA which contains a DNA binding element at one end, a scaffold segment for disabled CAS9 (dCAS9) binding and an epigenetic effector enzyme or an aptamer to capture an epigenetic effector enzyme at the other end. Epigenetic effector enzymes that can be used according to the methods of the invention include, but are not limited to, a transcription activation domain from VP64 or NF-κB p65; an enzyme that catalyzes DNA demethylation, such as Ten-Eleven Translocation (TET) protein, histone lysine demethylase (KDM) and other demethylases. For example, in one embodiment, the chimeric RNA binds near transcription elements for the H3K9me3-heterochromatin mark containing gene and the associated epigenetic effector enzyme demethylates local histones and thus activates, reactivates or de-represses the H3K9me3-heterochromatin mark containing gene.

In some embodiments, the associated epigenetic effector enzyme is linked to the N-terminus or C-terminus of the catalytically inactive Cas9 protein, optionally with an intervening linker, and the linker does not interfere with the activity of the fusion protein.

In some embodiments, the present invention provides nucleic acids encoding the epigenomic editors described herein, as well as expression vectors comprising the nucleic acids and host cells that express the epigenomic editors.

In some embodiments, the DNA binding element comprises an sgRNA specific for at least one H3K9me3-heterochromatin mark containing gene. In some embodiments, the DNA binding element comprises an sgRNA specific for FMR1, FMR1NB, FMR1-AS1, C5orf38, CTD-2194D22.4, LOC100506858, IRX2, LOC105374620, LINC01377, LINC01019, LINC01017, IRX1, LINC02114, DPP6, LINC01287, LOC101929998, CSMD1, FAM135B, LOC101927815, COL22A1, KCNK9, TRAPPC9, MYOM2, LOC101927845, LINC01591, LOC101927915, SPANXN4, SPANXN3, SLITRK4, SPANXN2, UBE2NL, SPANXN1, SLITRK2, TMEM257, MIR892C, MIR890, MIR888, MIR892A, MIR892B, MIR891B, MIR891A, CXorf51B, CXorf51A, MIR513C, MIR513B, MIR513A1, MIR513A2, MIR506, MIR507, MIR508, MIR514B, MIR509-2, MIR509-3, MIR509-1, MIR510, MIR514A1, MIR514A2, MIR514A3, TCERG1L, MIR378C, TCERG1L-AS1, LINC01164, TMEM132C, TMEM132D, LOC100996671, LOC101927592, LINC00508, LOC100996679, GLT1D1, RIMBP2, LINC00939, LOC101927464, LOC100128554, LINC00944, LINC00943, LOC440117, LOC101927616, LOC101927637, LOC105370068, FLJ37505, LINC00507, CRAT8, LOC101927694, MIR4419B, MIR3612, SLC15A4, LOC283352, LOC101927735, LOC100190940, FZD10-AS1, FZD10, PIWIL1, RBFOX1, MIR8065, TMEM114, DNAH9, SHISA6, PTPRT, LOC101927159, LINC01441, LINC01440, CBLN4, or MC3R.

Expression of FMR1 Pre-Mutation

In some embodiments, the invention includes transgenic compositions for overexpression of one or more H3K9me3-heterochromatin mark containing gene. In one embodiment, the H3K9me3-heterochromatin mark containing gene is Fmr1. In some embodiments, the Fmr1 gene comprises a pre-mutation length CGG tandem repeat. In one embodiment, the pre-mutation length of the CGG repeat comprises 40 to 200 tandem CGG repeats. In one embodiment, the pre-mutation length of the CGG repeat comprises 50 to 195 tandem CGG repeats. In some embodiments, the Fmr1 gene comprising a pre-mutation length CGG tandem repeat is expressed as a transgene to drive the presence of 190 CGG containing RNA to form inclusion bodies and sequester RNA away from the heterochromatin domains.

One of skill in the art, when armed with the disclosure herein, would appreciate that methods for overexpression of one or more H3K9me3-heterochromatin mark containing gene encompasses administering to a subject a nucleic acid molecule encoding FMR1 comprising a pre-mutation length CGG tandem repeat or a recombinant nucleic acid molecule encoding FMR1 comprising a pre-mutation length CGG tandem repeat.

The recombinant nucleic acid sequence construct described above can be placed in one or more vectors. The one or more vectors can contain an origin of replication. The one or more vectors can be a plasmid, bacteriophage, bacterial artificial chromosome or yeast artificial chromosome. The one or more vectors can be either a self-replication extra chromosomal vector, or a vector which integrates into a host genome.

Vectors include, but are not limited to, plasmids, expression vectors, recombinant viruses, any form of recombinant “naked DNA” vector, and the like. A “vector” comprises a nucleic acid which can infect, transfect, transiently or permanently transduce a cell. It will be recognized that a vector can be a naked nucleic acid, or a nucleic acid complexed with protein or lipid. The vector optionally comprises viral or bacterial nucleic acids and/or proteins, and/or membranes (e.g., a cell membrane, a viral lipid envelope, etc.). Vectors include, but are not limited to replicons (e.g., RNA replicons, bacteriophages) to which fragments of DNA may be attached and become replicated. Vectors thus include, but are not limited to RNA, autonomous self-replicating circular or linear DNA or RNA (e.g., plasmids, viruses, and the like, see, e.g., U.S. Pat. No. 5,217,879), and include both the expression and non-expression plasmids. In some embodiments, the vector includes linear DNA, enzymatic DNA or synthetic DNA. Where a recombinant microorganism or cell culture is described as hosting an “expression vector” this includes both extra-chromosomal circular and linear DNA and DNA that has been incorporated into the host chromosome(s). Where a vector is being maintained by a host cell, the vector may either be stably replicated by the cells during mitosis as an autonomous structure, or is incorporated within the host's genome.

The vector can be a heterologous expression construct, which is generally a plasmid that is used to introduce a specific gene into a target cell. Once the expression vector is inside the cell, polypeptide that is encoded by the recombinant nucleic acid sequence construct is produced by the cellular-transcription and translation machinery ribosomal complexes. The vector can express large amounts of stable messenger RNA, and therefore proteins.

Gene Editing

In some embodiments, the invention includes compositions for reducing a full mutation length CGG tandem repeat of Fmr1 to an intermediate or pre-mutation length to activate, re-activate, or de-repress one or more H3K9me3-heterochromatin mark containing gene. In some embodiments, the compositions and methods reduce a CGG tandem repeat of Fmr1 comprising at least 200 tandem CGG repeat units to an intermediate or pre-mutation length of between 40 and 200 tandem CGG repeat units. In some embodiments, the compositions and methods reduce a CGG tandem repeat of Fmr1 comprising at least 200 tandem CGG repeat units to a pre-mutation length of between 55 and 200 tandem CGG repeat units. In some embodiments, the compositions and methods reduce a CGG tandem repeat of Fmr1 comprising at least 200 tandem CGG repeat units to a pre-mutation length of between 60 and 200 tandem CGG repeat units. In some embodiments, the compositions and methods reduce a CGG tandem repeat of Fmr1 comprising at least 200 tandem CGG repeat units to a pre-mutation length of between 65 and 200 tandem CGG repeat units. In some embodiments, the compositions and methods reduce a CGG tandem repeat of Fmr1 comprising at least 200 tandem CGG repeat units to a pre-mutation length of between 70 and 200 tandem CGG repeat units. In some embodiments, the compositions and methods reduce a CGG tandem repeat of Fmr1 comprising at least 200 tandem CGG repeat units to a pre-mutation length of between 75 and 200 tandem CGG repeat units. In some embodiments, the compositions and methods reduce a CGG tandem repeat of Fmr1 comprising at least 200 tandem CGG repeat units to a pre-mutation length of between 80 and 200 tandem CGG repeat units. In some embodiments, the compositions and methods reduce a CGG tandem repeat of Fmr1 comprising at least 200 tandem CGG repeat units to a pre-mutation length of between 85 and 200 tandem CGG repeat units. In some embodiments, the compositions and methods reduce a CGG tandem repeat of Fmr1 comprising at least 200 tandem CGG repeat units to a pre-mutation length of between 90 and 200 tandem CGG repeat units. In some embodiments, the compositions and methods reduce a CGG tandem repeat of Fmr1 comprising at least 200 tandem CGG repeat units to a pre-mutation length of between 170 and 190 tandem CGG repeat units.

Compositions and methods that can be used to reduce a full mutation length CGG tandem repeat of Fmr1 to a pre-mutation length include, but are not limited to, gene editing compositions (e.g., CRISPR-Cas systems). CRISPR methodologies employ a nuclease, CRISPR-associated (Cas), that complexes with small RNAs as guides (gRNAs) to cleave DNA in a sequence-specific manner upstream of the protospacer adjacent motif (PAM) in any genomic location. CRISPR may use separate guide RNAs known as the crRNA and tracrRNA. These two separate RNAs have been combined into a single RNA to enable site-specific mammalian genome cutting through the design of a short guide RNA. Cas and guide RNA (gRNA) may be synthesized by known methods. Cas/guide-RNA (gRNA) uses a non-specific DNA cleavage protein Cas, and an RNA oligo to hybridize to target and recruit the Cas/gRNA complex. In one embodiment, a guide RNA (gRNA) targeted to the Fmr1 gene, and a CRISPR-associated (Cas) peptide form a complex to induce mutations within the targeted gene. In one embodiment, the composition comprises a gRNA or a nucleic acid molecule encoding a gRNA. In one embodiment, the composition comprises a Cas peptide or a nucleic acid molecule encoding a Cas peptide.

Inhibitors

In some embodiments, the present disclosure is directed to inhibitors of heterochromatin formation, inhibitors of RNA mediated heterochromatin formation, inhibitors of RNA-DNA interactions, inhibitors of the expression of one or more CGG tandem repeat containing gene, inhibitors of the expression of one or more histone H3-K9 methyltransferase gene, and compounds that disrupt heterochromatin domains. Exemplary inhibitory compositions include, but are not limited to, antisense oligonucleotides (ASOs), antibodies, small molecule chemical compounds and other inhibitory compositions as discussed elsewhere herein. Any inhibitor of RNA mediated heterochromatin formation, or compound which disrupts heterochromatic regions is encompassed in the invention.

It will be understood by one skilled in the art, based upon the disclosure provided herein, that a decrease in the level of RNA mediated heterochromatin formation encompasses a decrease in the expression, including transcription, translation, or both of one or more CGG tandem repeat containing gene. CGG tandem repeat containing genes that can be inhibited according to the methods of the invention include, but are not limited to, FMR1, SHISA6, IRX2, TCERG1L, PTPRT, DPP6, and TMEM257. The skilled artisan will also appreciate, once armed with the teachings of the present invention, that a decrease in the level of one or more CGG tandem repeat containing gene includes a decrease in the activity of one or more CGG tandem repeat containing gene product. Thus, a decrease in the level or activity of one or more CGG tandem repeat containing gene includes, but is not limited to, decreasing transcription, translation, or both, of a nucleic acid comprising one or more CGG tandem repeat containing gene; and it also includes decreasing any activity of one or more CGG tandem repeat containing gene product as well.

It will be understood by one skilled in the art, based upon the disclosure provided herein, that a decrease in the level of heterochromatin formation encompasses a decrease in the expression, including transcription, translation, or both of one or more gene involved in methylation of histones, wherein the methylation results in heterochromatin formation and gene silencing. Histone methylation genes that can be inhibited according to the methods of the invention include, but are not limited to, a histone H3-K9 methyltransferase, for example, ESET, G9a, Eu-HMTase, Suppressor Of Variegation 3-9 Homolog 1 (SUV39H1) and Suppressor Of Variegation 3-9 Homolog 2 (SUV39H2). The skilled artisan will also appreciate, once armed with the teachings of the present invention, that a decrease in the level of one or more histone H3-K9 methyltransferase gene includes a decrease in the activity of one or more histone H3-K9 methyltransferase gene product. Thus, a decrease in the level or activity of one or more histone H3-K9 methyltransferase gene includes, but is not limited to, decreasing transcription, translation, or both, of a nucleic acid comprising a histone H3-K9 methyltransferase gene; and it also includes decreasing any activity of a histone H3-K9 methyltransferase gene product as well.

In one embodiment, the composition of the invention comprises an inhibitor of the expression of one or more CGG tandem repeat containing gene, an inhibitor of the expression of one or more histone H3-K9 methyltransferase gene, a compound which disrupts heterochromatin domains, or any combination thereof. In one embodiment, the inhibitor is selected from the group consisting of a small interfering RNA (siRNA), a microRNA, an antisense nucleic acid, a ribozyme, an expression vector encoding a transdominant negative mutant, an antibody, a peptide and a small molecule.

In one embodiment, the composition of the invention comprises an inhibitor of CGG short tandem repeat (STR) containing RNA. In one embodiment, the inhibitor of CGG STR containing RNA decreases the half-life or stability of the CGG STR containing RNA. In one embodiment, the inhibitor comprises an antisense oligonucleotide directed against CGG STR containing RNA.

One skilled in the art will appreciate, based on the disclosure provided herein, that one way to decrease the mRNA and/or protein levels of one or more CGG tandem repeat containing gene and/or histone H3-K9 methyltransferase gene in a cell is by reducing or inhibiting expression of the nucleic acid comprising the one or more CGG tandem repeat containing gene and/or histone H3-K9 methyltransferase gene. Thus, the protein level of the protein encoded by one or more CGG tandem repeat containing gene and/or histone H3-K9 methyltransferase gene in a cell can be decreased using a molecule or compound that inhibits or reduces gene expression such as, for example, siRNA, an antisense molecule or a ribozyme. However, the invention should not be limited to these examples.

In one embodiment, siRNA is used to decrease the level of one or more CGG tandem repeat containing gene and/or histone H3-K9 methyltransferase gene. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., 1998, Nature 391(19):306-311; Timmons et al., 1998, Nature 395:854; Montgomery et al., 1998, TIG 14 (7):255-258; David R. Engelke, Ed., RNA Interference (RNAi) Nuts & Bolts of RNAi Technology, DNA Press, Eagleville, P A (2003); and Gregory J. Hannon, Ed., RNAi A Guide to Gene Silencing, Cold Spring Harbor Laboratory Press, Cold Spring Harbor, N.Y. (2003). Soutschek et al. (2004, Nature 432:173-178) describe a chemical modification to siRNAs that aids in intravenous systemic delivery. Optimizing siRNAs involves consideration of overall G/C content, C/T content at the termini, Tm and the nucleotide content of the 3′ overhang. See, for instance, Schwartz et al., 2003, Cell, 115:199-208 and Khvorova et al., 2003, Cell 115:209-216. Therefore, the present invention also includes methods of decreasing levels of host protein at the protein level using RNAi technology.

In other related aspects, the invention includes an isolated nucleic acid encoding an inhibitor, wherein an inhibitor such as an siRNA or antisense molecule, inhibits one or more CGG tandem repeat containing gene and/or histone H3-K9 methyltransferase gene, a derivative thereof, a regulator thereof, or a downstream effector, operably linked to a nucleic acid comprising a promoter/regulatory sequence such that the nucleic acid is preferably capable of directing expression of the protein encoded by the nucleic acid. Thus, the invention encompasses expression vectors and methods for the introduction of exogenous DNA into cells with concomitant expression of the exogenous DNA in the cells such as those described, for example, in Sambrook et al. (2012, Molecular Cloning: A Laboratory Manual, Cold Spring Harbor Laboratory, New York) and as described elsewhere herein.

In another aspect, the invention includes a vector comprising an siRNA or antisense polynucleotide. Preferably, the siRNA or antisense polynucleotide is capable of inhibiting the expression of one or more CGG tandem repeat containing gene and/or histone H3-K9 methyltransferase gene. In one embodiment, the siRNA or antisense polynucleotide inhibits the expression of FMR1, SHISA6, IRX2, TCERG1L, PTPRT, DPP6, or TMEM257. In one embodiment, the siRNA or antisense polynucleotide inhibits the expression of ESET, G9a, Eu-HMTase, SUV39H1 and SUV39H2. The incorporation of a desired polynucleotide into a vector and the choice of vectors is well-known in the art.

The siRNA or antisense polynucleotide can be cloned into a number of types of vectors as described elsewhere herein. For expression of the siRNA or antisense polynucleotide, at least one module in each promoter functions to position the start site for RNA synthesis.

In order to assess the expression of the siRNA or antisense polynucleotide, the expression vector to be introduced into a cell can also contain either a selectable marker gene or a reporter gene or both to facilitate identification and selection of expressing cells from the population of cells sought to be transfected or infected through viral vectors. In other embodiments, the selectable marker may be carried on a separate piece of DNA and used in a co-transfection procedure. Both selectable markers and reporter genes may be flanked with appropriate regulatory sequences to enable expression in host cells. Useful selectable markers are known in the art and include, for example, antibiotic-resistance genes, such as neomycin resistance and the like.

In one embodiment of the invention, an antisense nucleic acid sequence which is expressed by a plasmid vector is used to inhibit the expression of one or more CGG tandem repeat containing gene, inhibit the expression of one or more histone H3-K9 methyltransferase gene, disrupt heterochromatin domains, or any combination thereof. The antisense expressing vector is used to transfect a mammalian cell or the mammal itself, thereby causing reduced endogenous expression of one or more CGG tandem repeat containing gene and/or histone H3-K9 methyltransferase gene.

In some embodiments an antisense nucleic acid sequence specific for one or more CGG tandem repeat sequences may be used to specifically bind to a nucleic acid molecule comprising a CGG tandem repeat sequence and inhibit the interaction of the nucleic acid molecule comprising the CGG tandem repeat sequence with a distal CGG repeat or a CGG tandem repeat on a different chromosome.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules of the invention may be made synthetically and then provided to the cell. In some embodiments, the antisense oligomers are about 10 to about 30 nt, since they are easily synthesized and introduced into a target cell. Synthetic antisense molecules contemplated by the invention include oligonucleotide derivatives known in the art which have improved biological activity compared to unmodified oligonucleotides (see U.S. Pat. No. 5,023,243).

Ribozymes and their use for inhibiting gene expression are also well known in the art (see, e.g., Cech et al., 1992, J. Biol. Chem. 267:17479-17482; Hampel et al., 1989, Biochemistry 28:4929-4933; Eckstein et al., International Publication No. WO 92/07065; Altman et al., U.S. Pat. No. 5,168,053). Ribozymes are RNA molecules possessing the ability to specifically cleave other single-stranded RNA in a manner analogous to DNA restriction endonucleases. Through the modification of nucleotide sequences encoding these RNAs, molecules can be engineered to recognize specific nucleotide sequences in an RNA molecule and cleave it (Cech, 1988, J. Amer. Med. Assn. 260:3030). A major advantage of this approach is the fact that ribozymes are sequence-specific.

There are two basic types of ribozymes, namely, tetrahymena-type (Hasselhoff, 1988, Nature 334:585) and hammerhead-type. Tetrahymena-type ribozymes recognize sequences which are four bases in length, while hammerhead-type ribozymes recognize base sequences 11-18 bases in length. The longer the sequence, the greater the likelihood that the sequence will occur exclusively in the target mRNA species. Consequently, hammerhead-type ribozymes are preferable to tetrahymena-type ribozymes for inactivating specific mRNA species, and 18-base recognition sequences are preferable to shorter recognition sequences which may occur randomly within various unrelated mRNA molecules.

In one embodiment of the invention, a ribozyme is used to inhibit the expression of one or more CGG tandem repeat containing gene, inhibit the expression of one or more histone H3-K9 methyltransferase gene, disrupt heterochromatin domains, or any combination thereof. Ribozymes useful for inhibiting the expression of a target molecule may be designed by incorporating target sequences into the basic ribozyme structure which are complementary, for example, to the mRNA sequence of one or more CGG tandem repeat containing gene and/or histone H3-K9 methyltransferase gene of the present invention. Ribozymes targeting one or more CGG tandem repeat containing gene and/or histone H3-K9 methyltransferase gene may be synthesized using commercially available reagents (Applied Biosystems, Inc., Foster City, Calif.) or they may be genetically expressed from DNA encoding them.

When the inhibitor of the invention is a small molecule, a small molecule antagonist may be obtained using standard methods known to the skilled artisan. Such methods include chemical organic synthesis or biological means. Biological means include purification from a biological source, recombinant synthesis and in vitro translation systems, using methods well known in the art. Exemplary compounds that can function as inhibitors of heterochromatin formation, inhibitors of RNA-DNA interactions, which may induce heterochromatin, or inhibitors of one or more CGG tandem repeat containing gene include, but are not limited to compound 1a/1f (Disney et al., 2012, ACS Chem Biol. 7(10):1711-1718) and ETP69.

Combinatorial libraries of molecularly diverse chemical compounds potentially useful in treating a variety of diseases and conditions are well known in the art as are method of making the libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development.

In a general method for small library synthesis, an activated core molecule is condensed with a number of building blocks, resulting in a combinatorial library of covalently linked, core-building block ensembles. The shape and rigidity of the core determines the orientation of the building blocks in shape space. The libraries can be biased by changing the core, linkage, or building blocks to target a characterized biological structure (“focused libraries”) or synthesized with less structural bias using flexible cores.

In some embodiments, an antibody specific for one or more CGG tandem repeat containing gene (e.g., an antagonist to one or more CGG tandem repeat containing gene) may be used. In another embodiment, the antibody or antagonist is a protein and/or compound having the desirable property of interacting with one or more CGG tandem repeat containing gene and thereby sequestering the CGG tandem repeat containing gene.

Expression Constructs

In one embodiment, the invention relates to recombinant nucleic acid sequence construct comprising a pre-mutation length CGG repeat which functions as a competitive inhibitor to disrupt interactions between a mutation length CGG repeat and a distal CGG repeat containing site. In one embodiment, the pre-mutation length CGG repeat comprises at least 50, 55, 60, 65, 70, 75, 80, 85, 90, 95 or more than 95 CGG repeats. In one embodiment, the pre-mutation length CGG repeat comprises In one embodiment, the pre-mutation length CGG repeat comprises less than 200 CGG repeats. In one embodiment, the recombinant nucleic acid sequence construct comprises 99 CGG repeats.

The recombinant nucleic acid sequence construct described above can be placed in one or more vectors. The one or more vectors can contain an origin of replication. The one or more vectors can be a plasmid, bacteriophage, bacterial artificial chromosome or yeast artificial chromosome. The one or more vectors can be either a self-replication extra chromosomal vector, or a vector which integrates into a host genome.

Vectors include, but are not limited to, plasmids, expression vectors, recombinant viruses, any form of recombinant “naked DNA” vector, and the like. A “vector” comprises a nucleic acid which can infect, transfect, transiently or permanently transduce a cell. It will be recognized that a vector can be a naked nucleic acid, or a nucleic acid complexed with protein or lipid. The vector optionally comprises viral or bacterial nucleic acids and/or proteins, and/or membranes (e.g., a cell membrane, a viral lipid envelope, etc.). Vectors include, but are not limited to replicons (e.g., RNA replicons, bacteriophages) to which fragments of DNA may be attached and become replicated. Vectors thus include, but are not limited to RNA, autonomous self-replicating circular or linear DNA or RNA (e.g., plasmids, viruses, and the like, see, e.g., U.S. Pat. No. 5,217,879), and include both the expression and non-expression plasmids. In some embodiments, the vector includes linear DNA, enzymatic DNA or synthetic DNA. Where a recombinant microorganism or cell culture is described as hosting an “expression vector” this includes both extra-chromosomal circular and linear DNA and DNA that has been incorporated into the host chromosome(s). Where a vector is being maintained by a host cell, the vector may either be stably replicated by the cells during mitosis as an autonomous structure, or is incorporated within the host's genome.

The one or more vectors can be a plasmid. The plasmid may be useful for transfecting cells with the recombinant nucleic acid sequence construct. The plasmid may be useful for introducing the recombinant nucleic acid sequence construct into the subject. The plasmid may also comprise a regulatory sequence, which may be well suited for gene expression in a cell into which the plasmid is administered.

The plasmid may also comprise a mammalian origin of replication in order to maintain the plasmid extra-chromosomally and produce multiple copies of the plasmid in a cell.

In one embodiment, the plasmid expresses an RNA molecule comprising a pre-mutation length CGG repeat.

Cas13 Degradation of CGG Containing RNA

In certain example embodiments, the invention incudes compositions and methods for degrading mRNA of one or more CGG tandem repeat containing gene. In one embodiment, a CRISPR/Cas13 system can be used to degrade mRNA of one or more CGG tandem repeat containing gene. In some embodiments, the invention includes a CRISPR/Cas13 system comprising an sgRNA specific for mRNA for one or more of FMR1, SHISA6, IRX2, TCERG1L, PTPRT, DPP6, and TMEM257. In some embodiments, the invention includes a CRISPR/Cas13 system comprising an sgRNA specific for Fmr1 mRNA.

### Methods of Use

The invention provides methods of use of the compositions of the invention to modulate one or more epigenomic marker. In some embodiments, the methods of the invention reduce the level of epigenomic methylation of at least one H3K9me3-heterochromatin mark containing gene or H3K9me3-heterochromatin mark containing gene regulator. In one embodiment, the methods of the invention include activating, reactivating or de-repressing a H3K9me3-heterochromatin mark containing gene. In one embodiment, the methods of the invention include blocking RNA mediated heterochromatin formation. In one embodiment, the methods of the invention inhibit RNA-DNA interactions which may induce heterochromatin.

Methods of Diagnosing Fragile X Syndrome

The invention is based, in part, on the identification of multiple regions of heterochromatin in samples with a full mutation in the Fmr1 gene, comprising greater than 200 CGG tandem repeats. In one embodiment, the invention provides methods of detecting decreased levels of one or more H3K9me3-heterochromatin mark containing gene for the diagnosis of fragile X syndrome, or a disease or disorder associated with fragile X syndrome. In some embodiments, the invention includes detecting an increase in H3-K9 methylation in a sample from a subject. In some embodiments, the invention includes detecting an increase in the level of heterochromatin in a sample from a subject. In some embodiments, the invention includes detecting a decrease in the level of protein, or mRNA for one or more H3K9me3-heterochromatin mark containing gene products. In some embodiments, the invention includes detecting a decrease in the level of protein, or mRNA for one or more of FMR1, FMR1NB, FMR1-AS1, C5orf38, CTD-2194D22.4, LOC100506858, IRX2, LOC105374620, LINC01377, LINC01019, LINC01017, IRX1, LINC02114, DPP6, LINC01287, LOC101929998, CSMD1, FAM135B, LOC101927815, COL22A1, KCNK9, TRAPPC9, MYOM2, LOC101927845, LINC01591, LOC101927915, SPANXN4, SPANXN3, SLITRK4, SPANXN2, UBE2NL, SPANXN1, SLITRK2, TMEM257, MIR892C, MIR890, MIR888, MIR892A, MIR892B, MIR891B, MIR891A, CXorf51B, CXorf51A, MIR513C, MIR513B, MIR513A1, MIR513A2, MIR506, MIR507, MIR508, MIR514B, MIR509-2, MIR509-3, MIR509-1, MIR510, MIR514A1, MIR514A2, MIR514A3, TCERG1L, MIR378C, TCERG1L-AS1, LINC01164, TMEM132C, TMEM132D, LOC100996671, LOC101927592, LINC00508, LOC100996679, GLT1D1, RIMBP2, LINC00939, LOC101927464, LOC100128554, LINC00944, LINC00943, LOC440117, LOC101927616, LOC101927637, LOC105370068, FLJ37505, LINC00507, CRAT8, LOC101927694, MIR4419B, MIR3612, SLC15A4, LOC283352, LOC101927735, LOC100190940, FZD10-AS1, FZD10, PIWIL1, RBFOX1, MIR8065, TMEM114, DNAH9, SHISA6, PTPRT, LOC101927159, LINC01441, LINC01440, CBLN4, and MC3R. In some embodiments, a decreased level of protein, or mRNA for one or more H3K9me3-heterochromatin mark containing gene products is detected in a sample of a subject. In one embodiment, the sample is of a subject at risk for development of fragile X syndrome or a disease or disorder associated with fragile X syndrome. In one embodiment, the sample is of a subject previously identified as having a CGG pre-mutation in Fmr1.

In some embodiments, the sample is a biological sample, including but not limited to a blood sample, a serum sample, a saliva sample, and a tissue sample.

### Determining Effectiveness of Therapy or Prognosis

In one aspect, an increased level of heterochomatin, or a decreased level of protein, or mRNA for one or more H3K9me3-heterochromatin mark containing gene products in a biological sample of a subject is used to monitor the effectiveness of treatment or the prognosis of disease. In some embodiments, an increased level of heterochomatin, or a decreased level of protein, or mRNA for one or more H3K9me3-heterochromatin mark containing gene products in a test sample obtained from a treated subject can be compared to the level from a reference sample obtained from that patient prior to initiation of a treatment. Clinical monitoring of treatment typically entails that each subject serve as her own baseline control. In some embodiments, test samples are obtained at multiple time points following administration of the treatment. In these embodiments, measurement of the level of heterochomatin, or the level of protein, or mRNA for one or more H3K9me3-heterochromatin mark containing gene products in the test samples provides an indication of the extent and duration of in vivo effect of the treatment.

Measurement of biomarker levels allow for the course of treatment of a disease to be monitored. The effectiveness of a treatment regimen for a disease can be monitored by detecting one or more biomarkers in an effective amount from samples obtained from a subject over time and comparing the amount of biomarkers detected. For example, a first sample can be obtained prior to the subject receiving treatment and one or more subsequent samples are taken after or during treatment of the subject. Changes in biomarker levels across the samples may provide an indication as to the effectiveness of the therapy.

In one embodiment, the invention provides a method for monitoring the levels of heterochomatin, or level of protein, or mRNA for one or more H3K9me3-heterochromatin mark containing gene products in response to treatment. For example, in some embodiments, the invention provides for a method of determining the efficacy of treatment in a subject, by measuring the levels of heterochomatin, or level of protein, or mRNA for one or more H3K9me3-heterochromatin mark containing gene products. In one embodiment, the level of levels of heterochomatin, or level of protein, or mRNA for one or more H3K9me3-heterochromatin mark containing gene products can be measured over time, where the level at one timepoint after the initiation of treatment is compared to the level at another timepoint after the initiation of treatment. In one embodiment, the level of levels of heterochomatin, or level of protein, or mRNA for one or more H3K9me3-heterochromatin mark containing gene products can be measured over time, where the level at one timepoint after the initiation of treatment is compared to the level prior to the initiation of treatment.

In one embodiment, the invention provides a method for monitoring the level of heterochomatin, or level of protein, or mRNA for one or more H3K9me3-heterochromatin mark containing gene products after treatment. In one embodiment, the invention provides a method for assessing the efficacy of treatment for Fragile X Syndrome (FXS) or other severe clinical presentations of FXS including, but not limited to, reproductive, epithelial, neural adhesion, and synaptic plasticity defects.

For example, in one embodiment, the method indicates that the treatment is effective when the level of level of heterochomatin is decreased, or the level of protein, or mRNA for one or more H3K9me3-heterochromatin mark containing gene products is increased in a sample of a treated subject as compared to a control diseased subject or population not receiving treatment. In one embodiment, the method indicates that the treatment is effective when the level of heterochomatin is decreased, or the level of protein, or mRNA for one or more H3K9me3-heterochromatin mark containing gene products is increased in a sample of a treated subject as compared to a control sample from the subject prior to treatment. In one embodiment, the method indicates that the treatment is effective when the level of level of heterochomatin is decreased, or the level of protein, or mRNA for one or more H3K9me3-heterochromatin mark containing gene products is increased in a sample of a treated subject as compared to a sample from the subject obtained at an earlier time point during treatment.

To identify therapeutics or drugs that are appropriate for a specific subject, a test sample from the subject can also be exposed to a therapeutic agent or a drug, and the level of one or more biomarkers can be determined. Biomarker levels can be compared to a sample derived from the subject before and after treatment or exposure to a therapeutic agent or a drug, or can be compared to samples derived from one or more subjects who have shown improvements relative to a disease as a result of such treatment or exposure. Thus, in one aspect, the invention provides a method of assessing the efficacy of a therapy with respect to a subject comprising taking a first measurement of a biomarker panel in a first sample from the subject; effecting the therapy with respect to the subject; taking a second measurement of the biomarker panel in a second sample from the subject and comparing the first and second measurements to assess the efficacy of the therapy. In one embodiment, the biomarker panel measures the level of protein or mRNA for one or more H3K9me3-heterochromatin mark containing gene product. In one embodiment, the biomarker panel comprises measures the level of protein, or mRNA for one or more of FMR1, FMR1NB, FMR1-AS1, C5orf38, CTD-2194D22.4, LOC100506858, IRX2, LOC105374620, LINC01377, LINC01019, LINC01017, IRX1, LINC02114, DPP6, LINC01287, LOC101929998, CSMD1, FAM135B, LOC101927815, COL22A1, KCNK9, TRAPPC9, MYOM2, LOC101927845, LINC01591, LOC101927915, SPANXN4, SPANXN3, SLITRK4, SPANXN2, UBE2NL, SPANXN1, SLITRK2, TMEM257, MIR892C, MIR890, MIR888, MIR892A, MIR892B, MIR891B, MIR891A, CXorf51B, CXorf51A, MIR513C, MIR513B, MIR513A1, MIR513A2, MIR506, MIR507, MIR508, MIR514B, MIR509-2, MIR509-3, MIR509-1, MIR510, MIR514A1, MIR514A2, MIR514A3, TCERG1L, MIR378C, TCERG1L-AS1, LINC01164, TMEM132C, TMEM132D, LOC100996671, LOC101927592, LINC00508, LOC100996679, GLT1D1, RIMBP2, LINC00939, LOC101927464, LOC100128554, LINC00944, LINC00943, LOC440117, LOC101927616, LOC101927637, LOC105370068, FLJ37505, LINC00507, CRAT8, LOC101927694, MIR4419B, MIR3612, SLC15A4, LOC283352, LOC101927735, LOC100190940, FZD10-AS1, FZD10, PIWIL1, RBFOX1, MIR8065, TMEM114, DNAH9, SHISA6, PTPRT, LOC101927159, LINC01441, LINC01440, CBLN4, and MC3R.

Competitive Inhibition

In one embodiment, the invention relates to method of competitively inhibiting the interaction of a chromosome region comprising a full-length CGG repeat with one or more distal or trans chromosome region containing a CGG repeat. In one embodiment, the method comprises administering a CGG binding molecule to bind to the full-length CGG repeat and competitively inhibit the interaction of the chromosome region comprising the full-length CGG repeat with one or more distal or trans chromosome region containing a CGG repeat. In one embodiment, the competitive inhibitor is administered to a subject having at least 200 CGG repeats in the FMR1 gene. In one embodiment, the competitive inhibitor prevents heterochromatin formation, gene silencing, or a combination thereof at one or more of C5orf38, CTD-2194D22.4, LOC100506858, IRX2, LOC105374620, LINC01377, LINC01019, LINC01017, IRX1, LINC02114, DPP6, LINC01287, LOC101929998, CSMD1, FAM135B, LOC101927815, COL22A1, KCNK9, TRAPPC9, MYOM2, LOC101927845, LINC01591, LOC101927915, SPANXN4, SPANXN3, SLITRK4, SPANXN2, UBE2NL, SPANXN1, SLITRK2, TMEM257, MIR892C, MIR890, MIR888, MIR892A, MIR892B, MIR891B, MIR891A, CXorf51B, CXorf51A, MIR513C, MIR513B, MIR513A1, MIR513A2, MIR506, MIR507, MIR508, MIR514B, MIR509-2, MIR509-3, MIR509-1, MIR510, MIR514A1, MIR514A2, MIR514A3, TCERG1L, MIR378C, TCERG1L-AS1, LINC01164, TMEM132C, TMEM132D, LOC100996671, LOC101927592, LINC00508, LOC100996679, GLT1D1, RIMBP2, LINC00939, LOC101927464, LOC100128554, LINC00944, LINC00943, LOC440117, LOC101927616, LOC101927637, LOC105370068, FLJ37505, LINC00507, CRAT8, LOC101927694, MIR4419B, MIR3612, SLC15A4, LOC283352, LOC101927735, LOC100190940, FZD10-AS1, FZD10, PIWIL1, RBFOX1, MIR8065, TMEM114, DNAH9, SHISA6, PTPRT, LOC101927159, LINC01441, LINC01440, CBLN4, and MC3R.

Exemplary competitive inhibitors include, but are not limited to, a small molecule, an antisense oligonucleotide directed to CGG repeats, or a recombinant nucleic acid molecule comprising a pre-mutation length CGG repeat. Exemplary small molecule inhibitors include, but are not limited to, compound 1a, compound if and ETP69.

Therapeutic Compositions

In one embodiment, the invention relates to therapeutic composition comprising a composition of the invention to modulate one or more epigenomic marker. Such a molecule (e.g., epigenomic editor, ASO, etc.) and the encoding nucleic acid sequence may then serve as therapeutic agent for modulating one or more epigenomic marker in a subject in need thereof. In one embodiment, the therapeutic agent activates or reactivates one or more H3K9me3-heterochromatin mark containing gene. In one embodiment, the therapeutic agent reduces the level of epigenomic methylation of at least one H3K9me3-heterochromatin mark containing gene or H3K9me3-heterochromatin mark containing gene regulator. In one embodiment, the therapeutic agent blocks RNA mediated heterochromatin formation. In one embodiment, the therapeutic agent inhibits RNA-DNA interactions.

In one embodiment, the invention relates to vaccine compositions comprising a noncoding RNA molecule comprising a pre-mutation length CGG repeat. In one embodiment, the vaccine induces or restores expression of one or more silenced H3K9me3-heterochromatin mark containing gene.

In one embodiment, the invention relates to methods of treatment or prevention of a disease or disorder associated with genomic instability. In one embodiment, the invention relates to methods of treatment or prevention of fragile X syndrome or a disease or disorder associated with triplet repeat expansion or genome instability. Pathologies relating to triplet repeat expansion, include, but are not limited to, parkinsonism, ataxia, dementia, autonomic dysfunctions, myopathy, ubiquitin-positive inclusion bodies, middle cerebellar peduncle hyperintensity, leukoencephalopathy, myotonic dystrophy (DM), Huntington disease, spinocerebellar ataxia, Friedreich ataxia, and fragile X syndrome. In one embodiment, the pathology relating to genomic instability is fragile X syndrome, fragile X-associated primary ovarian insufficiency (FXPOI), fragile X-associated tremor/ataxia syndrome (FXTAS), syndromic and non-syndromic forms of intellectual disability (ID), autism, developmental delay, Jacobsen syndrome, and Baratela-Scott syndrome. In some embodiments, the genome instability associated disease or disorder is cancer or a disease or disorder associated therewith.

Administration of the therapeutic agent in accordance with the present invention may be continuous or intermittent, depending, for example, upon the recipient's physiological condition, whether the purpose of the administration is therapeutic or prophylactic, and other factors known to skilled practitioners. The administration of the agents of the invention may be essentially continuous over a preselected period of time or may be in a series of spaced doses. Both local and systemic administration is contemplated. The amount administered will vary depending on various factors including, but not limited to, the composition chosen, the particular disease, the weight, the physical condition, and the age of the subject, and whether prevention or treatment is to be achieved. Such factors can be readily determined by the clinician employing animal models or other test systems which are well known to the art.

### Excipients and Other Components of the Vaccine

The vaccine may further comprise a pharmaceutically acceptable excipient. The pharmaceutically acceptable excipient can be functional molecules such as vehicles, carriers, or diluents. The pharmaceutically acceptable excipient can include, but is not limited to, LPS analogs including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known vehicles, carriers, or diluents.

The pharmaceutically acceptable excipient can be an adjuvant. The adjuvant can be other genes that are expressed from a plasmid or are delivered as proteins in combination with the RNA vaccine. The adjuvant may be selected from the group consisting of: α-interferon (IFN-α), β-interferon (IFN-β), γ-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MHIC, CD80, CD86 including IL-15 having the signal sequence deleted and optionally including the signal peptide from IgE. The adjuvant can be IL-12, IL-15, IL-28, CTACK, TECK, platelet derived growth factor (PDGF), TNFα, TNF□, GM-CSF, epidermal growth factor (EGF), IL-1, IL-2, IL-4, IL-5, IL-6, IL-10, IL-12, IL-18, or a combination thereof.

Other genes that can be useful as adjuvants include those encoding: MCP-1, MIP-1a, MIP-1p, IL-8, RANTES, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, IL-22, nerve growth factor, vascular endothelial growth factor, Fas, TNF receptor, Flt, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, INK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof.

The vaccine can be formulated according to the mode of administration to be used. An injectable vaccine pharmaceutical composition can be sterile, pyrogen free and particulate free. An isotonic formulation or solution can be used. Additives for isotonicity can include sodium chloride, dextrose, mannitol, sorbitol, and lactose. The vaccine can comprise a vasoconstriction agent. The isotonic solutions can include phosphate buffered saline. Vaccines of the invention can further comprise stabilizers including gelatin and albumin. The stabilizers can allow the formulation to be stable at room or ambient temperature for extended periods of time, including LGS or polycations or polyanions.

### Method of Vaccination

Also provided herein is a method of treating, protecting against, and/or preventing disease in a subject in need thereof by administering the vaccine to the subject. Administration of the vaccine to the subject can induce or restore expression of one or more silenced gene in the subject. The induced or restored expression of one or more silenced gene can be used to treat, prevent, and/or protect against disease, for example, pathologies relating to genomic instability. The induced or restored expression of one or more silenced gene can be used to treat, prevent, and/or protect against disease, for example, pathologies relating to triplet repeat expansion, including, but not limited to, parkinsonism, ataxia, dementia, autonomic dysfunctions, myopathy, ubiquitin-positive inclusion bodies, middle cerebellar peduncle hyperintensity, leukoencephalopathy, myotonic dystrophy (DM), Huntington disease, spinocerebellar ataxia, Friedreich ataxia, and fragile X syndrome. In one embodiment, the pathology relating to genomic instability is fragile X syndrome, fragile X-associated primary ovarian insufficiency (FXPOI), fragile X-associated tremor/ataxia syndrome (FXTAS), syndromic and non-syndromic forms of intellectual disability (ID), autism, developmental delay, Jacobsen syndrome, and Baratela-Scott syndrome.

In some embodiments, the genome instability associated disease or disorder is cancer or a disease or disorder associated therewith. Cancers that can be treated using the compositions and methods of the invention include, but are not limited to, acute lymphoblastic leukemia, acute myeloid leukemia, adrenocortical carcinoma, appendix cancer, basal cell carcinoma, bile duct cancer, bladder cancer, bone cancer, brain and spinal cord tumors, brain stem glioma, brain tumor, breast cancer, bronchial tumors, burkitt lymphoma, carcinoid tumor, central nervous system atypical teratoid/rhabdoid tumor, central nervous system embryonal tumors, central nervous system lymphoma, cerebellar astrocytoma, cerebral astrocytoma/malignant glioma, cerebral astrocytotna/malignant glioma, cervical cancer, childhood visual pathway tumor, chordoma, chronic lymphocytic leukemia, chronic myelogenous leukemia, chronic myeloproliferative disorders, colon cancer, colorectal cancer, craniopharyngioma, cutaneous cancer, cutaneous t-cell lymphoma, endometrial cancer, ependymoblastoma, ependymoma, esophageal cancer, ewing family of tumors, extracranial cancer, extragonadal germ cell tumor, extrahepatic bile duct cancer, extrahepatic cancer, eye cancer, fungoides, gallbladder cancer, gastric (stomach) cancer, gastrointestinal cancer, gastrointestinal carcinoid tumor, gastrointestinal stromal tumor (gist), germ cell tumor, gestational cancer, gestational trophoblastic tumor, glioblastoma, glioma, hairy cell leukemia, head and neck cancer, hepatocellular (liver) cancer, histiocytosis, hodgkin lymphoma, hypopharyngeal cancer, hypothalamic and visual pathway glioma, hypothalamic tumor, intraocular (eye) cancer, intraocular melanoma, islet cell tumors, kaposi sarcoma, kidney (renal cell) cancer, langerhans cell cancer, langerhans cell histiocytosis, laryngeal cancer, leukemia, lip and oral cavity cancer, liver cancer, lung cancer, lymphoma, macroglobulinemia, malignant fibrous histiocytoma of bone and osteosarcoma, medulloblastoma, medulloepithelioma, melanoma, merkel cell carcinoma, mesothelioma, metastatic squamous neck cancer with occult primary, mouth cancer, multiple endocrine neoplasia syndrome, multiple myeloma, mycosis, myelodysplastic syndromes, myelodysplastic/myeloproliferative diseases, myelogenous leukemia, myeloid leukemia, myeloma, myeloproliferative disorders, nasal cavity and paranasal sinus cancer, nasopharyngeal cancer, neuroblastoma, non-hodgkin lymphoma, non-small cell lung cancer, oral cancer, oral cavity cancer, oropharyngeal cancer, osteosarcoma and malignant fibrous histiocytoma, osteosarcoma and malignant fibrous histiocytoma of bone, ovarian, ovarian cancer, ovarian epithelial cancer, ovarian germ cell tumor, ovarian low malignant potential tumor, pancreatic cancer, papillomatosis, paraganglioma, parathyroid cancer, penile cancer, pharyngeal cancer, pheochromocytoma, pineal parenchymal tumors of intermediate differentiation, pineoblastoma and supratentorial primitive neuroectodermal tumors, pituitary tumor, plasma cell neoplasm, plasma cell neoplasm/multiple myeloma, pleuropulmonary blastoma, primary central nervous system cancer, primary central nervous system lymphoma, prostate cancer, rectal cancer, renal cell (kidney) cancer, renal pelvis and ureter cancer, respiratory tract carcinoma involving the nut gene on chromosome 15, retinoblastoma, rhabdomyosarcoma, salivary gland cancer, sarcoma, sezary syndrome, skin cancer (melanoma), skin cancer (nonmelanoma), skin carcinoma, small cell lung cancer, small intestine cancer, soft tissue cancer, soft tissue sarcoma, squamous cell carcinoma, squamous neck cancer, stomach (gastric) cancer, supratentorial primitive neuroectodermal tumors, supratentorial primitive neuroectodermal tumors and pineoblastoma, T-cell lymphoma, testicular cancer, throat cancer, thymoma and thymic carcinoma, thyroid cancer, transitional cell cancer, transitional cell cancer of the renal pelvis and ureter, trophoblastic tumor, urethral cancer, uterine cancer, uterine sarcoma, vaginal cancer, visual pathway and hypothalamic glioma, vulvar cancer, waldenstrom macroglobulinemia, and wilms tumor.

The vaccine dose can be between 1 μg to 10 mg active component/kg body weight/time, and can be 20 μg to 10 mg component/kg body weight/time. The vaccine can be administered every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. The number of vaccine doses for effective treatment can be 1, 2, 3, 4, 5, 6, 7, 8, 9, or 10.

### Administration

The vaccine can be formulated in accordance with standard techniques well known to those skilled in the pharmaceutical art. Such compositions can be administered in dosages and by techniques well known to those skilled in the medical arts taking into consideration such factors as the age, sex, weight, and condition of the particular subject, and the route of administration. The subject can be a mammal, such as a human, a horse, a cow, a pig, a sheep, a cat, a dog, a rat, or a mouse.

The vaccine can be administered prophylactically or therapeutically. In prophylactic administration, the vaccines can be administered in an amount sufficient to induce or restore expression of one or more silenced gene. In therapeutic applications, the vaccines are administered to a subject in need thereof in an amount sufficient to elicit a therapeutic effect. An amount adequate to accomplish this is defined as “therapeutically effective dose.” Amounts effective for this use will depend on, e.g., the particular composition of the vaccine regimen administered, the manner of administration, the stage and severity of the disease, the general state of health of the patient, and the judgment of the prescribing physician.

The vaccine can be administered by methods well known in the art as described in Donnelly et al. (Ann. Rev. Immunol. 15:617-648 (1997)); Felgner et al. (U.S. Pat. No. 5,580,859, issued Dec. 3, 1996); Felgner (U.S. Pat. No. 5,703,055, issued Dec. 30, 1997); and Carson et al. (U.S. Pat. No. 5,679,647, issued Oct. 21, 1997), the contents of all of which are incorporated herein by reference in their entirety. The RNA of the vaccine can be complexed to or encapsulated within particles or beads that can be administered to an individual. One skilled in the art would know that the choice of a pharmaceutically acceptable carrier, including a physiologically acceptable compound, depends, for example, on the route of administration of the expression vector.

The vaccine can be delivered via a variety of routes. Typical delivery routes include parenteral administration, e.g., intradermal, intramuscular or subcutaneous delivery. Other routes include oral administration, intranasal, and intravaginal routes. The vaccine can also be administered to muscle, or can be administered via intradermal or subcutaneous injections, or transdermally, such as by iontophoresis. Epidermal administration of the vaccine can also be employed. Epidermal administration can involve mechanically or chemically irritating the outermost layer of epidermis to stimulate an immune response to the irritant (Carson et al., U.S. Pat. No. 5,679,647, the contents of which are incorporated herein by reference in its entirety).

The vaccine can also be formulated for administration via the nasal passages. Formulations suitable for nasal administration, wherein the carrier is a solid, can include a coarse powder having a particle size, for example, in the range of about 10 to about 500 microns which is administered in the manner in which snuff is taken, i.e., by rapid inhalation through the nasal passage from a container of the powder held close up to the nose. The formulation can be a nasal spray, nasal drops, or by aerosol administration by nebulizer. The formulation can include aqueous or oily solutions of the vaccine.

The vaccine can be a liquid preparation such as a suspension, syrup or elixir. The vaccine can also be a preparation for parenteral, subcutaneous, intradermal, intramuscular or intravenous administration (e.g., injectable administration), such as a sterile suspension or emulsion.

The vaccine can be incorporated into liposomes, microspheres or other polymer matrices (Felgner et al., U.S. Pat. No. 5,703,055; Gregoriadis, Liposome Technology, Vols. Ito III (2nd ed. 1993), the contents of which are incorporated herein by reference in their entirety). Liposomes can consist of phospholipids or other lipids, and can be nontoxic, physiologically acceptable and metabolizable carriers that are relatively simple to make and administer. In some embodiments, the RNA vaccine is formulated for administration using a lipid nanoparticle formulation (LNP).

The RNA vaccines contemplated herein-which may include various formats, such as, but not limited to, macromolecule complexes, nanocapsules, microspheres, beads, and lipid-based systems including oil-in-water emulsions, micelles, mixed micelles, liposomes, and lipid nanoparticles (LNPs)—may further comprise one or more targeting moieties (or equivalently “targeting domains” or “targeting ligands”) which function to target the RNA molecule to a locus of interest. In one embodiment, the noncoding RNA molecule of the invention comprises a RNA nuclear localization signal to target the RNA molecule of the invention to the nucleus of a cell.

Nanoparticles

In some embodiments, the present disclosure provides a nucleic acid vaccine comprising a noncoding RNA molecule comprising a CGG repeat tract formulated in a nanoparticle (e.g., a lipid nanoparticle). Lipid nanoparticle formulations typically comprise at least one lipid, a sterol and a molecule capable of reducing particle aggregation, for example a PEG or PEG-modified lipid.

Non-limiting examples of lipid nanoparticle compositions and methods of making them are described, for example, in Semple et al. (2010) Nat. Biotechnol. 28:172-176; Jayarama et al. (2012), Angew. Chem. Int. Ed., S1: 8529-8533; and Maier et al. (2013) Molecular Therapy 21, 1570-1578 (the contents of each of which are incorporated herein by reference in their entirety).

In some embodiments, the noncoding RNA molecule comprising a CGG repeat tract vaccines is formulated in a lipid-polycation complex, referred to as a cationic lipid nanoparticle. As a non-limiting example, the polycation may include a cationic peptide or a polypeptide such as, but not limited to, polylysine, polyornithine and/or polyarginine. In some embodiments, a noncoding RNA molecule comprising a CGG repeat tract is formulated in a lipid nanoparticle that includes a non-cationic lipid such as, but not limited to, cholesterol or dioleoyl phosphatidyl-ethanolamine (DOPE). In some embodiments, the lipid nanoparticle comprises at least one ionizable cationic lipid, at least one non-cationic lipid, at least one sterol, and/or at least one polyethylene glycol (PEG)-modified lipid.

In some embodiments, lipid nanoparticle formulations may comprise 35 to 45% cationic lipid, 40% to 50% cationic lipid, 50% to 60% cationic lipid and/or 55% to 65% cationic lipid. In some embodiments, the ratio of lipid to noncoding RNA in the lipid nanoparticles may be 5:1 to 20:1, 10:1 to 25:1, 15:1 to 30:1 and/or at least 30:1.

In some embodiments, the ratio of PEG in the lipid nanoparticle formulations may be increased or decreased and/or the carbon chain length of the PEG lipid may be modified from C14 to C18 to alter the pharmacokinetics and/or biodistribution of the lipid nanoparticle formulations. As a non-limiting example, lipid nanoparticle formulations may contain 0.5% to 3.0%, 1.0% to 3.5%, 1.5% to 4.0%, 2.0% to 4.5%, 2.5% to 5.0% and/or 3.0% to 6.0% of the lipid molar ratio of PEG-c-DOMG (R-3-[(o-methoxy-poly(ethyleneglycol)2000)carbamoyl)]-1,2-dimyristyloxypropyl-3-amine) (also referred to herein as PEG-DOMG) as compared to the cationic lipid, DSPC and cholesterol. In some embodiments, the PEG-c-DOMG may be replaced with a PEG lipid such as, but not limited to, PEG-DSG (1,2-Distearoyl-sn-glycerol, methoxypolyethylene glycol), PEG-DMG (1,2-Dimyristoyl-sn-glycerol) and/or PEG-DPG (1,2-Dipalmitoyl-sn-glycerol, methoxypolyethylene glycol). The cationic lipid may be selected from any lipid known in the art such as, but not limited to, DLin-MC3-DMA, DLin-DMA, C12-200 and DLin-KC2-DMA.

In some embodiments, the noncoding RNA molecule comprising a CGG repeat tract vaccines formulation is a nanoparticle that comprises at least one lipid selected from, but not limited to, DLin-DMA, DLin-K-DMA, 98N12-5, C12-200, DLin-MC3-DMA, DLin-KC2-DMA, DODMA, PLGA, PEG, PEG-DMG, PEGylated lipids and amino alcohol lipids. In some embodiments, the lipid may be a cationic lipid such as, but not limited to, DLin-DMA, DLin-D-DMA, DLin-MC3-DMA, DLin-KC2-DMA, DODMA and amino alcohol lipids. The amino alcohol cationic lipid may be the lipids described in and/or made by the methods described in U.S. Patent Publication No. US20130150625, herein incorporated by reference in its entirety. As a non-limiting example, the cationic lipid may be 2-amino-3-[(9Z,12Z)-octadeca-9,12-dien-1-yloxy]-2-{[(9Z,2Z)-octadeca-9,12-dien-1-yloxy]methyl}propan-1-ol (Compound 1 in US20130150625); 2-amino-3-[(9Z)-octadec-9-en-1-yloxy]-2{[(9Z)-octadec-9-en-1-yloxy]methyl}propan-1-ol (Compound 2 in US20130150625); 2-amino-3-[(9Z,12Z)-octadeca-9,12-dien-1-yloxy]-2-[(octyloxy)methyl]propan-1-ol (Compound 3 in US20130150625); and 2-(dimethylamino)-3-[(9Z,12Z)-octadeca-9,12-dien-1-yloxy]-2-{[(9Z,12Z)-octadeca-9,12-dien-1-yloxy]methyl}propan-lol (Compound 4 in US20130150625); or any pharmaceutically acceptable salt or stereoisomer thereof.

In some embodiments, a nanoparticle (e.g., a lipid nanoparticle) has a mean diameter of 10-500 nm, 20-400 nm, 30-300 nm, 40-200 nm. In some embodiments, a nanoparticle (e.g., a lipid nanoparticle) has a mean diameter of 50-150 nm, 50-200 nm, 80-100 nm or 80-200 nm.

### Combinations

In one embodiment, the methods of the present invention include combinations of any of the inhibitors and activators described herein. In certain embodiments, a combination of two or more of the inhibitors and/or activators described herein has an additive effect, wherein the overall effect of the combination is approximately equal to the sum of the effects of each individual composition. In other embodiments a combination of two or more of the inhibitors and/or activators described herein has a synergistic effect, wherein the overall effect of the combination is greater than the sum of the effects of each individual inhibitor.

In some embodiments, the composition of the present invention comprises a combination of one or more of the inhibitors and activators described herein and a second therapeutic agent. For example, in one embodiment the second therapeutic agents include, but are not limited to, a therapeutic agent for the treatment of fragile X syndrome or a genome instability associated disease or disorder. In some embodiments, the genome instability associated disease or disorder is cancer or a disease or disorder associated therewith.

Kits

The present invention also pertains to kits useful in the methods of the invention. Such kits comprise various combinations of components useful in any of the methods described elsewhere herein. For example, in one embodiment, the kit comprises components useful for modulating one or more host protein-microbial cell interaction as described herein. In one embodiment, the kit contains additional components. In one embodiment, an additional component includes but is not limited to instructional material. In one embodiment, instructional material for use with a kit of the invention may be provided electronically.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: Long-Range Heterochromatin Silencing Via Spatial Proximity Among Distal Unstable Short Tandem Repeat Tracts in Fragile X Syndrome

Recently severe local misfolding of the 3D genome was reported around the FMR1 gene in B cells and post-mortem brain tissue from FXS patients with a 450+ CGG STR expansion²⁴, suggests that silencing might occur via long-range mechanisms beyond local DNA methylation. Here, the extent to which 3D chromatin architecture and linear epigenetic marks are altered genome-wide is investigated as a function of a gradient of CGG STR tract lengths.

### Results

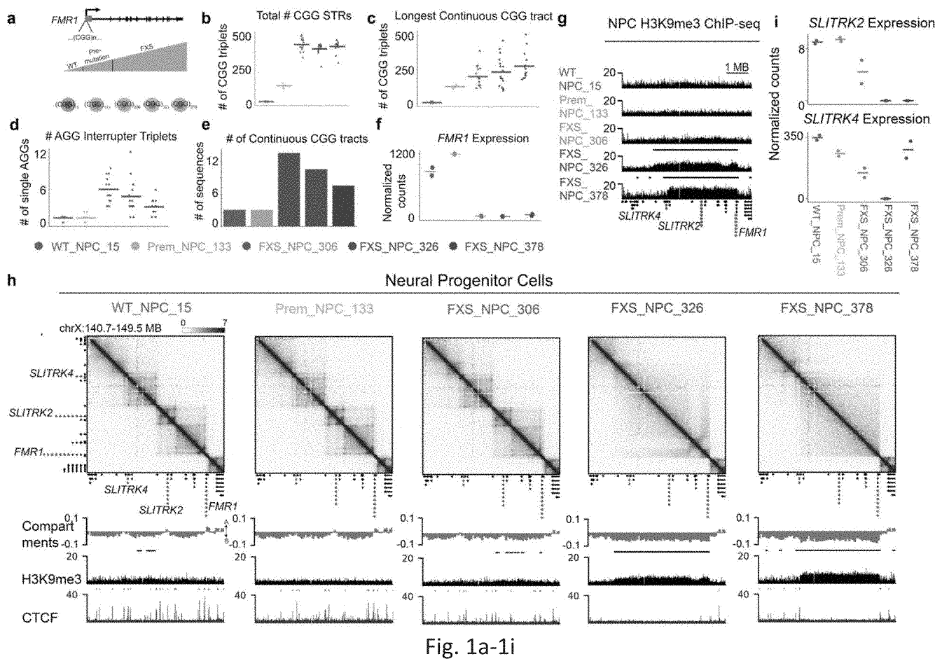

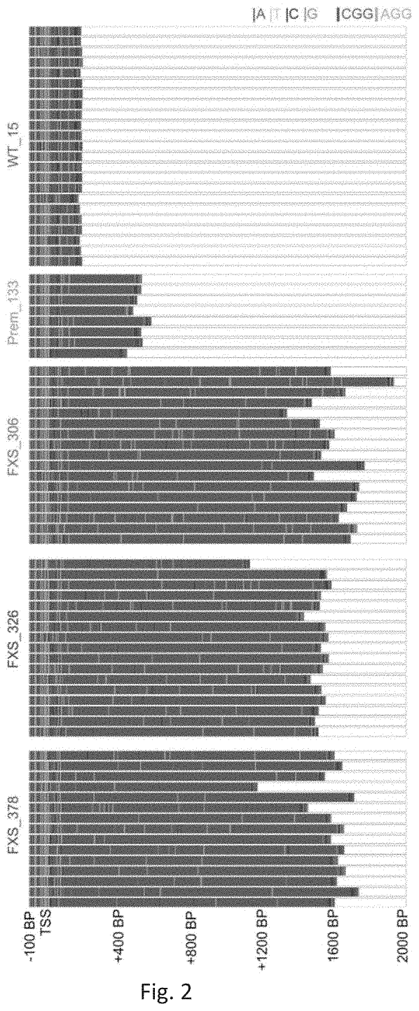

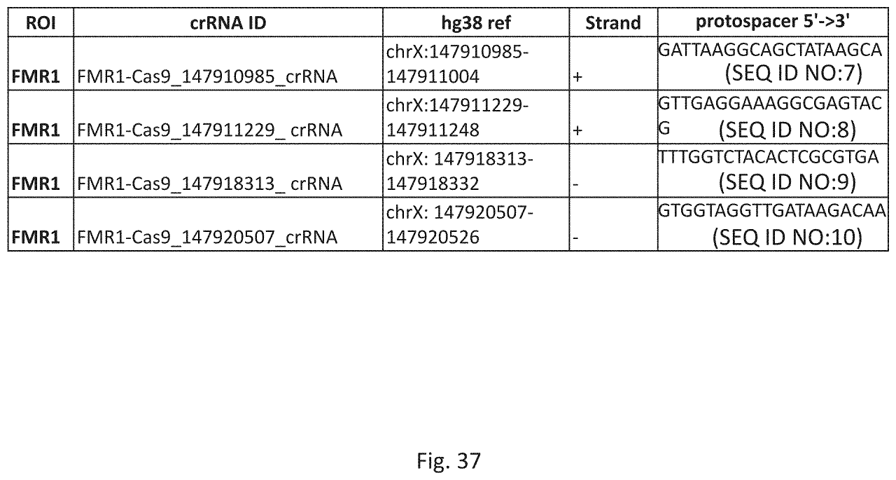

A series of human induced pluripotent stem cell lines differentiated to neural progenitor cells (iPS-NPCs) were examined in which the CGG STR tract is thought to expand from normal-length (5-30 CGG), pre-mutation (130-190 CGG), short mutation-length (200-300 CGG), and long mutation-length (450+ CGG Replicate 1; 450+ CGG Replicate 2) (FIG. 1a). To obtain precise estimates of CGG STR length, a customized assay was conducted coupling Nanopore long-read sequencing with guide RNA-directed Cas9 cutting around the transcription start site and 5′UTR of the FMR1 gene (FIG. 1b-e, FIG. 2, FIG. 37). Consistent with previous reports, wild type and pre-mutation lines had on average 34 and 160 total CGG STRs (FIG. 1b), respectively, with minimal interrupting sequences (FIG. 1c-e), and the expected increase in FMR1 mRNA upon expansion to the pre-mutation length (FIG. 1f). Unexpectedly, it was observed that both short and long mutation-length labeled F×S lines showed a similar total of 425-460 CGG triplets (FIG. 1b). However, the short mutation length line contained a high number of AGG interrupters, leading to shorter and more continuous CGG tracts compared to long mutation-length (green, FIG. 1c-e). To facilitate clarity, the three F×S lines are referred to by the sum of their top two longest continuous CGG tracts—306, 326, and 378 CGG triplets.

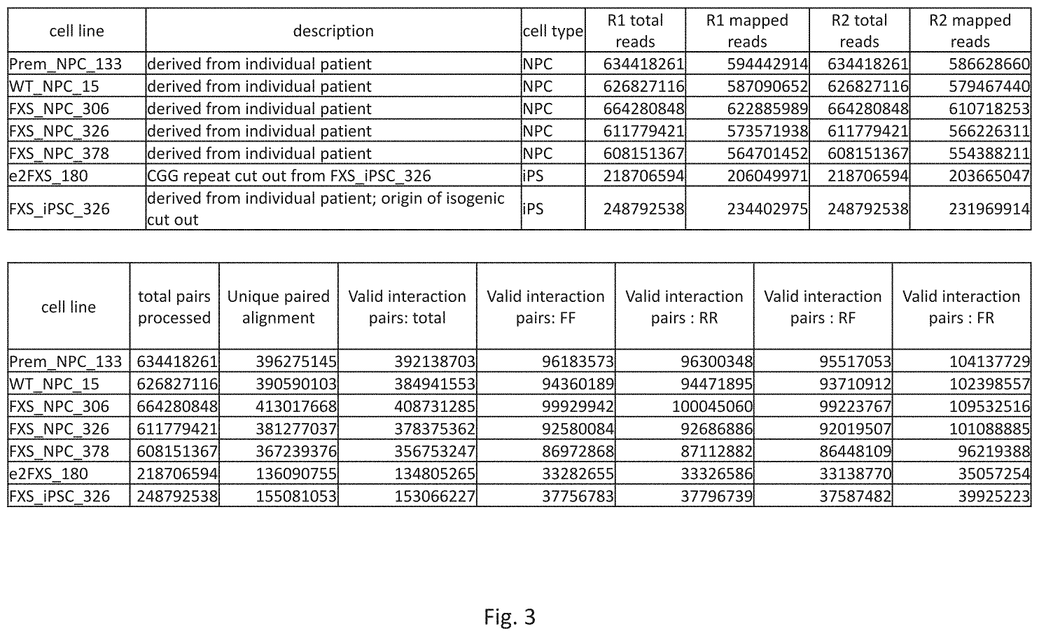

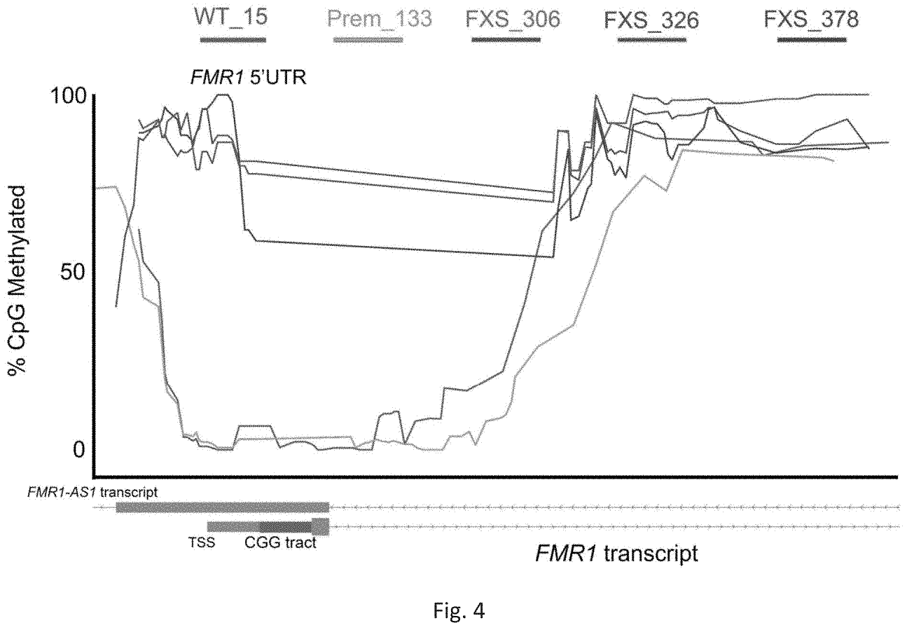

It is well established that AGG interrupters correlate with attenuated STR instability and decreased severity of disease (Eichler et al., 1994, Nat Genet 8, 88-94), therefore we hypothesized that the FXS_306 short mutation-length line with a high frequency of interrupters would have less severe pathological epigenetic defects than long mutation-length lines FXS_326 and FXS_378. It was observed that FMR1 gene expression decreased significantly to the same extent in all three F×S lines (FIG. 1f). Concomitant with decreased FMR1, increased DNA methylation was observed around the transcription start site and the 5′UTR-localized CGG STR in all three F×S lines, suggesting that local levels of DNA methylation correlate strongly with the mRNA levels of FMR1 (FIG. 4). In stark contrast to local DNA methylation, CGG-dependent acquisition of the repressive histone mark H3K9me3 (FIG. 1g) was also observed. The gained H3K9me3 signal was not only local to FMR1, but spread upstream over ˜3 Mb in FXS_306 and then increased in strength and/or spread further upstream to >5 Mb as the CGG tracts grew to 326 and 378 CGG triplets (FIG. 1g-h, FIG. 3). Thus, the spread and intensity of a large H3K9me3 repressive heterochromatin domain correlates with the length of the continuous CGG tract, whereas local DNA methylation of the FMR1 promoter silences the gene after the CGG passes short mutation-length.

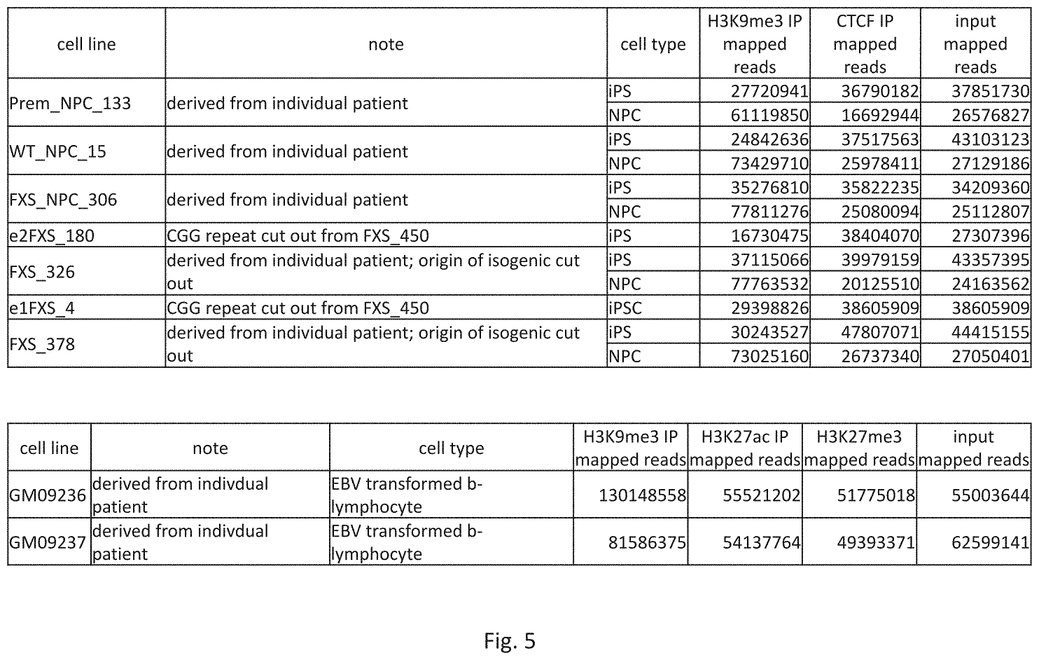

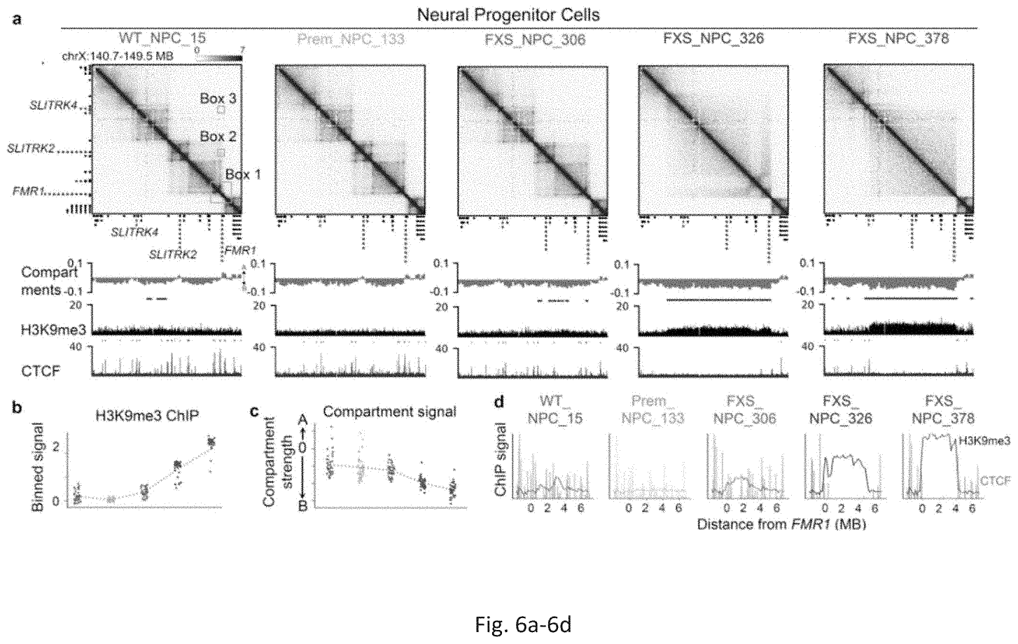

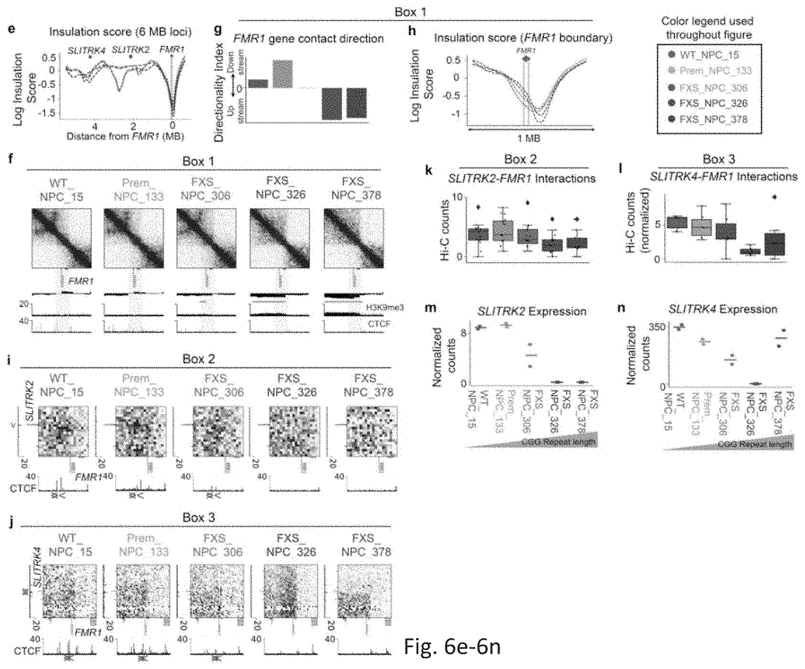

Next, the effect of H3K9me3 acquisition on the folding patterns of the 3D genome (FIG. 5) was studied. In parallel with gained H3K9me3 (FIG. 1h, FIG. 6a-b), we observed strengthening of B compartment signal (FIG. 1h, FIGS. 6a and 6c), loss of CTCF occupancy (FIG. 1h, FIG. 6d), and severe breakdown of TAD integrity (FIGS. 6a and 6e) across the broader 5 Mb-sized H3K9me3 domain. Destruction of the local subTAD boundary at FMR1 (FIG. 6f-h) was observed, as previously reported (Sun et al., 2018, Cell 175, 224-238 e215). These results demonstrate that heterochromatin silencing spreads more than 5 Mb upstream of FMR1 and is connected to severe large-scale misfolding of the 3D genome in FXS.

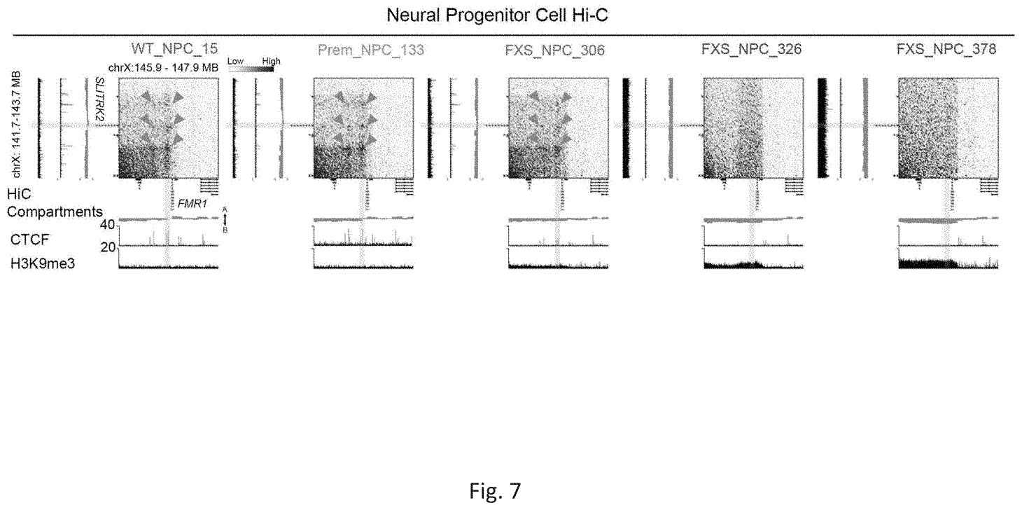

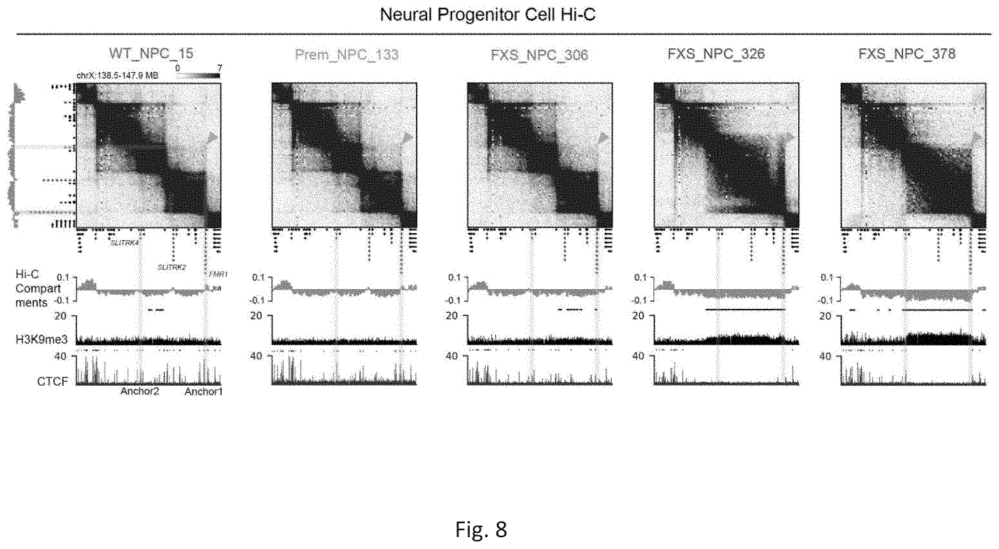

The FXS H3K9me3 domain spanned two additional genes, SLITRK2 and SLITRK4, encoding known neuronal cell adhesion proteins linked to synaptic plasticity (FIG. 1g-h). Expression of both SLITRK2 and SLITRK4 noticeably decrease in FXS in a manner that correlates with the spread of the H3K9me3 domain due to FMR1 CGG expansion (FIG. 1i). Using Hi-C maps, it was observed that FMR1 loops directly to SLITRK2 and SLITRK4 in wild type iPS-NPCs with a normal-length CGG STR tract (FIG. 6i-j). The long-range gene-gene cis interactions are abolished and SLITRK2 and SLITRK4 mRNA levels are decreased as H3K9me3 spreads over the locus (FIG. 6i-n, FIG. 7). It was observed that SLITRK2 SLITRK4 are downregulated but not fully off in the FXS_NPC_306 line, suggesting that FMR1 silencing is governed by local DNA methylation whereas distal gene silencing is governed by larger heterochromatin and 3D genome disruption (FIG. 6i-n). It was also noted that SLITRK4 is not silenced in one of the long mutation-length samples because the H3K9me3 domain does not extend up to the promoter of the gene, further emphasizing the likely functional role for H3K9me3 in distal gene silencing in FXS (FIG. 8). Together, these data suggest that the acquisition of a large 5 Mb sized H3K9me3 domain radiates outward from FMR1 to encompass and silence additional synaptic genes as a mutation-length CGG STR further expands. FXS is characterized by clinical presentation of cognitive decline and defects in synaptic plasticity (Telias, 2019, Front Mol Neurosci 12, 51), so the direct spatial connection between FMR1 and synaptic gene silencing is of critical importance toward understanding the onset of neural circuit pathology.

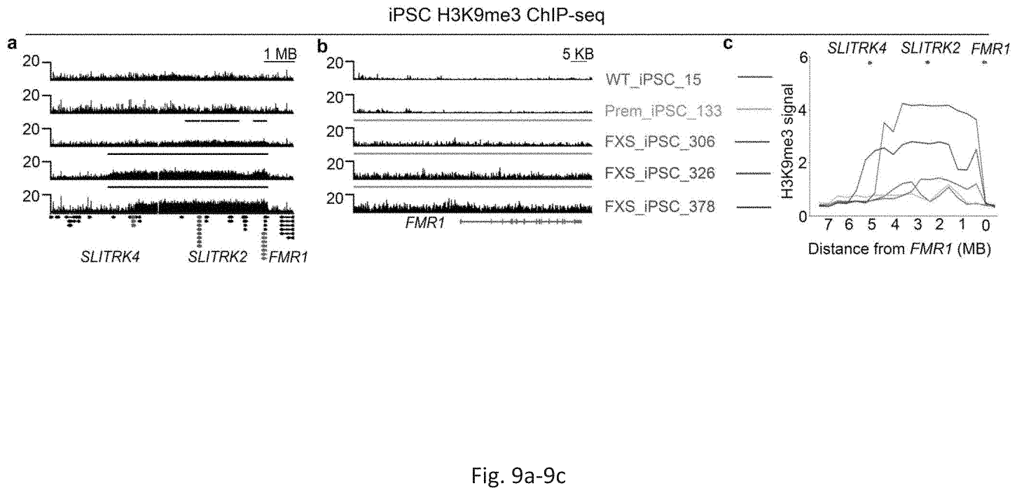

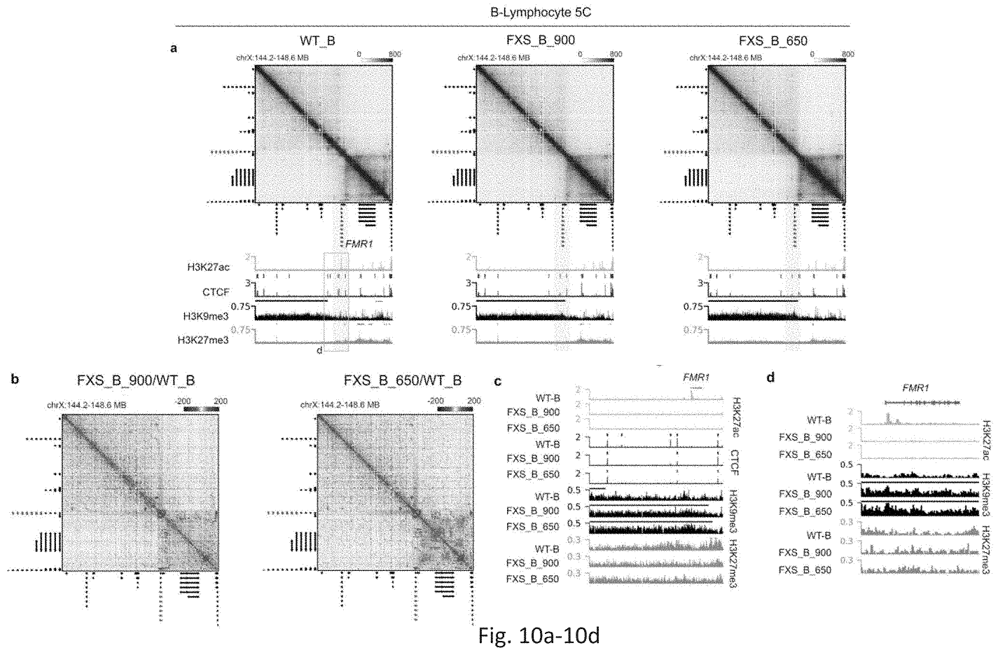

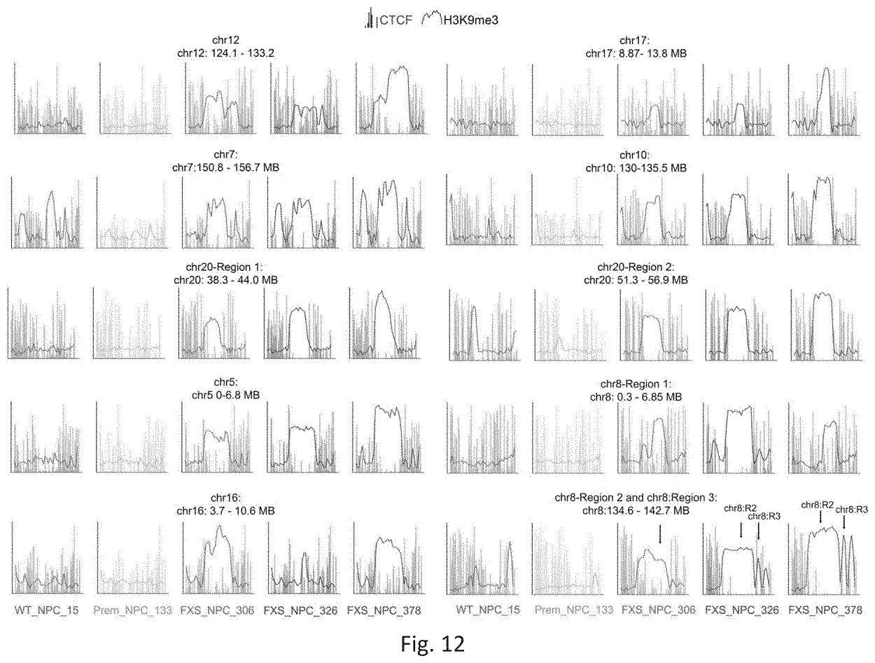

Next, whether the observations of large-scale 3D genome misfolding and heterochromatin silencing around the FMR1 locus were specific to the NPC state was explored. In pluripotent iPS cells, the same pattern of large-scale H3K9me3 deposition gained with CGG STR expansion was observed as in NPCs (FIG. 9). By contrast, in B cells, Hi-C analysis revealed that large scale genome folding disruptions did not occur upon mutation-length expansion (FIG. 10a-b). Importantly, the large H3K9me3 domain is pre-existing in wild type B cells with the normal-length CGG tract, but stops at the TAD boundary before FMR1 (FIGS. 10 and 12). In mutation-length FXS B cells, the pre-existing H3K9me3 domain spreads over FMR1, and local CTCF occupancy and TAD boundary integrity are disrupted as we have previously reported (FIG. 10c-d)²⁴. Thus, the genome folding, CTCF occupancy, and H3K9me3-based heterochromatin silencing defects are cell type-specific in FXS and most severe in cell types such as NPCs where no pre-existing H3K9me3 domain is present at the larger FMR1 locus.

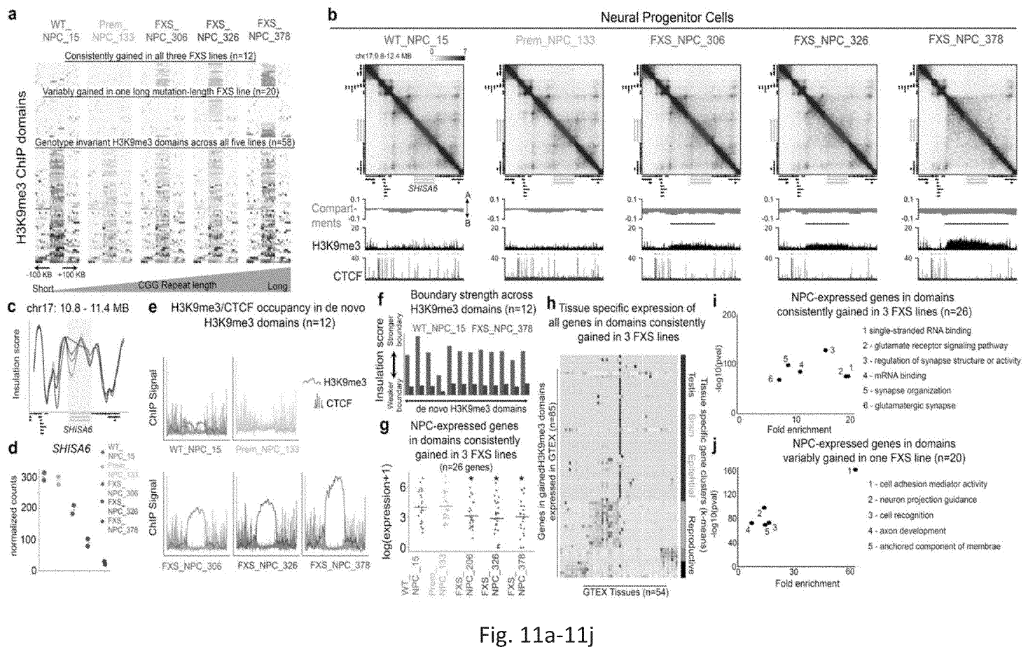

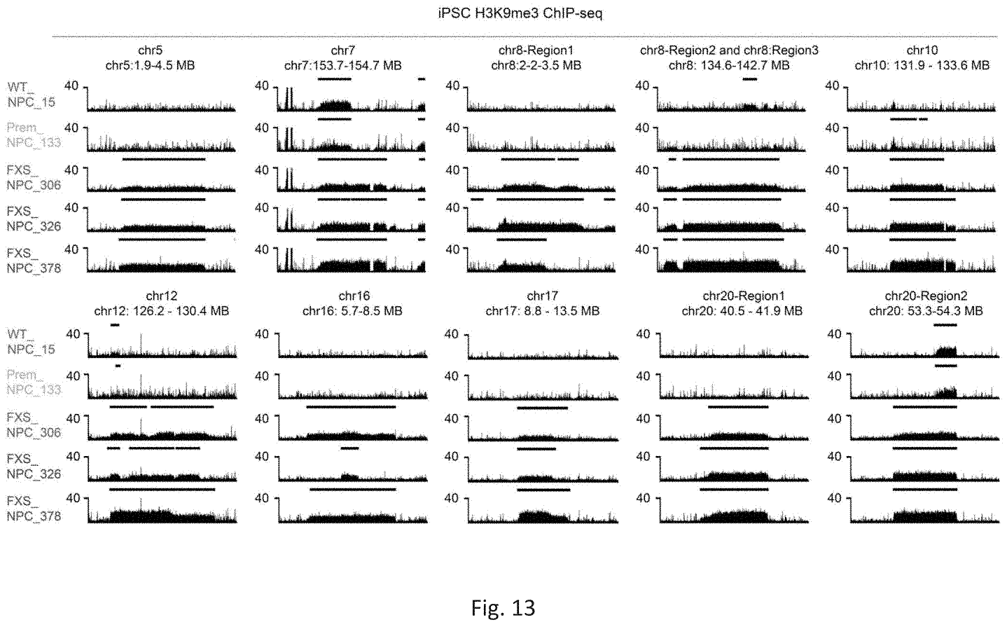

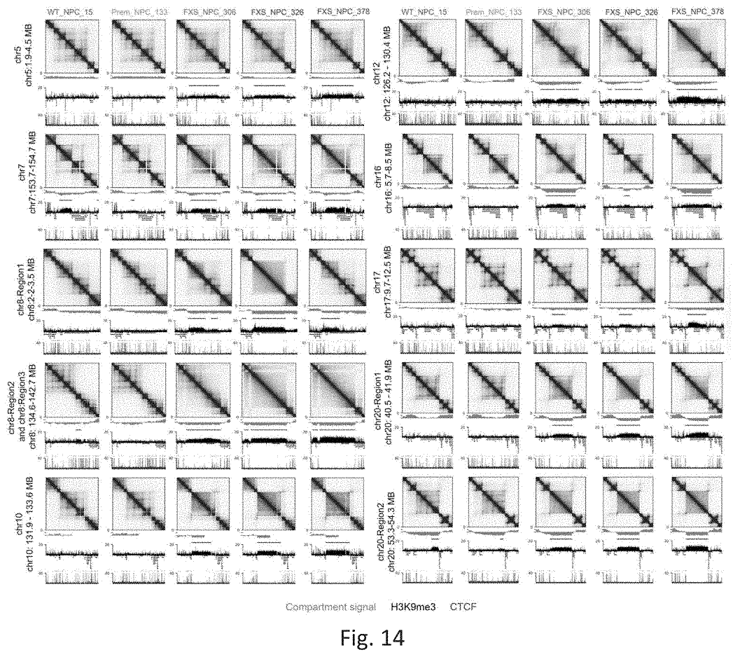

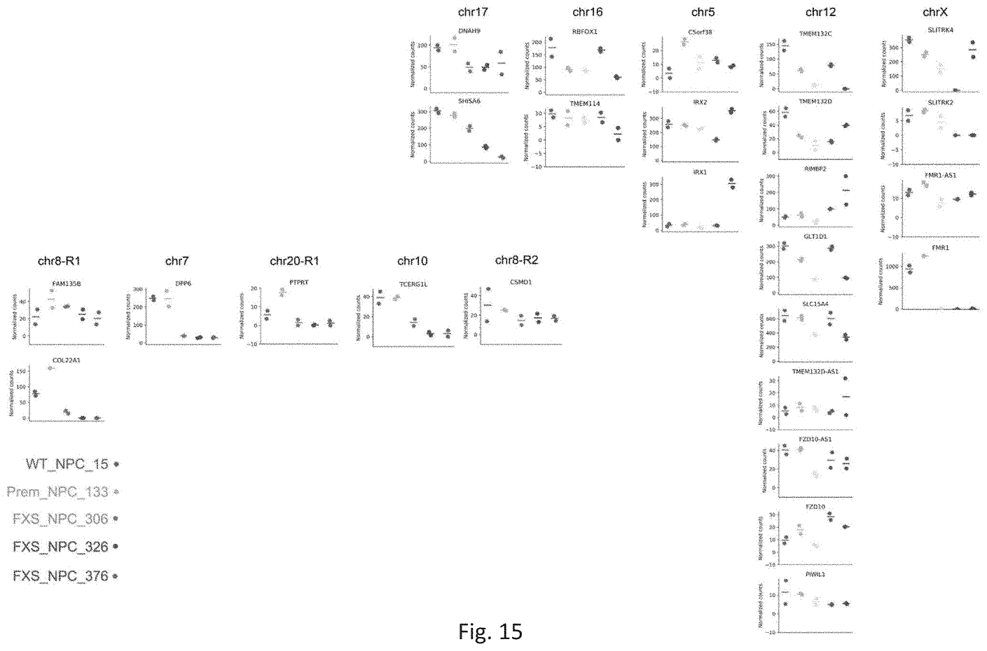

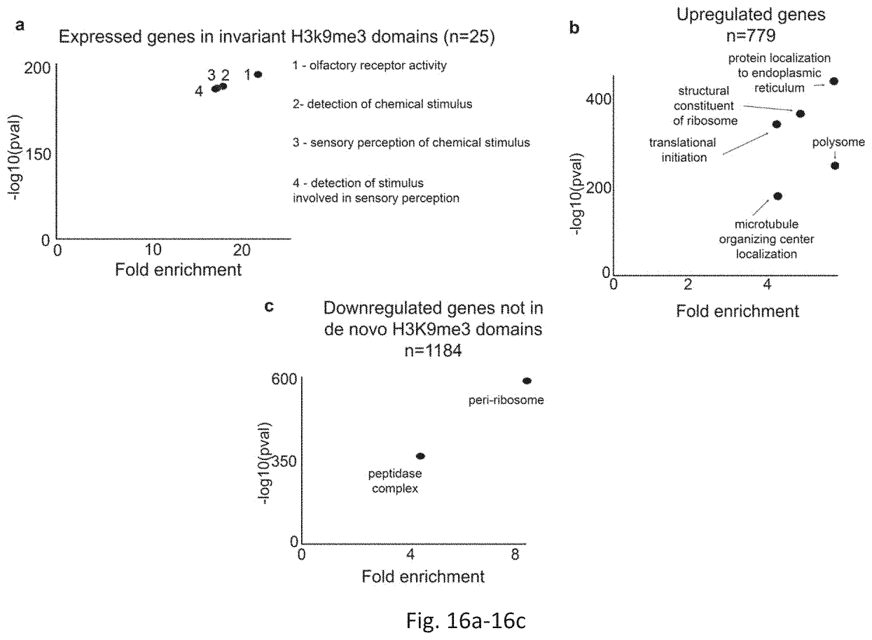

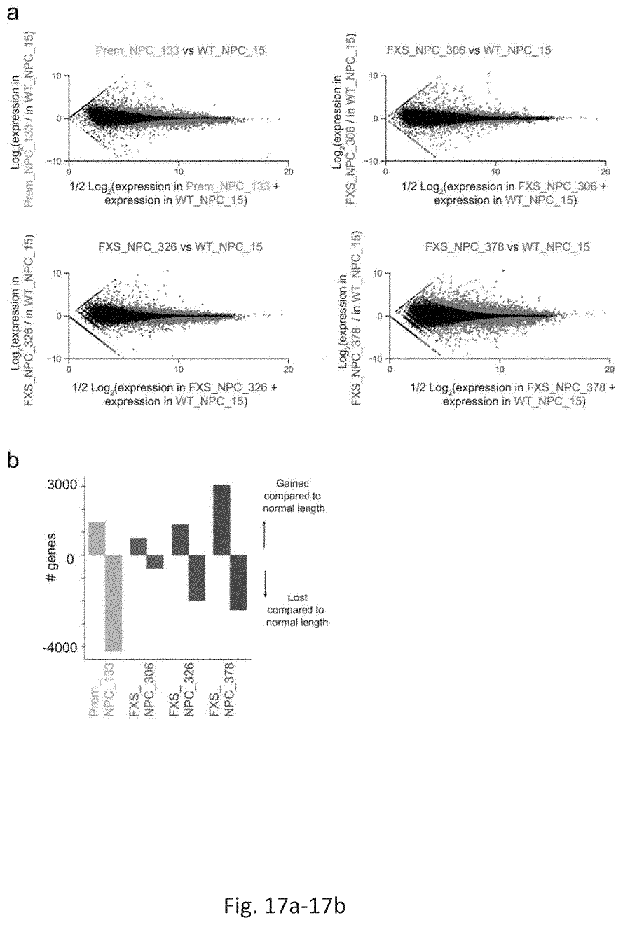

Next, it was sought to understand if H3K9me3 domains might be acquired on somatic chromosomes in FXS. Eleven additional genomic locations were identified in which large (>1 Mb) H3K9me3 domains were acquired with low signal in FXS_306 short mutation-length and subsequently strengthened and spread upon CGG expansion to long mutation-length in FXS_326 and FXS_378 (FIG. 11a, FIG. 12). The same domains were present in iPS cells (FIG. 13). One such domain encompasses the SHISA6 gene—a known fragile site on chromosome 17 (FIG. 11b). As seen at the broader FMR1 locus, acquisition of H3K9me3 upon mutation-length CGG, expansion occurs in parallel with TAD ablation and loss of CTCF occupancy (FIG. 11b-c). SHISA6 mRNA levels are decreased in a pattern that mirrors the intensity of the H3K9me3 domain (FIG. 11d). Indeed, for all 11 distal FXS domains, loss of CTCF occupancy (FIG. 11e, FIG. 14), TAD boundary disruption (FIG. 11f, FIG. 14), and a marked reduction in gene expression (FIG. 11g, FIG. 15) was observed. Gene ontology analysis confirmed that genes in the de novo FXS gained domains in NPCs are involved in synaptic plasticity and neural cell adhesion, and such synaptic genes are not enriched in the H3K9me3 domains that are invariant across all CGG lengths (FIG. 11i-j, FIG. 16a). It was noted that although both gain and loss of gene expression was see genome-wide in FXS (FIG. 17), it is only the downregulated genes in the NPC H3K9me3 domains that exhibit synaptic gene ontology (FIG. 16a-c). In addition to the twelve heterochromatin domains present across all FXS cell lines, 20 H3K9me3 domains were also identified specific to just one cell line (FIG. 11a, 11j), indicating that heterogeneity in clinical presentation in FXS patients may be due to different distributions of heterochromatinization in the FXS genomes. Together, the data reveal that large H3K9me3 domains also arise distal from the FMR1 locus in FXS and encompass genes critically linked to the synaptic plasticity defects characteristic of the disease (Pfeiffer et al., 2009, Neuroscientist 15, 549-567).

TABLE 1
H3K9me3 domains called using RSEG which are stable in FXS

| chr | start | end | chr | start | end | chr | start | end |
|---|---|---|---|---|---|---|---|---|
| chr1 | 2688950 | 2977150 | chr19 | 24256100 | 24603150 | chr19 | 53370350 | 53585950 |
| chr1 | 49789850 | 50482850 | chr19 | 27732100 | 28444350 | chr19 | 53636400 | 53836200 |
| chr1 | 248095100 | 248908000 | chr19 | 36801050 | 37752550 | chr19 | 54993500 | 55547250 |
| chr10 | 37181100 | 37503950 | chr19 | 37794350 | 38383400 | chr19 | 56211000 | 56577600 |
| chr10 | 37524850 | 38676000 | chr19 | 44656700 | 45056550 | chr19 | 56830500 | 57703000 |
| chr10 | 42770750 | 43123850 | chr19 | 52288500 | 52681750 | chr19 | 57944150 | 58168550 |
| chr10 | 135242250 | 135449600 | chr1 | 2688750 | 2977150 | chr19 | 58429800 | 58810500 |
| chr11 | 48197050 | 50073100 | chr1 | 49388000 | 50557500 | chr21 | 14919750 | 15624000 |
| chr11 | 50094550 | 50303000 | chr1 | 227708000 | 227915100 | chr22 | 16848000 | 17524800 |
| chr11 | 50323900 | 50783700 | chr1 | 247845750 | 248908050 | chr3 | 75676000 | 76016000 |
| chr11 | 51191250 | 51591650 | chr10 | 37100000 | 38709900 | chr4 | 10450 | 492300 |
| chr11 | 54794300 | 55587950 | chr10 | 42770750 | 43123850 | chr4 | 190153150 | 190958000 |
| chr12 | 14368750 | 14584900 | chr10 | 135241500 | 135450000 | chr5 | 140453000 | 140872950 |
| chr12 | 37857600 | 38708450 | chr12 | 14367750 | 14595300 | chr5 | 178074000 | 178563000 |
| chr12 | 133461900 | 133841400 | chr12 | 133460000 | 133841500 | chr6 | 57191250 | 58076000 |
| chr13 | 19357800 | 20196550 | chr14 | 20194000 | 20757000 | chr7 | 56160900 | 56443500 |
| chr14 | 20194350 | 20757000 | chr14 | 105970500 | 107289600 | chr7 | 61968000 | 62750250 |
| chr14 | 105973450 | 107289600 | chr15 | 22296750 | 22590000 | chr7 | 63207650 | 64345500 |
| chr15 | 22297000 | 22589600 | chr15 | 23613000 | 25594000 | chr7 | 137810500 | 138175950 |
| chr16 | 3241700 | 3494150 | chr16 | 3241700 | 3500500 | chr7 | 157227000 | 158392500 |
| chr16 | 32374100 | 32656800 | chr16 | 32355900 | 32657000 | chr8 | 134874450 | 135482400 |
| chr16 | 33370700 | 33631950 | chr16 | 33351500 | 33643800 | chr9 | 125159500 | 125570500 |
| chr16 | 33798050 | 34023000 | chr16 | 33795000 | 34023150 | chrX | 154933200 | 155235500 |
| chr16 | 34173150 | 35285800 | chr19 | 6774750 | 7019250 | chrY | 13798000 | 14743500 |
| chr17 | 21666700 | 22247500 | chr19 | 8897250 | 9147050 | chrY | 21910050 | 22357500 |
| chr19 | 6784800 | 7019100 | chr19 | 9192150 | 9728950 | chrY | 22507500 | 22735800 |
| chr19 | 9192150 | 9728950 | chr19 | 15582000 | 16174000 | chrY | 22760000 | 23656000 |
| chr19 | 11709500 | 12703350 | chr19 | 19775800 | 20504250 | chrY | 24332400 | 24546150 |
| chr19 | 15670600 | 16119400 | chr19 | 20639000 | 21198500 | chrY | 25848750 | 26162100 |
| chr19 | 20639850 | 21198100 | chr19 | 23592000 | 23966000 | chrY | 27799650 | 28113800 |
| chr19 | 22029700 | 23280950 | chr19 | 44656700 | 45057500 | chrY | 28408500 | 28819000 |
| chr19 | 23599400 | 24228600 | chr19 | 52751500 | 53158050 | chrY | 58967250 | 59337900 |

TABLE 2
H3K9me3 domains called using RSEG
which are variably gained in FXS

| chr | start | end |
|---|---|---|
| chr11 | 36774750 | 40146000 |
| chr14 | 24954300 | 25879050 |
| chr14 | 27498750 | 29996250 |
| chr15 | 54277500 | 55452750 |
| chr16 | 25289100 | 27184050 |
| chr18 | 68143500 | 70338000 |
| chr18 | 75371250 | 76730250 |
| chr22 | 34329000 | 35413500 |
| chr3 | 60300 | 3186450 |
| chr3 | 3224250 | 3837600 |
| chr3 | 3904200 | 4313700 |
| chr3 | 5298750 | 7314300 |
| chr6 | 64687500 | 67557750 |
| chr7 | 144744000 | 145782000 |
| chr7 | 158725350 | 159128550 |
| chr8 | 5547000 | 6256500 |
| chr8 | 20916450 | 21451500 |
| chr8 | 142825500 | 143352000 |
| chrX | 460500 | 1034250 |
| chrY | 362250 | 984000 |

TABLE 3
H3K9me3 domains called using
RSEG which are consistently gained in FXS

| chr | start | end |
|---|---|---|
| chr10 | 131986800 | 133677750 |
| chr12 | 126170250 | 131251050 |
| chr16 | 5668650 | 8615250 |
| chr17 | 10750950 | 11835000 |
| chr20 | 40337250 | 42074250 |
| chr20 | 53298900 | 54918000 |
| chr5 | 1899900 | 4869750 |
| chr7 | 152790750 | 154704750 |
| chr8 | 2030400 | 4851000 |
| chr8 | 135855000 | 136459800 |
| chr8 | 136671750 | 140779500 |
| chrX | 141905250 | 147118950 |

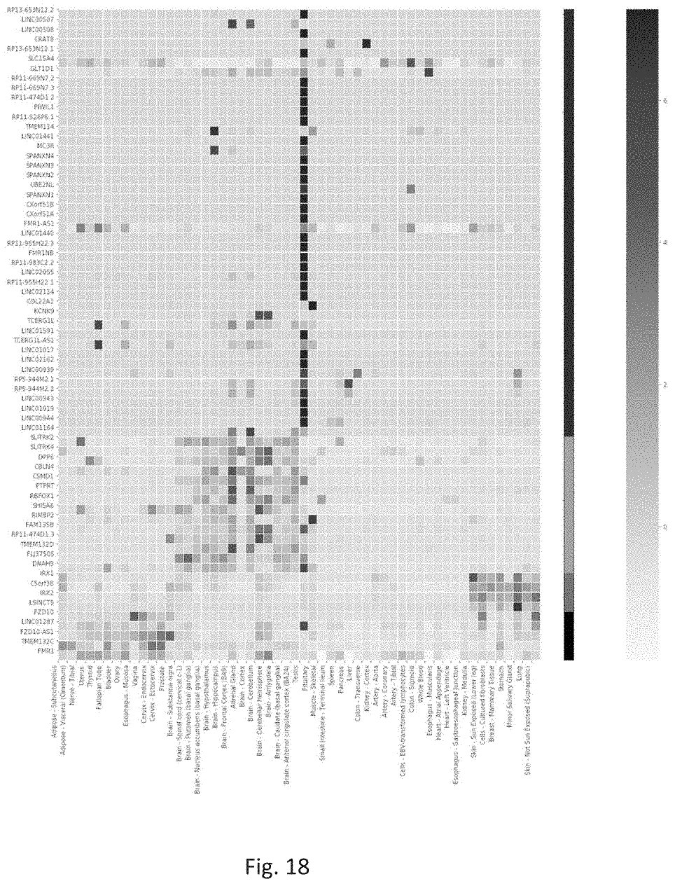

Macro-orchidism and soft skin are unexplained clinical presentations in FXS (Atkin, 1985, Am J Med Genet 21, 697-705), and expansion of the FMR1 CGG STR also causes severe ovary defects in Fragile X-associated primary ovarian insufficiency (FXPOI) (Tan et al., 2009, Neurosci Lett 466, 103-108). To understand the transcriptional profile of the H3K9me3-localized genes in tissues outside the brain, expression across 54 tissues from the GTEX consortium was examined. It was observed that genes localized to FXS heterochromatin domains largely exhibit tissue-specific expression profiles, including testis, female reproductive organs, epithelium, and (consistent with the NPC results) brain (FIG. 11h, FIG. 18). Given that the NPC FXS domains are also present in iPS cells, these results suggest that many of such domains will also be present in skin and reproductive tissues and thus relevant to the silencing of genes linked to non-brain pathology. These results bring to light a compelling hypothesis in which distal heterochromatinization and silencing of epithelial and testis genes on somatic chromosomes is a mechanism contributing to pathological features outside the brain in a broad range of clinical presentations due to FMR1 CGG instability.

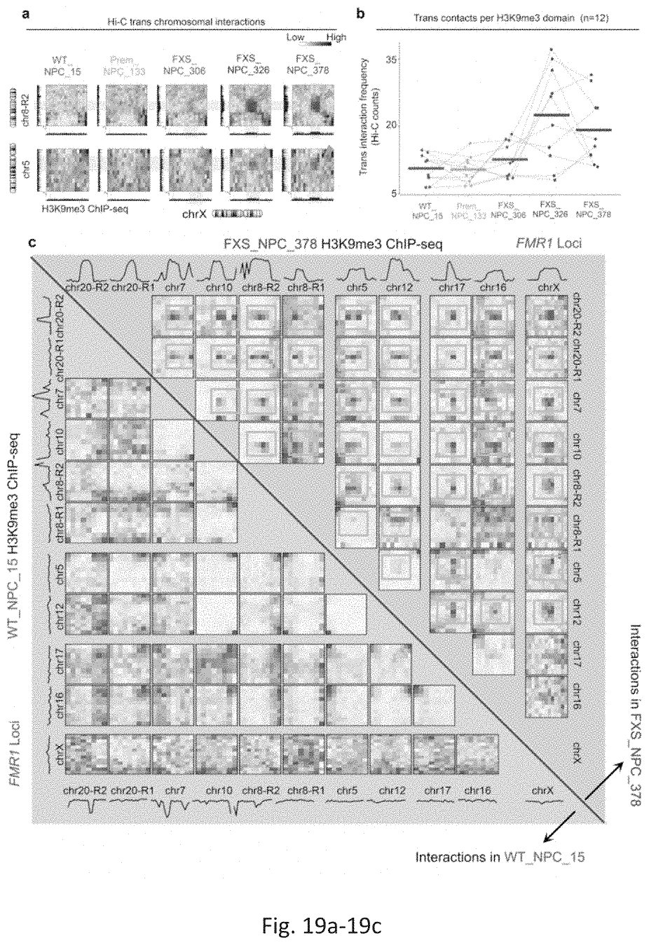

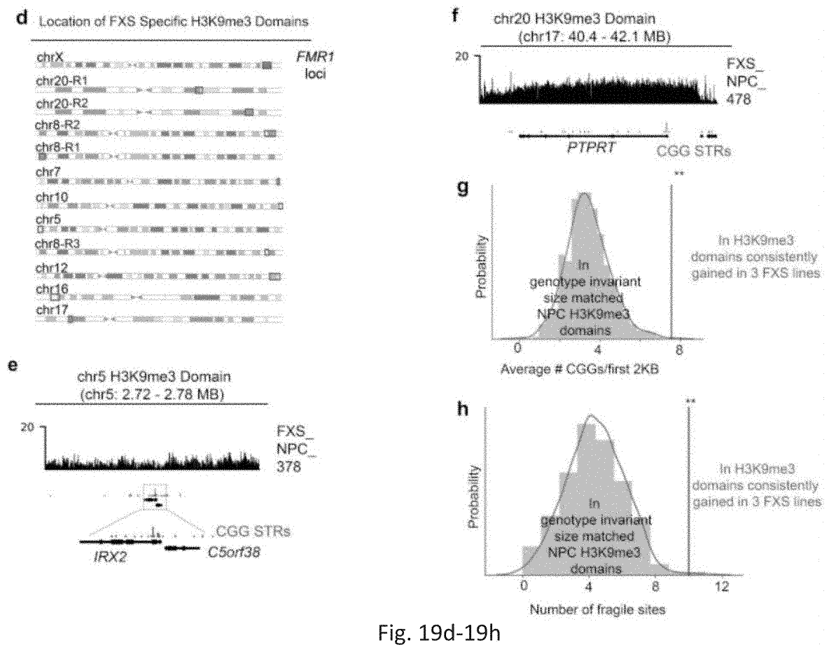

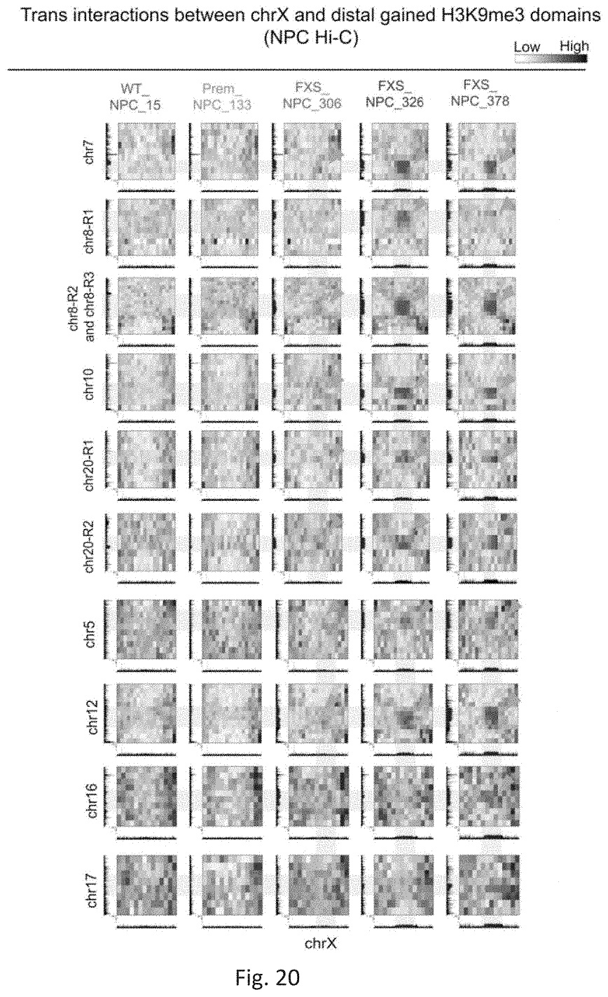

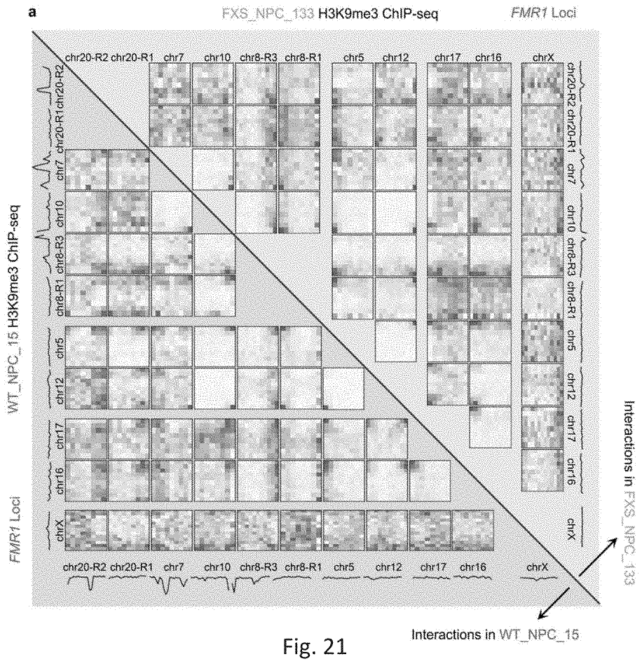

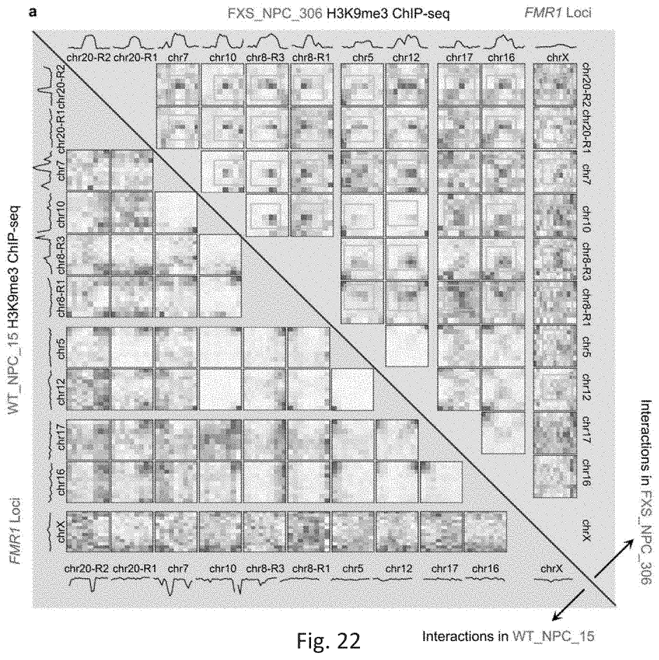

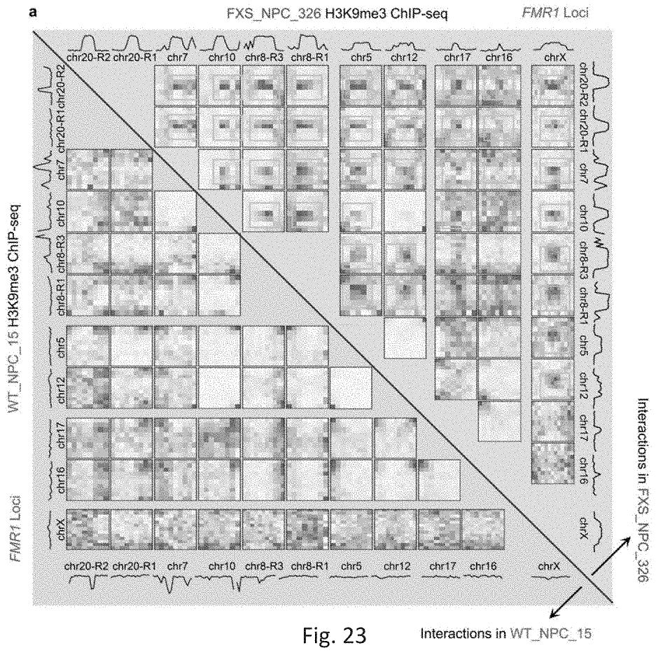

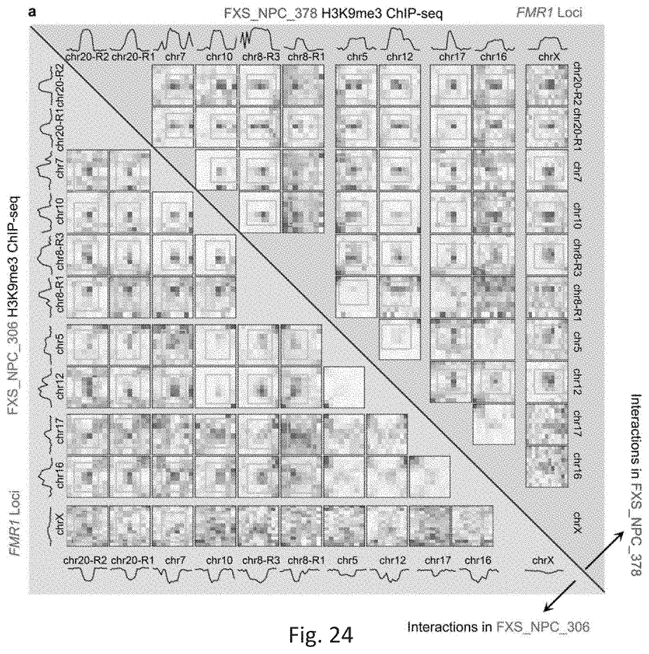

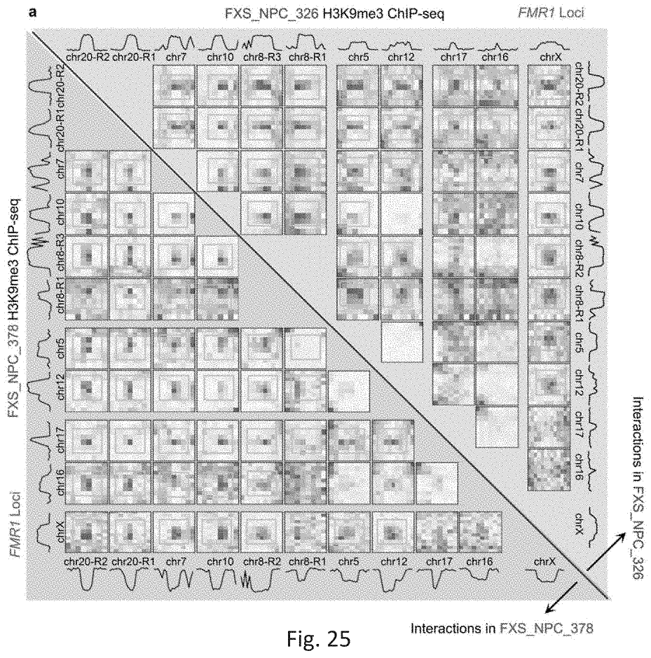

Given that the primary site of STR expansion is in the FMR1 gene on the X chromosome, it remains quite striking that distal loci on somatic chromosomes would be heterochromatinized in FXS. To understand how FMR1 communicates with distal loci, inter-chromosomal interactions with Hi-C were examined. Unexpectedly, trans (i.e. between-chromosome) interactions exhibiting unusually strong interaction frequency were observed connecting the FMR1 locus specifically to distal H3K9me3-marked domains (FIG. 19a-b, FIG. 20). Importantly, it was observed that all distal silenced H3K9me3 domains form a physical subnuclear hub in which all distal H3K9me3 domains are spatially proximal to FMR1 and to each other in FXS (FIG. 19c). It was noted that the formation of trans interactions occurs concomitant with the density of H3K9me3 acquired during disease progression (FIGS. 20-25). The subnuclear trans spatial silencing hub is not present in normal-length or pre-mutation length cells, initiates upon short mutation-length (FXS_306), and forms in full strength as the H3K9me3 domains spread and gain density of signal (FXS_326, FXS_378) (FIGS. 20-25). Together, these data show that the genome-wide gained FXS heterochromatin domains engage directly via spatial proximity with the unstable FMR1 locus upon mutation-length expansion of the CGG STR tract.

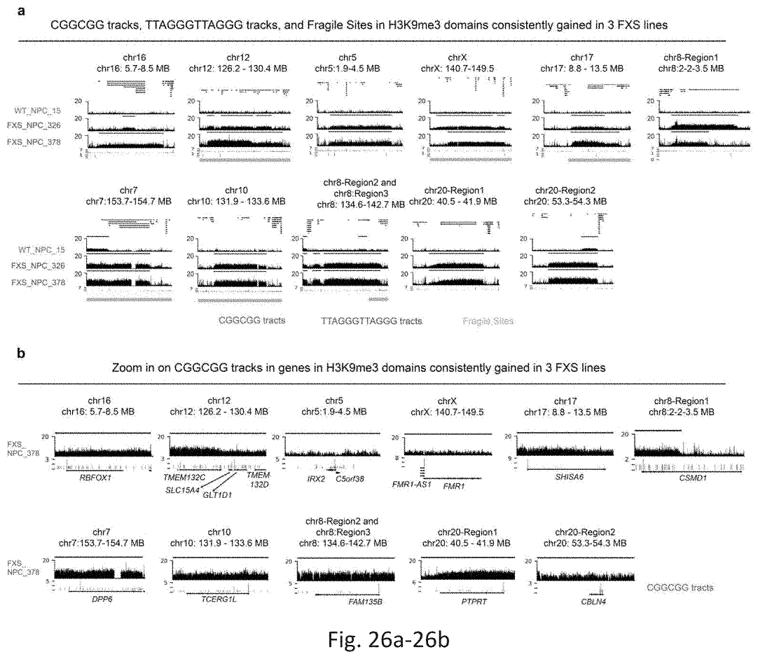

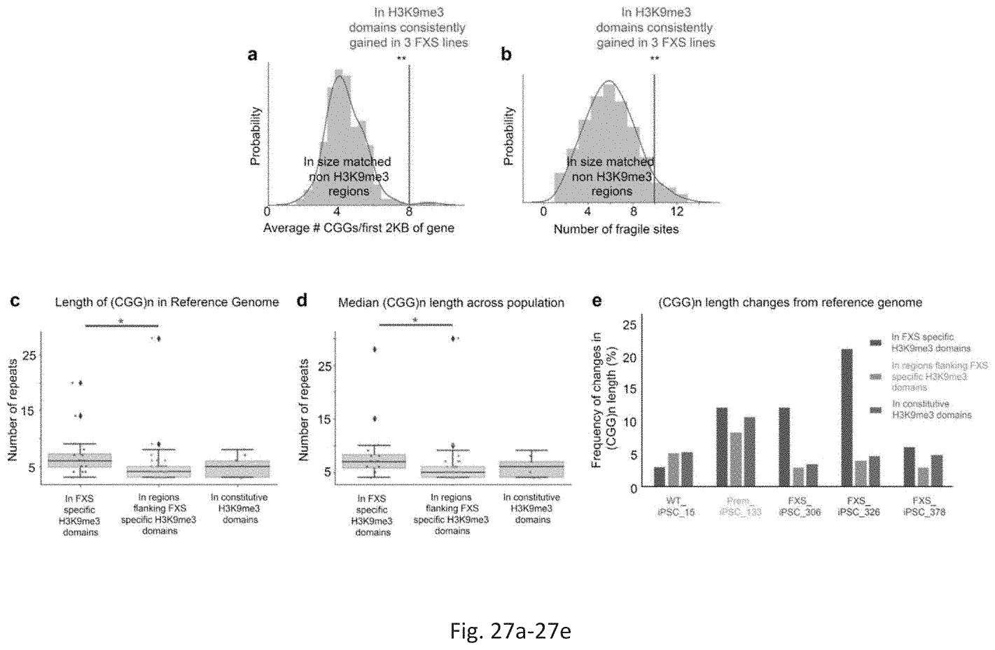

To understand why the unstable FMR1 locus would spatially contact and coordinate heterochromatinization with the specific distal locations and not with other locations in the genome, the unique genetic features of the FXS H3K9me3 domains were explored. It was first noticed that almost all the gained distal domains, like FMR1, are located at the ends of chromosomes adjacent to sub-telomeric regions (FIG. 19d). It was also observed that, like FMR1, genes localized in FXS H3K9me3 domains exhibit an extremely high density of normal-length CGG STR tracts (FIG. 19e-f, FIGS. 26-27). The density of CGG STR tracts in the 5′UTR of genes in the FXS H3K9me3 domains is significantly higher than expected in the rest of the genome, including null distributions of CGG STR density in random size-matched random regions or even genotype-invariant H3K9me3 domains present across all five lines (FIG. 19g, FIG. 27a-b). Together, this work demonstrates that regions of the genome silenced in FXS are similar to FMR1 loci in that they are at the ends of chromosomes and are enriched for CGGs STRs in the 5′UTR of genes. Without being bound by theory, it was posited that these features may predispose distal loci as targets of the mechanisms driving H3K9me3 at FMRL.

Heterochromatinization is known to protect the repetitive genome against instability (Janssen et al., 2018, Annu Rev Cell Dev Biol 34, 265-288). Without being bound by theory, it was hypothesized that CGG STR-rich genes in FXS H3K9me3 domains would require spatially coordinated heterochromatinization because they fall in genomic locations that are highly susceptible to instability. Consistent with this idea, it was noticed that the majority of the FXS domains also overlapped established human fragile sites (FIG. 19h, FIG. 26). Additionally, CGGs in FXS domains are longer than CGGs elsewhere in the genome, and longer repeats are associated with increased instability (FIG. 27a-b). Moreover, using the ExpansionHunter method, CGG tract length was quantified across the cell lines after whole genome PCR-free sequencing. It was observed that an increased rate of genes in H3K9me3 domains exhibited expanded or contracted CGG tracts in the F×S lines where FMR1 has a long mutation-length CGG, compared to normal and pre-mutation length cell lines where deviations in CGG length in H3K9me3 domains was observed at a rate consistent with non FXS specific-H3K9me3 domains, suggesting that normal-length STRs distal from FMR1 grown unstable in FXS (FIG. 27 c-d), Together, these data inspired a working model in which CGG tracts across the genome communicate with each other spatially via trans interactions as a surveillance mechanism that enables the heterochromatinization and silencing of genes at risk of instability.

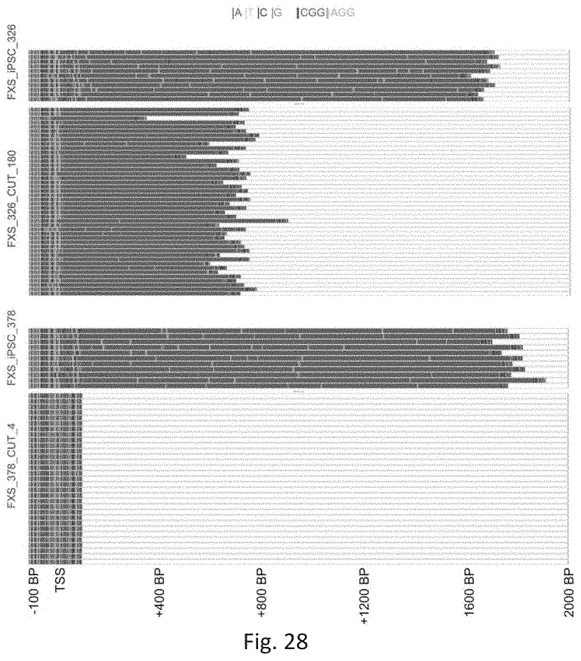

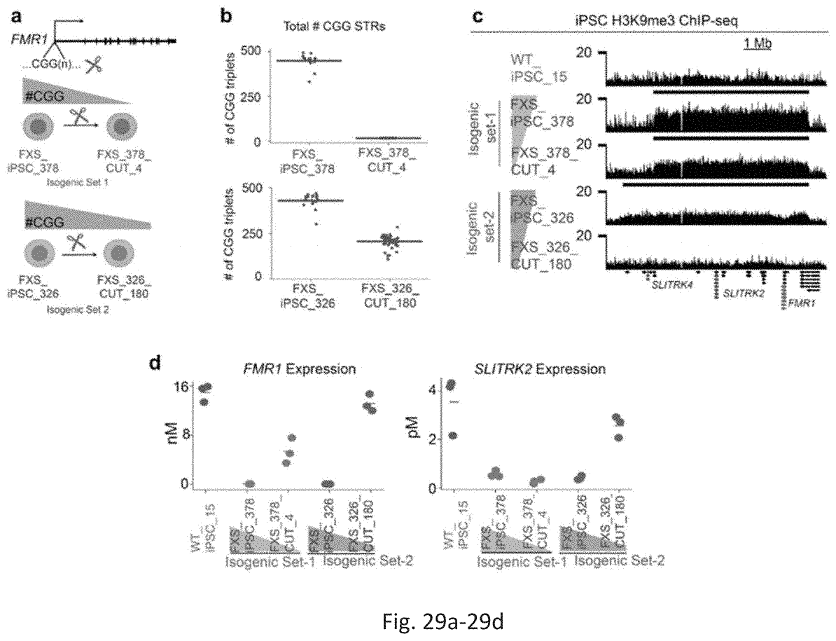

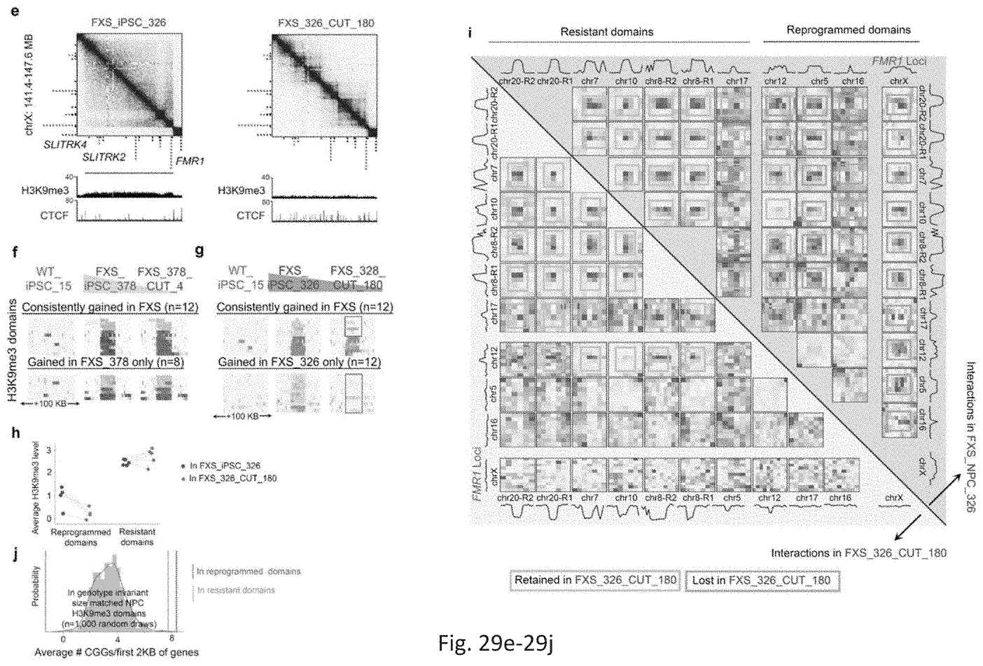

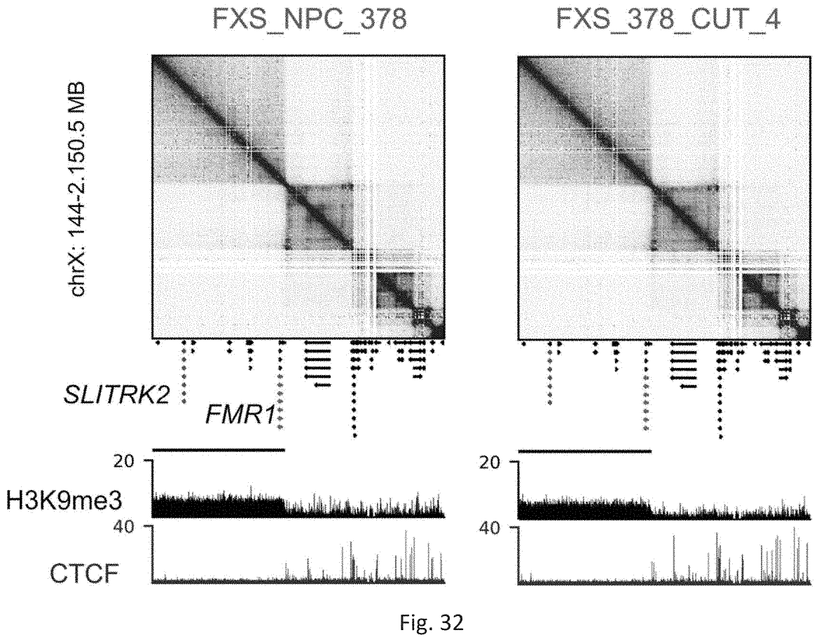

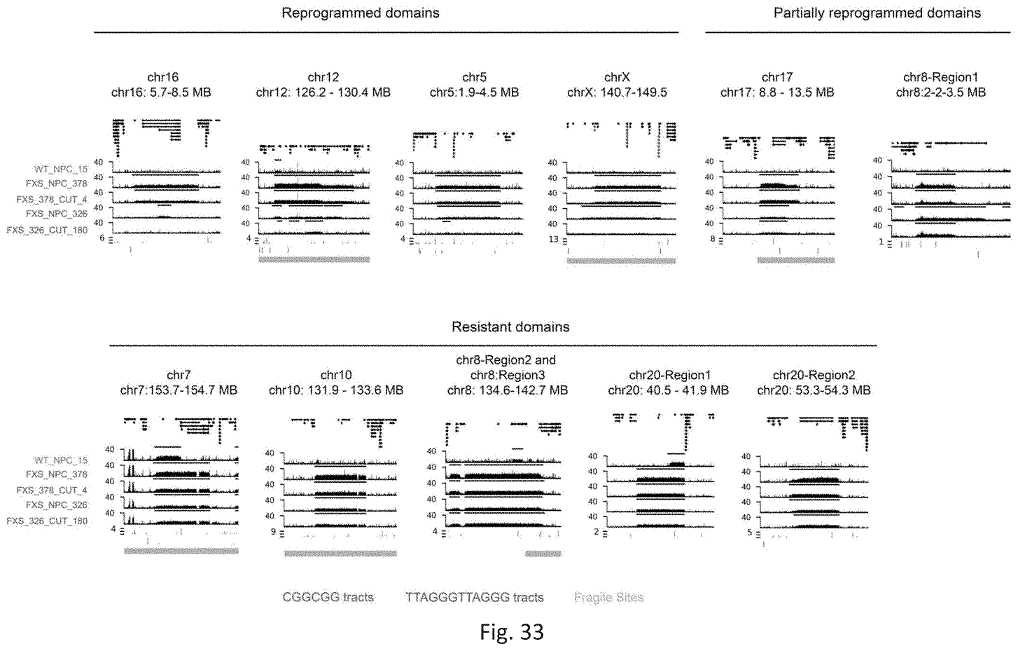

To understand the functional role of the FMR1 CGG STR in altering heterochromatin, the extent of H3K9me3 reversibility was examined after shortening the CGG to pre-mutation or normal-length with CRISPR (FIG. 29a). In the first IPSC cohort, the FMR1 CGG tract in the long mutation-length FXS_iPSC_378 line was cut back to the normal-length range of 4 CGG triplets (FIG. 29a-b, FIGS. 28, 30, and 31). It was observed that the large H3K9me3 domain spanning SLITRK4, SLITRK2, and FMR1 did not notably change after CGG STR cut-back to normal-length (FIG. 29c). CTCF binding was not re-gained and genome folding domains remained destroyed just as in the FXS_IPSC_378 parent line (FIG. 32). Consistent with previous reports, the FMR1 gene was partially de-repressed in the normal-length cut-out, however SLITRK2 remained silenced (FIG. 29d). It was also noticed that all distal heterochromatinized loci maintained a high level of H3K9me3 signal upon normal-length CGG cut-out (FIG. 33). The data indicate that engineering the CGG STR back to normal-length range does not markedly reprogram local or distal H3K9me3 domains genome-wide, suggesting that pathologically silenced synaptic, epithelial, testis, and female reproductive tissue genes will not be de-repressed with an FMR1 CGG normal-length cut out strategy in FXS.

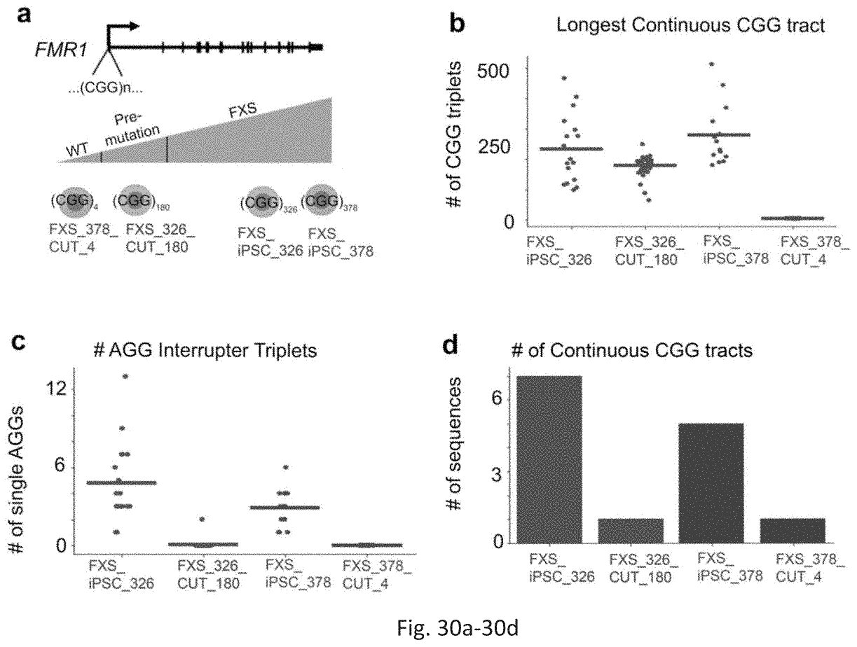

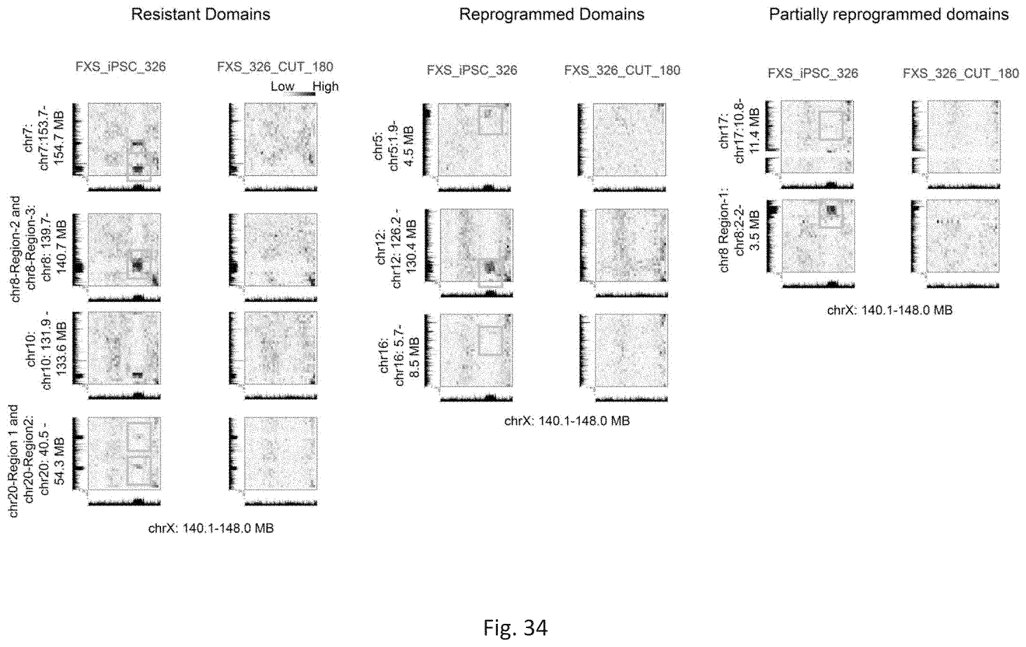

In the second IPSC cohort, the FMR1 CGG tract in the second long mutation-length FXS_iPSC_326 line was cut back to a pre-mutation length of 180 CGG triplets, as confirmed by Nanopore sequencing (FIG. 29a-b, FIGS. 28 and 30). It was observed that the H3K9me3 domain encompassing SLITRK4, SLITRK2, and FMR1 is fully reversible upon cut-out to pre-mutation-length (FIG. 29c). Corroborating the loss of the H3K9me3 domain, CTCF occupancy was re-gained and TAD boundaries were re-instated (FIG. 29e). Both SLITRK2 and FMR1 mRNA levels were nearly fully restored upon engineering to pre-mutation length (FIG. 29d), and the X chromosome H3K9me3 domain disconnected from its trans interactions with distal domains (FIG. 34). These results suggest that the reversal of the H3k9me3 heterochromatin domain around FMR1 might require a step back through the stage of disease acquisition involving the pre-mutation length CGG STR.

Next, the extent to which the distal H3K9me3 domains in FXS could be reversed upon local FMR1 CGG STR engineering was explored. By contrast to the cut-out to normal-length range where no distal H3K9me3 signal was altered, it was observed that a subset of distal H3K9me3 domains were fully reprogrammed upon only engineering of the FMR1 CGG STR precisely to 180 CGG pre-mutation length (FIG. 29f-g, FIG. 33). Distal domains with the lowest H3K9me3 density were the most susceptible to reprogramming after engineering the FMR1 CGG STR (FIG. 29h). Although the domains with high H3K9me3 density remain engaged in the subnuclear trans spatial hub, several distal domains lost their heterochromatinization and spatially disconnected upon engineering of the mutation-length CGG at FMR1 to pre-mutation (FIG. 29i). It was noted that reprogrammed domains had a higher density of CGG STRs per gene compared to resistant domains, suggesting that reversal potential is CGG density dependent (FIG. 29j). Together, these results highlight the remarkable ability of the FMR1 CGG STR to communicate spatially in trans with distal H3K9me3 domains, functionally contributing, at least in part, to the acquisition of their pathologic heterochromatinization. Importantly, reverse engineering of the FMR1 CGG to pre-mutation length can fully reverse the H3K9me3 domain locally at FMR1 and also attenuate a subset of distal H3K9me3 domains. The persistence of heterochromatin silencing at many reprogramming resistant H3K9me3 domains in FXS highlights the importance of additional clinical interventions beyond FMR1 CGG STR engineering, and suggests that many distal H3K9me3 domains in FXS may form through a mechanism that is independent of the FMR1 CGG.

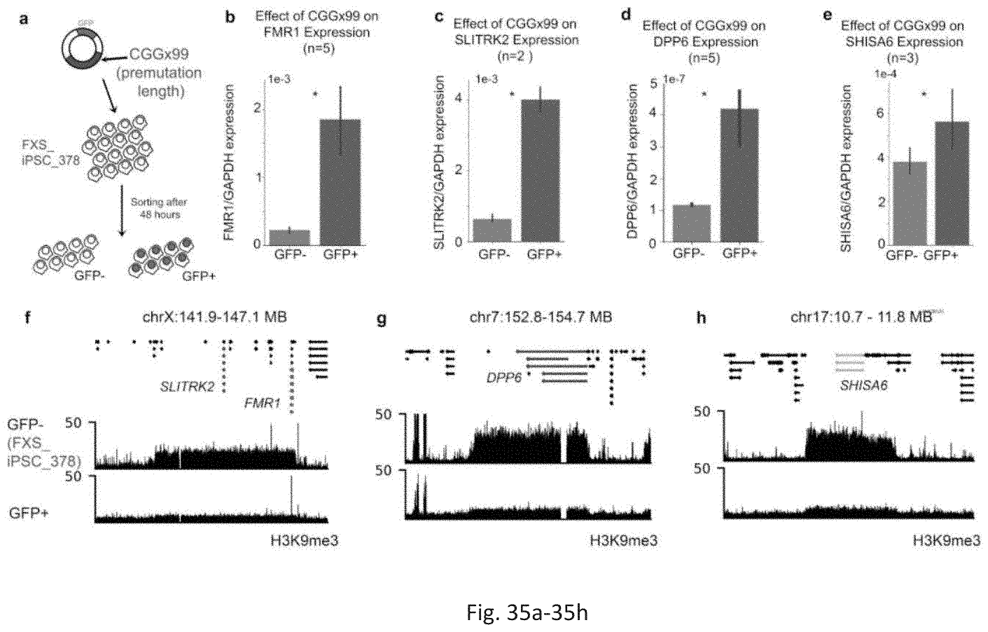

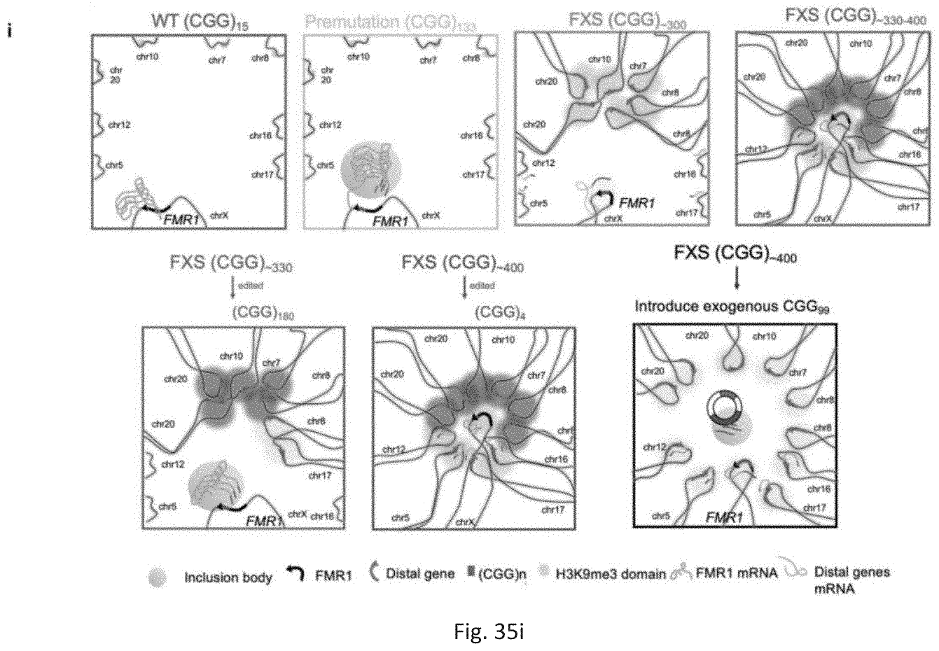

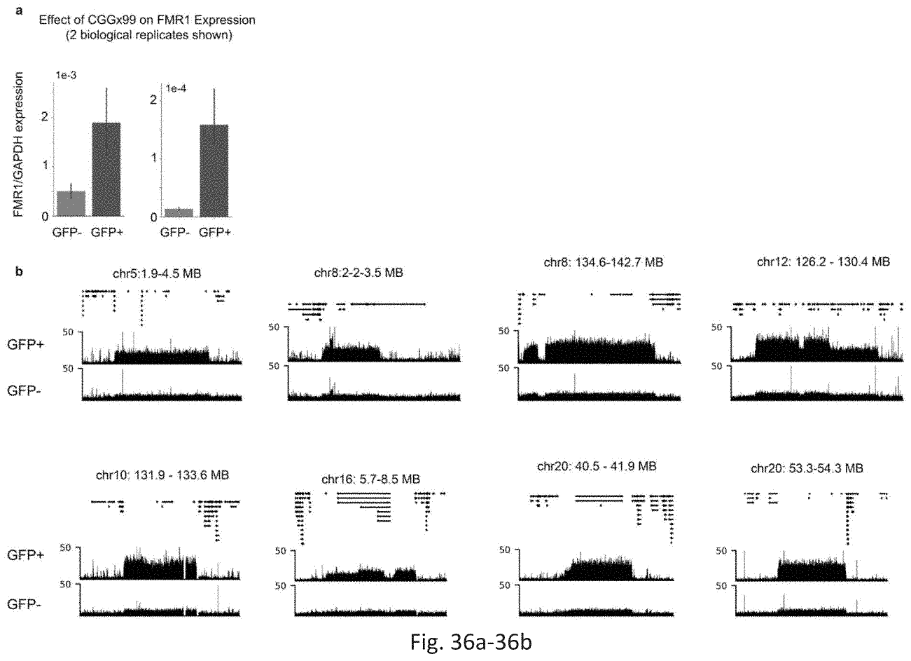

Finally, it was sought to understand if overexpression of a pre-mutation CGG STR sequence alone, independent from its placement in the FMR1 gene, was sufficient to attenuate local or distal FXS H3K9me3 domains. Gene expression and H3K9me3 was queried after overexpressing a transgene expressing 99 CGG triplets (pre-mutation) in long mutation-length FXS IPSCs for 48 hours (FIG. 35a). A striking de-repression of FMR1, SLITRK2, DPP6, and SHISA6 was observed, with a much higher effect size than that observed due to CRISPR CGG engineering to pre-mutation length within the endogenous FMR1 locus (FIG. 35b-e, FIG. 36). Using CUT&RUN for H3K9me3, which is amenable to assaying signal in low cell numbers, we observed complete ablation of nearly all distal H3K9me3 heterochromatin domains in FXS upon overexpression of the pre-mutation CGG STR (FIG. 35f-h). Altogether, these data reveal that both local and distal heterochromatin domain acquisition in FXS can be fully reversed by ectopic expression of a pre-mutation length CGG STR, suggesting that the spatial subnuclear hub of fragile repetitive regions in FXS is driven by a CGG-mediated DNA or RNA mechanism that transcends FMR1.

Altogether, the data support a model of pervasive long-range transcriptional silencing in FXS via the acquisition of a physically-connected subnuclear hub of more than ten Megabase-sized domains of the repressive histone modification H3K9me3. Such domains acquire low levels of H3K9me3 signal in the transition from pre-mutation to short mutation-length, and increase in severity and spread of H3K9me3 density as the FMR1 CGG STR expands to long mutation-length (FIG. 35i). Consistent with previous reports, local DNA methylation of the FMR1 gene correlates with its degree of silencing. By contrast, a large cohort of genes are repressed in FXS in a manner commensurate with the severity of H3K9me3 density in distal heterochromatin domains. It has long been thought that global gene expression disruption in FXS is due to the downstream effects of FMRP loss, however here we see that the CGG STR expansion in FMR1 activates a genome-wide surveillance system to deposit large H3K9me3 domains to directly silence CGG STR-rich genes localized at the ends of distal chromosomes. The FXS pathologic heterochromatin domains encompass and silence genes critical for synaptic plasticity, testis development, female reproductive system functioning, and epithelial tissue structure, which are precisely the pathologically disrupted tissues in FXS. These results suggest that pharmacological and RNA-based interventions to reverse distal H3K9me3 silencing may provide tangible therapeutic benefits to FXS patients as long as genome stability can be maintained.

It is difficult to envision how a CGG STR expansion event in FMR1 could coordinate heterochromatinization on 10 other chromosomes. Here, evidence of a physically-linked subnuclear hub of inter-chromosomal interactions among known human fragile sites in FXS is provided. Without being bound by theory, it was hypothesized that critical areas of the genome communicate to coordinate silencing when an instability event is detected. CRISPR engineering of the long mutation-length CGG tract to pre-mutation length provides evidence that at least a subset of distal domains are heterochromatinized and spatially connected as directed by the FMR1 STR. It is also likely that the DNA sequence or RNA encoded by additional CGG STR tracts will contribute to FXS heterochromatinization, as we demonstrate that overexpression of a generic CGG STR transgene results in complete attenuation of all distal H3K9me3 domains and full de-repression of distal genes. It is noteworthy that CRISPR shortening of the mutation length CGG STR to normal-length only slightly de-represses FMR1 and had no noticeable effect on distal heterochromatin domains. Other studies showing stronger FMR1 de-repression upon local CGG cut-out to normal-length may have started with a shorter mutation-length tract more amenable to reprogramming of epigenetic marks. These results suggest that genetically engineered CGG-based CRISPR therapeutic approaches targeting only FMR1 may not fully reverse the silencing of key genes contributing to persistent pathology in FXS patients. Full reversal of pathologic features across multiple tissues may require combination therapies coupling pharmacological intervention and STR engineering. Altogether, this work uncovers a pervasive genome-wide surveillance mechanism by which fragile sites in the genome spatially communicate over vast distances via pathologically expanding CGG STR tracts to heterochromatinize and silence the unstable genome.

### Methods

### Cell Culture

B-Lymphocytes

Patient-derived B-lymphocytes were cultured as previously described (Sun et al., 2018, Cell 175, 224-238 e215). In brief, cells were grown in suspension in RPMI 1640 media (Sigma, R8758) supplemented with 2 mM glutamine, 15% (v/v) Fetal Bovine Serum, 1% (v/v) penicillin-streptomycin (Thermo Fisher, 15140122) at 37° C. and 5% CO₂. Cells were passaged every 2-4 days, when they reached a density of approximately 5e5 cells/mL. All cell lines were male.

Induced Pluripotent Stem (iPS) Cells

All human iPS cells were obtained from Fulcrum Therapeutics (MA, USA). Cells were cultured in mTeSR plus (STEMCELL Technology, 05825) supplemented with 1% (v/v) penicillin-streptomycin (Thermo Fisher, 15140122) at 37° C. and 5% CO₂ on Matrigel coated plates. Cells were passaged by incubating in 5 ml of Versene Solution (Thermo Fisher, 15040066) at 37° C. for 3 min, after which Versene was inactivated by mixing with 10 ml of full growth media. Cells were passaged every 2-7 days. All iPS culture plates were coated with 1.2% (v/v) Matrigel hESC-Qualified Matrix (Corning, 354277) in DMEM/F-12 (Thermo Fisher, 11320033) for at least 1 hr at 37° C. All cell lines were male.

Neural Progenitor Cell Differentiation

Human iPSC were differentiated into NPCs using a previously established protocol (Xie et al 2013). Briefly, undifferentiated cells were maintained in mTESR Plus (STEMCELL Technology, 05825) on Matrigel coated plates. They were seeded onto fresh Matrigel plates in NPC media at a density of 16,000 cells/cm². NPC media was changed every day and cells were harvested at the end of day 8. The NPC differentiation medium consists of DMEM/F12 (Thermo Fisher, 11320033) with 5 μg/ml insulin, 64 g/ml L-ascorbic acid, 14 ng/ml sodium selenite, 10.7 ug/ml Holo-transferrin, 543 μg/ml sodium bicarbonate, 10 μM SB431542 and 100 ng/ml Noggin.

FMR1 CGG Cut-Out Isogenic iPSC Engineering

The FXS_378_CUT_4 isogenic iPS cell line (CGG cut-out from FXS_iPSC_378) was generated using CRISPR/Cas9 mediated targeted CGG deletion as described by Xie et al., 2016 (doi: 10.1371/journal.pone.0165499). To generate FXS_326_cut 180, the FXS iPSC_326 parental line was cultured in Geltrex coated T75 flask. The day before electroporation, cells were fed with fresh Stemflex™ medium with 1× RevitaCell supplements. Cells were dissociated with 5 ml Accutase™ cell dissociation reagent (STEMCELL technology, 07920). After washing once with PBS, cells were resuspended in Resuspension buffer R (Neon™ Transfection System 100 L Kit, Invitrogen, 10431915) to a final cell density ˜10⁸/ml. Dissociated iPSC were then incubated with 60 ug of a plasmid containing Cas9 and gRNA targeted to the 5′ end of exon 1 in FMR1 (sequence: 5′-TGACGGAGGCGCCGCTGCCA-3′; SEQ ID NO: 2). The resulting solution was electroporated with the following program: Pulse voltage 1,100v; Pulse width 30 ms; Pulse number 1; with cell density at 1×10⁸ cells/ml. After electroporation, cells were plated into a Geltrex coated T75 flask using Stemflex™ medium with 1× RevitaCell supplements. On day 3 post electroporation, cells were dissociated with Accutase for FACS sorting to enrich the GFP+ population, and re-plated onto Geltrex coated 10 cm Petri dish at ˜5 k/plate. 1× RevitaCell was supplemented in the Stemflex medium to enhance the cell viability. iPSC cell colonies were hand-picked and expanded in Stemflex medium from 96 wells to 12 wells, and further expanded for cryopreservation. Genotype were assessed using a pair of primers upstream and downstream to the CGG repeat expansion. Forward Primer: 5′-tcaggcgctcagctccgtttcggtttca-3′ (SEQ ID NO:3), Reverse Primer: 5′-AAGCGCCATTGGAGCCCCGCACTTCC-3′ (SEQ ID NO:4)

### Genomics Assays

Cell Fixation

Cells were fixed as previously described for all downstream ChIP-seq, Hi-C, and 5C¹⁻⁶ assays. Cell lines were fixed in 1% (v/v) formaldehyde for 10 min at room temperature in either RPMI 1640 (Sigma, R8758) or in DMEM/F-12 (Thermo Fisher, 11320033) for B-lymphocytes or iPSC/NPCs, respectively. The complete fixation media was 50 mM HEPES-KOH (pH 7.5), 100 mM NaCl, 1 mM EDTA, 0.5 mM EGTA, 11% formaldehyde. Fixation was quenched in 125 mM glycine for 5 min at room temperature, following by 15 min at 4° C. Crosslinked cells were washed in pre-chilled PBS before flash frozen and stored at −80° C.

Chromatin Immunoprecipitation (ChIP-Seq)

ChIP-seq was performed as previously described with minor modification (Sun et al., 2018, Cell 175, 224-238 e215; Kim ET AL., 2019, Nat Methods 16, 633-639; Kim et al., 2018, Methods 142, 39-46; Beagan et al., 2017, Genome Res 27, 1139-1152; Beagan et al., 2016, Cell Stem Cell 18, 611-624; Phillips-Cremins et al., 2013, Cell 153, 1281-1295). Briefly, crosslinked cell pellets (consisting of 10 million cells for CTCF ChIP-seq or 3 million cells for H3K9me3 ChIP-seq), were lysed in cell lysis buffer (10 mM Tris pH 8.0, 10 mM NaCl, 0.2% NP-40/Igepal, Protease Inhibitor, PMSF) on ice for 10 min. The suspension was then homogenized with pestle A 30 times. The nuclei were pelleted from the initial lysate at 2,500 g at 4C and the resulting nuclei were further lysed in 500 μl of nuclear lysis buffer (50 mM Tris pH 8.0, 10 mM EDTA, 1% SDS, Protease Inhibitor, PMSF) and incubated on ice for 20 min. Lysed nuclei were then sonicated by adding 300 μP IP Dilution Buffer (20 mM Tris pH 8.0, 2 mM EDTA, 150 mM NaCl, 1% Triton X-100, 0.01% SDS, Protease Inhibitor, PMSF) and transferring to sonication tubes. Samples were sonicated using a QSonica Q800R2 sonicator for 1 hour set at 100% amplitude, with pulse set to 30 seconds on and 30 seconds off. The sonicated lysate was then pelleted at 14,000 RPM in 4° C. and the supernatant was transferred to a reaction consisting of 3.7 ml IP Dilution Buffer, 500 μl Nuclear Lysis Buffer, 175 μl of a 1:1 ratio of ProteinA:ProteinG bead slurry (Thermofisher, 15918014 and 15920010, respectively) and 50 μg of rabbit IgG for preclearing. The preclearing reactions were rotated at 4° C. for 2 hours. 200 μl of the pre-clearing reactions was saved as the “input” control. The remaining solution was added to an immunoprecipitation reaction consisting of 1 ml cold PBS, 20 μl Protein A, 20 μl Protein G, and 1 μl/million cells of either CTCF or H3K9me3 antibody and rotated overnight at 4° C. The immunoprecipitation reactions were prepared one day before cell lysis and rotated overnight at 4° C. The next day, IP reactions were pelleted and the supernatant was discarded. The remaining pellet was washed once with IP Wash Buffer 1 (20 mM Tris pH 8, 2 mM EDTA, 50 mM NaCl, 1% Triton X-100, 0.1% SDS), twice with High Salt Buffer (20 mM Tris pH 8, 2 mM EDTA, 500 mM NaCl, 1% Triton X-100, 0.01% SDS), once with IP Wash Buffer 2 (10 mM Tris pH 8, 1 mM EDTA, 0.25 M LiCl, 1% NP-40/Igepal, 1% sodium deoxycholate) and twice with TE buffer (10 mM Tris pH 8, 1 mM EDTA pH 8). The IP DNA was eluted from the washed beads in Elution buffer (100 mM NaHCO₃, 1% SDS, prepared fresh) by resuspending and then spinning at 7,500 rpm. RNA was degraded with 2 μl RNAse A (Sigma, 10109142001) and incubated at 65° C. for 1 hour. To degrade residual DNA, 3 μl proteinase K (NEB P8107S) was added and all samples were incubated overnight at 65° C. DNA was extracted using phenol:chloroform and ethanol precipitation methods. Antibodies used in this study were: CTCF (Millipore 07-729), H3K9me3 (Abcam ab8898), H3K27ac (Abcam ab4729), H3K27me3 (Millipore 07-449), IgG (Sigma I8140).

Hi-C

Hi-C libraries were prepared using the Arima Genomics Hi-C kit (Arima Genomics, A510008) according to the manufacturer's protocol. Briefly, genomic DNA was enzymatically digested within nuclei of crosslinked cell pellets, and biotinylated ligation junctions were created between the digested ends at proximity. Then DNAs were extracted and sheared to an average size of ˜400 bp using a Covaris S220 sonicator at 140 W peak incident power, 10% duty factor, and 200 cycles per burst for 55 seconds. The sheared DNA were size selected to 200-600 bp using AgenCourt Ampure XP beads (Beckman Coulter, A63881) according to manufacturer's protocols. Biotin-tagged ligation junctions via pulldown using streptavidin breads from the Arima Hi-C kit (Arima Genomics, A510008) according to manufacturer's protocol. Streptavidin beads containing Hi-C libraries were stored at −20° C. for no more than 3 days before Illumina sequencing library preparation was performed.

Chromosome-Conformation-Capture-Carbon-Copy (5C) In Situ 3C

3C libraries were prepared as previously described (Sun et al., 2018, Cell 175, 224-238 e215; Kim ET AL., 2019, Nat Methods 16, 633-639; Kim et al., 2018, Methods 142, 39-46; Beagan et al., 2017, Genome Res 27, 1139-1152; Beagan et al., 2016, Cell Stem Cell 18, 611-624; Phillips-Cremins et al., 2013, Cell 153, 1281-1295). In brief, crosslinked cell pellets were lysed in cell lysis buffer (10 mM Tris pH8.0, 10 mM NaCl, 0.2% (v/v) NP-40) supplemented with 17% (v/v) Protease inhibitor cocktail (Sigma, P8340) in ice for 15 min. Nuclei were isolated by centrifuging cell lysate at 2,500 g for 5 min at 4° C. Pellets were washed once in cell lysis buffer and permeabilized in 0.5% (w/v) SDS at 65° C. for 10 min. SDS was quenched in 6.6% (v/v) TritonX-100 at 37° C. for 15 min. To create 3C ligation junctions, chromatin was digested using 100 U of HindIII in NEBuffer 2 (NEB, B7002S) at 37° C. overnight, then inactivated at 62° C. for 30 min. Digested ends at proximity were ligated using 1,000 U T4 DNA ligase (NEB, M0202S) in 1× T4 DNA ligase buffer supplemented with 0.83% (v/v) TritonX-100 and 0.1 mg/ml BSA at 16° C. for 2 hrs. The reaction was spun down at 2,500 g for 5 minutes, the supernatant was discarded, and the pellet was resuspended in nuclear lysis buffer (10 mM Tris-Hcl pH 8.0, 0.5 M NaCl, 1.0% SDS). Crosslinks were reversed with the addition of 25 μl of 20 mg/ml proteinase K (NEB, P8107) and incubated at 65° C. for 4 hours. An additional 25 uL of Proteinase K was then added and incubated at 65° C. overnight. RNA was degraded in 0.3 mg/ml of RNaseA at 37° C. for 30 min. DNA was extracted with 350 μl phenol:chloroform and precipitated with sodium acetate and ethanol. Excess salt was removed using Amicon Ultra centrifugal filter unit (Millipore, MFC5030BKS).

5C

5C libraries were prepared as previously described (Sun et al., 2018, Cell 175, 224-238 e215; Kim ET AL., 2019, Nat Methods 16, 633-639; Kim et al., 2018, Methods 142, 39-46; Beagan et al., 2017, Genome Res 27, 1139-1152; Beagan et al., 2016, Cell Stem Cell 18, 611-624; Phillips-Cremins et al., 2013, Cell 153, 1281-1295). In brief, previously designed double alternating 5C primers to a 6.4 Mb-sized region around the FMR1 locus (1) were used. 1 fmole of 5C primers were denatured at 95° C. for 5 min and then annealed to 600 ng of 3C template in 1×NEBuffer 4 (NEB, B7004S) at 55° C. for 16 hours. Annealed 5C primers were ligated by 10 U of Taq Ligase (NEB, M0208L) at 55° C. for 1 hour. Ligase was inactivated at 75° C. for 10 min, followed by PCR amplification in PCR mix (5 μl 5× HF buffer, 0.2 1 25 mM dNTP, 1.5 μl 80 μM emusion forward primers, 1.5 μl 80 μM emulsion phosphorylated reverse primers, 0.25 μl Phusion polymerase (NEB, M0530L), 10.55 μl nuclease-free water) in 3 stages: 1 cycle-95° C. for 5 min, 30 cycles—98° C. for 10 s, 62° C. for 30 s, 72° C. for 30 s, 1 cycle—72° C. for 10 min, 4° C. hold. 5C libraries were then prepared for sequencing.

Total RNA-Seq

Total RNA was isolated from NPC and iPS cells using the mirVana miRNA Isolation Kit (Thermo Fisher, AM1560) according to the manufacturer's protocol. 100 ng of isolated RNA was used for RNA-seq library preparation using TruSeq Stranded Total RNA Library Prep Gold (Illumina, 20020598) according to the manufacturer's instruction. In brief, rRNA was removed from the input RNA, followed by double stranded cDNA preparation using 0.8 U of SuperScript II RT (Thermo Fisher, 4376600) and A-tailing end repair. cDNA was ligated to TruSeq RNA Single Indexes Set A (Illumina, 20020492) to enable multiplex sequencing, followed by one round of size selection (selecting for 300 bp) and bead clean-up: 42.5 μL of sample was purified with 42 μL of Agencourt AMPure XP beads (Beckman Coulter, A63881), then, 50 μL sample was cleaned with 50 μL Agencourt AMPure XP beads (Beckman Coulter, A63881). The purified samples were amplified by 15 PCR cycles and further purified using Agencourt AMPure XP beads (Beckman Coulter, A63881). Library quality and quantities were assessed using the Agilent DNA 1000 reagent kit (Agilent, 5067-1504) on the Agilent Bioanalyzer 2100 (Agilent, 5067-4626) and Qubit high sensitivity RNA assay kit (Thermo Fisher, Q32852), respectively before sequencing on NextSeq500 (Illumina).

High Throughput DNA Sequencing Library Preparation

ChIP-seq and 5C libraries were prepared for sequencing using the NEBNext Ultra II DNA Library Prep Kit (NEB #7103) according to manufacturer's protocol. For ChIP-seq and 5C, size selection of adaptor-ligated libraries was performed using AgenCourt Ampure XP beads (Beckman Coulter, A63881) according to the manufacturer's protocol. For 5C, size selection targeted ˜230 bp fragment size and libraries were amplified using 5 PCR cycles. For ChIP-seq, size selection targeted <1 kb fragment size and libraries were amplified using 11 PCR cycles. Input amounts for library preparation using the NEBNext Ultra II DNA Library Prep Kit were 1 ng of purified ChIP-seq libraries and 100 ng of purified 5C libraries. Hi-C libraries were prepped for sequencing by first washing adaptor-ligated Hi-C libraries on streptavidin beads twice in 150 μL of wash buffer at 55° C. and once in 100 ml of elution buffer at room temperature using Hi-C kit (Arima Genomics, A510008). DNA was eluted from streptavidin beads by boiling at 98° C. for 10 min in 15 μL elution buffer. Subsequently the libraries were amplified using NEBNext Ultra II DNA Library Prep Kit for Illumina (NEB, E7645S) with 8 PCR cycles according to the manufacturer's protocol. RNA-seq libraries were prepared for sequencing using the TruSeq Stranded Total RNA Library Prep Gold (Illumina, 20020598).

Sequencing

Prior to sequencing, library quality and size distribution were analyzed with Agilent Bioanalyzer High Sensitivity DNA Analysis Kits (Agilent, 5067-4626) and quantified using Kapa Library Quantification Kit (KAPA biosysytem, KK4835) before sequencing on an Illumina NextSeq 500. ChIP-seq libraries were sequenced with 75 bp single end reads. 5C and Hi-C libraries were sequenced with reading length 37 bp paired end reads. RNA-seq libraries were sequenced with 75 bp paired end reads.

Gene Expression Quantification Using qRT-PCR

Genes of interest were quantified as previously described¹. Briefly, RNA isolation was performed on iPS cells and differentiated neural progenitor cells (NPC) by harvesting cells, snap freezing them in liquid nitrogen, and storing at −80° C. until RNA extraction. 1×10⁶ frozen cells were thawed on ice and total RNA were extracted using mirVana™ miRNA Isolation Kit (Thermo Fisher, AM1560) according to the manufacturer's protocol. RNA was converted into cDNA for each sample using the SuperScript® First-Strand Synthesis System for RT-PCR (Thermo Fisher, 11904018) according to the manufacturer's instruction. 100 ng of RNA was used as input for each sample and RNA was quantified using the Qubit RNA HS assay (Thermo Fisher, Q32852).

To perform qRT-PCR reactions, 2 ml of cDNA was mixed with 10 mM forward and reverse primers, respectively, for a final concentration of 400 nM, in 1× Power SYBR Green PCR Master Mix (Thermo Fisher, 4368706) and the reaction was completed on the Applied Biosystems StepOnePlus Real-Time PCR System (Thermo Fisher, 4376600) according to the manufacturer's instructions. qPCR conditions were 95° C. for 10 min, followed by 40 cycles of 95° C. for 15 s and 65° C. for 45 s. Primer pair specificity was validated by confirming single-peak melting curves at the end of PCR cycles.

For all genes quantified using qRT-PCR (FMR1, SLITRK2, and GAPDH), a standard curve was generated for each gene by amplifying cDNA with gene-specific primers. Standards were created with serial dilutions of 200-0.0002 μM. The resulting CT values of the standards were used to generate a standard curve and compute the absolute concentration of mRNA transcripts per condition using 100 ng of RNA in the cDNA reaction.

### Long Read Sequencing of CGG Repeats

High-Molecular-Weight DNA Preparation.

This protocol was modified from Giesselmann at el (Giesselmann at el., 2019, Nat Biotechnol 37, 1478-1481). Briefly, 1×10e7 hiPSCs were resuspended in 100 μl of 1×PBS. Cells were lysed by adding 10 ml of TLB solution composed of 10 mM Tris-Cl (pH 8), 25 mM EDTA (pH 8), 0.5% SDS (wt/vol) and 20 μg ml-1 RNase A (Sigma) for 1 h at 37° C. Then, proteins were digested at 50° C. for 3 hours using 50 μl of proteinase K (BIO-37084). The viscous solution was transferred into a 50-ml Falcon tube containing 5 g of phase-lock gel and 10 ml of ultrapure Phenol/Chloroform/Isoamyl Alcohol (Fisher) was added. Samples were mixed on a rotator at 40 r.p.m. for 10 min and phase separation was performed by centrifugation at 2,800 g for 10 min. The aqueous phase was then carefully poured into a fresh 50-ml Falcon tube containing 5 g of phase-lock gel followed by a second phase separation using 10 ml of ultrapure Phenol/Chloroform/Isoamyl Alcohol. Samples were mixed and centrifuged as described above. The aqueous phase was poured into a fresh 50-ml Falcon tube, and the genomic DNA was precipitated using 4 ml of 5 M ammonium acetate together with 30 ml of ice-cold ethanol (100%) and gently inverted ten to twenty times. Precipitated DNA was centrifuged at 12,000 g for 5 min and washed with 70% ethanol twice. Supernatant was removed and the DNA pellet was dried at room temperature (RT) for 2-5 min. Rehydration of DNA in 250 μl of 1×Tris-EDTA (pH 8) was performed at RT on a rotator for 20 r.p.m. overnight. Samples were stored at 4° C. for 2 days before use.

Cas9-Targeted Barcoding, Library Preparation, and Long Read Sequencing

To perform targeted sequencing of FMR1, we designed and synthesized CRISPR-Cas9 crRNAs targeting the genomic regions adjacent to the FMR1 CGG repeats with the ChopChop online tool. The crRNAs used are listed in FIG. 37. Preparation of the Cas9 nucleoprotein complex (Cas9 RNPs) was performed as follows: lyophilized Cas9 crRNA and tracrRNA (IDT) were suspended at 100 μM in TE (pH 7.5). The 4 crRNA probes (FIG. 37) were pooled for the cleavage reaction by combining equal volumes of each crRNA probe (0.25 μl/each) and 1 μl tracrRNA (100 μM stock) in 8 μl of water. The pooled crRNAs and tracrRNA were annealed with a thermal cycler at 95° C. for 5 mins, allowed to cool to room temperature, then spun down to collect any liquid in the bottom of the tube. To form Cas9 RNPs (for 10 reactions), components were assembled in a 1.5 ml Eppendorf DNA LoBind tube in the following order: annealed 10 μl crRNA⋅tracrRNA pool (10 μM), 10 μl 10×NEB CutSmart buffer, 79.2 μl Nuclease-free water, 0.8 μl HiFi Cas9 (62 μM, IDT). The tube was mixed thoroughly by flicking. RNPs were formed by incubating the tube at room temperature for 30 mins, then returned to ice until required. Meanwhile, dephosphorylated genomic DNA was prepared by assembling the components in a 1.5 ml Eppendorf DNA LoBind tube in the following order: 5 μg of high molecular weight (HMW) DNA in 24 μl, 3 μl NEB CutSmart Buffer (10×) and 3 μl of QuickCIP enzyme (NEBM0525S). The sample was then incubated in a thermocycler at 37° C. for 20 minutes, 80° C. for 2 minutes, then held at 20° C. (room temperature). The reaction was then mixed gently by flicking the tube, and spun down. 10 μl RNPs from the previous step was incubated with 5 μg of dephosphorylated HMW DNA, 1 μl of 10 mM dATP, and 1 μl of Taq polymerase (NEB) for 60 min at 37° C. on a thermocycler followed by 5 min at 72° C. 1 μl Proteinase K (Sigma, 20 mg/ml stock concentration) was added to each reaction, and samples were incubated at 43° C. for 30 mins to remove proteins for following size selection. The reaction was then purified to remove high concentration salt as follows: Cas9-cut genomic DNA (total volume is 42 μl) was precipitated using 16 μl of 5 M ammonium acetate together with 126 μl of ice-cold ethanol (absolute) and gently inverted ten times. Precipitated DNA was spun down at 16,000 g for 5 min. DNA was washed with 500 μl of 70% ethanol and centrifuged at 16,000 g for 5 min, and this step was repeated two times. The supernatant was removed and the DNA pellet was dried at RT for 2-5 min. Rehydration of DNA was performed at 50° C. for 1 hour using 200 μl of 10 mM Tris-HCl (pH 8). DNA was further homogenized on a rotator at 37° C. and 20 r.p.m overnight. Size selection was then performed with the Bluepippin BLF7510 (sagescience) using the “0.75DF 3-10kb Marker S1” cassette definition with size range at 5-12 kb.

To perform barcode ligation, the following was performed: 3 μl unique barcode (ONT EXP-NBD104) was added to 50 μl of Blunt/TA Ligase Master Mix (NEB) for each sample. The reactions were incubated at RT for 10 min, spun down, and then put on a magnet. The beads were washed with 200 μl of freshly-prepared 70% ethanol, without disturbing the pellet, twice, and allowed to dry for 30-60 seconds. The remaining pellet was then resuspended in 16 μl nuclease-free water and incubated for 10 minutes at room temperature. The reaction was then placed on a magnet and 16 μl of supernatant was removed into a clean 1.5 ml Eppendorf DNA LoBind tube. Samples were then quantified using a Qubit fluorometer, together with the Qubit dsDNA HS assay kit (Thermo Fisher Scientific). Adapters were then ligated by first adding 20 μl NEBNext® Quick Ligation Buffer (NEB #E6056S), 10 μl NEBNext Quick T4 DNA ligase (NEB #E6056S), and 5 μl Adapter Mix (AMII) at room temperature in a separate 1.5 ml Eppendorf DNA LoBind Tube. The ligation reaction was mixed thoroughly. 20 μl of the adapter ligation reaction was mixed with the pooled native barcode-ligated samples. Immediately after mixing, the remaining 15 μl of the adapter ligation mix was added to the native barcode-ligated sample, to yield a 100 μl ligation mix. The reaction was incubated for 10 minutes at room temperature. Then 1 volume (100 μl) of TE (pH 8.0) was added to the ligation mix, followed by 0.4× volume (80 μl) of AMPure XP Beads. The sample was then incubated for 10 minutes at room temperature, placed back on the magnet, and the supernatant was removed. The beads were then washed with 250 μl Long Fragment Buffer (LFB) twice and then air-dried for ˜30 seconds. The library was eluted off the beads in 14 μl Elution Buffer (EB). 13 μL of the library was then mixed with 37.5 μl sequencing buffer (SQB) and 25.5 μl loading beads (LB) and loaded onto the MinION flowcell.

PCR Free Whole Genome Sequencing

For PCR free whole genome sequencing, DNA for samples was extracted using the ThermoFisher GeneJET Genomic DNA Purification Kit (K0721), then sent to GeneWiz for Illumina PCR free, paired end sequencing.

### CGG Over-Expression Experiment

CGGx99 Vector Construction

A vector containing 99 CGG repeats within the FMR1 5′UTR was purchased from Addgene (63091). The CMV promoter in this vector was replaced by EF1a promoter as such: briefly, the CMV promoter in the vector was removed by RI and SalI digestion and replaced with a short fragment that contained two restriction cloning sites SpeI and BsiWI. The short fragment was generated from annealing two short oligos (5′-AATTCACTAGTGAATTCAGATCTGGTACCGTACG-3′ (SEQ ID NO:5); 5′-TCGACGTACGGTACCAGATCTGAATTCACTAGTG-3′ (SEQ ID NO: 6)). The EF1a promoter was isolated from another vector (Addgene, 104372) with NheI and BsiWI digestion and inserted to the CGG vector within SpeI and BsiWI restriction sites and generated the new expression vector EF1a-(CGG)x99-GFP.

CGGx99 Vector Transfection

iPS cells were cultured in a 10 cm dish. CGG vector transfection was carried out with Lipofectamine stem reagent (ThermoFisher, STEM00008) by following the vendor's instruction. 24 hours after transfection, cells were trypsinized and brought to the Children's Hospital of Philadelphia flow core for sorting for both GFP negative and GFP positive cells. Sorted cells were continued in culture for another 24 hours. The cells were then pelleted and used for RT-qPCR and CUT&RUN experiments.

CUT&RUN

CUT&RUN was completed as previously described (Epicypher). In brief, 300 k-600 k iPS cells were washed in phosphate-buffered saline (PBS) and harvested 24 hours after sorting (see: CGGx99 vector transfection). Harvested cells were then washed in wash buffer (20 M Hepes KOH pH 7.5, 150 M NaCl, 0.5 M Spermadine, 1 Roche Complete Protease Inhibitor EDTA-free mini tablet per 10 mL) and bound to Concanavalin A beads (BioMagPlus) that had been activated and washed with binding buffer (20 M Hepes KOH pH 8.0, 10 M KCl, 1 M CaC, 1 M MnC). The cells were then incubated with the Concanavalin A magnetic beads, primary antibody (either IgG (Sigma 18140) or H3K9me3 (Abcam ab 8898), and antibody buffer (digi-wash buffer—0.1% digitonin in wash buffer—with 2 M EDTA) overnight at 4 C. Cells were washed with digi-wash buffer and then incubated in a solution containing protein A-MNase and digi-wash buffer for one hour at 4 C. After incubation, the samples were washed in digi-wash buffer and 100 μL digi-wash buffer was added to the samples which were then placed on an ice block sitting in an ice bath to chill for five minutes. After chilling, 2 L of 100 M CaC was added to activate protein A-MNase chromatin digestion. After 30 minutes, 100 L of 2× stop buffer (340 M NaCl, 20 M EDTA, 4 M EGTA, 0.05% Digitonin, 50 ug/mL RNase A, 50 ug/mL Glycogen) was added to halt the reaction which was then incubated at 37C for 30 minutes to release chromatin fragments. Supernatant was collected and DNA was extracted using phenol-chloroform and ethanol precipitation. The resulting DNA was quantified on a Qubit Fluorometer and NEBNext Ultra II Library Prep Kit was performed using CUT&RUN specific PCR parameters as suggested by EpiCypher CUTANA CUT&RUN protocol to selectively amplify fragments of interest. Fragments were characterized using Qubit and BioAnalyzer. Libraries were pooled and paired-end sequencing was performed using the Nextseq 500 with the Nextseq 500/550 High Output Kit v2 (75 cycles).

Data Analysis

Nanopore Data Processing

All MinION sequencing reads were first processed using the base calling tool guppy_basecaller (Version 4.0.15), then the base called reads were sorted by guppy_barcoder (Version 4.0.15) into each barcoded sample respectively. Reads were then corrected with canu (version 2.1.1) using default parameters. All reads covering the FMR1 locus where the sequencing was done on the reverse orientation were extracted and used for further analysis. Nanopolish (0.13.2) was used to determine CpG methylation over the FMR1 loci from the basecalled long read data using default settings.

PCR Free Whole Genome Sequencing

PCR Free whole genome sequencing libraries were aligned to hg19 using bwa-mem and default parameters.

ChIP-Seq Mapping

ChIP-seq data was processed as previously described (Sun et al., 2018, Cell 175, 224-238 e215). In brief, 75 bp single end reads were mapped to the hg19 reference genome using Bowtie with parameters: --tryhard -m 2. Optical and PCR duplicates were removed using samtools. Reads were downsampled to achieve equal read numbers across samples being compared (FIG. 3). CTCF peaks were called using MACS2 with a cutoff of p<1×10⁻⁸. H3K9me3 domains were called using RSEG (see: H3K9me3 domain calling).

5C

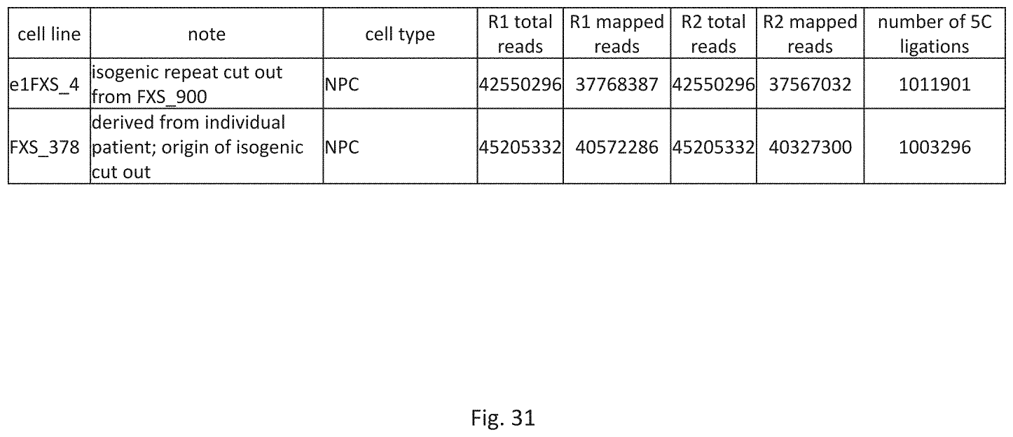

5C data was processed as previously described (Sun et al., 2018, Cell 175, 224-238 e215; Kim ET AL., 2019, Nat Methods 16, 633-639; Kim et al., 2018, Methods 142, 39-46). In brief, 37 bp paired-end reads were mapped to a pseudo-genome consisting of all possible 5C primer ligation junctions with Bowtie using the following parameters: --tryhard and -m 2 and --trim5 6 (FIG. 31). All 5C primer-primer counts were represented as 2-dimensional matrices of interaction frequencies between each pairwise combination of primers. Outlier entries in the matrices, those which were 8-fold greater than the local media of the 5 surrounding entries, were filtered out. The interaction frequency matrices corresponding to samples to be compared were then quantile normalized together. The primer-primer interaction frequencies were then converted to fragment interaction frequencies as described previously (Kim et al., 2018, Methods 142, 39-46). The fragment interaction frequencies were then binned into 4 kb resolution pixels, and a 6 kb smoothing window was applied to attenuate spacial noise. The binned and smoothed matrices were balanced using the ICED algorithm (Imakaev et al., 2012, Nat Methods 9, 999-1003).

Hi-C Data Processing Paired-end reads were aligned independently to the hg19 human genome using bowtie2 (global parameters: --verysensitive -L 30 -score-min L,-0.6,-0.2 -end-to-end --reorder; local parameters: --very-sensitive -L 20 -score-min L,-0.6,-0.2 -end-to-end --reorder) through the HiC-Pro software (Servant et al., 2015, Genome Biol 16, 259). Unmapped reads, non-uniquely mapped reads, and PCR duplicates were filtered and uniquely aligned reads were paired. Raw contact matrices for all samples were assembled into 10kb, 20kb, 40kb, and 100kb non-overlapping bins and balanced using the Knight-Ruiz algorithm. The balanced cis matrixes were then normalized across samples being directly compared using median-of-ratios size factors conditioned on genomic distance (Fernandez et al., 2020, bioRxiv 501056). For trans interactions, because trans interactions are too sparse to quantify at higher matrix resolutions, each trans m×n contact matrix was assembled using Juicer (Durand et al., 2016, Cell Syst;3(1):95-98) by binning hg19 aligned, in situ Hi-C paired-end reads into uniform 1-Mb bins and then balanced using the Knight Ruiz algorithm with default parameters. Data was then quantile normalized across samples.

CUT&RUN Data Processing

Sequencing data was analyzed using Bowtie2 (version 2.2.5) with parameters “--local --very-sensitive-local --no-unal --no-mixed --no-discordant --phred33 -I 10 -X 700”. Duplicates and unmapped reads were removed using Samtools (version 1.11) markdup command. After removing duplicates and unmapped reads, files were converted to bam files using Samtools, and then the resulting bam files were converted to bigwig format using BamCoverage from Deeptools (version 3.3.0). The “--normalizeUsing RPKM -extendReads” parameters for BamCoverage were used.

Gene Expression Analysis RNAA-Seq

RNA-seq reads were mapped to the hg19 ensembl reference transcriptome for both cDNA and ncRNA using kallisto quant (Nicolas et al., 2016, Nature Biotechnology 34, 525-527) with 100 bootstraps of transcript quantification. Reads were mapped to the ensembl cDNA and ncRNA transcriptomes as described in the kallisto documentation. The resulting quantifications were converted into DESEQ2 format, with transcript level counts mapped to gene level counts in R using the library (“tximportData”) according to DESEQ2 (Love et al., 2014, Genome Biology, 15, 550) documentation recommendations. Genes with total counts less than 60 across all samples were dropped from analysis. Differentially called transcripts across the 5 cell lines studied were determined in a pairwise manner using DeSEQ2 LRT with adjusted p<0.005.

H3K9Me3 Domain Calling

H3K9me3 domains were computationally identified using the RSEG program (Song et al., 2011, Bioinformatics. 27(6):870-871). RSEG version 0.4.9. RSEG was run with parameters -s 400000 and with -d deadzone flag, using RSEG provided deadzones for hg19. From the full list of domains calls, domains within 500 KB of centromeres were removed, and then domains located within 10kb of each other using BedTools (VERSION) were merged to get domains >200kb size only. When RSEG domain calls were interrupted by unmappable regions with 0 mapped reads from H3K9me3 ChIP-seq data, the RSEG domains flanking the unmappable region were merged. “Invariant” domains across WT, Pre-mutation, and short and long mutation cell lines were defined as domains present in 4/5 cell lines, where RSEG domain calls had to have boundaries within 300kb of each other to be considered the same domain. Domains “consistently gained” in FXS were defined as domains present in both long mutation cell lines and not present in WT or invariant domains.

Insulation Score Calculation

A 500 kb square window (50×50 bins on 10 KB binned data) with one bin offset from the diagonal was tiled across the genome on Knight-Ruiz balanced cis Hi-C maps on merged and individual replicates for all time points. Counts in the 50×50 bin window were summed, normalized by the chromosome-wide mean, log transformed, and recorded as the Insulation Score (IS).

Dimensionality Index Calculation

To determine the directional bias of the bins corresponding to the genome locations of FMR1, the Directionality Index (DI) was used as described previously (Dixon et al., 2012, Nature, 485(7398):376-380). Briefly, the directionality index is a weighted ratio between the number of Hi-C reads that map from a given 40kb bin to the upstream region and the downstream region. 2 MB upstream and downstream were used in the calculation.

Compartment Identification

To determine A/B compartment status genomewide, the eigenvector of the balanced, 100 KB binned cis Hi-C interaction matrix for each chromosome was calculated as such: The balanced matrix was first normalized by the expected distance dependence mean counts value, followed by removal of rows and columns that were composed of less than 2% non-zero counts. The off-diagonal counts were then z-scored, after which a Pearson correlation matrix for the cis-interaction matrixes was calculated. The eigenvector was the largest eigenvalue of the Pearson correlation matrix. The coordinates corresponding to transitions between positive and negative eigenvector values demarcate boundaries of compartments. To identify which sign corresponds to the A or B compartment for each chromosome, the resulting eigenvectors were correlated with the eigenvector from Lieberman-Aiden et al (Lieberman-Aiden et al., 2009, Science 326: 289-93). In that work, negative values were associated with closed chromatin. In this way, positive values correspond to the A compartment and negative values correspond to the B compartment.

Binning ChIP-Seq Signal Compartment Score

Binned H3K9me3 signal shown in FIG. 1f was generated by taking the H3K9me3 ChIP-seq signal across the loci of interest, splitting the loci into 40 evenly sized bins, and plotting one point for the average ChIP-seg signal of each bin. Similarly, compartment score across the loci of interest in FIG. 1g was calculated by taking the compartment score across the loci of interest, splitting the loci into 40 evenly sized bins, and plotting one point for the average compartment score of each bin.

Binning/Plotting H3K9me3 (FIG. 11)

To plot H3K9me3 domains in heatmap form as in FIG. 11a, each consistently gained, variably gained, or invariant domain (see above: H3K9me3 domain calling) was binned into 100 equally sized bins. The average H3K9me3 ChIP-seq signal in each bin was calculated and plotted. Effectively, this scales all the domains, which are different sizes, to be represented as the same width in the heatmaps. Then, the flanking 100 KB region around each domain was also binned into 100 equally sized bins, and the average H3K9me3 ChIP-seq signal in each bin was calculated and plotted.

Identification of Genes in H3K9Me3 Domains

Genes were defined to be “in” an H3K9me3 domain if the TSS of the gene was contained within the domain. The intersections were performed using BedTools.

Identification of Nested Hierarchy of TADs subTADs

To identify nested TADs, the DI+HMM method was used. The result of using DI window of 15, 25, and 50 were concatenated with goodness of fit with AIC criterion from 1 cluster to 10 clusters.

Determining Interactions Via Hi-C Counts (FIGS. 1k, 1i)

To determine the number of interactions between FMR1 and SLITRK2 as in FIG. 1k, normalized (see section Hi-C Data processing, above), Hi-C data binned at 20 KB resolution was used. The bins corresponding to interactions between the hg19 coordinates of FMR1 and SLITRK2 in the cis chrX interaction matrix were summed to determine the number of interactions between FMR1 and SLITRK2 across conditions. To determine the number of interactions between FMR1 and SLITRK4 as in FIG. 1i, Hi-C data binned at 40 KB resolution was used instead, as this was a much longer range interaction.

Determination of Locations of CTCF Motifs (FIGS. 1m, 1n)

The location of CTCF motifs in hg19 were obtained from the JASPER database using the following parameters: hg19 reference genome, JASPER 2018 consensus, motif: CTCF, allow overlapping motifs, pvalue=0.001, search both strands.

Ideograms and Domain Location

Ideograms for FIG. 18 were retrieved from the UCSC genome browser by using the UCSC Table Browser for hg19, and selecting Group=“All Tables” and Table=“cytoBand”. The location of the red boxes corresponding to gained H3K9me3 domains in FXS were determined by using the UCSC genome browser to locate the coordinates on the ideogram.

Gene Ontology Analysis

Gene ontology enrichment was performed using WebGestalt (Wang et al., 2017, Nucleic Acids Res. 45:W130-W137) (webgestalt.org) with the following settings: Organism of interest=Homo sapiens; Method of interest=overrepresentation enrichment, Functional database=geneontology, biological_process_noRedun. Gene name identifiers were uploaded for each set of classified genes. The genome_protein-coding set was used as the reference set. The enrichment ratios and -log 10p values for all gene ontology terms with an p of <0.01 and enrichment ratio >4 were plotted.

Identification of Genes for Gene Ontology Analysis

The input gene lists for gene ontology analysis (FIG. 11i-11j) were determined as such: In FIG. 11i, all genes which had their TSS reside in a consistently gained H3K9me3 domain in FXS (see above: “H3K9me3 domain calling”), which had expression greater than 0 in at least one of the cell lines where RNA-seq was performed, and which were protein coding (microRNAs and long non coding RNAs were excluded) were input into WebGESTALT. Only protein coding genes were included due to using the genome_protein-coding set as the reference set. Genes for FIG. 11j were selected in a similar manner, starting with genes in variable domains instead of consistently gained domains.

GTEX Tissue Data

Data of gene expression across tissues was obtained from GTEX consortium. The data used for the analyses described in this manuscript were obtained from gtexportal.org/home/datasets on the GTEx Portal on 04/2020. To generate the heatmap in FIG. 11h, the expression of all genes in n=12 consistently gained H3k9me3 domains in FXS was first retrieved. Then, genes which had 0 expression across all tissues were removed, resulting in a final list of n=67 genes. Then, gene expression data was z-scored across tissues (such that strong expression of one gene in one tissue type does not wash out signal in all other tissues). Finally, genes were clustered on the gene expression data using K-means clusters into 4 groups. Clusters were labelled based on the tissue types dominating each cluster.

Location of CGG Repeats in Hg19

Location of CGG repeats in hg19 were identified by string search from the hg19 reference genome. Any strings of more than two CGGs in a row were included in the analysis.

### Example 2: A Noncoding RNA-Based Vaccine for Reversing Pathologic Heterochromatin in Repeat Expansion Disorders

In fragile X syndrome, the long-time dogma is that instability of a single CGG short tandem repeat (STR) tract on the X chromosome represses FMR1 via local DNA methylation. MISHAPS—Megabase Inter-chromosomal interacting domainS of Heterochromatin After Pathologic inStability -were recently discovered in FXS, including ten on autosomes and a 5-8 Mb block encompassing FMR1 on the X chromosome. Nearly all H3K9me3 domains spatially connect via strong inter-chromosomal interactions concurrently with severe misfolding of topologically associating domains (TADs) and loops. Genes co-localized with autosomal H3K9me3 domains are pathologically silenced and encode synaptic plasticity, epithelial integrity, and reproductive development, which are clinical hallmarks of FXS. Unexpectedly, it was observed that overexpression of a noncoding RNA sequence encoding a pre-mutation length CGG tract resulted in full amelioration of all pathologic H3K9me3 domains. Moreover, CRISPR engineering the endogenous mutation-length FMR1 CGG tract to pre-mutation length (180-195 CGG triplets) resulted in de-repression of FMR1 and full reversal of a subset of the Mb-scale FXS H3K9me3 domains. Altogether, the data uncover that mutation-length expansion of the FMR1 CGG in FXS is accompanied by deposition of Mb-sized H3K9me3 domains to silence key synaptic genes on autosomes via inter-chromosomal interactions. Because the H3K9me3 domains are reversible upon delivery of a specific non-coding RNA to the nucleus, the development of RNA-based vaccines for FXS specifically and repeat expansion disorders generally is envisioned. Additionally, pharmacological and ASO-based strategies for the removal of heterochromatin in FXS is pursued. Local chromatin changes and transcriptional silencing have been reported in a number of repeat expansion disorders, therefore therapeutic strategies for the dissolution of heterochromatin-linked trans interactions may be generally applicable to a broad range of diseases outside the brain caused by genome instability.

### Example 3: Spatially Coordinated Heterochromatinization of Unstable Tandem Repeats in Fragile X Syndrome

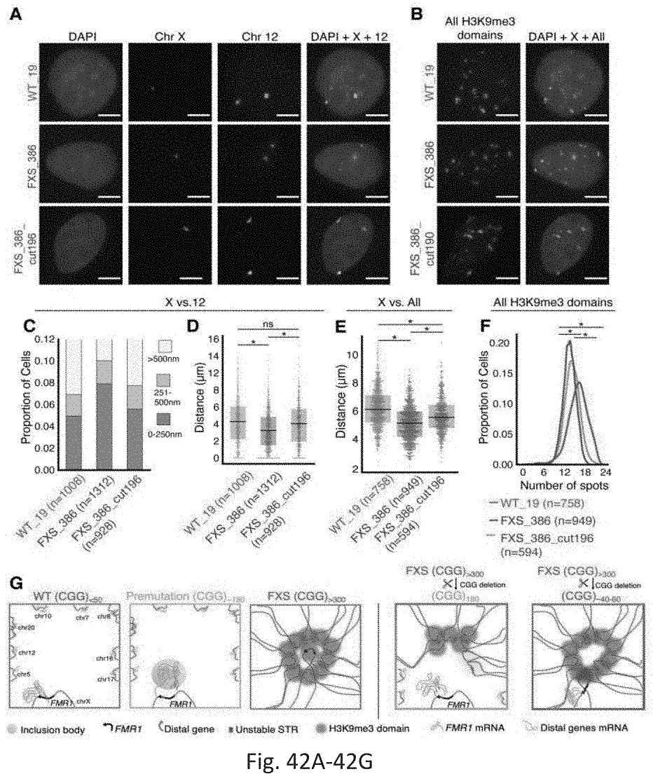

Classic models of FXS assert that the disease is a monogenic disorder in which CGG STR expansion causes local DNA methylation of the FMR1 promoter, leading to transcriptional silencing of FMR1 and loss of FMRP (15-17). Our data support a model of long-range, spatially-coordinated transcriptional silencing in FXS via the CGG-length-dependent acquisition of Megabase-sized domains of the repressive histone modification H3K9me3 on autosomes and the X chromosome (FIG. 42G).

When CGG STRs are normal-length, the FMR1 locus does not connect in trans with distal autosomes (FIG. 42G, panel 1). FMR1 mRNA levels increase as the CGG tract expands to pre-mutation length (FIG. 42G, panel 2). Upon mutation-length expansion, we see local promoter DNA methylation and FMR1 silencing as in traditional models. However, here we also identify many genes distal from FMR1 on the X chromosome and on autosomes which are encompassed by Mb-scale H3K9me3 domains and are repressed in FXS in a manner commensurate with the severity of H3K9me3 signal. Such Mb-scale FXS H3K9me3 domains cluster together spatially in trans, and the TADs, subTADs, and loops present in normal-length iPSC-NPCs are destroyed (FIG. 42G, panel 3). The H3K9me3-silenced genes are linked to synaptic plasticity, testis development, female reproductive system functioning, and epithelial tissue structure, which are known clinical presentations in FXS (34-36). Thus, by way of Mb-scale heterochromatin domains and trans interactions, we find several new candidate genes reproducibly silenced by direct heterochromatinization in FXS.

FMRP directly interacts with mRNA to negatively regulate their translation, and genome-wide disruption of gene expression in FXS has long been considered a secondary consequence downstream of FMRP loss (19). For example, in Fmr1 knock-out mice that lose FMRP but do not have a CGG STR expansion event, excess translation of chromatin readers, writers, and erasers has been linked to transcriptional activation (19), indicating the potential importance of FMRP loss alone in the pathogenesis of FXS. Our work complements these observations because it suggests that in addition to translation dysfunction, there is also direct transcriptional silencing in FXS coordinated by deposition of CGG STR-expansion-dependent H3K9me3 domains. A subset of H3K9me3 domains and trans interactions are dependent on the length of the FMR1 CGG STR, and thus could be coordinated independently of FMRP levels. Moreover, in the intermediate/normal-length CGG cutback experiments, FMR1 is de-repressed, presumably rescuing FMRP levels in FXS iPSCs, however the H3K9me3 domains persist. Future FMRP rescue experiments in our human FXS iPSC lines can be used to dissect the direct role for the CGG STR from the indirect role for downstream translational effects due to FMRP loss on H3K9me3 domains. Our data suggests that heterochromatin-based silencing in FXS would not be modeled only by FMRP loss alone and could not be rescued by simply replacing FMRP in samples with CGG STR expansion events.

A critical question arising from our work is whether engineering the FMR1 CGG STR tract could reverse heterochromatin domains. We use functional endogenous genome engineering with CRISPR to assess the role for the CGG STR tract in H3K9me3 levels. Unexpectedly, upon CGG STR cutout from mutation-length to long-pre-mutation, the X chromosome H3K9me3 domain is attenuated and a subset of distal H3K9me3 domains lose H3K9me3 signal and spatially disconnect from FMR1 (FIG. 42G, panel 4). By contrast, cutback of the CGG STR to intermediate/normal-length does not reverse autosomal heterochromatin domains and distal genes remain repressed (FIG. 42G, panel 5). Only local H3K9me3 signal is removed at the FMR1 promoter, which is consistent with previous reports of FMR1 de-repression upon normal-length cutback (43, 44).

Given that the cut-back to short-pre-mutation length of 100 CGG triplets had variable and partial effects on H3K9me3 domain reversal, our data indicate that the precise long-premutation length of ˜180-190 CGGs is important for reproducible attenuation of the X chromosome H3K9me3 domain in FXS. Overall, our data reveal that H3K9me3 domains on the X chromosome and a subset of autosomes are reversable and exquisitely sensitive to the pre-mutation, but not intermediate/normal CGG STR length.

The mechanism by which the pre-mutation length CGG STR DNA tract or CGG-containing RNA contributes to the establishment, maintenance and reversal of FXS heterochromatinization remains an open question. Mutation-length CGG-containing RNA has been implicated in the establishment of local FMR1 silencing (17), but this study left open the question of what mechanisms maintain FMR1 silencing over the long term. Our work identifies Mb-scale domains of the heterochromatin H3K9me3 modification in the maintenance of gene silencing in FXS on the X chromosome and on autosomes. Our observations bring to light the importance of future studies exploring the mechanistic interplay between long-range heterochromatin mediated silencing and other known molecular phenotypes in FXS, including CGG-RNA-DNA R loops (17, 45, 46), sequestration of specific proteins and the CGG-containing RNA in inclusion bodies (11), repeat-associated non-AUG (RAN) translation of the toxic protein FMRpolyG (12), alternative splicing defects (47), and the downstream effects of FMRP loss (19). The FMR1 CGG STR on the X chromosome is thought to be the only genetic mutation in FXS. Unexpectedly, we identified STR tracts on autosomes which exhibit expansions and contractions unique to our FXS iPSCs and significantly different than the STR length range expected in healthy individuals. Autosomal instability events in our F×S lines are reproducible, but significantly smaller in length than the severe CGG expansion event at FMR1, and thus would have been undetectable until the recent technological advances enabling single-molecule and bp-resolution query of STR lengths. The F×S unstable STRs are enriched in the H3K9me3 domains on autosomes, therefore we hypothesize a model in which critical areas of the genome vulnerable to instability might spatially contact each other to coordinate heterochromatinization when pathways amenable to genome instability are activated in disease. We find that our unstable STR tracts localize to key synaptic genes linked to Autism Spectrum Disorder in case-control studies, including CSDM1 (41) and RBFOX1 (42). Given the parallels between FXS and Autism, our genes containing unstable STRs that are also encompassed by H3K9me3 in our F×S lines may be relevant more broadly to understanding gene expression dysregulation in neurodevelopmental disease.

Altogether our data support a model in which unstable STRs and synaptic genes on autosomes acquire Mb-scale H3K9me3 domains in FXS. Autosomal and X chromosome heterochromatin domains physically contact each other via inter-chromosomal subnuclear hubs, a subset of which can be reversed upon engineering of the mutation-length CGG STR in FMR1 to pre-mutation length. Recently, an independent study reported boundary disruption at the CAG STR in Huntington's disease (48). Local chromatin changes and transcriptional silencing have been reported in a number of repeat expansion disorders, and we hypothesize that heterochromatin-linked trans interactions and TAD/loop dissolution may be generalized principles in diseases with genome instability (48, 49).

The Materials and Methods are Now Described

EBV-Transformed Lymphoblastoid Cell Culture

We cultured EBV-transformed lymphoblastoid cell lines as previously described (50). We grew suspension cells in RPMI 1640 media (Sigma, R8758) supplemented with 2 mM glutamine, 15% (v/v) Fetal Bovine Serum (ThermoFisher 16000044), and 1% (v/v) penicillin-streptomycin (Thermo Fisher, 15140122) at 37° C. and 5% CO₂. We passaged cells every 2-4 days, or when they reached a density of approximately 5×105 cells/ml.

Induced Pluripotent Stem Cell (iPSC) Culture

Prior to arrival, all iPSC lines were expanded, curated, and characterized by Fulcrum's standard operating procedures. At Fulcrum, iPSCs were routinely tested for karyotype instability, FMR1 expression, CGG length, morphology, and pluripotency markers. Upon receipt, we cultured all iPSC lines in mTeSR Plus media (STEMCELL Technology, 05825) supplemented with 1% (v/v) penicillin-streptomycin (Thermo Fisher, 15140122) at 37° C. and 5% CO2 on Matrigel-coated (Corning, 354277) plates for 10-20 passages. We dissociated iPSC by incubating in 5 ml of Versene Solution (Thermo Fisher, 15040066) at 37° C. for 3 min and then deactivated with 10 ml of mTeSR Plus media. All iPSC culture plates were coated with 1.2% (v/v) Matrigel hESC-Qualified Matrix (Corning, 354277) in DMEM/F-12 (Thermo Fisher, 11320033) for at least 1 hr at 37° C.

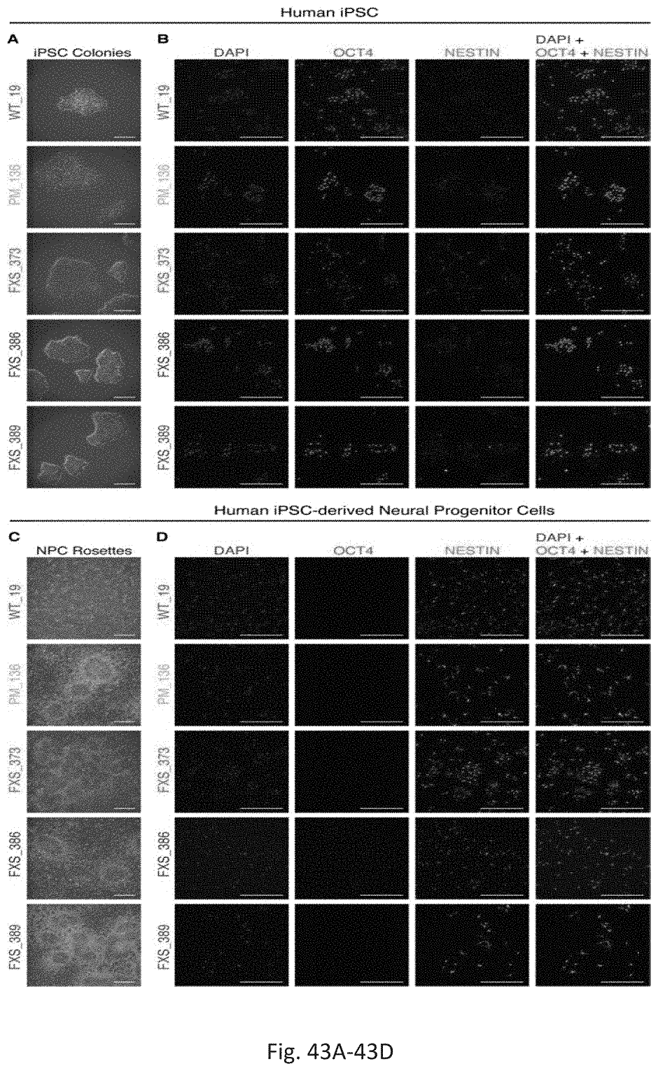

To allow the single-allele evaluation of the CGG STR on the X chromosome, we elected to use male iPSCs in this study. To verify the pluripotency cellular state of our clones, we conducted weekly visual and microscopy assessment of colony morphology and FMR1 expression as well as via immunofluorescence staining for the pluripotency marker OCT4. We used whole genome PCR-free sequencing to confirm that all iPSC lines were karyotypically normal (FIG. 62). We passaged all iPSC lines at 60-70% confluency every 2-5 days to ensure that single colonies remained independent without physical merging (FIG. 43).

iPSC Differentiation to Neural Progenitor Cells (iPSC-NPCs)

We differentiated human iPSC into NPCs using a well-established protocol (51). Briefly, we expanded undifferentiated cells in mTeSR Plus (STEMCELL Technology, 05825) on Matrigel-coated plates as described above. We seeded iPSCs onto fresh Matrigel plates in NPC media at a density of 16,000 cells/cm2. The NPC differentiation medium consisted of DMEM/F-12 (Thermo Fisher, 11320033) with 5 g/ml insulin (Sigma, I1882), 64 μg/ml L-ascorbic acid (Sigma, A8960), 14 ng/ml sodium selenite (Sigma, S5261), 10.7 ug/ml Holo-transferrin (Sigma, T0665), 543 μg/ml sodium bicarbonate (ThermoFisher S233), 10 μM SB431542 (StemCell Tech, 72234), and 100 ng/ml Noggin (R&D Systems, 6057-NG). We changed NPC media every day and harvested cells at the end of day 8. Only iPSC-NPC preparations with the expected rosette morphology and expressing the NPC-specific marker NESTIN were used for downstream genomics and imaging (FIG. 43).

FMR1 CGG Cut-Out Isogenic iPSC Engineering

We generated iPSC lines with CGG tract cut-outs from FXS_371, FXS_373, FXS_386, and FXS_389 iPSC parent lines using CRISPR/Cas9-mediated CGG deletion. We created a custom plasmid expressing Cas9, GFP, and a gRNA targeting the FMR1 5′UTR. To create this plasmid, we modified a previously published plasmid (Addgene #62988) containing Cas9 and a gRNA scaffold as follows: (1) replaced the CMV promoter in Addgene #62988 with an EF1alpha core promoter from Addgene plasmid #12255, (2) added GFP from Addgene plasmid #12255, (3) inserted the gRNA targeted to the FMR1 CGG STR using BbsI restriction digest (sgRNA sequence: 5′-TGACGGAGGCGCCGCTGCCA-3′ (SEQ ID NO: 2)). We verified the correct cloning outcome using the whole-plasmid plasmidosaurus sequencing service.

We transfected iPSCs cultured in Matrigel coated 10 cm dishes in mTeSR plus media with 30 μl of Lipofectamine Stem Transfection Reagent (ThermoFisher, STEM00008) and 15 μg of this custom plasmid according to the manufacturer's protocol. Four days post transfection, the iPSC were dissociated, resuspended in Hank's Balanced Salt Solution (HBSS buffer, ThermoFisher, 14025092) and filtered through a 70 m cell strainer (Corning, 431751) for fluorescence activated cell sorting (FACS) to select for the GFP+ population. Using a MoFlo Astrios cell sorter (Beckman Coulter), we sorted cells into individual wells of a 96-well plate coated with Matrigel. We grew single cells into clonal iPSC colonies in mTeSR Plus medium.

When cells grew into colonies and were ready for passaging, we split each clone into two 96-well plates each, one for screening and one for freezing down and storage.

To screen colonies for successful CGG editing, we extracted DNA from individual clones using QuickExtract™ DNA Extraction Solution (Lucigen QE09050) according to the manufacturer's protocol. We then performed a custom PCR (see below, FMR1 CGG PCR) which amplifies the CGG tract in the FMR1 5′UTR to screen for colonies that had PCR amplicons corresponding to normal, intermediate, or pre-mutation length CGG tracts. Clones that passed this initial screen were regrown from the storage plate by expanding from 96 wells to 12 wells in mTeSR Plus medium on Matrigel-coated plates. We re-screened all expanded clones using the same FMR1 CGG PCR assay to confirm that editing of the CGG tract had occurred. For all clones which passed this second screen and yielded normal, intermediate, or pre-mutation length amplicons, we gel extracted the amplicons using the Qiagen QIAquick Gel Extraction Kit (Qiagen 28706X4) and performed Sanger sequencing using both the forward and reverse PCR primers (Forward primer: 5′-ACGTGACGTGGTTTCAGTGTTTACACC-3′ (SEQ ID NO:26). Reverse primer: 5′-AGCCCCGCACTTCCACCACCAGCTCCT-3′ (SEQ ID NO:27)), utilizing services from the Genewiz company. Sanger sequencing was used to confirm that the amplicons from each clone contained the appropriate base pairs at both the 5′ and 3′ end of the CGG tract, indicating that only CGG STRs were deleted with no additional deletions affecting the FMR1 TSS or 5′UTR. All clones were karyotyped and grown in mTeSR Plus medium on Matrigel-coated plates for 5+ passages before harvesting for downstream assays.

FMR1 CGG PCR

We optimized a custom PCR reaction to amplify the CGGs within the FMR1 5′UTR. This PCR reaction includes additional reagents and extended amplification steps specifically designed to accurately amplify regions of 100% CG content up to 200 CGG triplets (52). The PCR amplification mixture consisted of, for each reaction, 14.5 μl of 2× Advantage GC-Melt Buffer,

0.5 μl of Advantage GC Genomic LA Polymerase (both from the Advantage® GC Genomic LA Polymerase Kit (TakaraBio 639153), 1 μl each of 10 μM forward and reverse primers, and 10 μl of freshly prepared 5M betaine (Sigma, 61962-50G). Samples were amplified with an initial heat denature step of 94° C. for 1 min, followed by 40 cycles of 94° C. for 30 sec, 64° C. for 30 sec and 72° C. for 2 min. After PCR, samples were analyzed by agarose gel electrophoresis. Primers used to amplify the CGGs were: Forward primer: 5′-ACGTGACGTGGTTTCAGTGTTTACACC-3′ (SEQ ID NO:26). Reverse primer: 5′-AGCCCCGCACTTCCACCACCAGCTCCT-3′ (SEQ ID NO:27).

Immunofluorescence Staining

We performed immunofluorescence staining by fixing iPSCs and NPCs using 4% paraformaldehyde for 12 min at room temperature (25° C.). We blocked and permeabilized samples in 0.3% Triton X-100 with 5% BSA in PBS at room temperature. We then incubated fixed cells with primary antibodies overnight at 4° C. in 0.3% Triton X-100 with 1% BSA in PBS followed by incubation with secondary antibodies for 2 hr at RT in 0.3% Triton X-100 with 1% BSA in PBS. Cells were mounted with VECTASHIELD® Antifade Mounting Medium with DAPI (Vector Laboratories, H-1200). The following antibodies were used in this study: rabbit anti-FMRP (1:150, Cell Signaling Technologies, #4317), mouse anti-SHISA6 (1:50, Novus, H00388336-BO1P-50ug), goat anti-rabbit IgG Alexa Fluor 488 (1:200, Thermo Fisher, A-11034), donkey anti-mouse IgG Alexa Fluor 594 (1:250, Thermo Fisher, A-21203), Human Nestin antibody (1:100, R&D Systems, MAB1259), OCT4 (1:200, Cell Signaling, #2740).

Oligopaint DNA FISH Probes

To visualize the twenty-three total loci (10 loci on 2 autosomes each and one locus on the X chromosome) that acquired H3K9me3 heterochromatin in FXS, we used OligoMiner (version 1.0.4) to design Oligopaint probes (53). We designed primary probes across each of N=12 total H3K9me3 domains consistently gained across all three FXS iPSC lines (FXS-consistent H3K9me3 domains). Although N=11 (10 autosomal, 1× chromosome) H3K9me3 domains were reported in FIG. 2, we divided one autosomal domain on chr8 (chr-8R2) into two (chr-8R2a and chr-8R2b) for imaging experiments due to a gap cause by a highly repetitive part of the genome. We ordered probes from Twist Biosciences with the following design features: (i) 80 bases of homology to a DNA sequence unique to a H3K9me3 domain, (ii) a 20 bp fiducial sequence, and (iii) a 20 bp barcode sequence unique to one specific H3K9me3 domain (hereafter referred to as a H3K9me3-locus-specific-barcode, one per each of N=12 domains). We used previously published sequences (54) for our fiducial sequence, 5′-AGTCCCGCGCAAACATTATT-3′ (SEQ ID NO:28), and loci-specific sequences.

We also designed bridge oligonucleotides with the following features: (i) a 20 bp sequence as the reverse complement to the H3K9me3-locus-specific-barcode in the primary Oligopaint probes and (ii) an adjacent 20 bp sequence which can hybridize to the secondary imaging probe. Finally, we designed a secondary fluorescent dye conjugated oligonucleotide imaging probe with a 20 bp sequence representing the reverse complement to the bridge probe (55). We ordered bridge oligonucleotides and dye-conjugated secondary imaging probes from Integrated DNA Technologies (IDT).

We synthesized primary DNA FISH probes from the stock of all Twist probes from all regions pooled at 20 ng/μL using two rounds of PCR as previously described (56). In the first PCR reaction, we used the KAPA HiFi HotStart ReadyMix (Roche, #7958927001), an initial template concentration of 0.04 ng/pL, and primers at a concentration of 0.6 M: F: 5′-ATACGGACGGATCAGGGTAC-3′ (SEQ ID NO: 29) and R:5′-AACGAACTGGCCTTACCAGT-3 (SEQ ID NO: 30), targeting complementary sequences designed for PCR amplification universal to all DNA FISH probes. We implemented a 3 min initial denaturing step at 98° C. and then 20 cycles consisting of 20 seconds of denaturing at 98° C., 15 seconds of annealing at 60° C., 15 seconds of extension at 72° C., concluded by a final extension of 1 minute at 72° C. In the second PCR, we implemented the same settings, but with an amplified template concentration of 0.004 ng/μL and 0.6 μM primers: F: 5′-AGTCCCGCGCAAACATTATTATACGGACGGATCAGGGTAC-3′(SEQ ID NO: 31) and R: 5′-TAATACGACTCACTATAGGGAACGAACTGGCCTTACCAGT-3′ (SEQ ID NO: 32) targeting the complementary sequences designed for PCR amplification universal to all DNA FISH probes. To all DNA probes, the second round of PCR facilitated the addition of (i) a 20 bp fiducial sequence via the forward primer (underlined and italicized above) for the common labelling of all primary probes during imaging and (ii) a T7 promoter sequence via the reverse primer (underlined and italicized above) for subsequent in vitro transcription.

We performed in vitro transcription with an input of 0.75 ng of the amplified primary DNA FISH probe pool using the T7 HiScribe Kit (NEB, E2040S) per manufacturer's instructions. We next performed reverse transcription using the entirety of the in vitro transcribed probe pool RNA produced by the T7 reaction, 2U of Maxima H Minus Reverse Transcriptase (ThermoFisher, EP0751) per 75 μL of reaction, and a custom mix of dNTPs (12.5 mM of dATP, dCTP and dGTP and 6.25 mM of dTTP and amino allyl UTP). After incubation for 2 hr at 50° C., we degraded the RNA:DNA hybrids and excess RNA not converted to cDNA with an alkaline hydrolysis mix (0.25M EDTA, 0.5 M NaOH, and 0.625 μg/μl RNase A), followed by purifying the single-stranded cDNA using a plasmid purification kit (Clontech 740588.250). The single-stranded cDNA probe pool was quantified using a Nanodrop and resuspended in water at a stock concentration of 1.2 μg/μl for imaging.

DNA FISH

We performed Oligopaint DNA FISH as previously described (57) with some modifications for iPSCs. We disassociated iPSC into single cells and plated them on Corning™ Matrigel™ hESC-Qualified Matrix (Fisher Scientific) coated 40 mm glass coverslips (Bioptechs) for 4 hr. We then fixed the samples by incubating the coverslips in 4% formaldehyde and 0.1% Triton in 1×PBS at room temperature. We washed the coverslips three times in 1×PBS for 5 min at room temperature (20-25° C.), and then performed a series of washes at room temperature to prepare the sample for denaturation: (1) a 10 min wash with 0.5% Triton in 1×PBS, (2) a 2 min wash in 70% ethanol, (3) a 2 min wash in 90% ethanol, (4) a 2 minute wash in 100% ethanol followed by 2 min of drying, (5) a 5 min wash in 2×SSCT buffer (0.3 M NaCl, 0.03 M sodium citrate, 0.1% Tween-20 in water), and (6) 5 min wash in a 1:1 mixture of 4×SSCT and 100% formamide. We next incubated coverslips in a 1:1 mixture of 4×SSCT buffer and 100% formamide at 37° C. We diluted 175 pmol of the stock single-stranded Oligopaint probe pool into a final volume of 55 μl of primary hybridization buffer (50% formamide, 10% dextran sulfate, 4% polyvinylsulfonic acid (PVSA) and 0.4 μg/μl RNaseA in nuclease free water) for a final working concentration of 175 μM. We pipetted the Oligopaint probe pool onto 2″×3″ glass slides, placed the coverslips on top, and sealed them with rubber cement. We then heat-denatured the samples by placing the slides on a heat block in a water bath set to 80° C. for 30 minutes. After heat denaturation, we incubated slides in a humidified chamber overnight at 37° C.

The following day, we removed the coverslips from the slides and washed them in (1) 2×SSCT buffer at 60° C. for 15 minutes, (2) 2×SSCT at room temperature for 10 minutes, and (3) 0.2×SSC (0.3 M NaCl, 0.03 M sodium citrate in water) at room temperature for 10 minutes. We used secondary hybridization buffer (50% formamide, 10% dextran sulfate, and 4% polyvinylsulfonic acid (PVSA) in water) to dilute the bridge oligonucleotides and secondary fluorescent dye conjugated imaging probes to final working concentrations of 0.1 μM of each bridge oligonucleotide and 0.2 μM of each secondary dye conjugated imaging probe. As described above, we used a bridge probe and secondary probe unique to each of N=11 FXS-consistent H3K9me3 domains. We pipetted 0.1 μM bridge probes and 0.2 μM secondary imaging probes onto 2″×3″ glass slides, placed the coverslips on top, and sealed them with rubber cement. Slides were incubated in a dark humidified chamber for 2 hr at room temperature. Following the incubation, we removed the coverslips from the slides and washed them in multiple steps: (1) 2×SSCT at 60° C. for 15 min, (2) 2×SSCT at room temperature for 10 min, and (3) 0.2×SSC (0.3 M NaCl, 0.03 M sodium citrate in water) at room temperature for 10 min. To stain nuclei, we incubated coverslips in Hoechst 33342 (1:10,000 in 2×SSC, Thermo Scientific) for five min at room temperature, and subsequently mounted coverslips on 2″×3″ glass slides using SlowFade™ Diamond Antifade Mountant (Thermo Fisher, S36967).

Immunofluorescence and DNA FISH Imaging

We imaged our immunofluorescence and DNA FISH samples on a Leica DMi8 microscope using 10× (phase contrast), 20× (OCT4/Nestin IF), 63× oil-immersion objective (NA 1.4) (DNA FISH), and 100× oil-immersion objective (NA 1.4) (FMRP/SHISA6 IF). We processed the immunofluorescence images with ImageJ (NIH). All DNA FISH images were deconvolved with Huygens Essential deconvolution software v20.04 (Scientific Volume Imaging) using the Classic MLE algorithm with a signal to noise ratio of 40 and 50 iterations (DNA FISH) or signal to noise ratio of 40 and 2 iterations (DAPI stain). We subsequently analyzed our DNA FISH data with TANGO (v0.94) (58). We used TANGO to segment nuclei and perform DNA FISH signal calling using the “Hysteresis” algorithm. We manually curated the segmentation to remove merged multiple nuclei. To measure the distance between the domains on chromosomes X (chrX) and 12 (chr12), we removed nuclei where the number of H3K9me3 domains on chrX and chr12 did not equal one and two respectively, and then took the smallest of the distances between the chrX spot and the two spots representing chr12. For chrX to all domain measurements, we first removed nuclei that that had more than 23 foci (11 autosomal domains * 2+1 domain on chrX), and where the domain on chrX did not co-localize with any of these foci. For the remaining nuclei, we measured the edge-to-edge spatial distance between the spot representing chrX and the spots representing all other distal domains using the “Distance” algorithm in TANGO (border-to-border). We performed two-tailed Mann-Whitney-U tests to evaluate the difference between the distributions of each measurement among the iPSC lines.

Cell Fixation for ChIP-Seq and Hi-C

We fixed cells as previously described for all downstream ChIP-seq, Hi-C, and 5C experiments (1, 4-9). For EBV-transformed lymphoblastoid cells in suspension, we pelleted the appropriate number of cells, resuspended in serum-free RPMI 1640 (Sigma, R8758), and added 1 ml of formaldehydes fixation solution for a final concentration of 1% (v/v) formaldehyde. For adherent iPSC and iPSC-derived NPC, we replaced growth medium with 10 ml DMEM/F-12 (Thermo Fisher, 11320033) and added 1 mL of formaldehyde fixation solution for a final concentration of in 1% (v/v). The stock formaldehyde fixation solution consisted of 50 mM HEPES-KOH (pH 7.5), 100 mM NaCl, 1 mM EDTA, 0.5 mM EGTA, and 11% formaldehyde (Sigma F8775). We quenched the fixation reaction in 125 mM glycine for 5 min at room temperature and 15 min at 4° C. For EBV transformed lymphoblastoid cells in suspension, we pelleted the crosslinked cells. For adherent iPSC and iPSC-derived NPC, we used a cell scraper (Fisher Scientific 02-683-197) to remove crosslinked cells from the dish and then pelleted the cells. We washed the pelleted cells in pre-chilled PBS, flash froze pellets in liquid nitrogen, and stored at −80° C.

Chromatin Immuno-Precipitation and Sequencing (ChIP-Seq)

We performed ChIP-seq as previously described with minor modifications (50, 59-64). Briefly, we lysed crosslinked pellets (consisting of 10 million cells for CTCF ChIP-seq or 3 million cells for H3K9me3 ChIP-seq) in cell lysis buffer (10 mM Tris pH 8.0, 10 mM NaCl, 0.2% NP-40/Igepal, Protease Inhibitor, PMSF) on ice for 10 min. We then homogenized the suspension with pestle A 30 times. We pelleted nuclei at 2,500×g at 4° C. and subsequently lysed them in 500 μl of nuclear lysis buffer (50 mM Tris pH 8.0, 10 mM EDTA, 1% SDS, Protease Inhibitor, PMSF) on ice for 20 min.

We sonicated lysed nuclei in 300 μl IP Dilution Buffer (20 mM Tris pH 8.0, 2 mM EDTA, 150 mM NaCl, 1% Triton X-100, 0.01% SDS, Protease Inhibitor, PMSF) using a QSonica Q800R2 sonicator (settings: 1 hour set, 100% amplitude, 30 seconds pulse, 30 seconds off). After pelleting nuclear membranes at 14,000 RPM and 4° C., we resuspended 800 μl of supernatant-containing chromatin in a pre-clearing solution consisting of 3.7 ml IP Dilution Buffer, 500 μl Nuclear Lysis Buffer, 175 μl of a 1:1 ratio of ProteinA:ProteinG bead slurry (Thermofisher #15918014 and #15920010, respectively), and 50 μg of rabbit IgG on a rotator at 4° C. for 2 hours.

Antibodies used in this study include: CTCF (Millipore, 07-729), H3K9me3 (Abcam, ab8898), H3K27ac (Abcam, ab4729), H3K27me3 (Millipore, 07-449), and IgG (Sigma, I8140). After preclearing, we saved 200 μl as the “input” control and added the remaining solution to an immunoprecipitation (IP) reaction consisting of 1 ml cold PBS, 20 μl Protein A, 20 μl Protein G, and 1 μl/million cells of either CTCF or H3K9me3 antibody and rotated overnight at 4° C. The IP solution was pre-incubated overnight at 4° C. before incubating with chromatin. The next day, we pelleted the IP reactions and discarded the supernatant. We washed the remaining pellet once with IP Wash Buffer 1 (20 mM Tris pH 8, 2 mM EDTA, 50 mM NaCl, 1% Triton X-100, 0.1% SDS), twice with High Salt Buffer (20 mM Tris pH 8, 2 mM EDTA, 500 mM NaCl, 1% Triton X-100, 0.01% SDS), once with IP Wash Buffer 2 (10 mM Tris pH 8, 1 mM EDTA, 0.25 M LiCl, 1% NP-40/Igepal, % sodium deoxycholate), and twice with TE buffer (10 mM Tris pH 8, 1 mM EDTA pH 8). We eluted the IP DNA from the washed beads in Elution buffer (100 mM NaHCO₃, 1% SDS, prepared fresh) by resuspending and then spinning at 7,500 RPM, for a final volume of 200 μL.

We degraded RNA with 60 μg RNase A (Sigma, 10109142001) at 65° C. for 1 hour. We degraded residual protein by incubating the 200 μl solution with 60 μg proteinase K (NEB, P8107S) overnight at 65° C. After extracting DNA using phenol:chloroform and ethanol precipitation, we prepared ChIP-seq libraries for sequencing using the NEBNext Ultra II DNA Library Prep Kit (NEB, #7103) according to the manufacturer's protocol. We performed size selection of adaptor-ligated libraries using AgentCourt Ampure XP beads (Beckman Coulter, A63881), selecting from fragments under 1kb, according to the manufacturer's protocol.

Hi-C

We prepared Hi-C libraries using the Arima Genomics Hi-C kit (Arima Genomics, A510008) according to the manufacturer's protocol. We crosslinked 2 million cells with 1% formaldehyde as described above. Cells were lysed with Lysis buffer (Arima Genomics, A510008) and nuclei were lysed with Conditioning solution (Arima Genomics, A510008). We then enzymatically digested genomic DNA within nuclei of crosslinked cell pellets and created biotinylated ligation junctions between the digested ends according to the manufacturer's protocols. We extracted DNA and sheared to an average size of ˜400 bp using a Covaris S220 sonicator at 140 W peak incident power, 10% duty factor, and 200 cycles per burst for 55 seconds. We further size selected the sheared DNA to 200-600 bp using AgenCourt Ampure XP beads (Beckman Coulter, A63881). Biotin-tagged ligation junctions were pulled down using streptavidin beads from the Arima Hi-C kit according to the manufacturer's protocol. Streptavidin beads containing Hi-C libraries were stored at −20° C. for no more than 3 days before library preparation for sequencing was performed. We prepared Hi-C libraries for sequencing by eluting DNA from streptavidin beads by boiling at 98° C. for 10 min in a 15 μl elution buffer (Arima Genomics, A510008). Subsequently, we amplified the libraries using NEBNext Ultra II DNA Library Prep Kit for Illumina (NEB, E7645S) with 8 PCR cycles according to the manufacturer's protocol.

### Chromosome-Conformation-Capture-Carbon-Copy (5C)

In Situ 3C

3C libraries were prepared as described (50, 59-64). We lysed crosslinked pellets in cell lysis buffer (10 mM Tris pH8.0, 10 mM NaCl, 0.2% (v/v) NP-40) supplemented with 17% (v/v) Protease inhibitor cocktail (Sigma, P8340) on ice for 15 min. We pelleted the remaining nuclei by centrifuging the cell lysate at 2,500×g for 5 min at 4° C. To permeabilize nuclei for in situ restriction digestion of chromatin, we washed the pelleted nuclei once in cell lysis buffer, and incubated nuclei in 0.5% (w/v) SDS at 65° C. for 10 min. We quenched SDS in 6.6% (v/v) TritonX-100 at 37° C. for 15 min. To create 3C ligation junctions within the nuclei, we digested chromatin using 100 U of HindIII in NEBuffer 2 (NEB, B7002S) at 37° C. overnight and then inactivated the restriction enzymes at 62° C. for 30 min. We ligated digested ends in spatial proximity using 1,000 U T4 DNA ligase (NEB, M0202S) in 1× T4 DNA ligase buffer supplemented with 0.83% (v/v) TritonX-100 and 0.1 mg/ml BSA at 16° C. for 2 hrs. We pelleted nuclei at 2,500×g for 5 min, discarded the supernatant, and resuspended the pellet in nuclear lysis buffer (10 mM Tris-Hcl pH 8.0, 0.5 M NaCl, 1.0% SDS). We reversed crosslinks with the addition of 1.7 μg/μl Proteinase K (NEB, P8107) at 65° C. for 4 hrs. We then doubled the concentration of Proteinase K and incubated at 65° C. overnight. We degraded RNA in 0.3 mg/mL of RNase A at 37° C. for 30 min, extracted DNA with phenol:chloroform, and precipitated with sodium acetate and ethanol. We removed excess salt using Amicon Ultra centrifugal filter units (Millipore, MFC5030BKS).

5C

5C libraries were prepared as previously described (50, 59-64). We used previously designed double alternating 5C primers to a 6.4 Mb-sized region around the FMR1 locus (50). We denatured 1 fmole of 5C primers at 95° C. for 5 min and then annealed to 600 ng of 3C template in 1×NEBuffer 4 (NEB, B7004S) at 55° C. for 16 hrs. We ligated annealed 5C primers by 10 U of Taq Ligase (NEB, M0208L) at 55° C. for 1 hr. We inactivated the ligase at 75° C. for 10 min, followed by PCR amplification in PCR mix (5 μl 5× HF buffer, 0.2 1 25 mM dNTP, 1.5 μl 80 μM emulsion forward primers, 1.5 μl 80 μM emulsion phosphorylated reverse primers, 0.25 μl Phusion polymerase (NEB, M0530L), 10.55 μl nuclease-free water) in 3 stages: 1 cycle 95° C. for 5 min; 30 cycles—98° C. for 10 seconds, 62° C. for 30 seconds, 72° C. for 30 seconds; 1 cycle 72° C. for 10 min; and 4° C. hold. We prepared 5C libraries for sequencing using NEBNext Ultra II DNA Library Prep Kit for Illumina (NEB, E7645S) according to the manufacturer's protocol.

Total RNA-Seq

We isolated total RNA from iPSCs and iPSC-derived NPCs using the mirVana miRNA Isolation Kit (Thermo Fisher, AM1560) according to the manufacturer's protocol. All RNA samples had an RNA Integrity Number >9 as assessed by Agilent BioAnalyzer using the RNA 6000 kit (Agilent, 5067-1511). We treated RNA samples with rDNAse I (Ambion, 1906) according to the manufacturer's protocol to remove residual genomic DNA. We used 100 ng of DNAse-treated total RNA for RNA-seq library preparation using the TruSeq Stranded Total RNA Library Prep Gold kit (Illumina, 20020598) according to the manufacturer's instructions. Briefly, we removed rRNA from the input RNA, generated double stranded cDNA using 0.8 U of SuperScript II RT (Thermo Fisher, 4376600), and performed A-tailing and end repair. We ligated the resulting cDNA to TruSeq RNA Single Indexes Set A (Illumina, 20020492) to enable multiplex sequencing. After one round of size selection (selecting for 300 bp) and two rounds of bead clean-up (42.5 μl of sample with 42 μl of Agencourt AMPure XP beads (Beckman Coulter, A63881), we amplified the purified samples using 15 PCR cycles.

CUT&Run

We performed CUT&Run as previously described (65). We harvested 300,00-600,000 iPSC using Versene (ThermoFisher, 15040066) and washed iPSC pellets in phosphate-buffered saline (PBS). We then washed harvested cells in wash buffer (20 M Hepes KOH pH 7.5, 150 μM NaCl, 0.5 μM Spermadine, 1 Roche Complete Protease Inhibitor EDTA-free mini tablet per 10 ml) and bound them to Concanavalin A beads (BioMagPlus) that had been activated with binding buffer (20 μM Hepes KOH pH 8.0, 10 μM KCl, 1 μM CaCl₂), 1 μM MnCl2). We incubated the cells bound to the Concanavalin A magnetic beads in 100 μl antibody buffer (consisting of 0.1% digitonin (Millipore 300410) in wash buffer with 2 μM EDTA) and a final concentration of 1:100 of antibody (either IgG (Sigma, 18140) or H3K9me3 (Abcam ab 8898)) overnight at 4° C. with rotation. Addition of the digitonin at these concentrations reliably permeabilizes the cellular and nuclear membrane without destroying the integrity of either. This allows for diffusion of antibodies, protein A/G-MNase fusion protein, and cleaved chromatin in and out of both membranes in a controlled manner.

We washed cells in digi-wash buffer (0.1% digitonin in wash buffer) and then incubated with 2.5 μl of CUTANA™ pAG-MNase (EpiCypher, #15-1016) in 50 μl digi-wash buffer for 10 min at room temperature. After incubation, we washed the samples in digi-wash buffer and placed them on an ice block sitting in an ice bath to chill for 5 min in 100 μl digi-wash buffer. After chilling, we added 2 μl of 100 μM CaCl₂) and incubated for 30 min to activate the pAG-MNase chromatin digestion. We then added 100 μl of 2× stop buffer (340 μM NaCl, 20 μM EDTA, 4 μM EGTA, 0.05% Digitonin, 50 μg/ml ml RNase A, 50 μg/ml ml Glycogen) and incubated at 37° C. for 30 min to halt the reaction and release chromatin fragments. Samples were placed on a magnet stand to separate immobilized beads and cells from the supernatant containing the cleaved chromatin fragments. We collected the supernatant and extracted DNA using phenol:chloroform and ethanol precipitation. We prepared the library for sequencing using the NEBNext Ultra II Library Prep Kit (NEB, E7645S).

Sequencing

We sequenced all libraries on an Illumina NextSeq 500. Prior to sequencing, we analyzed library quality and size distribution with Agilent Bioanalyzer High Sensitivity DNA Analysis Kits (Agilent, 5067-4626). We quantified library concentration using the Qubit high sensitivity DNA assay kit (Thermo Fisher, Q32852) and the Kapa Library Quantification Kit (KAPA Biosystems, KK4835). We sequenced ChIP-seq libraries with 75 bp single-end reads, CUT&Run and Hi-C libraries with 37 bp paired-end reads, and RNA-seq libraries with 75 bp paired-end reads.

Gene Expression Quantification Using qRT-PCR

We quantified genes of interest as previously described (50). Briefly, we isolated RNA on iPSCs and NPCs by harvesting cells, flash freezing them in liquid nitrogen, and storing at −80° C. until RNA extraction. We thawed 1 million frozen cells on ice and extracted total RNA using the mirVana™ miRNA Isolation Kit (Thermo Fisher, AM1560) according to the manufacturer's protocol. We digested any remaining genomic DNA using rDNAseI (ThermoFisher, AM1906). We quantified RNA using the Qubit RNA HS assay (Thermo Fisher, Q32852) and converted 100 ng RNA into cDNA using the SuperScript® First-Strand Synthesis System for RT-PCR (Thermo Fisher, 11904018) with final concentrations of 500 uM dNTPs, 5 mM MgCl2, 10 mM DTT, and 2.5 ng/μl of random hexamers in the first stranding reaction.

To perform qRT-PCR reactions, we mixed 2 μl of cDNA with 10 uM forward and 10 uM reverse primers for a final concentration of 400 nM, in 1× Power SYBR Green PCR Master Mix (Thermo Fisher, 4368706), and completed the reaction on the Applied Biosystems StepOnePlus Real-Time PCR System (Thermo Fisher, 4376600). Cycle conditions were 95° C. for 10 min, followed by 40 cycles of 95° C. for 15 seconds and 65° C. for 45 seconds. We validated primer pair specificity with single-peak melting curves at the end of PCR cycles. For all mRNA levels quantified using qRT-PCR (FMR1, SLITRK2, SHISA6, DPP6, and GAPDH), we generated a standard curve by amplifying cDNA with gene-specific primers (FMR1: CAAAGGACAGCATCGCTAATGCC (SEQ ID NO:11), GCTCCAATCTGTCGCAACTGCT (SEQ ID NO:12), DPP6: GACCGACAGATGCCTAAAGTGG (SEQ ID NO:13), TGTCGGTGAAGGTTGCTGGCTT (SEQ ID NO:14), SLITRK2: GAGAAATCGTCCAACTCCTCGAG (SEQ ID NO:15), TCTGAGAGGTGCAGACACAGCT (SEQ ID NO:16), SHISA6: GGATGCTTACCGAAGTGGAGGA (SEQ ID NO:17), GGTAACACTGCTCAAAATCGGATG (SEQ ID NO:18), GAPDH: GTCTCCTCTGACTTCAACAGCG (SEQ ID NO:19), ACCACCCTGTTGCTGTAGCCAA (SEQ ID NO:20)). We created standards with serial 10 fold dilutions of cDNA starting at 2 μM. We used the resulting CT values to generate a standard curve and computed the concentration of mRNA transcripts per condition using 100 ng of RNA in the cDNA reaction. We validated the specificity of our amplicons by running the PCR reaction on a gel to verify a single band and confirming a single peak while running a melting curve at the end of each qRT-PCR run.

High-Molecular-Weight (HMW) DNA Isolation for Genome-Wide Long-Read Sequencing

We isolated HMW DNA for genome-wide long-read sequencing using the Gentra Puregene Cell Kit (Qiagen, 158767) with some minor modifications. Briefly, we lysed cells using 1.5 ml of Cell Lysis Solution per 5 million cells, followed by incubation at 37° C. for 1 hour. We then added 10 μl of Proteinase K (provided in the kit) and incubated at 55° C. for 1 hour. We removed RNA by adding 10 μl of RNase A (provided in the kit) and incubating at 37° C. for 1 hour. 500 μl of protein precipitation solution ((provided in the kit) was added to each tube and vortexed for 10 sec. Samples were centrifuged at 12,000×g for 5 min. The supernatant from each tube was added to a new tube containing 1.5 ml of isopropanol and inverted 50 times. We extracted high-molecular weight DNA using a disposable inoculation loop, pelleted the DNA, and washed by dipping into ice-cold 70% ethanol. The DNA pellet was resuspended in 100 μl of elution buffer (Qiagen, 19086). The samples were incubated at 50° C. for 30 min and then at room temperature overnight to allow full resuspension of the DNA. We submitted the resulting HMW DNA to the Cold Spring Harbor Laboratory core facility for genome-wide PCR-free long-read sequencing on a PromethION.

### Nanopore Long-Read Sequencing of CGG Short Tandem Repeat Tract in FMR1

High-Molecular-Weight DNA Preparation for Targeted Long-Read Sequencing

To prepare DNA for targeted long read sequencing at the FMR1 locus, we developed an assay based on previous targeted Cas9 technology development (66, 67). We lysed ˜10 million iPSCs by resuspending in 100 μl of 1×PBS and then adding 10 ml of Tris-Lysis-Buffer solution composed of 10 mM Tris-Cl (pH 8), 25 mM EDTA (pH 8), 0.5% SDS (w/v), and 20 μg/ml RNase A (Sigma, 10109142001) for 1 hour at 37° C. We digested proteins using 1 mg of Proteinase K (Bioline, BIO-37084) at 50° C. for 3 hours. We transferred the solution into a 50 ml Falcon tube containing 5 grams of phase-lock gel and added 10 ml of ultrapure Phenol/Chloroform/Isoamyl Alcohol (Fisher, BP1752I100). We mixed samples on a rotator at 40 RPM for 10 min then centrifuged at 2800 g for 10 minutes. We then poured the aqueous phase into a fresh 50 ml Falcon tube containing 5 g of phase-lock gel and performed a second phase separation using 10 ml of ultrapure Phenol/Chloroform/Isoamyl Alcohol, mixing and centrifuging samples as described above. We poured the aqueous phase into a fresh 50 ml Falcon tube and precipitated the genomic DNA using 4 ml of 5 M ammonium acetate together with 30 ml of ice-cold 100% ethanol and gently inverted ten to twenty times. We centrifuged precipitated DNA at 12,000×g for 5 min, washed with 70% ethanol twice, and dried the DNA pellet at room temperature for 5 min. We resuspended the DNA in 250 μl of 1× Tris-EDTA (pH 8) at room temperature on a rotator at 20 RPM overnight. DNA was stored at 4° C. for up to 2 days before use.

Cas9-Targeted Barcoding, Library Preparation, and Long Read Sequencing

To perform targeted long-read sequencing of FMR1, we designed and synthesized CRISPR-Cas9 crRNAs with the ChopChop online tool (version 3.0.0) using parameters (Target: FMR1, In: Homo sapiens(hg38/GRCh38), Using: CRISPR/Cas9, For: nanopore enrichment) to selectively isolate the FMR1 CGG STR, we designed four crRNAs to specific PAM sequences upstream and downstream of the 5′UTR CGG STR. We ordered 2 nmol of lyophilized customized single-stranded crRNAs (IDT) and 2 nmol of single-stranded tracrRNA (IDT, cat #1072532). We resuspended all RNA to 100 μM in 1× Tris-EDTA (pH 7.5) and created a crRNA-tracrRNA pool consisting of 2.5 μM of each crRNA and 10 μM of the tracrRNA in duplex buffer (30 mM HEPES, pH 7.5; 100 mM potassium acetate). The crRNA and tracrRNAs were annealed to each other via the common complementary sequence by incubating at 95° C. for 5 min and cooling to room temperature.

To assemble Cas9 ribonucleoproteins in vitro, we created a working stock of 1 μM crRNA⋅tracrRNA pool and 0.5 μM HiFi Cas9 by incubating the following on ice for 30 minutes (10 μl crRNA⋅tracrRNA pool (10 μM), 10 μl 10×NEB CutSmart buffer, 79.2 μl Nuclease-free water, and 0.8 μl HiFi Cas9 (62 μM, IDT)). We dephosphorylated genomic DNA by incubating 24 ul of high molecular weight DNA (5 μg), 3 μl NEB CutSmart Buffer (10×), and 3 μl of QuickCIP enzyme (NEBM0525S) at 37° C. for 20 min, 80° C. for 2 min, and 20° C. for 15 minutes.

To specifically cut the target genomic DNA at the FMR1 locus with CRISPR-Cas9 in vitro and dA-tail the cleaved target DNA, we incubated 10 μL of RNPs assembled from the previous step with 30 ul of dephosphorylated high molecular weight DNA (5 μg), 1 μL dATP (10 mM), and 1 μL Taq polymerase (NEB #M0273). We incubated this reaction at 37° C. for 60 minutes to cleave the DNA and produce blunt ended fragments, followed by incubation at 72° C. for 5 min, during which the blunt ends are dA-tailed. To remove protein, we added 1 μl Proteinase K (20 mg/ml, Bioline, BIO-37084) to 42 μl of digested genomic DNA reaction and at 43° C. for 30 min. We purified Cas9-cut genomic DNA (42 μl) with 16 μl of 5 M ammonium acetate together with 126 μl of ice-cold ethanol, spinning down at 16,000×g for 5 minutes, and washing with 70% ethanol. The wash step was repeated 2-3× to remove excessive salts. We removed the supernatant, dried DNA pellet at room temperature for 5 min, and resuspended DNA in 200 μl Tris-HCl (10 mM, pH=8.0) at 50° C. for 1 hr. After incubation on a rotator at 20 RPM overnight, we performed size selection for Cas9-cut DNA with the Bluepippin (Sage Science, BLF7510) using the “0.75DF 3-10kb Marker S1” cassette definition and size range mode at 5-12 kb.

To perform barcode ligation to the DNA library, we added 3 μl of a barcode (Oxford Nanopore Technologies, EXP-NBD104) and 50 μl of Blunt/TA Ligase Master Mix (NEB, M0367) to each sample. We incubated the reactions at room temperature for 10 min and performed cleanup using 50 μl of Agencourt AMPure XP beads (Beckman Coulter, A63881), eluting the library in a final volume of 16 μl nuclease-free water. We quantified samples using a Qubit fluorometer and Qubit dsDNA HS assay kit (Thermo Fisher Scientific, #Q32851).

To prepare the library for sequencing, we used the NEBNext® Quick Ligation Module (NEB #E6056S). We first prepared an adapter ligation solution consisting of 20 μl NEBNext® Quick Ligation Buffer (NEB, #E6056S), 10 μl NEBNext Quick T4 DNA ligase (NEB, #E6056S), and 5 μl Adapter Mix (AMII) (Oxford Nanopore Technologies, SQK-LSK109). We then mixed 20 μl of this adapter ligation solution with the 16 μl barcode-ligated library. Immediately after mixing, we added the remaining 15 μl of the adapter ligation reaction and incubated the reaction for 10 min at room temperature. We added 51 μl nuclease free water for a total volume of 100 μl. We then added 100 μl of TE (pH 8.0) to the ligation mix, followed by 80 μl of AMPure XP Beads. We incubated the sample for 10 min at room temperature, separated the beads using a magnet, and discarded the supernatant. We washed the beads with 250 μl Long Fragment Buffer (Oxford Nanopore Technologies, SQK-LSK109) twice and then air-dried for ˜30 seconds. We eluted the library off the beads in 14 μl Elution Buffer (Oxford Nanopore Technologies SQK-LSK109). Finally, we mixed 13 μl of the library with 37.5 μl sequencing buffer (Oxford Nanopore Technologies SQK-LSK109) and 25.5 μl loading beads (Oxford Nanopore Technologies SQK-LSK109) and loaded the library onto the MinION flowcell for sequencing.

PCR Free Whole Genome Sequencing

We extracted genomic DNA from all iPSC lines using the GeneJet Genomic DNA purification kit (ThermoFisher, #K0721). We used Genewiz for library preparation and sequencing on the HiSeqX platform with 150 bp paired-end reads.

Targeted Nanopore Long-Read Sequencing—Single-Molecule CGG Triplet Counts

We performed base-calling of raw nanopore fast5 using either Guppy (Version 4.4.2+9623c16) or bonito (version 0.3.5a0). We aligned the output files (fastq and fasta, respectively) to hg38 using minimap2 (version 2.21-r1071). We performed several quality-control steps to ensure only high-quality reads were used in downstream analysis: (1) filtering out reads that did not align to the FMR1 gene, (2) using only reads that mapped to the reverse strand because the forward strand cast errors for the ultra-high CG content CGG STR, (3) filtering out truncated reads that did not contain an upstream sequence to the CGG tract “ACCAAACCAA” (SEQ ID NO:21) and at least four consecutive CGGs, 4) removing reads that contained more than nine consecutive “TA” nucleotides within the CGG repeats, as these reflect base calling errors. We then created a custom script to count the number of CGGs in the remaining high-quality reads by finding the first and last instances of the string “CGGCGGCGG”, counting the number of CGGs between them and subtracting five CGGs from the total sum. These five CGGs were excluded because they reflect CGGs located within the FMR1 5′UTR but upstream and external to the continuous CGG tract.

Targeted Nanopore Long-Read Sequencing—DNA Methylation

We called DNA methylation from the Nanopore long-reads using two different methods. We used nanopolish (version 0.13.2) to call methylation in the 19 CpG dinucleotides in the 500 bp FMR1 promoter (chrX:147911419-147911919 (hg38)). Because nanopolish cannot call DNA methylation over a variable number of CGG triplets, we used STRique (version 0.4.2) to call methylation over the CGG tract itself across our normal-length, pre-mutation, and FXS iPSCs.

For the FMR1 promoter, we first indexed the fast5 files using the nanopolish command ‘index’. We called CpG methylation using the command ‘call-methylation’ in the window ‘chrX:147,902,117-147,960,927’. We considered Log 2 likelihood >0.1 as methylated and <−0.1 as un-methylated. For every single-molecule read in every iPSC line, we computed the proportion of 19 CpGs that were methylated.

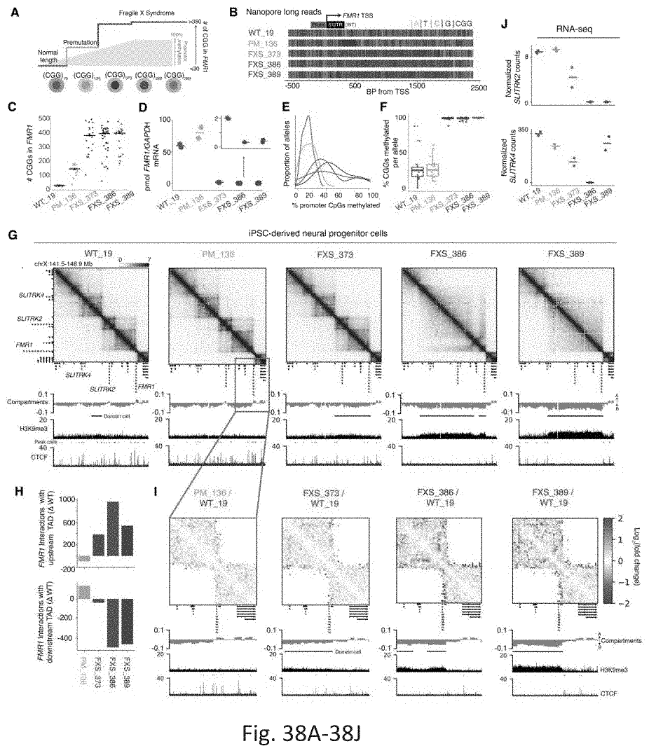

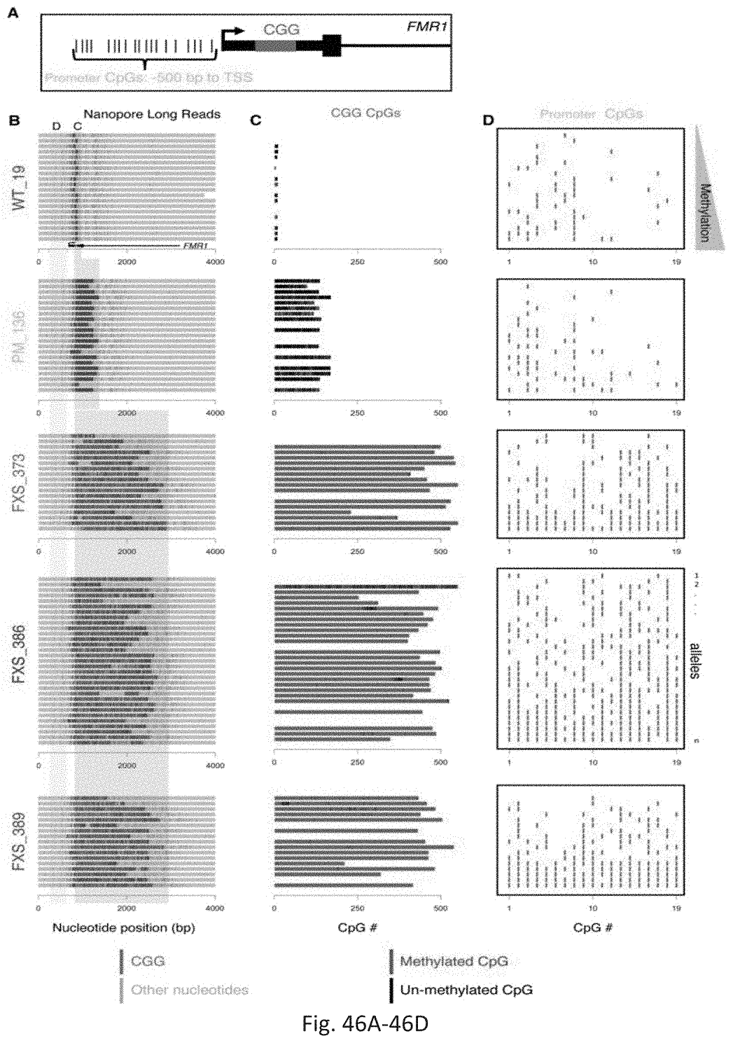

To determine CpG methylation specifically at the CGG STR in the 5′UTR of FMR1, we first indexed the fast5 files using the STRique command ‘index’. We then computed methylation status and CGG counts using the STRique command ‘count’ with the respective models ‘r9_4_450bps_mCpG.model’ and ‘r9_4_450bps.model’. We only used reads with prefix and suffix scores greater than 4 for further analyses as the reads with <4 were of low-quality mapping scores to the upstream and downstream regions of the CGG tract. We calculated the percentage of methylated CpGs over CGG and plotted methylated (1) and unmethylated (0) nucleotides as red and black stripes along the repeats, respectively (FIG. 38, FIG. 46).

PCR-Free Whole Genome Sequencing Read Alignment

For mappability and coverage calculations, we aligned libraries to hg38 using bwa-mem (v0.7.10-r789) and default parameters. Prior to mapping, we checked read quality using FastQC (v0.11.9). We converted the files to the bam format and sorted using Samtools (v1.11) and quality checked the bam files using deeptools (v3.30) and Samtools flagstat before proceeding to downstream analyses.

PCR-Free Whole Genome Sequencing Coverage Calculations

Genome coverage for all iPSC lines was calculated from PCR-free whole genome sequencing data using the published command line tool “goleft indexcov” (version 0.2.3) on aligned bam files with parameters --sex “X,Y”--excludepatt “KI” (68). Copy number variation on all iPSC-NPC lines was calculated using Neoloop (version 0.2.3), a published method to assess genome-wide copy number variation at 5 kb matrix resolution Hi-C map (69). We ran Neoloopfinder (version 0.2.4) with the sub-program calculate-cnv with default parameters on “allValidPairs” output files from HiC-Pro (see: ‘Hi-C data processing’).

ChIP-Seg Mapping

We processed ChIP-seq data as previously described (50, 59-64). Briefly, we mapped 75 bp single-end reads to the hg38 reference genome using bowtie with parameters: --tryhard -m 2. We removed optical and PCR duplicates using samtools (version 1.11). We downsampled reads to achieve equal read numbers across samples. We called CTCF peaks using MACS2 with a cutoff of p<1×10⁻⁸ using input samples as control files. For bigwig visualization, we performed input subtract using deepTools bamCompare with the flag “-o subtract”. We called H3K9me3 domains using RSEG (see: ‘H3K9me3 domain calling’).

Hi-C Data Processing

We aligned paired-end reads independently to the hg38 human genome using bowtie2 (global parameters: --verysensitive -L 30 -score-min L, -0.6,-0.2 -end-to-end --reorder; local parameters: --very-sensitive -L 20 -score-min L,-0.6, -0.2 -end-to-end --reorder) using HiC-Pro version 2.7.7. We filtered out unmapped reads, non-uniquely mapped reads, and PCR duplicates, and paired the remaining uniquely aligned reads. We assembled raw cis contact matrices for all samples into 10kb, 20kb, 40kb, and 100kb non-overlapping bins and balanced using the Knight-Ruiz algorithm. We normalized the balanced cis matrices across all iPSC-NPC lines using median-of-ratios size factors conditioned on genomic distance as we have previously described (70). We assembled trans m×n contact matrices by binning hg38 aligned, in situ Hi-C paired-end reads into uniform 1 Mb-sized non-overlapping bins and balancing using the Knight Ruiz algorithm with default parameters. We quantile normalized trans matrices across samples to facilitate direct comparison.

5C Analysis

5C data was processed as previously described (50, 59-64, 71-73). We mapped 37 bp paired-end reads to a pseudo-genome consisting of all possible 5C primer ligation junctions with Bowtie using the following parameters: --tryhard and -m 2 and --trim5. All 5C primer-primer counts were represented as 2-dimensional matrices of interaction frequencies between each pairwise combination of primers. Outlier entries in the matrices, those which were 8-fold greater than the local media of the 5 surrounding entries, were filtered out. We quantile normalized the interaction frequency matrices from the normal-length and FXS EBV-transformed lymphoblastoid cells. We converted the primer-primer interaction frequencies to fragment interaction frequencies and binned into a 4 kb interaction frequency matrix as described previously (61). We applied a 6 kb smoothing window to attenuate spatial noise and balanced the binned and smoothed matrices using the ICED algorithm.

CUT&Run Data Processing

We analyzed CUT&Run sequencing data using Bowtie2 (version 2.2.5) with parameters “local --very-sensitive-local --no-unal --no-mixed --no-discordant --phred33 -110 -X 700”. We removed duplicates and unmapped reads using Samtools (version 1.11) markdup command. After removing duplicates and unmapped reads, we converted files to bam format files using Samtools. We downsampled mapped reads for IgG and H3K9me3 samples to the lowest number of mapped reads for each comparison group. We converted the resulting bam files to bigwig format using BamCoverage from Deeptools (version 3.3.0) using the “--normalizeUsing RPKM -extendReads -binSize 10 -smoothLength 30” parameters. We input normalized tracks using BamCompare from Deeptools (version 3.3.0) using the “-extendReads -binSize 10 -smoothLength 30 -operation subtract” parameters.

H3K9Me3 Domain Calling

We computationally identified H3K9me3 domains using the RSEG program (version 0.4.9) (74). We ran RSEG with parameters -s 400000 and with -d, deadzone flag, using RSEG deadzone package with default parameters to generate deadzones for hg38. From the full list of domains calls, we removed domains within 500 kb of centromeres, and then merged domains located within 10 kb of each other using BedTools v2.29.2. To focus our analysis on large H3K9me3 domains, we filtered the full list of domains for those greater than 200 kb in size. When RSEG domain calls were interrupted by unmappable regions with 0 mapped reads from H3K9me3 ChIP-seq data, we merged the RSEG domains flanking the unmappable region. We defined “Genotype-invariant H3K9me3 domains” as those present in 4/5 of normal-length, pre-mutation, and full-mutation length FXS iPSC-NPCs, where RSEG domain calls had to have boundaries within 300kb of each other to be considered the same domain. We defined 11 Mb-sized “FXS-consistent H3K9me3 domains” (N=10 on autosomes, N=1 on the X chromosome) as those present in FXS_373, FXS_386, and FXS_389 and not present in WT_19 nor PM_136. We defined Mb-sized “FXS-variable H3K9me3 domains” as those present in only one of the three FXS iPSC-NPCs (FXS_373, FXS_386, and FXS_389) and not present in WT_19 nor PM_136.

RNA-Seg Gene Expression Analysis

We mapped RNA-seq reads to the hg38 ensembl reference transcriptome for both cDNA and ncRNA using kallisto quant with 100 bootstraps of transcript quantification (75) as described in the kallisto documentation. We converted the resulting quantifications into DESEQ2 format and mapped transcript level counts to gene level counts in R using the package “tximportData” according to DESEQ2 documentation recommendations (76). We filtered out genes with total counts less than 60 across all samples from analysis. We normalized data using the DESEQ2 median of ratios based method. We determined differentially called transcripts across the iPSC-NPC lines studied in a pairwise manner using DESEQ2 LRT with adjusted p<0.005.

Insulation Score and Boundary Strength Calculation

To calculate insulation score, we tiled a 200 kb square window (10×10 bins on 20 kb binned data) with one bin offset from the diagonal across the genome on Knight-Ruiz-balanced cis Hi-C maps (77, 78). We then summed, normalized by the chromosome-wide mean, and log transformed counts in the 20×20 bin window to obtain the Insulation Score (IS) of that window. We characterize “boundary strength” within a domain by calculating to difference between the window with the lowest insulation score in the domain and the average insulation score across a 200kb neighboring region.

Directionality Index Calculation

To determine the directional bias of the bins corresponding to FMR1, we calculated the Directionality Index (DI) as described previously (79). Briefly, DI is a weighted ratio between the number of Hi-C reads that map from a given 40 kb bin to the upstream region and the downstream region. We used 2 Mb upstream and downstream regions in the DI calculation.

A/B Compartment Identification

To determine A/B compartment status genome-wide, we calculated the eigenvector of 100 kb Knight-Ruiz-balanced cis Hi-C matrices for each chromosome (80, 81). We first normalized the balanced matrix by the expected distance dependence mean counts value, followed by removal of rows and columns that were composed of less than 2% non-zero counts. We then z-scored the off-diagonal counts and calculated a Pearson correlation matrix for the cis-interaction matrixes. We selected the largest eigenvalue of the Pearson correlation matrix computed from the Hi-C matrix as the eigenvector. The coordinates corresponding to transitions between positive and negative eigenvector values demarcate boundaries of compartments. Using the established pattern of gene density in A/B compartments, we assigned positive eigenvector values to the gene-dense A compartment, and negative values to the gene-poor B compartment.

Binning ChIP-Seq & A/B Compartment Signal

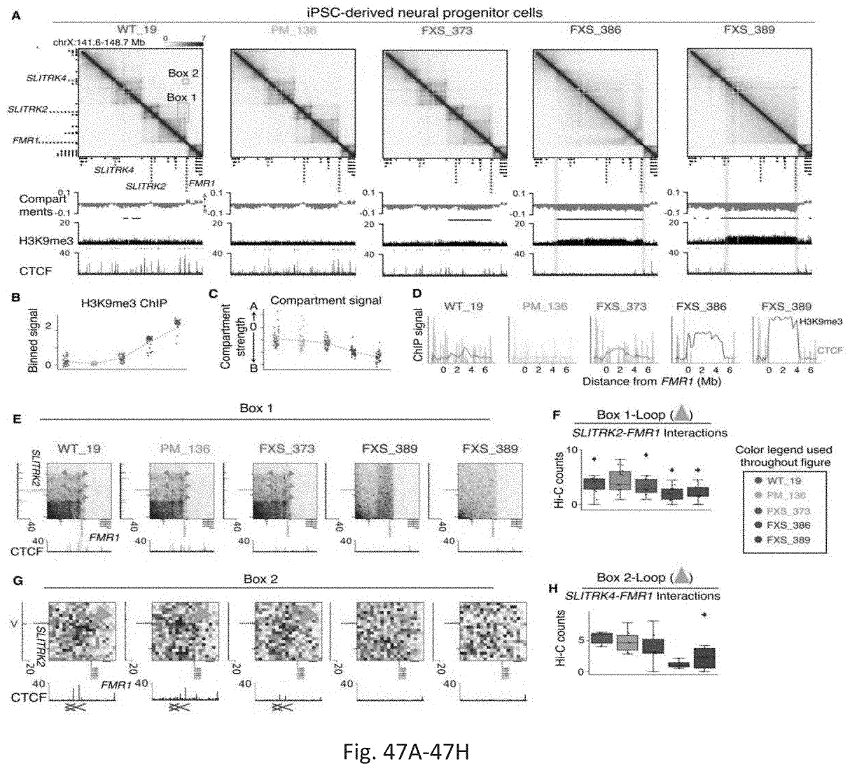

We binned the H3K9me3 signal shown in FIG. 47 by taking the input normalized H3K9me3 ChIP-seq signal across the loci of interest, splitting the loci into 40 evenly sized bins, and plotting one point for the average ChIP-seg signal of each bin. Similarly, we calculated compartment score in FIG. 47 by splitting the locus of interest into 40 evenly sized bins and plotting one point for the average compartment score of each bin. For FIG. 2A, we plotted H3K9me3 signal in heatmap form for “genotype-invariant H3K9me3 domains”, “FXS-consistent H3K9me3 domains”, and “FXS-variable H3K9me3 domains” by binning ChIP-seq signal in each domain into 100 equally sized bins and calculating the average H3K9me3 ChIP-seq signal in each bin. The flanking 100 kb regions around each domain were also binned into 100 equally sized bins, and the average H3K9me3 ChIP-seq signal in each bin was calculated and plotted.

Identification of Genes in H3K9Me3 Domains

We identified genes as co-localized to H3K9me3 domains if the TSS of the gene was contained within the domain. We performed the intersections using the BedTools function ‘intersect’.

Quantifying Long-Range Interaction Frequency Among Key Genes from Hi-C

To determine the interaction frequency between FMR1 and SLITRK2, we used normalized Hi-C data binned at 20 kb and summed the normalized counts in bins corresponding to interactions between the hg38 coordinates of the two genes in the cis X chromosome interaction matrix. To determine the interaction frequency between FMR1 and SLITRK4, we used normalized Hi-C data binned at 40 kb and summed the normalized counts in bins corresponding to interactions between the hg38 coordinates of the two genes in the cis X chromosome interaction matrix.

Hi-C Contact Matrix Difference Maps

To directly compare Hi-C contact matrixes between two iPSC-NPC lines, difference heatmaps were created by taking the log 2 ratio of the two contact matrixes for the region of interest. Any values in the contact matrix that were less than 10 were dropped.

CTCF Motif Identification

We obtained the location of CTCF motifs in hg38 from the JASPER database using the following parameters: hg38 reference genome, JASPER 2018 consensus, motif: CTCF, allow overlapping motifs, pvalue=0.001, search both strands.

Ideograms and Domain Location

We retrieved Ideograms from the UCSC genome browser by using the Table Browser for hg38 and selecting Group=“All Tables” and Table=“cytoBand”. We determined the location of the red boxes corresponding to gained H3K9me3 domains in FXS by using the UCSC genome browser to locate the coordinates on the ideogram.

Gene Ontology Analysis

We performed gene ontology enrichment using WebGestalt (www_webgestalt_org) with the following settings: Organism of interest=Homo sapiens; Method of interest=overrepresentation enrichment, Functional database=geneontology, biological_process_noRedun. We identified gene name identifiers for each set of classified genes and used the genome_protein-coding set as the reference set. We plotted the enrichment ratios and -log 10(p-values) for all gene ontology terms with an p of <0.01 and enrichment ratio >4. All protein-coding genes with TSSs co-localized to “FXS-consistent H3K9me3 domains” or “FXS-variable H3K9me3 domains” or “genotype-invariant H3K9me3 domains” were input into WebGESTALT. Only protein coding genes were included using the genome_protein-coding set as the reference set.

GTEX Gene Expression Data

We obtained gene expression across human tissues from the GTEX consortium. We obtained the data used for the analyses described in this manuscript from https://www.gtexportal.org/home/datasets from the GTEx Portal on 04/2020. To generate the heatmap in FIG. 2, we first retrieved the expression of all genes in n=11 “FXS-consistent H3K9me3 domains”. We removed genes which had 0 expression across all tissues, resulting in a final list of n=68 genes. We then z-scored gene expression data across tissues to ensure that strong expression of one gene in one tissue type does not wash out signal in all other tissues. Finally, we clustered genes on the gene expression data using K-means clusters into 4 groups. We labelled clusters based on the tissue types dominating each cluster.

Identification of FXS H3K9me3 domain as reprogrammed vs resistant to CGG STR editing We categorized FXS specific H3K9me3 domains as either reprogrammed or resistant to CGG deletion based on if the length of the RSEG domain call in the edited iPSC line was less than half the size of that in the parent disease cell line (reprogrammed) or not (resistant).

De Novo Genome Assembly

We constructed de novo assembly using PCR-free WGS as previously described (82). Briefly, we removed any adapter sequences and quality trimmed ends of reads using cutadapt (v 1.18) with parameters “-j 16 -a AGATCGGAAGAGCACACGTCTGAACTCCAGTCA (SEQ ID NO:22) -A AGATCGGAAGAGCGTCGTGTAGGGAAAGAGTGT (SEQ ID NO:23) -q 20,20 --minimum-length 60”. Reads less than 60 bp were removed from further analysis and quality checked using FastQC (v 0.11.9). After filtering reads, we analyzed the k-mer distribution using kat (v 2.4.1). Next, we used W2rapContigger (v 0.1) with parameters “-t 48 -m 600 --min_freq 4 -d 16 -K 136” to create a draft assembly from only raw reads using a 60-mer de bruijn graph and an expanded de bruijn graph up to a k-mer size of 136. Parameters for W2rapContigger were chosen based on our analysis of k-mer distributions and the raw reads. Next, we adapter trimmed, and quality trimmed the ends of our raw Hi-C reads using cutadapt (v 1.18) with parameters “-j 16 -a AGATCGGAAGAGCACACGTCTGAACTCCAGTCA (SEQ ID NO:24)-A AGATCGGAAGAGCGTCGTGTAGGGAAAGAGTGT (SEQ ID NO:25) --nextseq-trim=20 -q 20,20 --minimum-length 10”. We used Juicer (v 1.5) with parameters “-s Arima -p assembly -S early” to map Hi-C reads onto our W2rapContigger draft assembly. We used the output from Juicer and the W2rapContigger draft assembly as inputs to 3D-DNA (v180922) with default parameters. We viewed the output candidate assembly in Juicebox (v 1.11.08), made manual corrections to address assembly errors, and input the edited assembly into 3D-DNA again to finalize the assembly. All sequences over 500kb were extracted as the final assembly. We mapped our final assembly to hg38 and visualized syntenic regions using JupiterPlots (v 3.8.2).

STR Tract Genotyping with GangSTR

We performed STR genotyping on the PCR-free whole genome sequencing data from the N=3 FXS iPSC lines used in our study as well as from three non-diseased populations, including: (i) N˜150 ancestry-matched, European, non-diseased male, PCR-free, blood cell libraries from the 1000 Genomes consortium (83, 84), (ii) N=70 ancestry-, sex-, sequencing depth, and cell type-matched non-diseased individuals from the HipSci Consortium (85), and (iii) N=90 mixed-ancestry, non-diseased, male, PCR-free, blood cell libraries from the ### consortium (83, 84). All lines received nearly half billion reads per sample, with the downsampling target of >400 million equivalently mapped reads per sample.

We aligned PCR-free whole genome sequencing data to hg38 using bwa-mem (version 0.7.10) with default parameters and an additional parameter ‘-M’. We downsampled the aligned reads of comparable sequencing depth (˜400 million reads per sample) and ran GangSTR (version 2.5.0) with the STR input file “hg38_ver13.bed” from GangSTR GitHub page (github_com/gymreklab/GangSTR). Default parameters with one additional parameter declaring sex as males (--samp-sex M) were used. We then filtered out low quality GangSTR predictions by using DumpSTR (version 4.0.0) with the following parameters ‘--gangstr-min-call-DP 12 --gangstr-filter-spanbound-only --gangstr-filter-badCI --gangstr-max-call-DP 1000 -- gangstr-min-call-Q 0.8’. Since DumpSTR was limited by the quality score from a haploid X chromosome, we focused only on the autosomes. The resulting data consisted of an allele-specific STR tract length estimate for more than 800,000 STRs genome-wide in WT_19, PM_136, FXS_373, FXS_386, FXS_389, and N=90 non-diseased samples.

The N=90 PCR free whole genome sequencing samples afforded us the ability to assess the distribution of lengths for a given STR tract across a set of non-diseased individuals. We created nearly 800,000 STR length distributions, one per each STR tract, and employed them as the expected background distribution of lengths for non-diseased individuals. For STRs on autosomes, we used both alleles of each individual in the background distribution. We considered STRs to be candidate “unstable expansions” in our full-mutation FXS iPSC lines if either of the allele lengths, as determined by PCR-free whole genome sequencing, was in the top 6.5th percentile of the 180 alleles (N=90 individuals) non-diseased length distribution in at least one allele 2/3 of our FXS iPSC lines and not in both alleles of our normal-length and pre-mutation iPSC lines. Similarly, we considered STRs to be candidate “unstable contractions” in our full-mutation FXS iPSC lines if either of the allele lengths, as determined by PCR-free whole genome sequencing, was in the bottom 6.5th percentile of the 180 alleles (N=90 individuals) non-diseased length distribution in at least one allele 2/3 of our FXS iPSC lines and not in both allele of our normal-length and pre-mutation iPSC lines. Thus, we identify a candidate list of STRs on autosomes that exhibit evidence of reproducible expansion or contraction in our FXS iPSC lines and not in our normal-length or pre-mutation iPSC lines.

To test the hypothesis that the autosomal unstable STRs in our FXS iPSC lines are enriched in our FXS H3K9me3 domains, we formulated our null and alternative hypotheses as:

>>Ho: The proportion of FXS H3K9me3 domains co-localized with an autosomal unstable Zhou et al FXS STR is no different than the proportion found in size-matched random intervals

>>Ha: The proportion of FXS H3K9me3 domains co-localized with an autosomal unstable Zhou et al FXS STR is greater than the proportion found in size-matched random intervals

We defined an STR as “colocalized” if it was located within an H3K9me3 domain where the domains were expanded to include their 300 kb flanking region. We formulated an empirical statistical test in which we randomly sampled N=10 size-matched genomic intervals without replacement and computed a test statistic of the proportion of intervals co-localized with an F×S unstable STR tract. We resampled 10,000 times, generating a distribution of the proportion of intervals co-localized with an F×S unstable STR tracts under the assumption that the null hypothesis is true. We then computed the same test statistic using our N=10 FXS H3K9me3 domains and computed a one-tailed empirical P-value as the percentage of the null distribution that is greater than or equal to the test statistic in our N=10 FXS H3K9me3 domains. We repeated the randomization test n=100 times and report the average P-value obtained over these 100 iterations. P<0.05 was considered statistically significant. We also repeated the statistical test using random sampling of size-matched, genotype-invariant H3K9me3 domains, with similar results. Finally, we repeated this statistical test using an additional test statistic to assess for STR localization with domain boundaries: the proportion of intervals whose boundary regions (defined as the +/−350kb flanks of each domain) contained an STR.

Statistics Overview

FIG. 38E: For every single-molecule read in every iPSC line, we computed the proportion of 19 CpGs that were methylated. We compared the distributions of single-molecule DNA methylation using a one tailed Mann-Whitney U test (FIG. 38, FIG. 46).

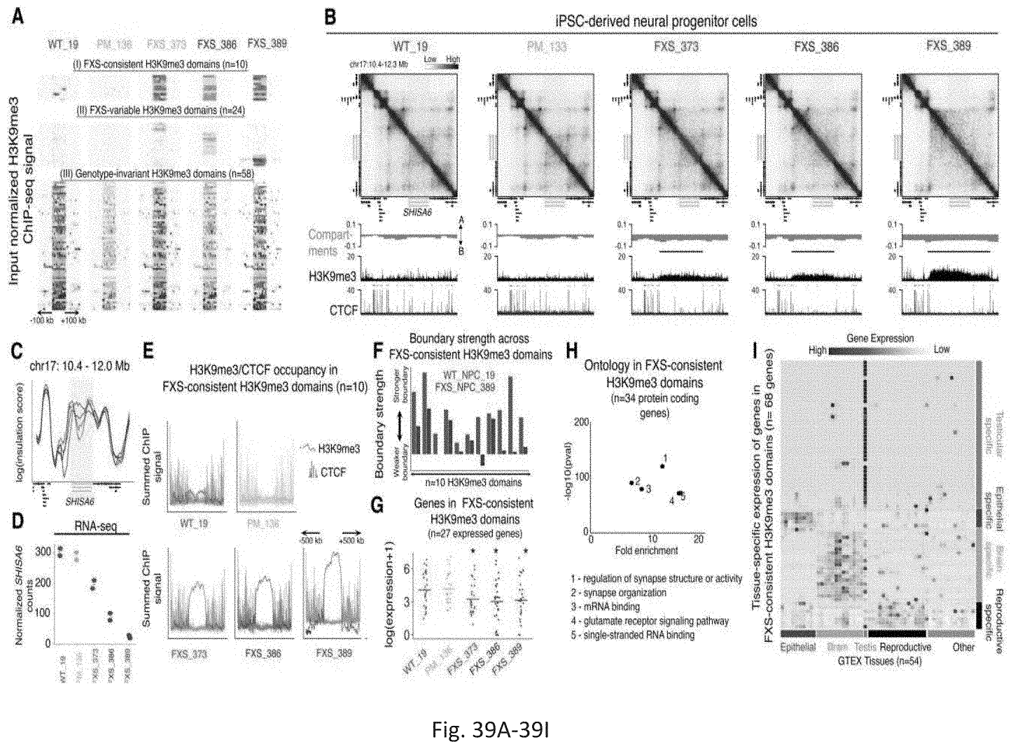

FIG. 39: We generated RNA-seq counts using DEseq (see above) across all iPSCs for each of the n=27 expressed protein-coding genes in FXS H3K9me3 domains. We averaged counts across biological replicates per condition and log-transformed the averages after adding a pseudocount. We compared the distribution of log transformed counts for PM 136, FXS_373, FXS_386, and FXS_389 to WT_19 using a non-parametric, one-tailed Mann Whitney U test. An alpha value of 0.05 was selected a priori, and P-values were as follows: PM_136 vs WT_19: 0.4313 (not significant) FXS_373 vs WT_19: 0.0346 (significant) FXS_386 vs WT_19: 0.0233 (significant) FXS_389 vs WT_19: 0.0449 (significant)

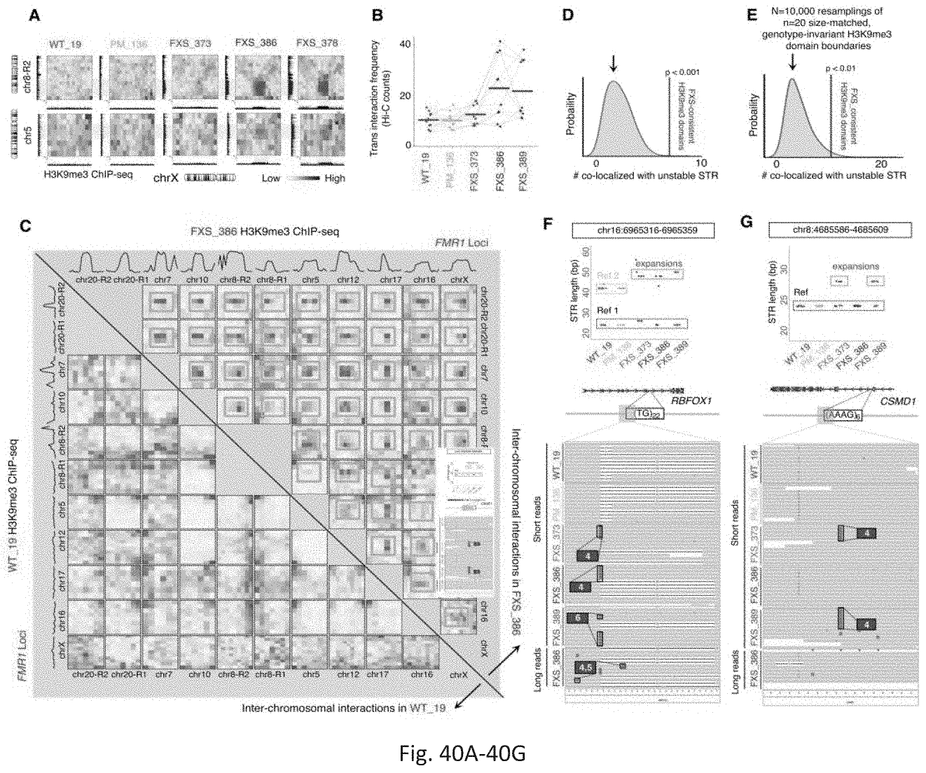

FIG. 40: We developed an empirical randomization test to assess the enrichment of unstable STRs in FXS H3K9me3 domains. A one-tailed empirical P-value was computed as detailed in ‘STR tract genotyping with GangSTR’.

FIG. 42: We performed non-parametric, two-tailed Mann-Whitney-U tests to evaluate the difference between the distributions of each DNA FISH measurement among the different iPSC lines. In FIG. 42D, we plot the distances between the H3K9me3 domain on chromosome X and the H3K9me3 on chromosome 12 from individual nuclei: WT_19_iPSC (N=1008, mean: 6.94 m, IQR: 4.00-9.77 m), FXS_386_iPSC (N=1312, mean: 5.55 m, IQR: 2.94-7.80 m) and FXS_386_cut190_iPSC (N=928, mean: 6.68 m, IQR: 3.89-9.22 m). In FIG. 42E, we plot the average distance between the H3K9me3 domain on chromosome X and all other H3K9me3 domains from individual nuclei: WT_19_iPSC (N=758, mean: 6.33 m, IQR: 5.33-7.22 m), FXS_386_iPSC (N=949, mean: 5.29 m, IQR: 4.35-6.08 m) and FXS_386_cut190_iPSC (N=594, mean: 5.82 m, IQR: 4.96-6.55 m). In FIG. 42F, we plot kernel density estimate plots of the number of individual foci (green in B) from the Oligopaints probes for all twelve H3K9me3 domains in WT_19_iPSC (N=758), FXS_386_iPSC (N=949) and FXS_386_cut190_iPSC nuclei (N=594).

| | | | P value from |
|---|---|---|---|
| | | | two-tailed |
| | | iPSC lines | Mann-Whitney-U |
| Plot | Measurement | compared | tests |
| 42D | distance between | WT_19 vs FXS_386 | 2.34856e−18 |
| | the H3K9me3 domain | FXS_386_cut190 | 5.78003e−12 |
| | on chromosome X | vs FXS_386 | |
| | and the H3K9me3 | WT_19 vs | 0.055089 |
| | on chromosome 12 | FXS_386_cut190 | |
| 42E | average distance | WT_19 vs FXS_386 | 5.14363e−51 |
| | between the | FXS_386_cut190 | 1.27527e−14) |
| | H3K9me3 domain | vs FXS_386 | |
| | on chromosomeX | WT_19 vs | 7.16146e−13 |
| | and all other | FXS_386_cut190 | |
| | H3K9me3 domains | | |
| 42F | number of | WT_19 vs FXS_386 | 1.23424e−88 |
| | individual all | FXS_386_cut190 | 7.19643e−13 |
| | H3K9me3 foci | vs FXS_386 | |
| | | WT_19 vs | 4.99803e−36 |
| | | FXS_386_cut190 | |

The Experimental Results are now described

Severe Genome Misfolding and Acquisition of a Mb-Scale H3K9Me3 Domain Upon Full-Mutation CGG STR Expansion

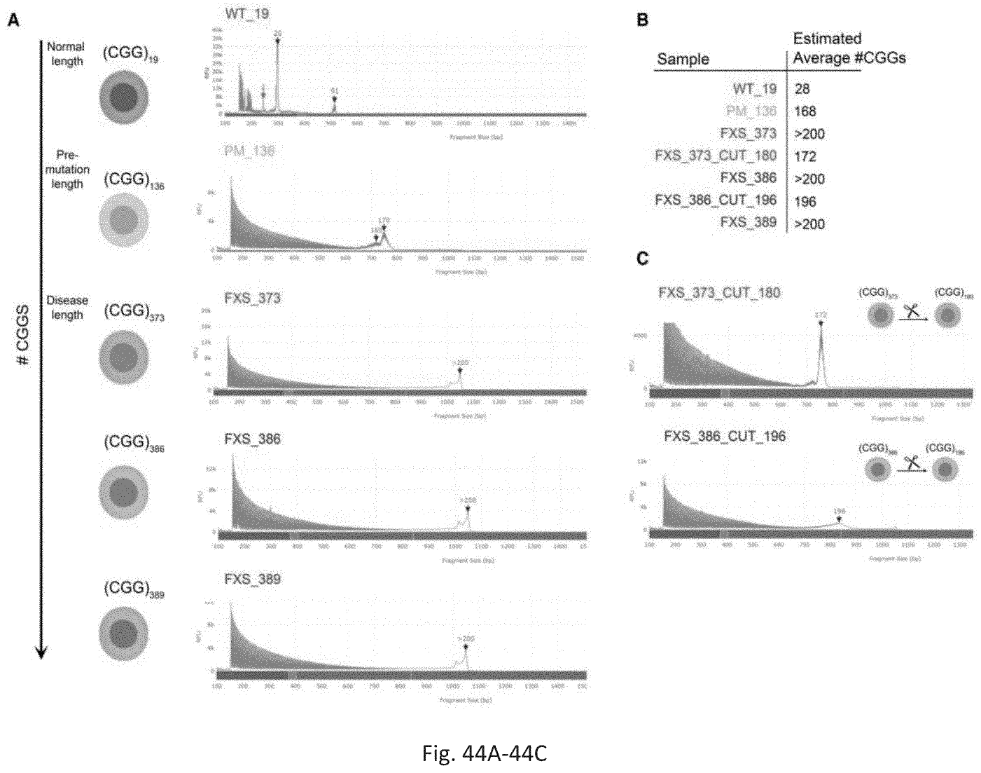

We previously reported misfolding of the topologically associating domain (TAD) boundary around FMR1 in lymphoblastoid cell lines and post-mortem brain tissue from FXS patients with a 450+ CGG STR expansion (26), suggesting that silencing might occur via long-range chromatin mechanisms beyond local DNA methylation. Here, we investigate the extent to which higher-order chromatin folding and the repressive histone modification H3K9me3 is altered genome-wide upon expansion of the CGG STR across a range of tract lengths. We analyzed a series of human induced pluripotent stem cell (iPSC) lines in which the CGG STR tract expands from normal-length (5-40 GG) to pre-mutation (61-199 CGG) and full mutation-length (200+ FXS Replicates 1, 2, 3) (FIG. 38A and FIG. 43A). Using an established clinical-grade PCR assay, we confirmed the CGG tract length status on bulk cellular populations (FIG. 44).

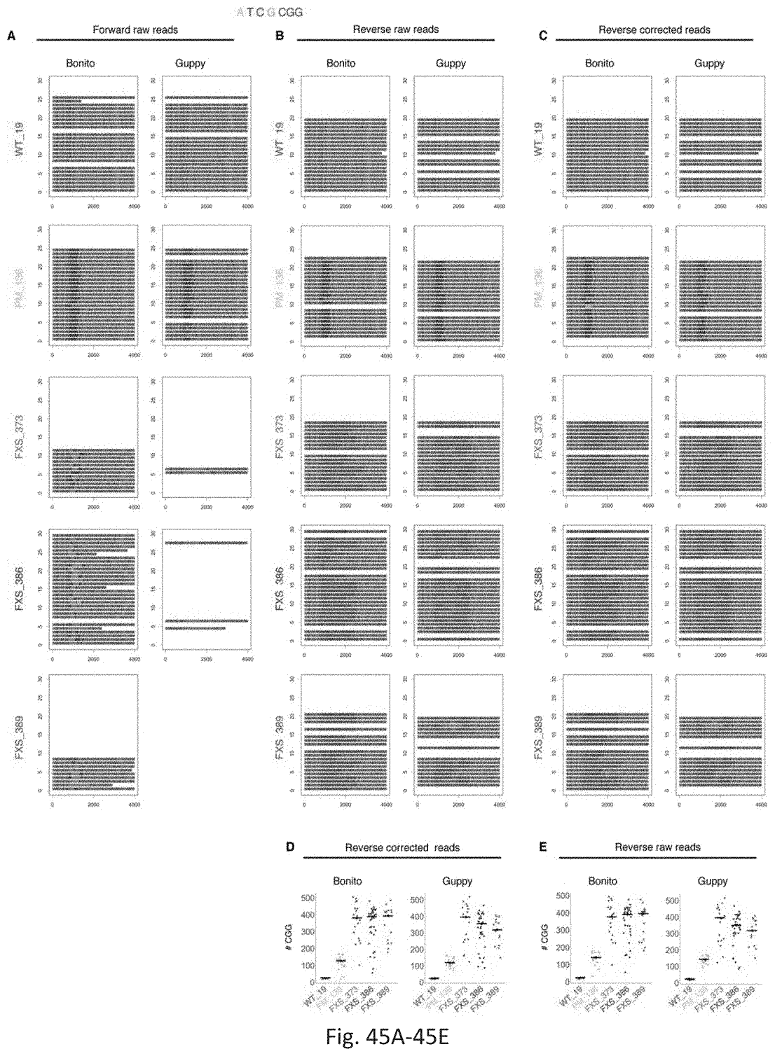

To obtain precise estimates of CGG STR length, we developed a customized assay coupling Nanopore long-read sequencing with guide RNA-directed Cas9 cutting around the transcription start site and 5′UTR of the FMR1 gene (FIGS. 38B-E and FIG. 45) (31). Consistent with previous reports, normal-length and pre-mutation iPSCs had on average 19 and 136 CGG triplets, respectively (FIG. 38B-C). All three independent full mutation-length iPSC lines showed a similar average of -370-380 CGG triplets and thus represent three biological replicates of FXS (FIG. 38B-C). CGG tract lengths were similar using Guppy and Bonito base callers and with or without read correction (FIG. 45). Consistent with previous reports (8), we observed that FMR1 mRNA increased upon CGG expansion to pre-mutation length and decreased significantly in all three F×S lines (FIG. 38D). Concomitant with decreased FMR1 mRNA, we observed DNA methylation at the promoter and CGG tract in all three F×S lines (FIG. 38E-F, FIG. 46). Thus, using single-molecule Nanopore long reads, we have precisely estimated CGG tract length and verified known molecular hallmarks of FXTAS and FXS in our iPSC lines, including depleted DNA methylation and increased FMR1 mRNA levels in pre-mutation iPSCs, as well as local DNA methylation and FMR1 silencing in three independent lines with full-mutation CGG expansion.

To study folding patterns of the 3D genome in FXS, we differentiated our iPSC lines to homogenous populations of neural progenitor cells (iPSC-NPCs) (FIG. 43B) and generated genome-wide high-resolution Hi-C libraries. We observed severe genome misfolding in all three full mutation-length CGG expansion FXS iPSC-NPCs, including the dissolution of TADs, subTADs, and loops for up to 8 Megabases (Mbs) upstream of the ˜1200 bp CGG STR (FIG. 38G and FIG. 47A). We also observed destruction of the local TAD boundary at FMR1 (FIG. 38H-I, box 1 FIG. 47A) as we previously reported in lymphoblastoid cell lines and post-mortem brain tissue using targeted Chromosome-Conformation-Capture-Carbon-Copy (5C) analysis (26). Thus, chromatin misfolding is severe in FXS and encompasses many more Mb of the X chromosome than only the FMR1 CGG STR.

To gain insight into the underlying mechanisms governing genome misfolding, we used ChIP-seq to map genome-wide patterns of the repressive histone mark H3K9me3 and the architectural protein CTCF. We observed a striking acquisition of H3K9me3, and signal was not only local to FMR1 as in previous reports (32). H3K9me3 spread in a domain-like pattern 5-8 Mb upstream FMR1 in all three mutation-length FXS iPSC-NPC lines (FIG. 38G-I). Upon gain of H3K9me3 in FXS, we observed loss of occupancy of the majority of CTCF sites (FIG. 38G, FIG. 47A-E). Boundaries of the Mb-scale H3K9me3 domain co-localize with the limits of genome misfolding (FIG. 38G, FIG. 47A). These results indicate that heterochromatin spreads 5-8 Mb upstream of FMR1 and correlates with large-scale misfolding of the genome on the X chromosome in FXS.

H3K9me3 extends 5-8 Mb upstream of FMR1 to silence essential synaptic genes in FXS

FXS is characterized by defects in synaptic plasticity and cognitive ability (33). We noticed that the FXS H3K9me3 domain spanned two additional genes, SLITRK2 and SLITRK4, linked to neuronal cell adhesion and synaptic plasticity (FIG. 38G). Using our Hi-C maps, we observed that FMR1 loops directly to SLITRK2 and SLITRK4 in normal-length and pre-mutation-length iPSC-NPCs (FIG. 47F-I). The long-range gene-gene cis interactions are abolished and SLITRK2 and SLITRK4 mRNA levels are decreased as H3K9me3 spreads over the locus in FXS (FIG. 38J, FIG. 47F-I). We note that the H3K9me3 domain does not encompass the promoter of SLITRK4 in FXS_389 iPSC-NPCs, and the gene is not silenced in this line. Moreover, SLITRK2 and SLITRK4 are only partially repressed in FXS_373 iPS-NPCs with lower H3K9me3 signal intensity, further emphasizing the likely role for H3K9me3 in distal gene silencing in FXS (FIG. 38I). Together, these data suggest that a H3K9me3 domain radiates outward from FMR1 to encompass and silence additional synaptic and neural cell adhesion genes in FXS.

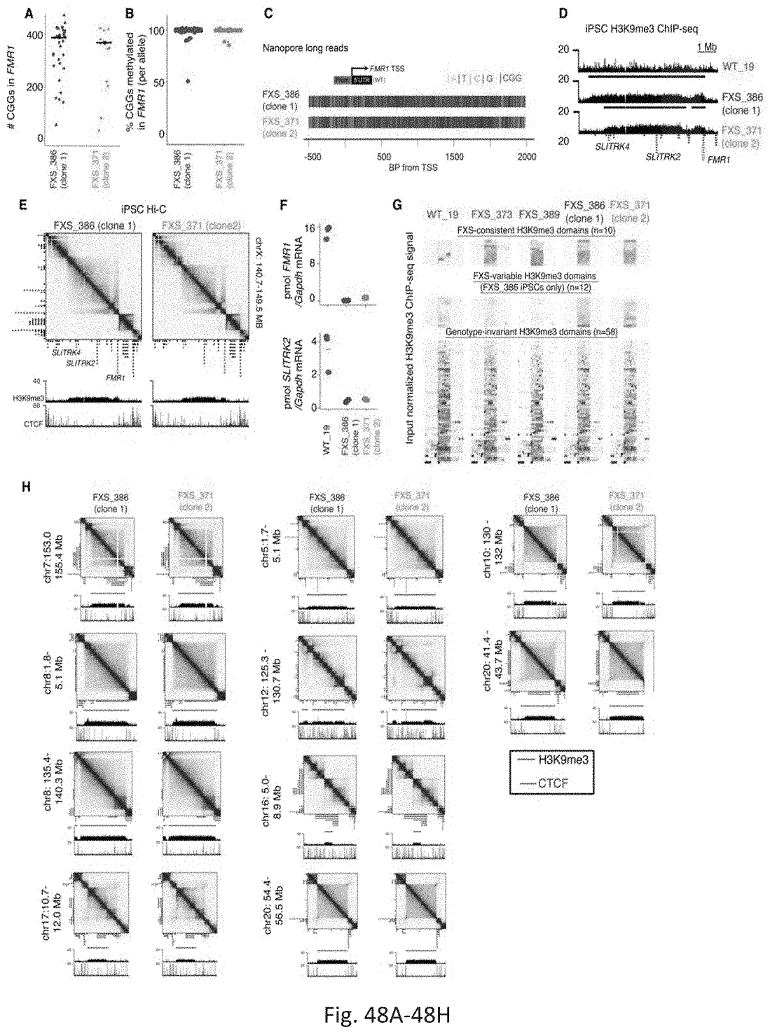

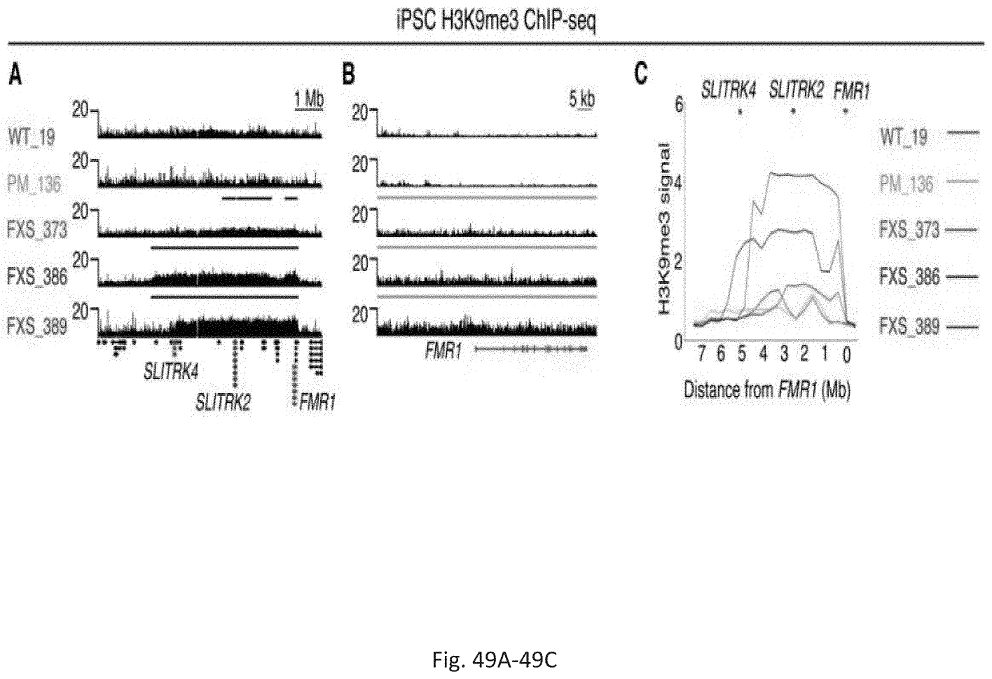

We tested if large-scale genome misfolding and heterochromatin silencing around the FMR1 locus would vary by cellular state or in subclones from the same parent line. We derived a second iPSC line, FXS_371, from the parent line FXS_386, and we observed similar CGG tract length, STR DNA methylation, genome misfolding, and H3K9me3 signal (FIG. 48). To test the role for cellular state, we created H3K9me3 ChIP-seq and Hi-C libraries in pluripotent iPSCs and EBV-transformed lymphoblastoid B-cell lines. We observed similar H3K9me3 deposition patterns in iPSCs and iPSC-NPCs from the same genetic backgrounds (FIG. 49). In lymphoblastoid B-cell lines with a normal-length CGG tract, the SLITRK2/4 genes are already silenced and FMR1 lowly expressed. Therefore, in mutation-length FXS B-cells there is only slight repression of FMR1 from already low expression levels (FIG. 50A). Consistent with gene expression patterns, the X chromosome H3K9me3 domain already encompasses silenced SLITRK2/4 in normal-length B-cells, and it spreads downstream to encompass and further silence FMR1 upon CGG expansion (FIG. 50B-D). It is noteworthy that at the location of H3K9me3 domain spread over FMR1, the TAD boundary and local CTCF occupancy is disrupted in FXS B-cells (FIG. 50B, E-F). These data suggest that H3K9me3 silencing in FXS will be most severe in cell types which strongly express SLITRK2, 4, and FMR1 and lack a pre-existing H3K9me3 domain in their normal-length state.

Mb-Scale H3K9Me3 Domains are Acquired on Autosomes in FXS

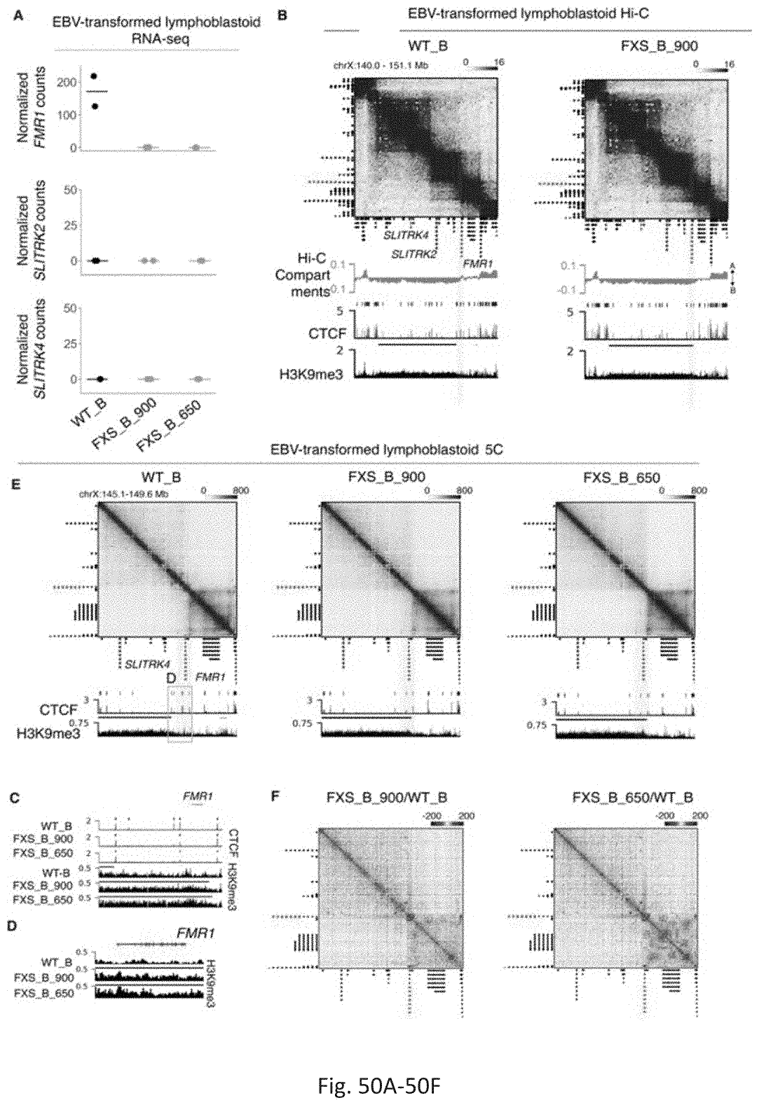

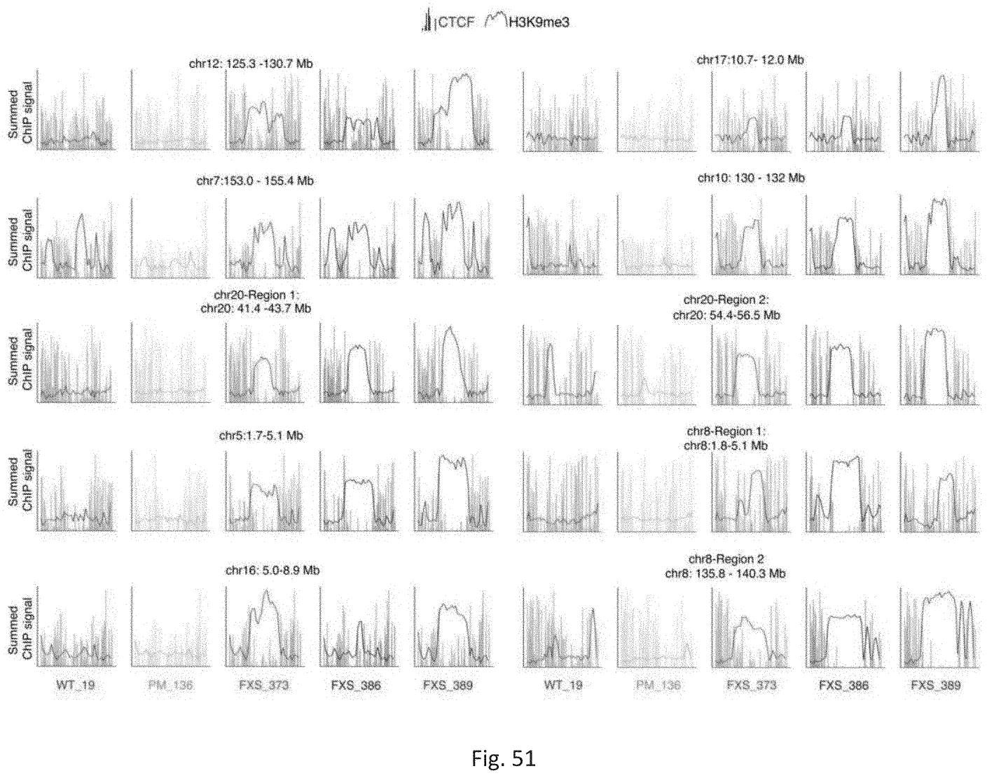

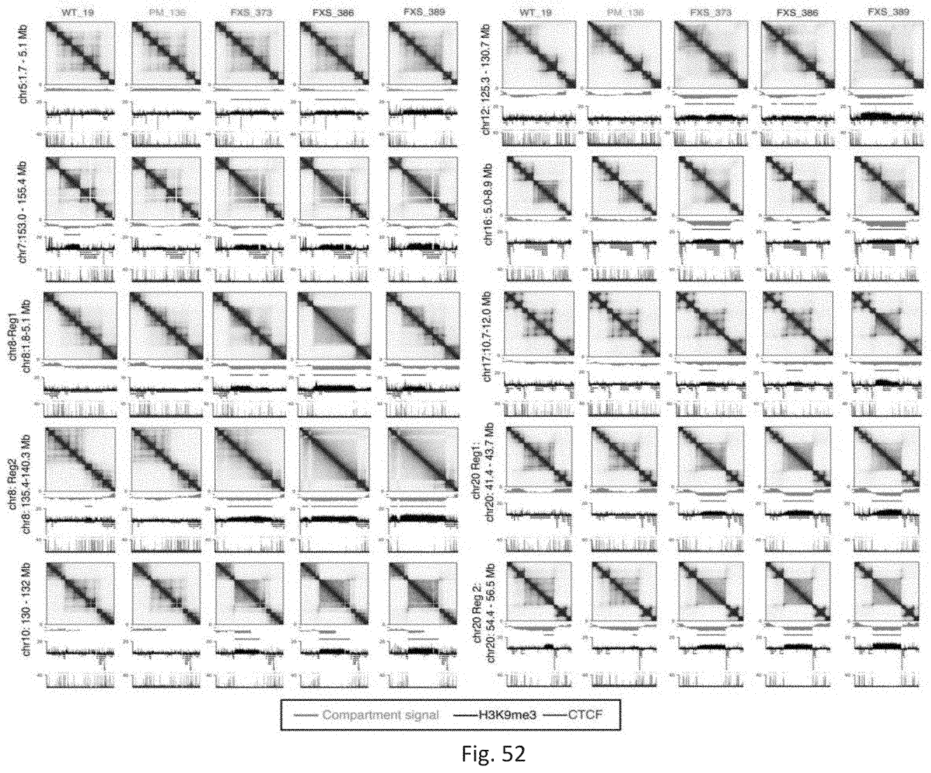

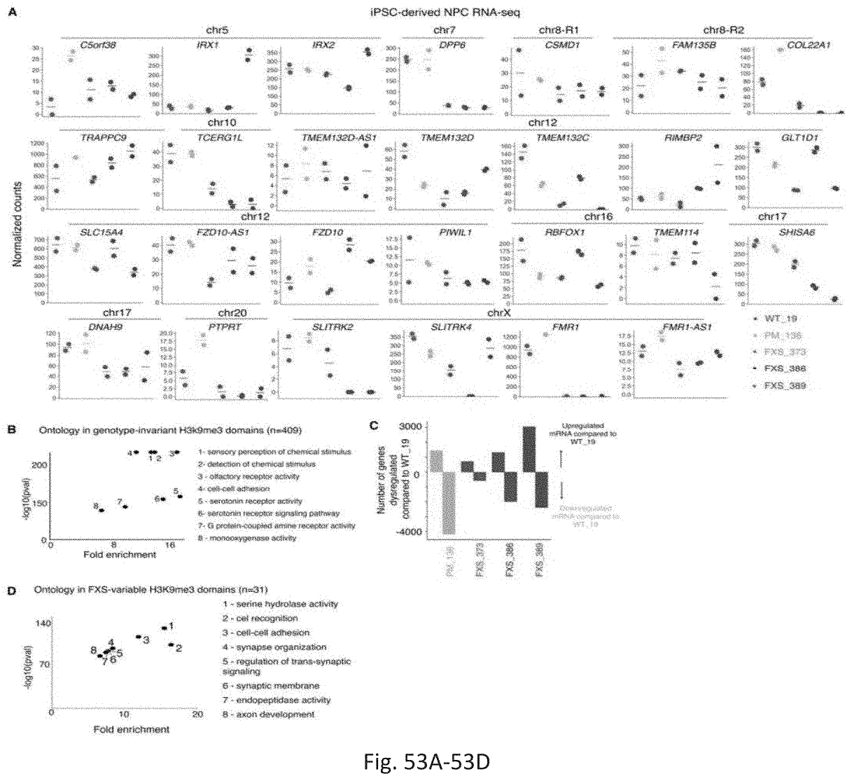

We unexpectedly identified ten additional genomic locations on autosomes in which large (>1 Mb) H3K9me3 domains were acquired in all three of our mutation-length FXS iPSC-NPCs (FIG. 39A and FIG. 51). This observation is particularly unexpected given that the CGG STR expansion event driving FXS is on the X chromosome. One such domain encompasses the synaptic gene SHISA6 located within a known fragile site on chromosome 17 (FIG. 39B). Similar to the broader FMR1 locus, we observe H3K9me3 deposition, TAD ablation, and loss of CTCF occupancy on chr17 in all three F×S lines (FIG. 39B, C). SHISA6 mRNA levels decrease proportionately to the intensity of the H3K9me3 signal (FIG. 39D). In aggregate for all 10 autosomal FXS domains, we observed loss of CTCF occupancy (FIG. 39E, and FIG. 51), TAD boundary disruption (FIG. 39F and FIG. 52), and a marked reduction in gene expression (FIG. 39G, FIG. 53A). Ontology analysis indicated that genes in FXS H3K9me3 domains are linked to synaptic plasticity and neural cell adhesion, and such gene classes are not enriched in genotype-invariant H3K9me3 domains (FIG. 39H, FIG. 53B). We note that although we see both gain and loss of expression genome-wide in FXS (FIG. 53C), the synaptic genes in our iPSC-NPC H3K9me3 domains are largely downregulated (FIG. 39G and FIG. 53A). We also identified H3K9me3 domains present in only one F×S line (so-called FXS-variable H3K9me3 domains, FIG. 39A). Genes co-localized with FXS-variable H3K9me3 domains were also enriched for synaptic and neural cell adhesion ontology (FIG. 53D). Together, our data suggest that Mb-scale H3K9me3 domains are present on autosomes and encompass repressed synaptic genes in FXS, which is of particular interest given the synaptic and cognitive defects reported in FXS patients (34).

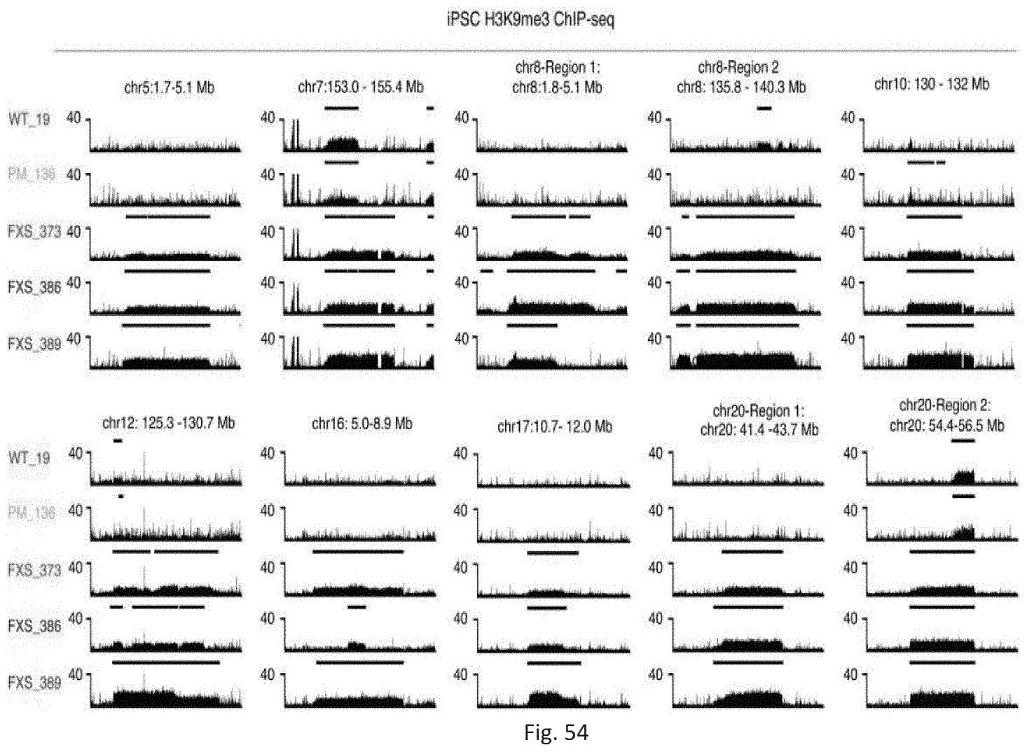

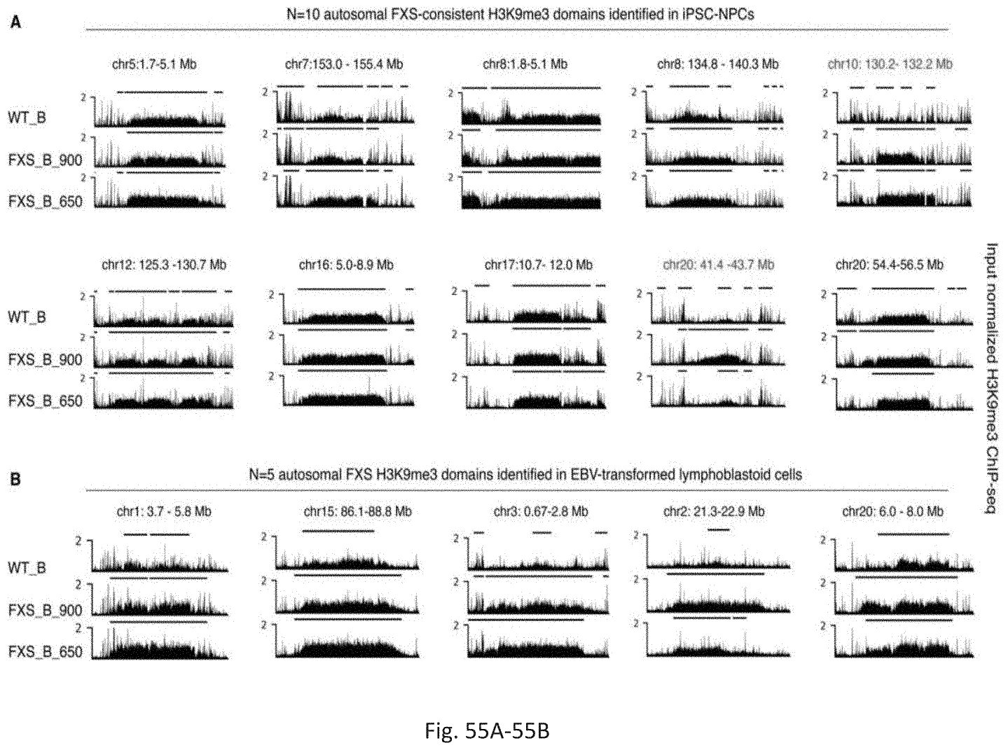

Macro-orchidism and soft skin are lesser known clinical presentations in FXS (35), and expansion of the FMR1 CGG STR also causes severe ovary defects in Fragile X-associated primary ovarian insufficiency (FXPOI) (36). We examined the transcriptional profile of H3K9me3-localized genes across 54 tissues from the GTEX consortium (37). We observed that genes localized to FXS heterochromatin domains exhibit tissue-specific expression profiles, including in the testis, female reproductive organs, epithelium, and (consistent with our NPC results) brain (FIG. 39I). Given that our iPSC-NPC H3K9me3 domains are also present in iPSCs and B cells (FIG. 54, FIG. 55), these results suggest that they also may be present in skin and reproductive tissues and thus might be relevant to understanding the silencing of genes linked to non-brain clinical presentations in FXS.

Autosomal FXS H3K9Me3 Domains Spatially Co-Localize with FMR1 Via Inter-Chromosomal Interactions

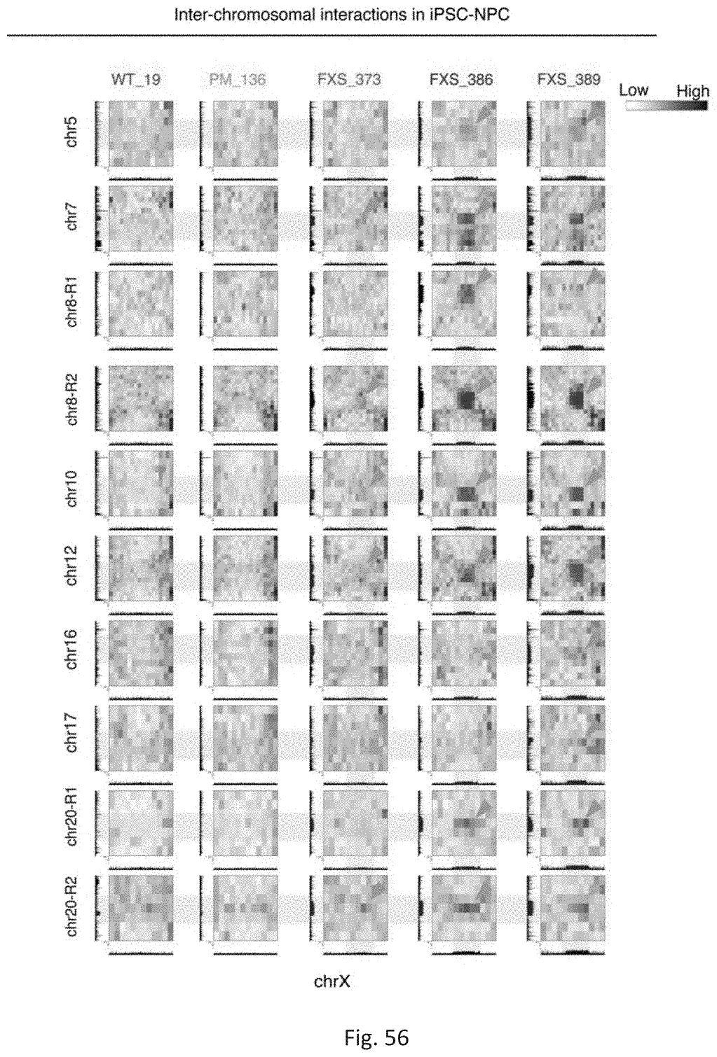

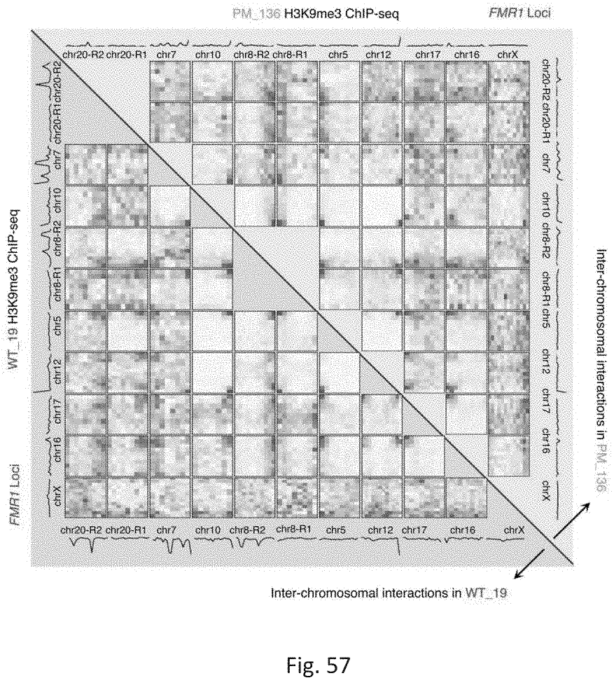

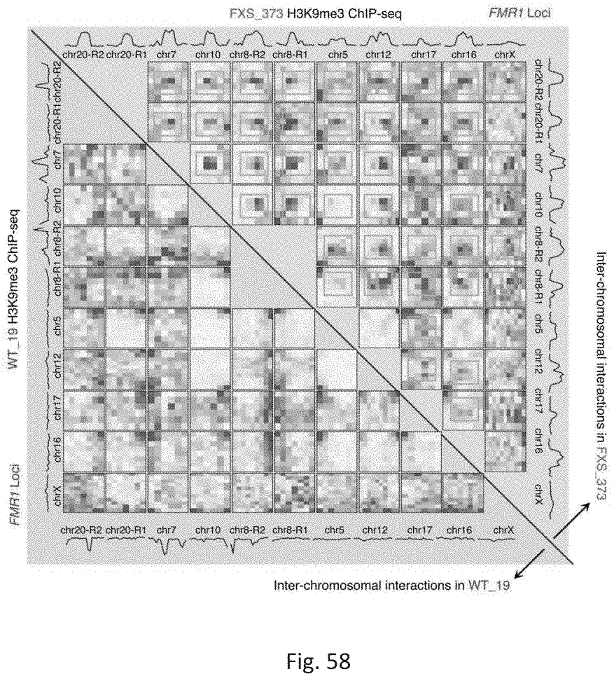

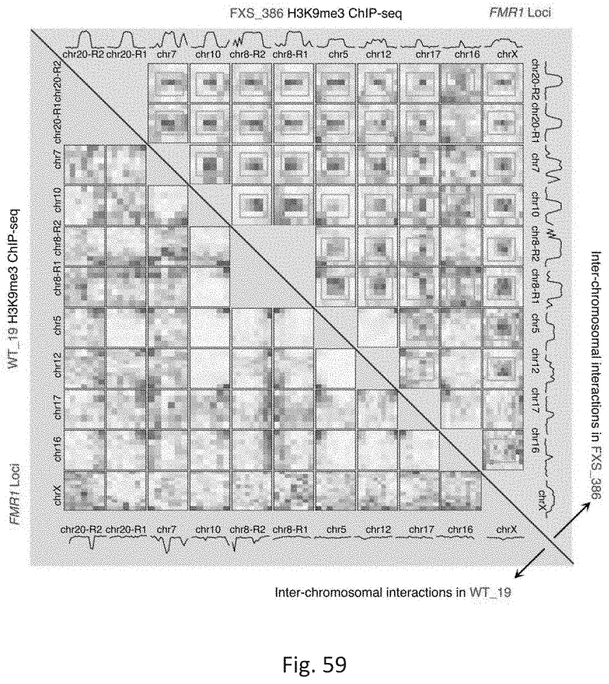

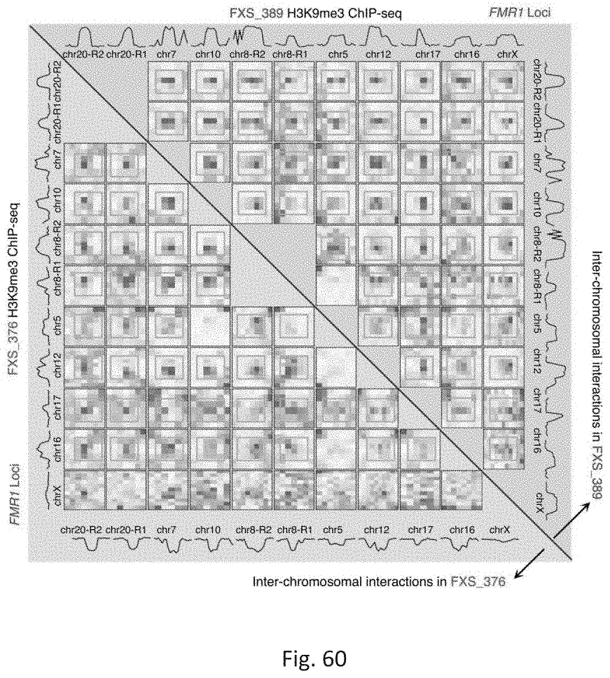

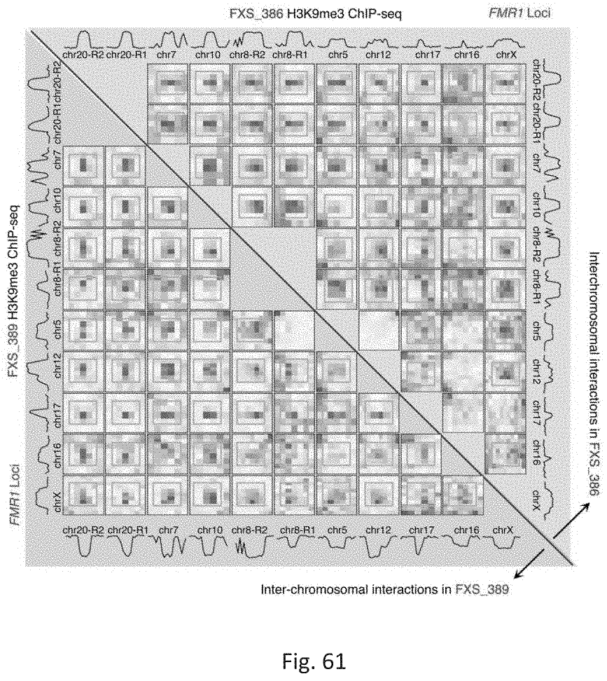

Given that the primary site of STR expansion is on the X chromosome, we sought to gain insight into how large genomic loci on autosomes are heterochromatinized in parallel with FMR1 CGG STR expansion in FXS. Using Hi-C, we queried trans interactions between chromosomes. We unexpectedly observed unusually strong inter-chromosomal interactions connecting the FMR1 locus to distal H3K9me3 domains (FIG. 40A and FIG. 56). Trans interactions are not present in normal- or pre-mutation-length iPSC-NPCs, and form concomitantly with increased density of H3K9me3 in full mutation-length CGG expansions (FIG. 40B, FIG. 57, FIG. 58, FIG. 59, FIG. 60, FIG. 61). Importantly, the distal silenced H3K9me3 domains contact each other as well as the X chromosome, suggesting they form multi-way subnuclear hubs with FMR1 in FXS (FIG. 40C). Our iPSC lines exhibit largely normal karyotype, and do not display structural issues that artifactually cause trans interaction signal (FIG. 62). These data indicate that autosomal FXS heterochromatin domains engage via spatial proximity with the unstable FMR1 locus upon mutation-length expansion of the CGG STR tract.

Autosomal FXS H3K9Me3 Domains are Enriched for STRs Prone to Instability in FXS iPSCs

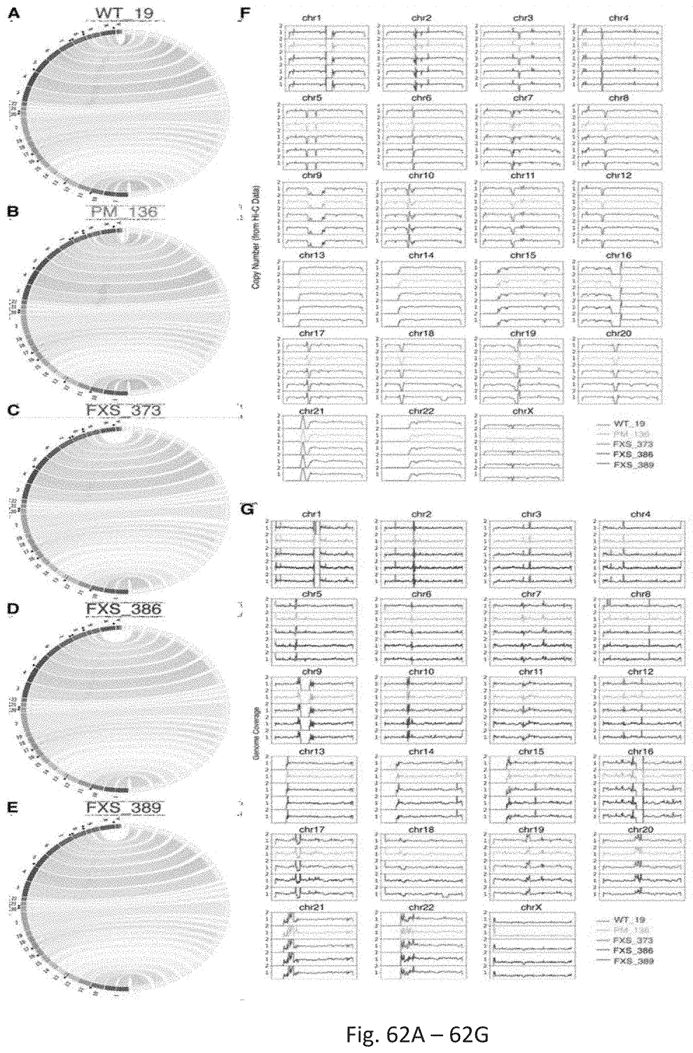

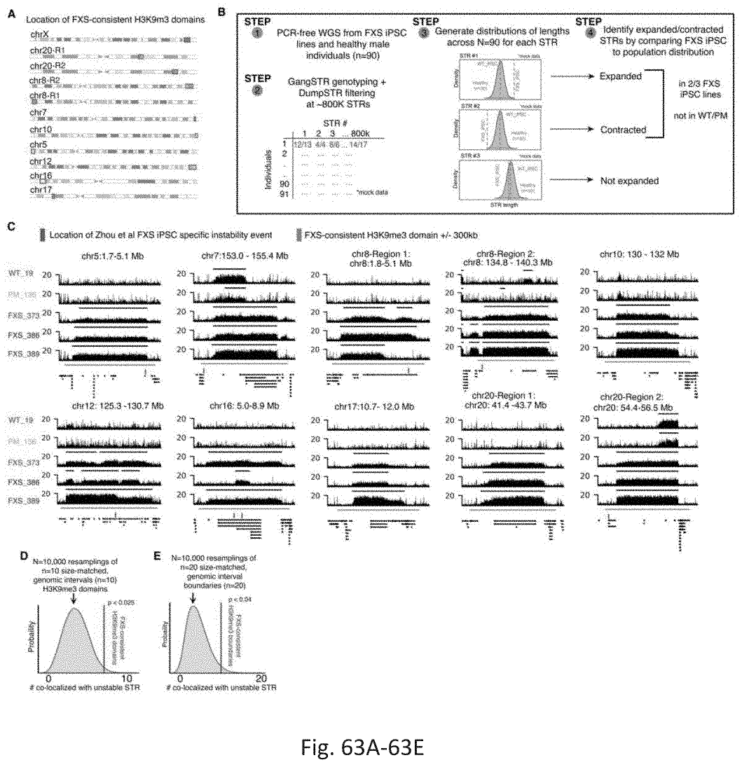

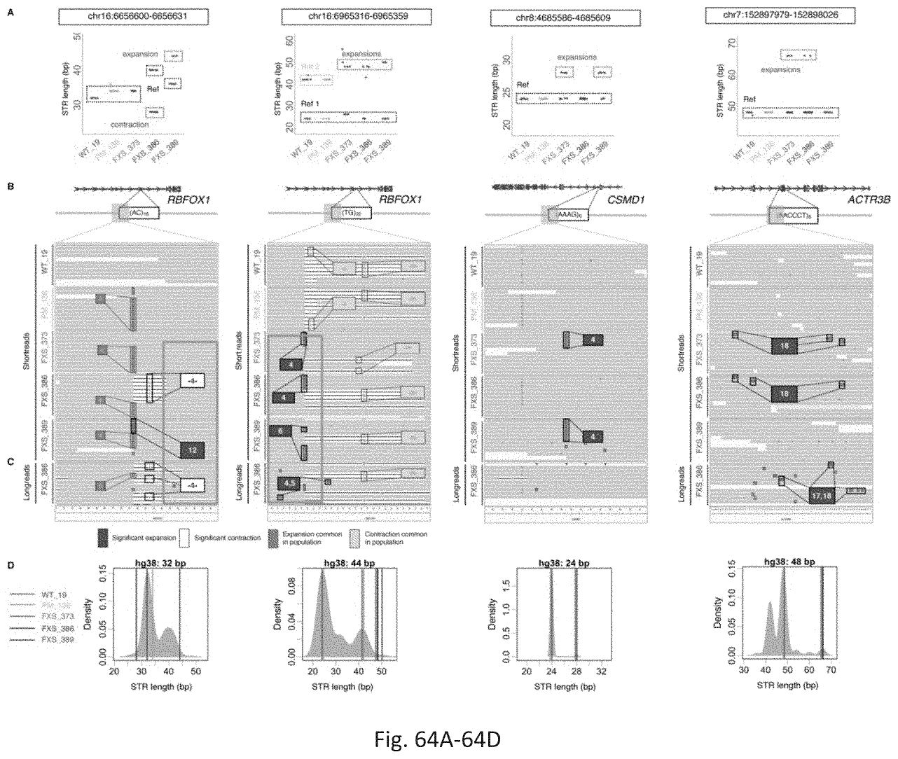

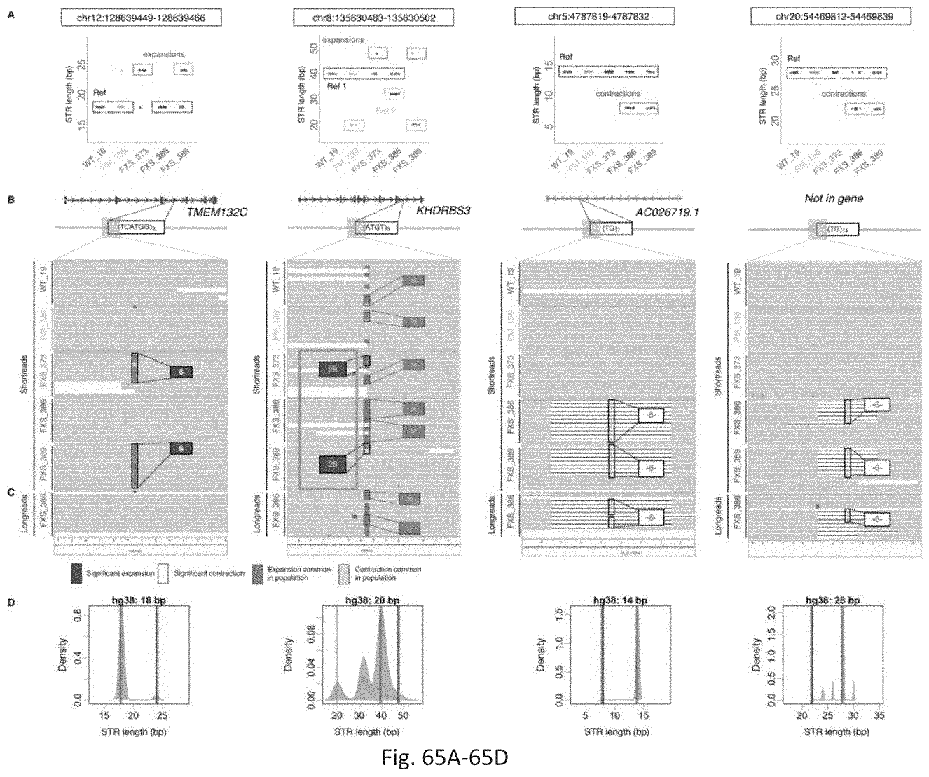

Heterochromatinization protects the repetitive genome against instability (38). We hypothesized that genomic loci in FXS H3K9me3 domains might spatially coordinate heterochromatinization because they encompass STRs susceptible to instability. We noticed that, like FMR1, nearly all of the FXS-specific distal H3K9me3 domains are located at the ends of chromosomes adjacent to sub-telomeric regions (FIG. 63a). Using high-coverage whole genome PCR-free sequencing and the GangSTR computational method (39), we computed the length of 800,000 STR tracts genome-wide in our FXS iPSC lines as well as in N=70 ancestry-, sex-, sequencing depth, and cell type-matched non-diseased individuals from the HipSci Consortium (40). We computed a null distribution of expected lengths across the N=70 non-diseased individuals for every STR tract and formulated a statistical test (˜800,000 tests, 1 per STR tract) in which we required that the STR length was significantly different than the null S1 (FIG. 63B). We identified a small set of STRs exhibiting reproducible FXS-specific expansion or contraction on autosomes in at least 2/3 our FXS iPSC lines. We validated our observed expansion/contraction events using genome-wide, single-molecule Nanopore long-read sequencing (FIG. 64-FIG. 65). Moreover, we also confirmed that we could identify the same candidate autosomal unstable STRs as significantly expanded/contracted using a null distribution of N=153 ancestry-, sex-, and sequencing depth-matched non-diseased individuals from the 1000 Genomes consortium (FIG. 63C).

Our data reveal the existence of STR expansion/contraction events on autosomes in our FXS iPSCs. We next sought to understand the relationship between our FXS iPSC unstable STRs and H3K9me3 domains. Similar to the CGG STR tract in FMR1 on the X chromosome, we observed that the majority of our FXS H3K9me3 domains or their boundaries co-localized with an STR tract exhibiting instability in our FXS iPSC lines (FIG. 63D). We find that the FXS H3K9me3 domains and their boundaries are significantly enriched for FXS iPSC-specific unstable STR tracts compared to random size-matched genomic intervals or genotype-invariant H3K9me3 domains (FIG. 40D-E, FIG. 63E-F) (26). It is particularly noteworthy that synaptic genes linked to Autism Spectrum Disorder in case-control studies, including CSDM1 (41) and RBFOX1 (42), co-localize with unstable STR tracts and are encompassed by H3K9me3 in our F×S lines (FIG. 40F-G, FIG. 64, FIG. 65). Together, our data suggest that regions of the genome silenced in FXS are similar to FMR1 in that they are at the ends of chromosomes adjacent to sub-telomeres and can co-localize with unstable STRs. The autosomal instability events in our F×S lines are reproducible, but significantly smaller in length change than the severe CGG expansion event at FMR1. Thus, they would have been undetectable until the recent availability of single-molecule long-read sequencing and computational technologies to glean STR length information from short-read sequencing.

Engineering the CGG STR to Pre-Mutation Length Reverses the FMR1 H3K9Me3 Domain and a Subset of Trans Interactions with Autosomal FXS Heterochromatin Domains

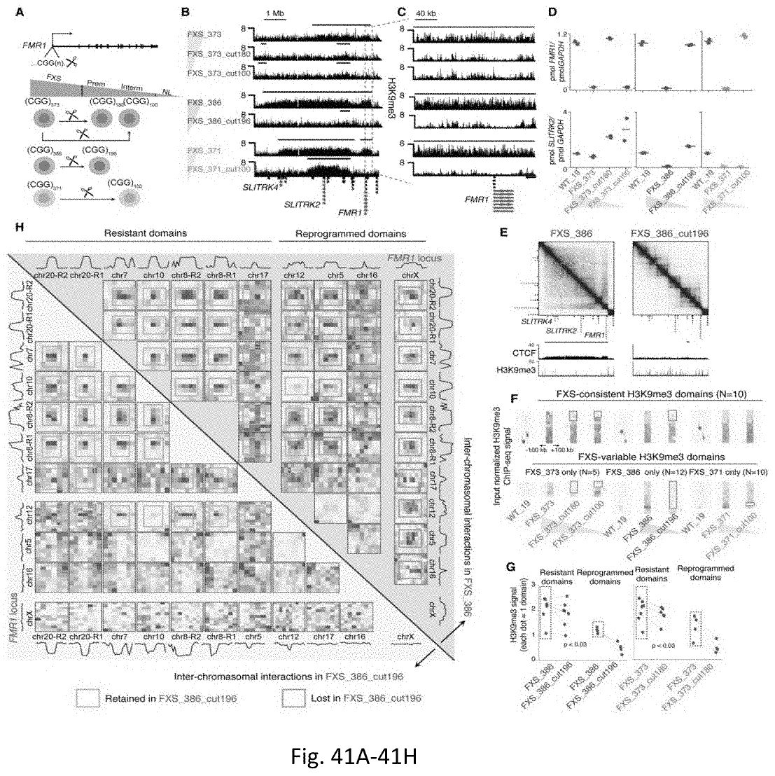

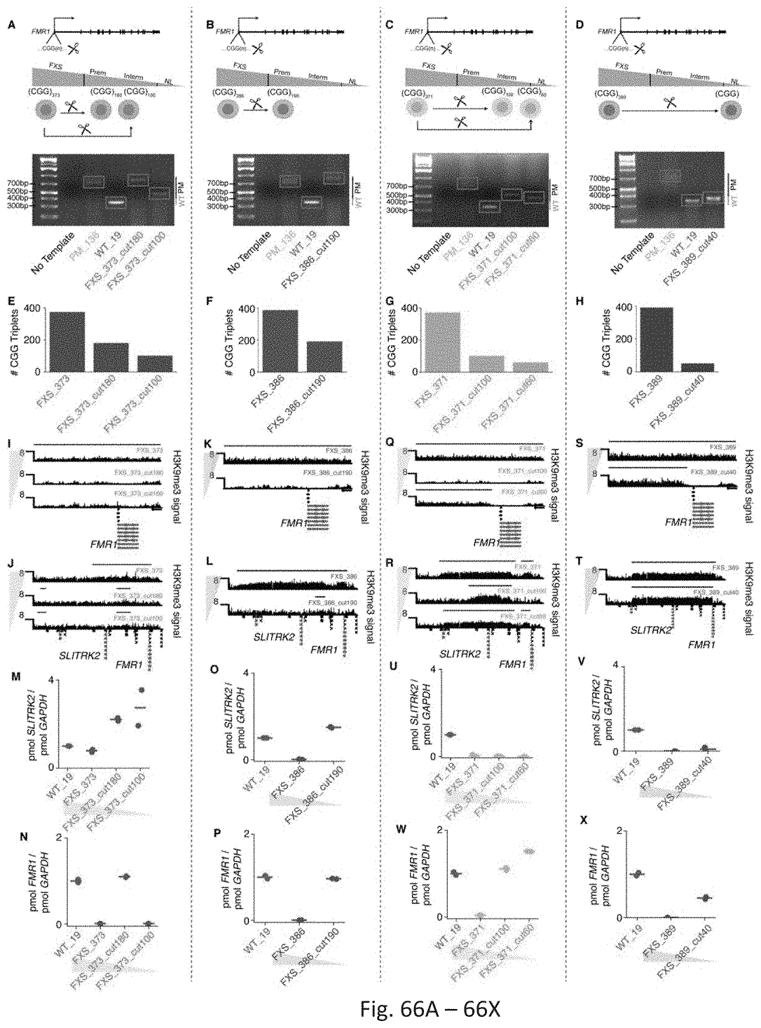

To understand the functional role of FMR1 CGG STR length on heterochromatin deposition in cis and trans, we examined if H3K9me3 could be reversed by shortening the CGG to long-pre-mutation (170-199 CGGs), short-pre-mutation (80-110 CGGs), or intermediate/normal-length (40-60 CGGs) with CRISPR (FIG. 41A, FIG. 66A-H). First, in two independent CRISPR clones from two independent full-mutation F×S lines (FXS_386, FXS_373), we cut back the FMR1 CGG STR to long-pre-mutation length CGG triplets (FXS_386_cut190, FXS_373_cut180) (FIG. 41A, FIG. 66A-B, E-F). We unexpectedly observed that the full 5-8 Mb-sized H3K9me3 domain encompassing SLITRK4, SLITRK2, and FMR1 is reversible upon cut-out to long-premutation-length (FIG. 41B, FIG. 66I-L). Both SLITRK2 and FMR1 mRNA levels were restored (FIG. 41C, FIG. 66M-P). Corroborating the loss of H3K9me3, CTCF occupancy was re-gained and TAD boundaries were re-instated at the broader FMR1 locus upon full-mutation to long-pre-mutation cut back (FIG. 41D). These results reveal that endogenous cut-back of the full-mutation length CGG STR to a length of 180-190 CGGs is sufficient to fully reverse the pathologic heterochromatin and genome misfolding around FMR1 in FXS iPSCs.

We sought to define the CGG cut-back length range that is permissible to reversal of the X chromosome H3K9me3 domain. We cut back the FMR1 CGG STR to intermediate/normal-length (40-60 CGG triplets; FXS_371_cut60, FXS_389_cut40) as well as short-pre-mutation (100 CGG triplets; FXS_371_cut100, FXS_373_cut100) in two independent CRISPR clones from two independent full-mutation F×S lines (FIG. 41A, FIG. 66A-H). Cut-back to 100 triplets had a partial and inconsistent effect on H3K9me3, with domain removal but residual local FMR1 repression in one iPSC clone (FIGS. 41B-C, FIG. 66A, I-J, M-N) and de-repression of FMR1 and partial H3K9me3 domain shortening in the other line (FIG. 41B-C, FIG. 66A, K-L, O-P). Strikingly, after the CGG STR was cutback to 40-60 intermediate/normal-length triplets, the H3K9me3 domain on the X chromosome remained largely intact (FIGS. 66Q-T) and SLITRK2 remained silenced (FIGS. 66U-V).

Consistent with previous reports, we observed a slight local reduction in H3K9me3 only over the FMR1 gene (FIGS. 66Q-T) and FMR1 de-repression (FIGS. 66W-X) in both intermediate/normal-length cut-out iPSC clones (43, 44). Our data indicate that engineering the CGG STR to intermediate/normal-length does not markedly reprogram the FXS H3K9me3 domain on the X chromosome.

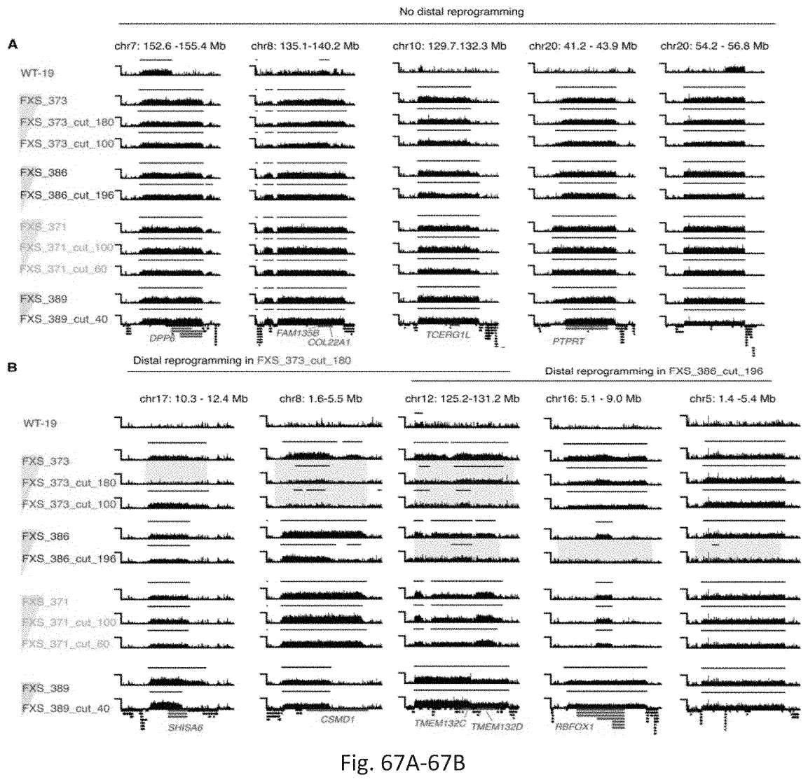

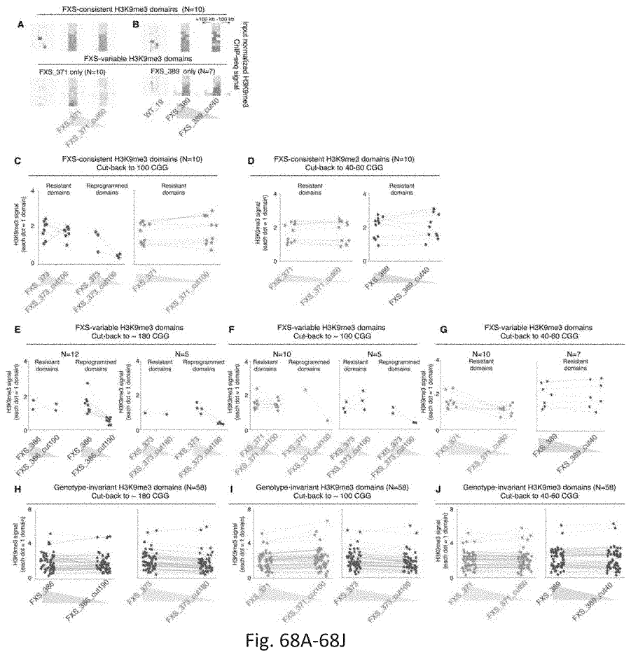

We next queried the extent to which the distal H3K9me3 domains in FXS could be reversed upon local FMR1 CGG engineering. Distal heterochromatinized loci maintained a high level of H3K9me3 signal upon intermediate/normal-length CGG cut-out (FIG. 67, FIG. 68A-B). By contrast, we observed that a subset of distal H3K9me3 domains were reprogrammed upon engineering of the FMR1 CGG STR long-pre-mutation length (FIG. 41E-F, FIG. 67, FIG. 68C-D). Distal domains with the lowest H3K9me3 density were the most susceptible to reprogramming after engineering the FMR1 CGG STR (FIG. 41F). Although the majority of autosomal H3K9me3 loci remained tethered in a trans interaction hub, the FMR1 locus and several distal domains lost their heterochromatinization and spatially disconnected upon engineering of the mutation-length CGG at FMR1 to long-pre-mutation (FIG. 41G). Together, these results indicate that reverse engineering of the FMR1 CGG to pre-mutation length can ameliorate the FMR1 H3K9me3 domain on the X chromosome and attenuate a subset of distal H3K9me3 domains. The persistence of heterochromatin silencing at many autosomal FXS H3K9me3 domains suggests that many pathologically silenced synaptic, epithelial, and reproductive tissue genes may not be de-repressed with a normal-length FMR1 CGG cut-out strategy in FXS.

Autosomal and X Chromosome H3K9Me3 Domains Form Trans Interactions in Single FXS Cells

Finally, we used Oligopaints DNA FISH probes to image the trans interactions among H3K9me3 domains in single cells (FIG. 42A-F). We observed that chromosome X and 12 H3K9me3 domains are closer together in a higher proportion of FXS iPSCs compared to normal-length iPSCs (FIG. 42A-C). Moreover, the chromosome X H3K9me3 domain is closer on average to all autosomal H3K9me3 domains (FIG. 42D-E) with fewer distinguishable domain-like dots per cell (FIG. 42F) in FXS iPSCs compared to normal-length iPSCs. Consistent with our Hi-C results, we observe that engineering the CGG tract to 180 triplets resumes spatial distances in single FXS iPSCs that resemble the normal-length iPSC distances (FIG. 42A-F). Thus, with both ensemble Hi-C as well as single-cell imaging methods, we demonstrate that autosomal H3K9me3 domains form CGG-length-dependent pathological trans interactions with the FMR1 H3K9me3 domain in FXS.

### REFERENCES

1. M. R. Santoro, S. M. Bray, S. T. Warren, Molecular mechanisms of fragile X syndrome: a twenty-year perspective. Annu Rev Pathol 7, 219-245 (2012).
2. A. R. La Spada, H. L. Paulson, K. H. Fischbeck, Trinucleotide repeat expansion in neurological disease. Ann Neurol 36, 814-822 (1994).
3. S. M. Mirkin, Expandable DNA repeats and human disease. Nature 447, 932-940 (2007).
4. D. L. Nelson, H. T. Orr, S. T. Warren, The unstable repeats--three evolving faces of neurological disease. Neuron 77, 825-843 (2013).
5. A. R. La Spada, J. P. Taylor, Repeat expansion disease: progress and puzzles in disease pathogenesis. Nat Rev Genet 11, 247-258 (2010).
6. C. T. McMurray, Mechanisms of trinucleotide repeat instability during human development. Nat Rev Genet 11, 786-799 (2010).
7. C. E. Pearson, K. Nichol Edamura, J. D. Cleary, Repeat instability: mechanisms of dynamic mutations. Nat Rev Genet 6, 729-742 (2005).
8. R. J. Hagerman, P. Hagerman, Fragile X-associated tremor/ataxia syndrome—features, mechanisms and management. Nat Rev Neurol 12, 403-412 (2016).
9. R. I. Richards et al., Evidence of founder chromosomes in fragile X syndrome. Nat Genet 1, 257-260 (1992).
10. H. T. Orr, H. Y. Zoghbi, Trinucleotide repeat disorders. Annu Rev Neurosci 30, 575-621 (2007).
11. F. Tassone, C. Iwahashi, P. J. Hagerman, FMR1 RNA within the intranuclear inclusions of fragile X-associated tremor/ataxia syndrome (FXTAS). RNA Biol 1, 103-105 (2004).
12. P. K. Todd et al., CGG repeat-associated translation mediates neurodegeneration in fragile X tremor ataxia syndrome. Neuron 78, 440-455 (2013).
13. H. Y. Zoghbi, M. F. Bear, Synaptic dysfunction in neurodevelopmental disorders associated with autism and intellectual disabilities. Cold Spring Harb Perspect Biol 4, (2012).
14. A. Contractor, V. A. Klyachko, C. Portera-Cailliau, Altered Neuronal and Circuit Excitability in Fragile X Syndrome. Neuron 87, 699-715 (2015).
15. J. S. Sutcliffe et al., DNA methylation represses FMR-1 transcription in fragile X syndrome. Hum Mol Genet 1, 397-400 (1992).
16. Y. Zhou, D. Kumari, N. Sciascia, K. Usdin, CGG-repeat dynamics and FMR1 gene silencing in fragile X syndrome stem cells and stem cell-derived neurons. Mol Autism 7, 42 (2016).
17. D. Colak et al., Promoter-bound trinucleotide repeat mRNA drives epigenetic silencing in fragile X syndrome. Science 343, 1002-1005 (2014).
18. R. S. Alisch et al., Genome-wide analysis validates aberrant methylation in fragile X syndrome is specific to the FMR1 locus. BMC Med Genet 14, 18 (2013).
19. E. Korb et al., Excess Translation of Epigenetic Regulators Contributes to Fragile X Syndrome and Is Alleviated by Brd4 Inhibition. Cell 170, 1209-1223 e1220 (2017).
20. R. Dahlhaus, Of Men and Mice: Modeling the Fragile X Syndrome. Front Mol Neurosci 11, 41 (2018).
21. S. A. Musumeci et al., Audiogenic seizure susceptibility is reduced in fragile X knockout mice after introduction of FMR1 transgenes. Exp Neurol 203, 233-240 (2007).
22. A. M. Peier et al., (Over)correction of FMR1 deficiency with YAC transgenics: behavioral and physical features. Hum Mol Genet 9, 1145-1159 (2000).
23. S. Gholizadeh, J. Arsenault, I. C. Xuan, L. K. Pacey, D. R. Hampson, Reduced phenotypic severity following adeno-associated virus-mediated Fmr1 gene delivery in fragile X mice. Neuropsychopharmacology 39, 3100-3111 (2014).
24. Z. Zeier et al., Fragile X mental retardation protein replacement restores hippocampal synaptic function in a mouse model of fragile X syndrome. Gene Ther 16, 1122-1129 (2009).
25. J. Arsenault et al., FMRP Expression Levels in Mouse Central Nervous System Neurons Determine Behavioral Phenotype. Hum Gene Ther 27, 982-996 (2016).
26. J. H. Sun et al., Disease-Associated Short Tandem Repeats Co-localize with Chromatin Domain Boundaries. Cell 175, 224-238 e215 (2018).
27. B. Coffee, F. Zhang, S. T. Warren, D. Reines, Acetylated histones are associated with FMR1 in normal but not fragile X-syndrome cells. Nat Genet 22, 98-101 (1999).
28. B. Coffee, F. Zhang, S. Ceman, S. T. Warren, D. Reines, Histone modifications depict an aberrantly heterochromatinized FMR1 gene in fragile x syndrome. Am J Hum Genet 71, 923-932 (2002).
29. X. S. Liu et al., Rescue of Fragile X Syndrome Neurons by DNA Methylation Editing of the FMR1 Gene. Cell 172, 979-992 e976 (2018).
30. J. M. Haenfler et al., Targeted Reactivation of FMR1 Transcription in Fragile X Syndrome Embryonic Stem Cells. Front Mol Neurosci 11, 282 (2018).
31. Zhou et al. Supplementary Materials
32. D. Kumari, K. Usdin, The distribution of repressive histone modifications on silenced FMR1 alleles provides clues to the mechanism of gene silencing in fragile X syndrome. Hum Mol Genet 19, 4634-4642 (2010).
33. M. Telias, Molecular Mechanisms of Synaptic Dysregulation in Fragile X Syndrome and Autism Spectrum Disorders. Front Mol Neurosci 12, 51 (2019).
34. B. E. Pfeiffer, K. M. Huber, The state of synapses in fragile X syndrome. Neuroscientist 15, 549-567 (2009).
35. J. F. Atkin, K. Flaitz, S. Patil, W. Smith, A new X-linked mental retardation syndrome. Am J Med Genet 21, 697-705 (1985).
36. H. Tan, H. Li, P. Jin, RNA-mediated pathogenesis in fragile X-associated disorders. Neurosci Lett 466, 103-108 (2009).
37. M. Mele et al., Human genomics. The human transcriptome across tissues and individuals. Science 348, 660-665 (2015).
38. A. Janssen, S. U. Colmenares, G. H. Karpen, Heterochromatin: Guardian of the Genome.

Annu Rev Cell Dev Biol 34, 265-288 (2018).
39. N. Mousavi, S. Shleizer-Burko, R. Yanicky, M. Gymrek, Profiling the genome-wide landscape of tandem repeat expansions. Nucleic Acids Res 47, e90 (2019).
40. I. Streeter et al., The human-induced pluripotent stem cell initiative-data resources for cellular genetics. Nucleic Acids Res 45, D691-D697 (2017).
41. H. N. Cukier et al., Exome sequencing of extended families with autism reveals genes shared across neurodevelopmental and neuropsychiatric disorders.

Mol Autism 5, 1 (2014).
42. A. J. Griswold et al., Targeted massively parallel sequencing of autism spectrum disorder-associated genes in a case control cohort reveals rare loss-of-function risk variants. Mol Autism 6, 43 (2015).
43. N. Xie et al., Reactivation of FMR1 by CRISPR/Cas9-Mediated Deletion of the Expanded CGG-Repeat of the Fragile X Chromosome. PLoS One 11, e0165499 (2016).
44. C. Y. Park et al., Reversion of FMR1 Methylation and Silencing by Editing the Triplet Repeats in Fragile X iPSC-Derived Neurons. Cell Rep 13, 234-241 (2015).
45. M. Groh, M. M. Lufino, R. Wade-Martins, N. Gromak, R-loops associated with triplet repeat expansions promote gene silencing in Friedreich ataxia and fragile X syndrome. PLoS Genet 10, e1004318 (2014).
46. E. W. Loomis, L. A. Sanz, F. Chedin, P. J. Hagerman, Transcription-associated R-loop formation across the human FMR1 CGG-repeat region. PLoS Genet 10, e1004294 (2014).
47. C. Sellier et al., Sam68 sequestration and partial loss of function are associated with splicing alterations in FXTAS patients. EMBO J 29, 1248-1261 (2010).
48. R. Alcala-Vida et al., Age-related and disease locus-specific mechanisms contribute to early remodelling of chromatin structure in Huntington's disease mice. Nat Commun 12, 364 (2021).
49. G. K. Griffin et al., Epigenetic silencing by SETDB1 suppresses tumour intrinsic immunogenicity. Nature 595, 309-314 (2021).
50. J. H. Sun et al., Disease-Associated Short Tandem Repeats Co-localize with Chromatin Domain Boundaries. Cell 175, 224-238 e215 (2018).
51. W. Xie et al., Epigenomic analysis of multilineage differentiation of human embryonic stem cells. Cell 153, 1134-1148 (2013).
52. A. Saluto et al., An enhanced polymerase chain reaction assay to detect pre- and full mutation alleles of the fragile X mental retardation 1 gene. J Mol Diagn 7, 605-612 (2005).
53. B. J. Beliveau et al., OligoMiner provides a rapid, flexible environment for the design of genome-scale oligonucleotide in situ hybridization probes. Proc Natl Acad Sci USA 115, E2183-E2192 (2018).
54. J. H. Su, P. Zheng, S. S. Kinrot, B. Bintu, X. Zhuang, Genome-Scale Imaging of the 3D Organization and Transcriptional Activity of Chromatin. Cell 182, 1641-1659 e1626 (2020).
55. G. Nir et al., Walking along chromosomes with super-resolution imaging, contact maps, and integrative modeling. PLoS Genet 14, e1007872 (2018).
56. J. R. Moffitt, X. Zhuang, RNA Imaging with Multiplexed Error-Robust Fluorescence In Situ Hybridization (MERFISH). Methods Enzymol 572, 1-49 (2016).
57. L. F. Rosin, S. C. Nguyen, E. F. Joyce, Condensin II drives large-scale folding and spatial partitioning of interphase chromosomes in Drosophila nuclei. PLoS Genet 14, e1007393 (2018).
58. J. Ollion, J. Cochennec, F. Loll, C. Escude, T. Boudier, TANGO: a generic tool for high-throughput 3D image analysis for studying nuclear organization. Bioinformatics 29, 1840-1841 (2013).
59. J. A. Beagan et al., Three-dimensional genome restructuring across timescales of activity-induced neuronal gene expression. Nat Neurosci 23, 707-717 (2020).
60. J. H. Kim et al., LADL: light-activated dynamic looping for endogenous gene expression control. Nat Methods 16, 633-639 (2019).
61. J. H. Kim et al., 5C-ID: Increased resolution Chromosome-Conformation-Capture-Carbon-Copy with in situ 3C and double alternating primer design. Methods 142, 39-46 (2018).
62. J. A. Beagan et al., YY1 and CTCF orchestrate a 3D chromatin looping switch during early neural lineage commitment. Genome Res 27, 1139-1152 (2017).
63. J. A. Beagan et al., Local Genome Topology Can Exhibit an Incompletely Rewired 3D—Folding State during Somatic Cell Reprogramming. Cell Stem Cell 18, 611-624 (2016).
64. J. E. Phillips-Cremins et al., Architectural protein subclasses shape 3D organization of genomes during lineage commitment. Cell 153, 1281-1295 (2013).
65. M. P. Meers, T. D. Bryson, J. G. Henikoff, S. Henikoff, Improved CUT&RUN chromatin profiling tools. Elife 8, (2019).
66. P. Giesselmann et al., Analysis of short tandem repeat expansions and their methylation state with nanopore sequencing. Nat Biotechnol 37, 1478-1481 (2019).
67. T. Gilpatrick et al., Targeted nanopore sequencing with Cas9-guided adapter ligation. Nat Biotechnol 38, 433-438 (2020).
68. B. S. Pedersen, R. L. Collins, M. E. Talkowski, A. R. Quinlan, Indexcov: fast coverage quality control for whole-genome sequencing. Gigascience 6, 1-6 (2017).
69. X. Wang et al., Genome-wide detection of enhancer-hijacking events from chromatin interaction data in rearranged genomes. Nat Methods 18, 661-668 (2021).
70. H. Zhang et al., Chromatin structure dynamics during the mitosis-to-G1 phase transition. Nature 576, 158-162 (2019).
71. L. R. Fernandez, T. G. Gilgenast, J. E. Phillips-Cremins, 3DeFDR: statistical methods for identifying cell type-specific looping interactions in 5C and Hi-C data. Genome Biol 21, 219 (2020).
72. T. G. Gilgenast, J. E. Phillips-Cremins, Systematic Evaluation of Statistical Methods for Identifying Looping Interactions in 5C Data. Cell Syst 8, 197-211 e113 (2019).
73. J. E. Phillips-Cremins, T. G. Gilgenast, Systematic evaluation of statistical methods for identifying looping interactions in 5C data. bioRxiv, (2017).
74. Q. Song, A. D. Smith, Identifying dispersed epigenomic domains from ChIP-Seq data. Bioinformatics 27, 870-871 (2011).
75. N. L. Bray, H. Pimentel, P. Melsted, L. Pachter, Near-optimal probabilistic RNA-seq quantification. Nat Biotechnol 34, 525-527 (2016).
76. M. I. Love, W. Huber, S. Anders, Moderated estimation of fold change and dispersion for RNA-seq data with DESeq2. Genome Biol 15, 550 (2014).
77. J. A. Beagan, J. E. Phillips-Cremins, On the existence and functionality of topologically associating domains. Nat Genet 52, 8-16 (2020).
78. H. K. Norton et al., Detecting hierarchical genome folding with network modularity. Nat Methods 15, 119-122 (2018).
79. J. R. Dixon et al., Topological domains in mammalian genomes identified by analysis of chromatin interactions. Nature 485, 376-380 (2012).
80. M. J. Rowley, V. G. Corces, Organizational principles of 3D genome architecture. Nat Rev Genet 19, 789-800 (2018).
81. M. J. Rowley et al., Evolutionarily Conserved Principles Predict 3D Chromatin Organization. Mol Cell 67, 837-852 e837 (2017).
82. O. Dudchenko et al., De novo assembly of the Aedes aegypti genome using Hi-C yields chromosome-length scaffolds. Science 356, 92-95 (2017).
83. C. Genomes Project et al., A global reference for human genetic variation. Nature 526, 68-74 (2015).
84. X. Zheng-Bradley et al., Alignment of 1000 Genomes Project reads to reference assembly GRCh38. Gigascience 6, 1-8 (2017).
85. I. Streeter et al., The human-induced pluripotent stem cell initiative-data resources for cellular genetics. Nucleic Acids Res 45, D691-D697 (2017).

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A composition for modulating heterochomatin levels or activating, reactivating or de-repressing at least one H3K9me3-heterochromatin mark containing gene, wherein the gene is repressed or silenced in a heterochromatic genomic region, wherein the composition increases the level of at least one of the transcription of the silenced gene, the translation of the silenced gene and the level of gene product for the silenced gene, the composition selected from the group consisting of:
a) a composition comprising an epigenomic editor comprising catalytically dead Cas9 (dCas9) operably linked to a composition for removing a methylation mark;
b) a composition for overexpression of one or more H3K9me3-heterochromatin mark containing gene, wherein the gene is repressed or silenced in a heterochromatic genomic region;
c) a composition for reducing a full mutation length CGG tandem repeat of Fmr1 to an intermediate or pre-mutation length; and
d) a composition for reducing the level of Fmr1 mRNA, wherein the Fmr1 mRNA comprises a full mutation length CGG tandem repeat; and
e) a composition comprising a noncoding RNA molecule comprising a pre-mutation length CGG repeat.

2. The composition of claim 1a, wherein the composition for removing a methylation mark is selected from the group consisting of 5-aza-2′-deoxycytidine, VP64, NF-κB p65, Ten-Eleven Translocation (TET) protein, histone lysine demethylase (KDM) and a DNA demethylase.

3. The composition of claim 1a, wherein the composition further comprises a guide RNA specific for at least one silenced gene in a heterochromatin comprising genomic region.

4. The composition of claim 3, wherein the silenced gene in a heterochromatin comprising genomic region is selected from the group consisting of FMR1, FMR1NB, FMR1-AS1, C5orf38, CTD-2194D22.4, LOC100506858, IRX2, LOC105374620, LINC01377, LINC01019, LINC01017, IRX1, LINC02114, DPP6, LINC01287, LOC101929998, CSMD1, FAM135B, LOC101927815, COL22A1, KCNK9, TRAPPC9, MYOM2, LOC101927845, LINC01591, LOC101927915, SPANXN4, SPANXN3, SLITRK4, SPANXN2, UBE2NL, SPANXN1, SLITRK2, TMEM257, MIR892C, MIR890, MIR888, MIR892A, MIR892B, MIR891B, MIR891A, CXorf51B, CXorf51A, MIR513C, MIR513B, MIR513A1, MIR513A2, MIR506, MIR507, MIR508, MIR514B, MIR509-2, MIR509-3, MIR509-1, MIR510, MIR514A1, MIR514A2, MIR514A3, TCERG1L, MIR378C, TCERG1L-AS1, LINC01164, TMEM132C, TMEM132D, LOC100996671, LOC101927592, LINC00508, LOC100996679, GLT1D1, RIMBP2, LINC00939, LOC101927464, LOC100128554, LINC00944, LINC00943, LOC440117, LOC101927616, LOC101927637, LOC105370068, FLJ37505, LINC00507, CRAT8, LOC101927694, MIR4419B, MIR3612, SLC15A4, LOC283352, LOC101927735, LOC100190940, FZD10-AS1, FZD10, PIWIL1, RBFOX1, MIR8065, TMEM114, DNAH9, SHISA6, PTPRT, LOC101927159, LINC01441, LINC01440, CBLN4, and MC3R.

5. The composition of claim 1b comprising a heterologous nucleic acid molecule encoding at least one selected from the group consisting of FMR1, FMR1NB, FMR1-AS1, C5orf38, CTD-2194D22.4, LOC100506858, IRX2, LOC105374620, LINC01377, LINC01019, LINC01017, IRX1, LINC02114, DPP6, LINC01287, LOC101929998, CSMD1, FAM135B, LOC101927815, COL22A1, KCNK9, TRAPPC9, MYOM2, LOC101927845, LINC01591, LOC101927915, SPANXN4, SPANXN3, SLITRK4, SPANXN2, UBE2NL, SPANXN1, SLITRK2, TMEM257, MIR892C, MIR890, MIR888, MIR892A, MIR892B, MIR891B, MIR891A, CXorf51B, CXorf51A, MIR513C, MIR513B, MIR513A1, MIR513A2, MIR506, MIR507, MIR508, MIR514B, MIR509-2, MIR509-3, MIR509-1, MIR510, MIR514A1, MIR514A2, MIR514A3, TCERG1L, MIR378C, TCERG1L-AS1, LINC01164, TMEM132C, TMEM132D, LOC100996671, LOC101927592, LINC00508, LOC100996679, GLT1D1, RIMBP2, LINC00939, LOC101927464, LOC100128554, LINC00944, LINC00943, LOC440117, LOC101927616, LOC101927637, LOC105370068, FLJ37505, LINC00507, CRAT8, LOC101927694, MIR4419B, MIR3612, SLC15A4, LOC283352, LOC101927735, LOC100190940, FZD10-AS1, FZD10, PIWIL1, RBFOX1, MIR8065, TMEM114, DNAH9, SHISA6, PTPRT, LOC101927159, LINC01441, LINC01440, CBLN4, and MC3R.

6. The composition of claim 1b, wherein the composition comprises a nucleic acid molecule comprising a nucleotide sequence of an Fmr1 gene comprising an intermediate or pre-mutation length CGG tandem repeat, wherein the intermediate or pre-mutation length CGG tandem repeat comprises 40 to 200 tandem CGG repeats.

7. The composition of claim 1c, comprising a complex comprising a guide RNA targeted to the Fmr1 gene, and a CRISPR-associated (Cas) protein.

8. The composition of claim 1d, comprising a complex comprising a guide RNA targeted to the Fmr1 mRNA, and a CRISPR-associated (Cas) protein.

9. The composition of claim 1e, wherein the composition comprises an RNA vaccine.

10. A composition comprising an inhibitor of at least one of heterochromatin formation, RNA mediated heterochromatin formation and RNA-DNA interactions, wherein the inhibitor is selected from the group consisting of a small interfering RNA (siRNA), a microRNA, antisense oligonucleotide (ASO), a ribozyme, an expression vector encoding a transdominant negative mutant, an antibody, an antibody fragment, a peptide, a chemical compound and a small molecule, wherein inhibitor decreases the level of at least one selected from the group consisting of:
a) the level of mRNA or protein of at least one CGG tandem repeat containing gene; and
b) the level of mRNA or protein of at least one histone H3-K9 methyltransferase gene.

11. The composition of claim 10, wherein the inhibitor is selected from the group consisting of: compound 1a, compound if and ETP69.

12. The composition of claim 10, wherein the inhibitor is an antisense oligonucleotide targeting at least one of FMR1, SHISA6, IRX2, TCERG1L, PTPRT, DPP6, and TMEM257.

13. The composition of claim 10, wherein the histone H3-K9 methyltransferase gene is selected from the group consisting of ESET, G9a, Eu-HMTase, SUV39H1 and SUV39H2.

14. A method of activating, reactivating or de-repressing at least one H3K9me3-heterochromatin mark containing gene, wherein the gene is repressed or silenced in a heterochromatic genomic region, the method comprising contacting a sample comprising a heterochromatic nucleic acid molecule with a composition of claim 1.

15. A method of inhibiting at least one of heterochromatin formation, RNA mediated heterochromatin formation and RNA-DNA interactions, the method comprising contacting a sample with a composition of claim 10.

16. A method of treating or preventing a disease or disorder associated with genomic instability or a triplet repeat expansion in a subject in need thereof, the method comprising administering a composition of claim 1 for activating, reactivating or de-repressing at least one H3K9me3-heterochromatin mark containing gene, wherein the gene is repressed or silenced in a heterochromatic genomic region, to a subject in need thereof.

17. The method of claim 16, wherein the disease or disorder associated with genomic instability or a triplet repeat expansion is selected from the group consisting of cancer, parkinsonism, ataxia, dementia, autonomic dysfunctions, myopathy, ubiquitin-positive inclusion bodies, middle cerebellar peduncle hyperintensity, leukoencephalopathy, myotonic dystrophy (DM), Huntington disease, spinocerebellar ataxia, Friedreich ataxia, fragile X syndrome, fragile X-associated primary ovarian insufficiency (FXPOI), fragile X-associated tremor/ataxia syndrome (FXTAS), syndromic and non-syndromic forms of intellectual disability (ID), autism, developmental delay, Jacobsen syndrome, and Baratela-Scott syndrome.

18. A method of treating or preventing a disease or disorder associated with genomic instability or a triplet repeat expansion in a subject in need thereof, the method comprising administering a composition of claim 10 for inhibiting at least one of heterochromatin formation, RNA mediated heterochromatin formation and RNA-DNA interactions, to a subject in need thereof.

19. A composition for inhibiting an interaction between a nucleic acid molecule comprising a Fmr1 full-mutation length CGG repeat and at least one distal or trans nucleic acid molecule comprising a CGG repeat, comprising a recombinant nucleic acid molecule selected from the group consisting of:
a) a recombinant nucleic acid molecule comprising a pre-mutation length CGG repeat that binds to a CGG repeat, wherein the pre-mutation length CGG repeat comprises 99 CGG repeats and
b) a recombinant nucleic acid molecule for expression of an antisense oligonucleotide that directly hybridizes to a nucleic acid molecule comprising a CGG repeat.

20. A method of inhibiting an interaction between a nucleic acid molecule comprising a Fmr1 full-mutation length CGG repeat comprises at least 200 CGG repeats, and at least one distal or trans nucleic acid molecule comprising a CGG repeat, the method comprising administering to a subject in need thereof at least one inhibitor selected from the group consisting of:
a) a composition of claim 19;
b) an inhibitor of heterochromatin formation;
c) an inhibitor of RNA mediated heterochromatin formation;
d) an inhibitor of RNA-DNA interactions;
e) a recombinant nucleic acid molecule comprising a pre-mutation length CGG repeat comprising about 99 CGG repeats;
f) a recombinant nucleic acid molecule for expression of an antisense oligonucleotide that directly hybridizes to a nucleic acid molecule comprising a CGG repeat; and
g) a small molecule inhibitor selected from the group consisting of compound 1a, compound if and ETP69.
